SEQUENCE LISTING (36 sequences)

SEQ ID NO: 1 - protein, 11 aa, Homo sapiens
SASQGISNYLN

SEQ ID NO: 2 - protein, 7 aa, Homo sapiens
YTSNLHS

SEQ ID NO: 3 - protein, 9 aa, Homo sapiens
QQYSELPWT

SEQ ID NO: 4 - protein, 10 aa, Homo sapiens
GFSLTGYSVN

SEQ ID NO: 5 - protein, 16 aa, Homo sapiens
MIWGDGSTDYNSALKS

SEQ ID NO: 6 - protein, 12 aa, Homo sapiens
DYYVNYASWFAY

SEQ ID NO: 7 - protein, 107 aa, Homo sapiens
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIK

SEQ ID NO: 8 - protein, 120 aa, Homo sapiens
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSS

SEQ ID NO: 9 - protein, 214 aa, Homo sapiens
DIQMTQSPSSLSASVGDRVTITCSASQGISNYLNWYQQKPGKTVKLLIYYTSNLHSGVPSRFSGSGSGTDYTLTISSLQPEDFATYYCQQYSELPWTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 10 - protein, 450 aa, Homo sapiens
EVQLVESGPALVKPTQTLTLTCTVSGFSLTGYSVNWIRQPPGKALEWLGMIWGDGSTDYNSALKSRLTISKDTSKNQVVLTMTNMDPVDTATYYCARDYYFNYASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 10 aa, Homo sapiens
GFSFSDFAMS

SEQ ID NO: 12 - protein, 18 aa, Homo sapiens
ATIGRVAFHTYYPDSMKG

SEQ ID NO: 13 - protein, 13 aa, Homo sapiens
ARHRGFDVGHFDF

SEQ ID NO: 14 - protein, 16 aa, Homo sapiens
RSSETLVHSSGNTYLE

SEQ ID NO: 15 - protein, 7 aa, Homo sapiens
RVSNRFS

SEQ ID NO: 16 - protein, 9 aa, Homo sapiens
FQGSFNPLT

SEQ ID NO: 17 - protein, 120 aa, Homo sapiens
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 18 - protein, 113 aa, Homo sapiens
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKR

SEQ ID NO: 19 - protein, 16 aa, Homo sapiens
RSSQSIVHSVGNTFLE

SEQ ID NO: 20 - protein, 7 aa, Homo sapiens
KVSNRFS

SEQ ID NO: 21 - protein, 9 aa, Homo sapiens
FQGSQFPYT

SEQ ID NO: 22 - protein, 10 aa, Homo sapiens
GYEFSRSWMN

SEQ ID NO: 23 - protein, 17 aa, Homo sapiens
RIYPGDGDTNYSGKFKG

SEQ ID NO: 24 - protein, 11 aa, Homo sapiens
DGSSWDWYFDV

SEQ ID NO: 25 - protein, 113 aa, Homo sapiens
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKR

SEQ ID NO: 26 - protein, 120 aa, Homo sapiens
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSS

SEQ ID NO: 27 - protein, 219 aa, Homo sapiens
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 28 - protein, 450 aa, Homo sapiens
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 445 aa, synthetic construct
  REGION 1..445: note = anti-huTfR1.hIgG1.LC.K149C.HC.L174C.Y373C.NG2LH heavy chain
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVCQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFCPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, synthetic construct
  REGION 1..214: note = anti-huTfR1.hIgG1.LC.K149C.HC.L174C.Y373C.NG2LH light chain
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 31 - protein, 445 aa, synthetic construct; identical to SEQ ID NO: 29
  REGION 1..445: note = anti-huTfR2.hIgG1.LC.K149C.HC.L174C.Y373C.NG2LH heavy chain

SEQ ID NO: 32 - protein, 214 aa, synthetic construct
  REGION 1..214: note = anti-huTfR2.hIgG1.LC.K149C.HC.L174C.Y373C.NG2LH light chain
DIQMTQSPSSLSASVGDRVTITCRASDNLYSNLAWYQQKPGKSPKLLVYDATNLADGVPSRFSGSGSGTDYTLTISSLQPEDFATYYCQHFAGTPLTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 33 - protein, 445 aa, synthetic construct
  REGION 1..445: note = anti-huTfR1.hIgG1.LC.K149C.NG2LH heavy chain
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 214 aa, synthetic construct; identical to SEQ ID NO: 30
  REGION 1..214: note = anti-huTfR1.hIgG1.LC.K149C.NG2LH light chain

SEQ ID NO: 35 - protein, 445 aa, synthetic construct; identical to SEQ ID NO: 33
  REGION 1..445: note = anti-huTfR2.hIgG1.LC.K149C.NG2LH heavy chain

SEQ ID NO: 36 - protein, 214 aa, synthetic construct; identical to SEQ ID NO: 32
  REGION 1..214: note = anti-huTfR2.hIgG1.LC.K149C.NG2LH light chain

(19) United States
(12) Patent Application Publication
(10) Pub. No.: US 2023/0330249 A1
(43) Pub. Date: Oct. 19, 2023

(54) ANTIBODY-CONJUGATED CHEMICAL INDUCERS OF DEGRADATION OF BRM AND METHODS THEREOF

(71) Applicant: Genentech, Inc., South San Francisco, CA (US)

(72) Inventors: Peter Scott DRAGOVICH, San Diego, CA (US); Summer A. BAKER DOCKREY, Millbrae, CA (US); Thomas Harden PILLOW, Millbrae, CA (US); Donglu ZHANG, San Mateo, CA (US)

(73) Assignee: Genentech, Inc., South San Francisco, CA (US)

(21) Appl. No.: 18/156,744

(22) Filed: Jan. 19, 2023

Related U.S. Application Data
(63) Continuation of application No. PCT/US2021/042280, filed on Jul. 20, 2021.
(60) Provisional application No. 63/054,757, filed on Jul. 21, 2020.

(51) Int. Cl. A61K 47/68; A61P 35/00

(52) U.S. Cl. CPC A61K 47/6811; A61P 35/00

(57) ABSTRACT

The subject matter described herein is directed to antibody-CIDE conjugates (Ab-CIDEs) that target BRM for degradation, to pharmaceutical compositions containing them, and to their use in treating diseases and conditions where BRM degradation is beneficial.

### CROSS REFERENCE TO RELATED APPLICATIONS

This application is a Continuation of International Application No. PCT/US2021/042280, filed Jul. 20, 2021, which claims the benefit of U.S. Provisional Patent Application No. 63/054,757, filed Jul. 21, 2020, the contents of each of which is incorporated by reference herein in its entirety.

### SEQUENCE LISTING

This application incorporates by reference a Sequence Listing electronically submitted in a XML file entitled “P36010-US-1_SL.xml”, created on May 18, 2023 and having a size of 39,855 bytes.

### FIELD

The subject matter described herein relates generally to degrader conjugates comprising antibody-proteolysis-targeting chimera molecules that are useful for facilitating intracellular degradation of target BRM proteins.

### BACKGROUND

Cell maintenance and normal function requires controlled degradation of cellular proteins. For example, degradation of regulatory proteins triggers events in the cell cycle, such as DNA replication, chromosome segregation, etc. Accordingly, such degradation of proteins has implications for the cell's proliferation, differentiation, and death.

While inhibitors of proteins can block or reduce protein activity in a cell, protein degradation in a cell can also reduce activity or remove altogether the target protein. Utilizing a cell's protein degradation pathway can, therefore, provide a means for reducing or removing protein activity. One of the cell's major degradation pathways is known as the ubiquitin-proteasome system. In this system, a protein is marked for degradation by the proteasome by ubiquitinating the protein. The ubiqitinization of the protein is accomplished by an E3 ubiquitin ligase that binds to a protein and adds ubiquitin molecules to the protein. The E3 ubiquitin ligase is part of a pathway that includes E1 and E2 ubiquitin ligases, which make ubiquitin available to the E3 ubiquitin ligase to add to the protein.

To harness this degradation pathway, molecular constructs known as chemical inducers of degradation (CIDEs) bring together an E3 ubiquitin ligase with a protein that is to be targeted for degradation. To facilitate a protein for degradation by the proteasome, the CIDE is comprised of a group that binds to an E3 ubiquitin ligase and a group that binds to the protein target for degradation. These groups are typically connected with a linker. This CIDE can bring the E3 ubiquitin ligase in proximity with the protein so that it is ubiquitinated and marked for degradation. However, the relatively large size of the CIDE can be problematic for targeted delivery, as well as contribute to undesirable properties, such as fast metabolism/clearance, short half-life, and low bioavailability.

There is an ongoing need in the art for improving CIDEs, including enhancing targeted delivery of CIDEs to cells that contain the protein target. The subject matter described herein addresses this and other shortcomings in the art.

### BRIEF SUMMARY

In one aspect, the subject matter described herein is directed to conjugated or covalently linked Ab-CIDEs, wherein the positions of the covalent bonds that connect the components of the Ab-CIDE: Antibody (Ab), Linker 1 (L1), Linker 2 (L2), protein binding group (PB) and the E3 ligase binding group (E3LB), can be tailored as desired to prepare Ab-CIDEs having desirable properties, such as potency, in vivo pharmacokinetics, stability and solubility.

In one aspect, the subject matter described herein is directed to an Ab-CIDE having the chemical structure:

Ab-(L1-D)ₚ,
wherein,
Ab is an antibody;
D is a CIDE, or prodrug thereof, having the structure:

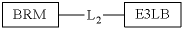

wherein,
BRM is a residue of a BRM-binding compound,
E3LB is a residue of an E3 ligase-binding compound, and
L2 is a moiety covalently linking BRM with E3LB;
L1 is a linker-1 covalently linking Ab to one of BRM, E3LB or L2; and
p is 1 to 16.

In another aspect, the subject matter described herein is directed to an Ab-CIDE having the chemical structure:

Ab-(L1-D)ₚ,
wherein,
Ab is an antibody;
D is a CIDE, or prodrug thereof, having the structure:

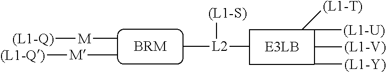

wherein L1 is attached at one attachment point selected from L1-Q, L1-Q′, L1-S, L1-T, and optionally L1-U, L1-V and L1-Y, if present, wherein
L1Q is at

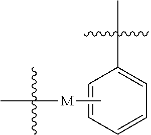

on BRM, wherein M is O;
L1-Q′ is at

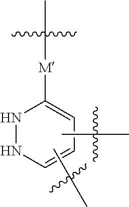

on BRM, wherein M′ is —NH;
L1-S is at

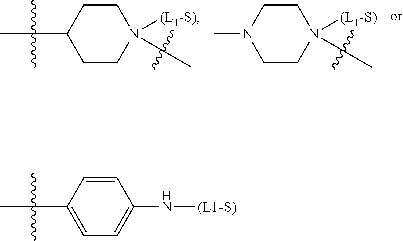

### on L2;

L1-T is at

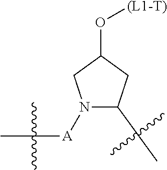

on E3LB, wherein, A is a group covalently bound to L2;
L1-U and L1-V are at

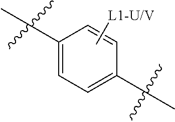

### on E3LB; and

L1-Y is at

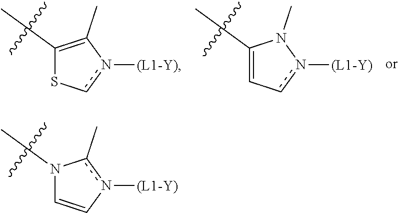

on E3LB, wherein, [symbol] is a single or double bond.

In another aspect, the subject matter described herein is directed to an Ab-CIDE having the chemical structure:

Ab-(L1-D)ₚ,
wherein,
Ab is an antibody;
D is a CIDE, or prodrug thereof, having the structure:

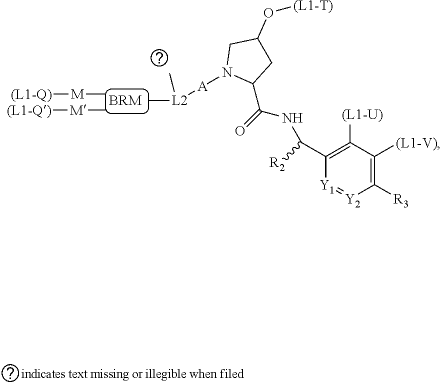

wherein:
   R₃ is cyano,

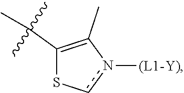

or

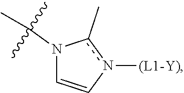

wherein, [symbol] is a single or double bond.

In another aspect, the subject matter described herein is directed to an Ab-CIDE having the chemical structure:

Ab-(L1-D)ₚ,
wherein,
Ab is an antibody;
D is a CIDE, or prodrug thereof, having the structure:

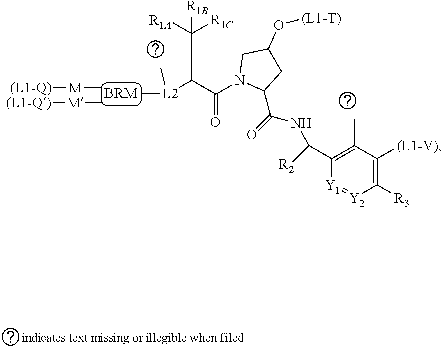

wherein, R_{1A}, R_{1B} and R_{1C} are each independently hydrogen, or C₁₋₅ alkyl; or two of R_{1A}, R_{1B} and R_{1C} together with the carbon to which each is attached form a C₁₋₅ cycloalkyl.

In another aspect, the subject matter described herein is directed to an Ab-CIDE having the chemical structure:

Ab-(L1-D)ₚ,
wherein,
   D is a CIDE having the structure E3LB-L2-PB;
   E3LB is covalently bound to L2, said E3LB having the formula:

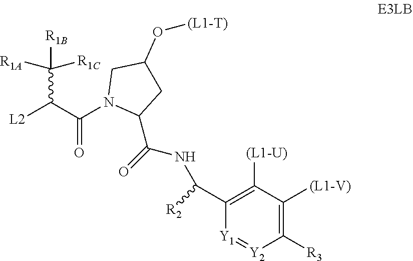

wherein,
   R_{1A}, R_{1B} and R_{1C} are each independently hydrogen, or C₁₋₅ alkyl; or two of R_{1A}, R_{1B} and R_{1C} together with the carbon to which each is attached form a C₁₋₅ cycloalkyl;
   R₂ is a C₁₋₅ alkyl;
   R₃ is selected from the group consisting of cyano,

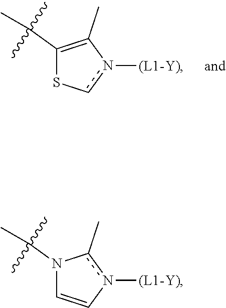

wherein, [symbol] is a single or double bond;
one of Y₁ and Y₂ is —CH, the other of Y₁ and Y₂ is —CH or N;
L2 is a linker covalently bound to E3LB and PB, said L2 having the formula:

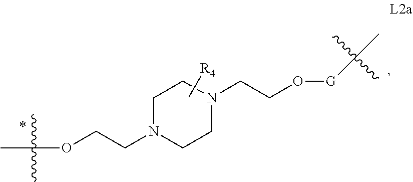

wherein,
   R₄ is hydrogen or methyl,

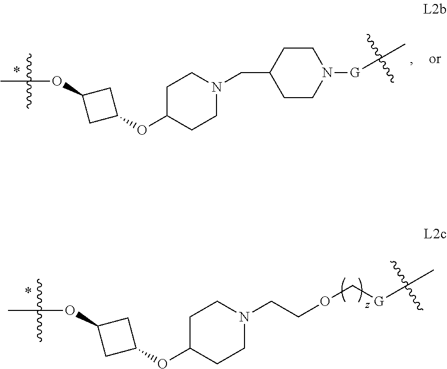

wherein,
   z is one or zero,
   G is

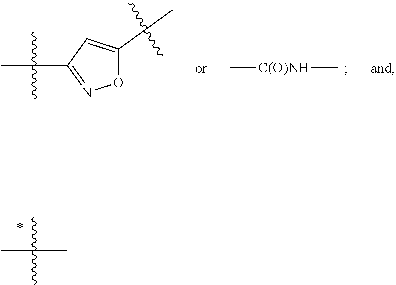

is the point of attachment to PB;
PB is a protein binding group covalently bound to L2, having the structure:

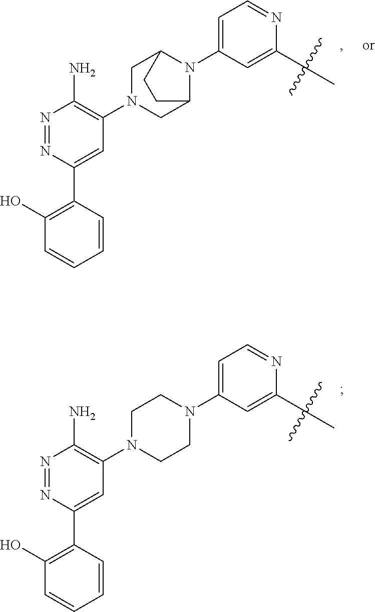 [SMILES: *c1cc(N2C3CCC2CN(c2cc(-c4ccccc4O)nnc2N)C3)ccn1 ; *c1cc(N2CCN(c3cc(-c4ccccc4O)nnc3N)CC2)ccn1]

Ab is an antibody covalently bound to at least one L1 that is a linker;
L1-T, L1-U, and L1-V are each independently hydrogen or a L1 linker covalently bound to Ab and D;
L1-Y is hydrogen or a L1 linker covalently bound to Ab and D;
q is 1 or zero;
and,
p has a value from about 1 to about 8.

Another aspect of the subject matter described herein is a pharmaceutical composition comprising an Ab-CIDE, and one or more pharmaceutically acceptable excipients.

Another aspect of the subject matter described herein is the use of an Ab-CIDE in methods of treating conditions and diseases by administering to a subject a pharmaceutical composition comprising an Ab-CIDE.

Another aspect of the subject matter described herein is a method of making an Ab-CIDE.

Another aspect of the subject matter described herein is an article of manufacture comprising a pharmaceutical composition comprising an Ab-CIDE, a container, and a package insert or label indicating that the pharmaceutical composition can be used to treat a disease or condition.

Yet other embodiments are also fully described herein.

### BRIEF DESCRIPTION OF THE FIGURES

FIGS. 1A and 1B shows an exemplary Ab-CIDE Ab-L1a-CIDE-BRM1-1 is active in cell-based assays.

FIGS. 2A and 2B shows an exemplary Ab-CIDE Ab-L1a-CIDE-BRM1-3 is active in cell-based assays.

FIG. 3A-3L shows dose and antigen-dependent anti-tumor activity of an exemplary Ab-CIDE Ab-L1a-CIDE-BRM1-1.

FIG. 4A-4L shows dose and antigen-dependent anti-tumor activity of an exemplary Ab-CIDE Ab-L1a-CIDE-BRM1-3. The data are in relative contrast to those of Ab-CIDE Ab-L1a-CIDE-BRM1-1.

FIG. 5 shows that BRM and BRG1 degradation correlate with anti-tumor activity of an exemplary Ab-CIDE Ab-L1a-CIDE-BRM1-1.

FIG. 6 shows that BRM and BRG1 degradation with anti-tumor activity of an exemplary Ab-CIDE Ab-L1a-CIDE-BRM1-3 is less correlative. All lanes are for Ab-CIDE Ab-L1a-CIDE-BRM1-3 except ** indicates the lane for Ab-CIDE-L1a-BRM1-1.

FIG. 7 shows that the antibody linking strategy can modulate the activity of the CIDE. The data show that the exemplary Ab-CIDE Ab-L1a-CIDE-BRM1-1 provides stronger BRM degradation than unconjugated CIDE-BRM1-3, although CIDE-BRM1-3 is generally more potent than CIDE-BRM1-1. All lanes are for Ab-CIDE Ab-Lia-CIDE-BRM1-1 except ** indicates the lane for Ab-CIDE-L1a-BRM1-3.

FIGS. 8-12 depict some of the antibody linking strategies described herein.

### DETAILED DESCRIPTION

Disclosed herein, are antibody-Chemical Inducers of Degradation (“CIDE”) conjugates, referred to herein as “Ab-CIDEs,” that are useful in targeted protein degradation of BRM, also known as SMARCA2, and the treatment of related diseases and disorders. In particular, the present disclosure is directed to antibody-conjugated CIDES, which contain on one end a ligand that binds to the Von Hippel-Lindau E3 ubiquitin ligase, and on the other end a moiety which binds BRM (target protein), such that the target protein is placed in proximity to the ubiquitin ligase to effect degradation, thus, modulating BRM, As described herein, the linking strategy and types of linkers were modulated and data are reported that show the modulations can have advantageous effects on the activity of the CIDE towards BRM.

The subject matter described herein utilizes antibody targeting to direct a CIDE to a target cell or tissue. As described herein, connecting an antibody to a CIDE to form an Ab-CIDE has been shown to deliver the CIDE to a target cell or tissue. As shown herein, e.g. in the Examples, a cell that expresses an antigen can be targeted by an antigen specific Ab-CIDE, whereby the CIDE portion of the Ab-CIDE is delivered intracellularly to the target cell. CIDEs that comprise an antibody directed to an antigen that is not found on the cell do not result in significant intracellular delivery of the CIDE to the cell.

Accordingly, the subject matter described herein is directed to Ab-CIDE compositions that result in the ubiquitination of a target protein and subsequent degradation of the protein. The compositions comprise an antibody covalently linked to a Linker 1 (L1), which is covalently linked at any available point of attachment to a CIDE, in which the CIDE comprises an E3 ubiquitin ligase binding (E3LB) moiety, wherein the E3LB moiety recognizes a E3 ubiquitin ligase protein that is VHL, a Linker 2 (L2) covalently connecting the E3LB moeity to the protein binding moiety (PB), which is the moeity that recognizes a target protein that is BRM or SMARCA2. The subject matter described herein is useful for degrading, and thus regulating protein activity, and treating diseases and conditions related to protein activity.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

The term “CIDE” refers to Chemical Inducers of DEgradation that are proteolysis-targeting chimera molecules having generally three components, an E3 ubiquitin ligase binding group (E3LB), a linker L2, and a protein binding group (PB).

The terms “residue,” “moiety,” “portion,” or “group” refers to a component that is covalently bound or linked to another component. The term “component” is also used herein to described such a residue, moiety, portion or group. By way of example, a residue of a compound will have an atom or atoms of the compound, such as a hydrogen or hydroxy, replaced with a covalent bond, thereby binding the residue to another component of the CIDE, L1-CIDE or Ab-CIDE. For example a “residue of a CIDE” refers to a CIDE that is covalently linked to one or more groups such as a Linker L2, which itself can be optionally further linked to an antibody.

The term “covalently bound” or “covalently linked” refers to a chemical bond formed by sharing of one or more pairs of electrons.

The term “peptidomimetic” or PM as used herein means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety includes non-amino acid chemical moieties. A peptidomimetic chemical moiety may also include one or more amino acid that are separated by one or more non-amino acid chemical units. A peptidomimetic chemical moiety does not contain in any portion of its chemical structure two or more adjacent amino acids that are linked by peptide bonds.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs (complementary determining regions) on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment(s)” as used herein comprises a portion of a full length antibody, generally the antigen binding or variable region thereof. Examples of antibody fragments include Fab, Fab′, F(ab′)₂, and Fv fragments; diabodies; linear antibodies; minibodies (Olafsen et al (2004) Protein Eng. Design & Sel. 17(4):315-323), fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the subject matter described herein may be made by the hybridoma method first described by Kohler et al (1975) Nature, 256:495, or may be made by recombinant DNA methods (see for example: U.S. Pat. Nos. 4,816,567; 5,807,715). The monoclonal antibodies may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape, etc.) and human constant region sequences.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “intact antibody” as used herein is one comprising a VL and VH domains, as well as a light chain constant domain (CL) and heavy chain constant domains, CH1, CH2 and CH3. The constant domains may be native sequence constant domains (e.g., human native sequence constant domains) or amino acid sequence variant thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

The term “Fc region” as used herein means a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

The term “framework” or “FR” as used herein refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310).

The term “human consensus framework” as used herein refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “variable region” or “variable domain” as used herein refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR,” as used herein, refers to each of the regions of an antibody variable domain that are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “epitope” refers to the particular site on an antigen molecule to which an antibody binds.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following. In certain embodiments, an antibody as described herein has dissociation constant (Kd) of ≤1 μM, ≤100 nM, ≤10 nM, ≤5 nm, ≤4 nM, ≤3 nM, ≤2 nM, ≤1 nM, ≤0.1 nM, ≤0.01 nM, or ≤0.001 nM (e.g., 10⁻⁸M or less, e.g. from 10⁻⁸ M to 10⁻¹³ M, e.g., from 10⁻⁹ M to 10⁻¹³ M).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “vector” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “free cysteine amino acid” as used herein refers to a cysteine amino acid residue which has been engineered into a parent antibody, has a thiol functional group (—SH), and is not paired as an intramolecular or intermolecular disulfide bridge. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

The term “Linker”, “Linker Unit”, or “link” as used herein means a chemical moiety comprising a chain of atoms that covalently attaches a CIDE moiety to an antibody, or a residue, portion, moiety, group or component of a CIDE to another residue, portion, moiety, group or component of the CIDE. In various embodiments, a linker is a divalent radical, specified as Linker 1, Linker 2, L1 or L2.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human. In some embodiments, the patient may be a “cancer patient,” i.e. one who is suffering or at risk for suffering from one or more symptoms of cancer.

A “patient population” refers to a group of cancer patients. Such populations can be used to demonstrate statistically significant efficacy and/or safety of a drug.

The terms “cancer” and “cancerous” refer to or describe the physiological condition in mammals that is typically characterized by unregulated cell growth/proliferation. A “tumor” comprises one or more cancerous cells. Examples of cancer are provided elsewhere herein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the subject matter described herein are used to delay development of a disease or to slow the progression of a disease.

A drug that is administered “concurrently” with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

An “effective amount” of an agent, e.g., a pharmaceutical formulation, refers to an amount effective, at dosages and for periods of time necessary, to achieve the desired therapeutic or prophylactic result. For example, an effective amount of the drug for treating cancer may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

As used herein, the term “therapeutically effective amount” means any amount which, as compared to a corresponding subject who has not received such amount, results in treatment of a disease, disorder, or side effect, or a decrease in the rate of advancement of a disease or disorder. The term also includes within its scope amounts effective to enhance normal physiological function. For use in therapy, therapeutically effective amounts of an Ab-CIDE, as well as salts thereof, may be administered as the raw chemical. Additionally, the active ingredient may be presented as a pharmaceutical composition.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The phrase “pharmaceutically acceptable salt,” as used herein, refers to pharmaceutically acceptable organic or inorganic salts of a molecule. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

Other salts, which are not pharmaceutically acceptable, may be useful in the preparation of compounds of described herein and these should be considered to form a further aspect of the subject matter. These salts, such as oxalic or trifluoroacetate, while not in themselves pharmaceutically acceptable, may be useful in the preparation of salts useful as intermediates in obtaining the compounds described herein and their pharmaceutically acceptable salts.

The term “alkyl” as used herein refers to a saturated linear or branched-chain monovalent hydrocarbon radical of any length from one to five carbon atoms (C₁-C₅), wherein the alkyl radical may be optionally substituted independently with one or more substituents described below. In another embodiment, an alkyl radical is one, two, three, four or five carbon atoms. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), and the like.

The term “alkylene” as used herein refers to a saturated linear or branched-chain divalent hydrocarbon radical of any length from one to twelve carbon atoms (C₁-C₁₂), wherein the alkylene radical may be optionally substituted independently with one or more substituents described below. In another embodiment, an alkylene radical is one to eight carbon atoms (C₁-C₅), or one to six carbon atoms (C₁-C₆). Examples of alkylene groups include, but are not limited to, methylene (—CH₂—), ethylene (—CH₂CH₂—), propylene (—CH₂CH₂CH₂—), and the like.

The terms “carbocycle”, “carbocyclyl”, “carbocyclic ring” and “cycloalkyl” refer to a monovalent non-aromatic, saturated or partially unsaturated ring having 3 to 5 carbon atoms (C₃-C₅) as a monocyclic ring. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, and the like. Carbocyclyl groups can be optionally substituted independently with one or more alkyl groups.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties. Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like. A heterocyclyl group can be substituted as described in WO2014/100762.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space. “Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Other terms, definitions and abbreviations herein include: Wild-type (“WT”); Cysteine engineered mutant antibody (“thio”); light chain (“LC”); heavy chain (“HC”); 6-maleimidocaproyl (“MC”); maleimidopropanoyl (“MP”); valine-citrulline (“val-cit” or “ve”), alanine-phenylalanine (“ala-phe”), p-aminobenzyl (“PAB”), and p-aminobenzyloxycarbonyl (“PABC”); A118C (EU numbering)=A121C (Sequential numbering)=A114C (Kabat numbering) of heavy chain K149C (Kabat numbering) of light chain. Still additional definitions and abbreviations are provided elsewhere herein.

### II. Chemical Inducers of Degradation

Chemical Inducers of Degradation (CIDE) molecules can be conjugated with an antibody to form an “Ab-CIDE” conjugate. The antibody is conjugated via a linker (L1) to a CIDE (“D”), wherein the CIDE comprises a ubiquitin E3 ligase binding group (“E3LB”), a linker (“L2”) and a protein binding group (“PB”). The general formula of an Ab-CIDE molecule is:

Ab-(L1-D)ₚ,

wherein, D is CIDE having the structure E3LB-L2-PB; wherein, E3LB is an E3 ligase binding group covalently bound to L2; L2 is a linker covalently bound to E3LB and PB; PB is a protein binding group covalently bound to L2; Ab is an antibody covalently bound to L1; L1 is a linker, covalently bound to Ab and to D; and p has a value from about 1 to about 50.

The variable p reflects that an antibody can be connected to one or more L1-D groups. In one embodiment, p is from about 1 to 8. In another embodiment, p is about 2.

The following sections describe the components that comprise the Ab-CIDE. To obtain an ab-CIDE having potent efficacy and a desirable therapeutic index, the following components are provided.

1. Antibody (Ab)

As described herein, antibodies, e.g., a monoclonal antibodies (mABs) are used to deliver a CIDE to target cells, e.g., cells that express the specific protein that is targeted by the antibody. The antibody portion of an Ab-CIDE can target a cell that expresses an antigen whereby the antigen specific Ab-CIDE is delivered intracellularly to the target cell, typically through endocytosis. While Ab-CIDEs that comprise an antibody directed to an antigen that is not found on the cell surface may result in less specific intracellular delivery of the CIDE portion into the cell, the Ab-CIDE may still undergo pinocytosis. The Ab-CIDEs and method of their use described herein advantageously utilize antibody recognition of the cellular surface and/or endocytosis of the Ab-CIDE to deliver the CIDE portion inside cells.

In particular embodiments, the antibody is a thiomab, described fully below. Thiomabs can have modulated Fe effector, e.g., LALAPG or NG2LH mutations. Further, combinations are contemplated, such that any antibody target (CD71, Trop2, MSLN, NaPi2b, Ly6E, EpCAM, and CD22) can be combined with any suitable combination of thiomab mutations with any Fc effector modulation including LALAPG or NG2LH mutations.

a. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

b. Library-Derived Antibodies

Antibodies for use in an Ab-CIDE may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

c. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

d. Multispecific Antibodies

In certain embodiments, an antibody provided herein is a multispecific antibody, e.g. a bispecific antibody. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules).

In some embodiments, multispecific antibodies are monoclonal antibodies that have binding specificities for at least two different antigen binding sites (such as a bispecific antibody). In some embodiments, the first antigen-binding domain and the second antigen-binding domain of the multispecific antibody may bind the two epitopes within one and the same molecule (intramolecular binding). For example, the first antigen-binding domain and the second antigen-binding domain of the multispecific antibody may bind to two different epitopes on the same protein molecule. In certain embodiments, the two different epitopes that a multispecific antibody binds are epitopes that are not normally bound at the same time by one monospecific antibody, such as e.g. a conventional antibody or one immunoglobulin single variable domain. In some embodiments, the first antigen-binding domain and the second antigen-binding domain of the multispecific antibody may bind epitopes located within two distinct molecules (intermolecular binding). For example, the first antigen-binding domain of the multispecific antibody may bind to one epitope on one protein molecule, whereas the second antigen-binding domain of the multispecific antibody may bind to another epitope on a different protein molecule, thereby cross-linking the two molecules.

In some embodiments, the antigen-binding domain of a multispecific antibody (such as a bispecific antibody) comprises two VH/VL units, wherein a first VH/VL unit binds to a first epitope and a second VH/VL unit binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, and antibody fragments (such as Fab, Fv, dsFv, scFv, diabodies, bispecific diabodies and triabodies, antibody fragments that have been linked covalently or non-covalently). A VH/VL unit that further comprises at least a portion of a heavy chain variable region and/or at least a portion of a light chain variable region may also be referred to as an “arm” or “hemimer” or “half antibody.” In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

In certain embodiments, a multispecific antibody provided herein may be a bispecific antibody. The term “bispecific antibody” as used herein refers to a multispecific antibody comprising an antigen-binding domain that is capable of binding to two different epitopes on one molecule or is capable of binding to epitopes on two different molecules. A bispecific antibody may also be referred to herein as having “dual specificity” or as being “dual specific.” Exemplary bispecific antibodies may bind both protein and any other antigen. In certain embodiments, one of the binding specificities is for protein and the other is for CD3. See, e.g., U.S. Pat. No. 5,821,337. In certain embodiments, bispecific antibodies may bind to two different epitopes of the same protein molecule. In certain embodiments, bispecific antibodies may bind to two different epitopes on two different protein molecules. Bispecific antibodies may also be used to localize cytotoxic agents to cells which express protein. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9). The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, Zhu et al., 1997, Protein Science 6:781-788, and WO2012/106587). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009. A mutation to introduce a “protuberance” may be referred to as a “knob mutation.”

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan. A mutation to introduce a “cavity” may be referred to as a “hole mutation.”

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgG1 constant region is T366W (EU numbering). In some embodiments, a hole mutation in an IgG1 constant region comprises one or more mutations selected from T366S, L368A and Y407V (EU numbering). In some embodiments, a hole mutation in an IgG1 constant region comprises T366S, L368A and Y407V (EU numbering).

In some embodiments, a knob mutation in an IgG4 constant region is T366W (EU numbering). In some embodiments, a hole mutation in an IgG4 constant region comprises one or more mutations selected from T366S, L368A, and Y407V (EU numbering). In some embodiments, a hole mutation in an IgG4 constant region comprises T366S, L368A, and Y407V (EU numbering).

Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies” or “dual-variable domain immunoglobulins” (DVDs) are also included herein (see, e.g., US 2006/0025576A1, and Wu et al. Nature Biotechnology (2007)).). The antibody or fragment herein also includes a “Dual Acting FAb” or “DAF” comprising an antigen binding site that binds to a target protein as well as another, different antigen (see, US 2008/0069820, for example).

e. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

f. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

g. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977); baby hamster kidney cells (BHK); mouse sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

Referring now to antibody affinity, in embodiments, the antibody binds to one or more tumor-associated antigens or cell-surface receptors. In embodiments, the tumor-associated antigen or cell surface receptor is selected from CD71, Trop2, MSLN, NaPi2b, Ly6E, EpCAM, and CD22.

As described herein, an Ab-CIDE may comprise an antibody, e.g., an antibody selected from:

i. Anti-Ly6E Antibodies

Ly6E (lymphocyte antigen 6 complex, locus E; Ly67, RIG-E, SCA-2, TSA-1); NP_002337.1; NM_002346.2; de Nooij-van Dalen, A. G. et al (2003) Int. J. Cancer 103 (6), 768-774; Zammit, D. J. et al (2002) Mol. Cell. Biol. 22 (3):946-952; WO 2013/17705.

In certain embodiments, an Ab-CIDE can comprise anti-Ly6E antibodies. Lymphocyte antigen 6 complex, locus E (Ly6E), also known as retinoic acid induced gene E (RIG-E) and stem cell antigen 2 (SCA-2). It is a GPI linked, 131 amino acid length, ˜8.4 kDa protein of unknown function with no known binding partners. It was initially identified as a transcript expressed in immature thymocyte, thymic medullary epithelial cells in mice (Mao, et al. (1996) Proc. Natl. Acad. Sci. U.S.A. 93:5910-5914). In some embodiments, the subject matter described herein provides an Ab-CIDE comprising an anti-Ly6E antibody described in PCT Publication No. WO 2013/177055.

In some embodiments, the subject matter described herein provides an Ab-CIDE comprising an anti-Ly6E antibody comprising at least one, two, three, four, five, or six HVRs selected from (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 1; (e) HVR-L2 comprising the amino acid sequence of SEQ ID NO: 2; and (f) HVR-L3 comprising the amino acid sequence of SEQ ID NO: 3.

In one aspect, the subject matter described herein provides an Ab-CIDE comprising an antibody that comprises at least one, at least two, or all three VH HVR sequences selected from (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO: 6. In a further embodiment, the antibody comprises (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO: 6.

In another aspect, the subject matter described herein provides an Ab-CIDE comprising an antibody that comprises at least one, at least two, or all three VL HVR sequences selected from (a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3. In one embodiment, the antibody comprises (a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3.

In another aspect, an Ab-CIDE comprises an antibody comprising (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 5, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 1, (ii) HVR-L2 comprising the amino acid sequence of SEQ ID NO: 2, and (c) HVR-L3 comprising the amino acid sequence of SEQ ID NO: 3.

In another aspect, the subject matter described herein provides an Ab-CIDE comprising an antibody that comprises (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 1; (e) HVR-L2 comprising the amino acid sequence of SEQ ID NO: 2; and (f) HVR-L3 comprising the amino acid sequence of SEQ ID NO: 3.

In any of the above embodiments, an anti-Ly6E antibody of an Ab-CIDE is humanized. In one embodiment, an anti-Ly6E antibody comprises HVRs as in any of the above embodiments, and further comprises a human acceptor framework, e.g. a human immunoglobulin framework or a human consensus framework.

In another aspect, an anti-Ly6E antibody of an Ab-CIDE comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 8. In certain embodiments, a VH sequence having at least 90%, 91%, 92%, 93%, 94%, 95%, 96%, 97%, 98%, or 99% identity to the amino acid sequence of SEQ ID NO:8 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 8. In certain embodiments, a total of 1 to 5 amino acids have been substituted, inserted and/or deleted in SEQ ID NO: 8. In certain embodiments, substitutions, insertions, or deletions occur in regions outside the HVRs (i.e., in the FRs). Optionally, the anti-Ly6E antibody comprises the VH sequence of SEQ ID NO: 8, including post-translational modifications of that sequence. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 5, and (c) HVR-H3 comprising the amino acid sequence of SEQ ID NO: 6.

In another aspect, an anti-Ly6E antibody of an Ab-CIDE is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 7. In certain embodiments, a VL sequence having at least 90%, 91%, 92%, 93%, 94%, 95%, 96%, 97%, 98%, or 99% identity to the amino acid sequence of SEQ ID NO:7 contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO: 7. In certain embodiments, a total of 1 to 5 amino acids have been substituted, inserted and/or deleted in SEQ ID NO: 7. In certain embodiments, the substitutions, insertions, or deletions occur in regions outside the HVRs (i.e., in the FRs). Optionally, the anti-Ly6E antibody comprises the VL sequence of SEQ ID NO: 7, including post-translational modifications of that sequence. In a particular embodiment, the VL comprises one, two or three HVRs selected from (a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3.

In another aspect, an Ab-CIDE comprising an anti-Ly6E antibody is provided, wherein the antibody comprises a VH as in any of the embodiments provided above, and a VL as in any of the embodiments provided above.

In one embodiment, an Ab-CIDE is provided, wherein the antibody comprises the VH and VL sequences in SEQ ID NO: 8 and SEQ ID NO: 7, respectively, including post-translational modifications of those sequences.

In a further aspect, provided herein are Ab-CIDEs comprising antibodies that bind to the same epitope as an anti-Ly6E antibody provided herein. For example, in certain embodiments, an Ab-CIDE is provided comprising an antibody that binds to the same epitope as an anti-Ly6E antibody comprising a VH sequence of SEQ ID NO: 8 and a VL sequence of SEQ ID NO: 7, respectively.

In a further aspect, an anti-Ly6E antibody of an Ab-CIDE according to any of the above embodiments is a monoclonal antibody, including a human antibody. In one embodiment, an anti-Ly6E antibody of an Ab-CIDE is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein. In some embodiments, an Ab-CIDE comprises an anti-Ly6E antibody comprising a heavy chain and a light chain comprising the amino acid sequences of SEQ ID NO: 10 and 9, respectively.

ii. Anti-NaPi2b Antibodies

Napi2b (Napi3b, NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1.

In certain embodiments, an Ab-CIDE comprises anti-NaPi2b antibodies.

In some embodiments, described herein are Ab-CIDEs comprising an anti-NaPi2b antibody comprising at least one, two, three, four, five, or six HVRs selected from (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15 and (f) HVR-L3 comprising the amino acid sequence of SEQ ID NO: 16.

In one aspect, described herein are Ab-CIDEs comprising an antibody that comprises at least one, at least two, or all three VH HVR sequences selected from (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13. In a further embodiment, the antibody comprises (a) HVR-H1 comprising the amino acid sequence of SEQ ID NO: 11; (b) HVR-H2 comprising the amino acid sequence of SEQ ID NO: 12; (c) HVR-H3 comprising the amino acid sequence of SEQ ID NO: 13.

In another aspect, described herein are Ab-CIDEs comprising an antibody that comprises at least one, at least two, or all three VL HVR sequences selected from (a) HVR-L1 comprising the amino acid sequence of SEQ ID NO: 14; (b) HVR-L2 comprising the amino acid sequence of SEQ ID NO: 15; and (c) HVR-L3 comprising the amino acid sequence of SEQ ID NO: 16. In one embodiment, the antibody comprises (a) HVR-L1 comprising the amino acid sequence of SEQ ID NO: 14; (b) HVR-L2 comprising the amino acid sequence of SEQ ID NO: 15; and (c) HVR-L3 comprising the amino acid sequence of SEQ ID NO: 16.

In another aspect, an Ab-CIDE comprises an antibody comprising (a) a VH domain comprising at least one, at least two, or all three VH HVR sequences selected from (i) HVR-H1 comprising the amino acid sequence of SEQ ID NO: 11, (ii) HVR-H2 comprising the amino acid sequence of SEQ ID NO: 12, and (iii) HVR-H3 comprising an amino acid sequence selected from SEQ ID NO: 13; and (b) a VL domain comprising at least one, at least two, or all three VL HVR sequences selected from (i) HVR-L1 comprising the amino acid sequence of SEQ ID NO: 14, (ii) HVR-L2 comprising the amino acid sequence of SEQ ID NO: 15, and (c) HVR-L3 comprising the amino acid sequence of SEQ ID NO: 16.

In another aspect, described herein are Ab-CIDEs comprising an antibody that comprises (a) HVR-H1 comprising the amino acid sequence of SEQ ID NO: 11 (b) HVR-H2 comprising the amino acid sequence of SEQ ID NO: 12; (c) HVR-H3 comprising the amino acid sequence of SEQ ID NO: 13; (d) HVR-L1 comprising the amino acid sequence of SEQ ID NO: 14; (e) HVR-L2 comprising the amino acid sequence of SEQ ID NO: 15; and (f) HVR-L3 comprising the amino acid sequence of SEQ ID NO: 16.

In any of the above embodiments, an anti-NaPi2b antibody of an Ab-CIDE is humanized. In one embodiment, an anti-NaPi2b antibody comprises HVRs as in any of the above embodiments, and further comprises a human acceptor framework, e.g. a human immunoglobulin framework or a human consensus framework.

In another aspect, an anti-NaPi2b antibody of an Ab-CIDE comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 17 In certain embodiments, a VH sequence having at least 90%, 91%, 92%, 93%, 94%, 95%, 96%, 97%, 98%, or 99% identity to the amino acid sequence of SEQ ID NO: 54 contains substitutions (e.g., conservative substitutions), insertions, or deletions relative to the reference sequence, but an anti-NaPi2b antibody comprising that sequence retains the ability to bind to NaPi2b. In certain embodiments, a total of 1 to 10 amino acids have been substituted, inserted and/or deleted in SEQ ID NO: 17. In certain embodiments, a total of 1 to 5 amino acids have been substituted, inserted and/or deleted in SEQ ID NO: 17. In certain embodiments, substitutions, insertions, or deletions occur in regions outside the HVRs (i.e., in the FRs). Optionally, the anti-NaPi2b antibody comprises the VH sequence of SEQ ID NO: 17, including post-translational modifications of that sequence. In a particular embodiment, the VH comprises one, two or three HVRs selected from: (a) HVR-H1 comprising the amino acid sequence of SEQ ID NO: 11, (b) HVR-H2 comprising the amino acid sequence of SEQ ID NO: 12, and (c) HVR-H3 comprising the amino acid sequence of SEQ ID NO: 13.

In another aspect, an anti-NaPi2b antibody of an Ab-CIDE is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 18. In certain embodiments, a VL sequence having at least 90%, 91%, 92%, 93%, 94%, 95%, 96%, 97%, 98%, or 99% identity to the amino acid sequence of SEQ ID NO: 18 contains substitutions (e.g., conservative substitutions), insertions, or deletions relative to the reference sequence, but an anti-NaPi2b antibody comprising that sequence retains the ability to bind to anti-NaPi2b. In certain embodiments, a total of 1 to 10 amino acids have been substituted, inserted and/or deleted in SEQ ID NO: 18. In certain embodiments, a total of 1 to 5 amino acids have been substituted, inserted and/or deleted in SEQ ID NO: 18. In certain embodiments, the substitutions, insertions, or deletions occur in regions outside the HVRs (i.e., in the FRs). Optionally, the anti-NaPi2b antibody comprises the VL sequence of SEQ ID NO: 18, including post-translational modifications of that sequence. In a particular embodiment, the VL comprises one, two or three HVRs selected from (a) HVR-L1 comprising the amino acid sequence of SEQ ID NO: 14; (b) HVR-L2 comprising the amino acid sequence of SEQ ID NO: 15; and (c) HVR-L3 comprising the amino acid sequence of SEQ ID NO: 16.

In another aspect, an Ab-CIDE comprising an anti-NaPi2b antibody is provided, wherein the antibody comprises a VH as in any of the embodiments provided above, and a VL as in any of the embodiments provided above.

In one embodiment, an Ab-CIDE is provided, wherein the antibody comprises the VH and VL sequences in SEQ ID NO: 17 and SEQ ID NO: 18, respectively, including post-translational modifications of those sequences.

In a further aspect, provided herein are Ab-CIDEs comprising antibodies that bind to the same epitope as an anti-NaPi2b antibody provided herein. For example, in certain embodiments, an Ab-CIDE is provided comprising an antibody that binds to the same epitope as an anti-NaPi2b antibody comprising a VH sequence of SEQ ID NO: 17 and a VL sequence of SEQ ID NO: 18, respectively.

In a further aspect, an anti-NaPi2b antibody of an Ab-CIDE according to any of the above embodiments is a monoclonal antibody, including a human antibody. In one embodiment, an anti-NaPi2b antibody of an Ab-CIDE is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein.

iii. Anti-CD22 Antibodies

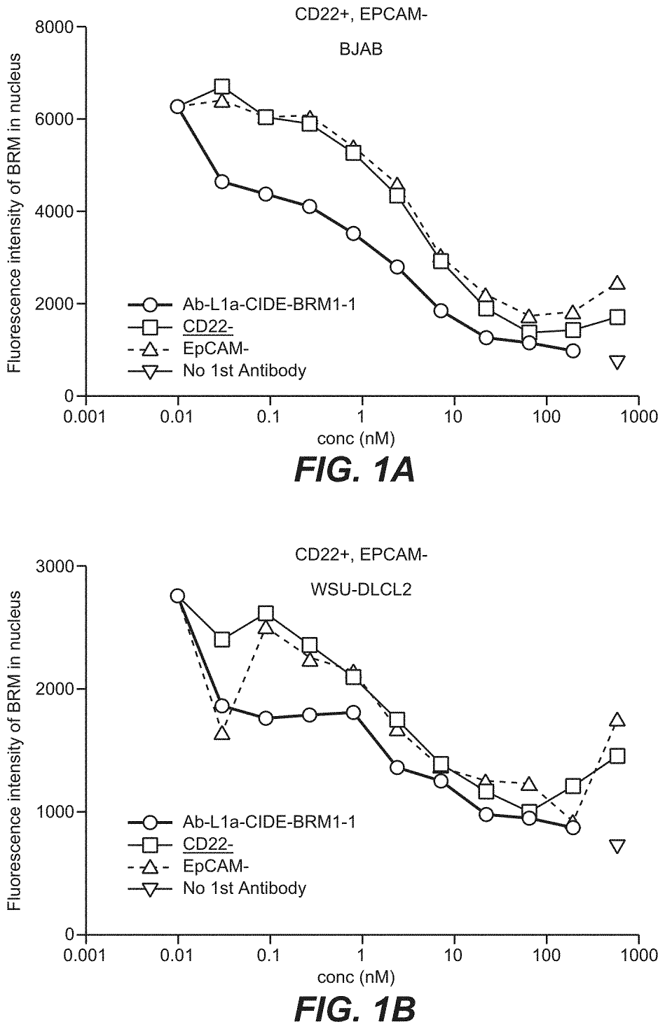

CD22 (B-cell receptor CD22-B isoform, BL-CAM, Lyb-8, Lyb8, SIGLEC-2, FLJ22814, Genbank accession No. AK026467); Wilson et al (1991) J. Exp. Med. 173:137-146; WO2003072036 (Claim 1; FIG. 1); Cross-references: MIM:107266; NP_001762.1; NM_001771_1

In certain embodiments, an Ab-CIDE can comprise anti-CD22 antibodies, which comprise three light chain hypervariable regions (HVR-L1, HVR-L2 and HVR-L3) and three heavy chain hypervariable regions (HVR-H1, HVR-H2 and HVR-H3). In one embodiment, the anti-CD22 antibody of an Ab-CIDE comprises three light chain hypervariable regions and three heavy chain hypervariable regions (SEQ ID NO: 19-24), the sequences of which are shown below. In one embodiment, the anti-CD22 antibody of an Ab-CIDE comprises the variable light chain sequence of SEQ ID NO: 25 and the variable heavy chain sequence of SEQ ID NO: 26. In one embodiment, the anti-CD22 antibody of Ab-CIDEs of the present invention comprises the light chain sequence of SEQ ID NO: 27 and the heavy chain sequence of SEQ ID NO: 28.

iv. Anti-CD71 Antibodies

In certain embodiments, an Ab-CIDE can comprise anti-CD71 antibodies. CD71 (transferrin receptor) is an integral membrane glycoprotein that plays an important role in cellular uptake of iron. It is well known as a marker for cell proliferation and activation. Although all proliferating cells in hematopoietic system express CD71, however, CD71 has been considered as a useful erythroid-associated antigen. In any of the above embodiments, an anti-CD71 antibody of an Ab-CIDE is humanized.

In one embodiment, the anti-CD71 antibody comprises a NG2LH modification which is a combination of an N297G mutation plus the IgG2 Lower Hinge region that reduces/eliminates IgG1 mAb effector function. In another embodiment, the anti-CD71 antibody comprises engineered Cys residues used for conjugation to the linker. In one embodiment, the parent IgG1 mAb lacking all of these changes is described in: WO2016081643 which is incorporated by reference in its entirety.

In embodiments, the anti-CD71 antibody is anti huTfR1.hIgG1.LC. K149C.HC.L174C.Y373C.NG2LH ABP1AA25970 (high affinity DAR6). In one embodiment, the anti-CD71 antibody of an Ab-CIDE comprises the light chain sequence of SEQ ID NO: 30 and the heavy chain sequence of SEQ ID NO: 29.

In embodiments, the anti-CD71 antibody is anti-huTfR2.hIgG1.LC.K149C.HC.L174C.Y373C.NG2LH ABP1AA25969 (low affinity DAR6). In one embodiment, the anti-CD71 antibody of an Ab-CIDE comprises the light chain sequence of SEQ ID NO: 32 and the heavy chain sequence of SEQ ID NO: 31.

In embodiments, the anti-CD71 antibody is anti-huTfR1.hIgG1.LC.K149C.NG2LH ABP1AA30139 (high affinity DAR2). In one embodiment, the anti-CD71 antibody of an Ab-CIDE comprises the light chain sequence of SEQ ID NO: 34 and the heavy chain sequence of SEQ ID NO: 33.

In embodiments, the anti-CD71 antibody is anti-huTfR2.hIgG1.LC.K149C.NG2LH ABP1AA30140 (low affinity DAR2). In one embodiment, the anti-CD71 antibody of an Ab-CIDE comprises the light chain sequence of SEQ ID NO: 36 and the heavy chain sequence of SEQ ID NO: 35.

v. Anti-Trop2 Antibodies

In certain embodiments, an Ab-CIDE can comprise anti-Trop2 antibodies. Trop2 (trophoblast antigen 2) is a transmembrane glycoprotein that is an intracellular calcium signal transducer that is differentially expressed in many cancers. It signals cells for self-renewal, proliferation, invasion, and survival. Trop 2 is also known as cell surface glycoprotein Trop-2/Trop2, gastrointestinal tumor-associated antigen GA7331, pancreatic carcinoma marker protein GA733-1/GA733, membrane component chromosome 1 surface marker 1 M1S1, epithelial glycoprotein-1, EGP-1, CAA1, Gelatinous Drop-Like Corneal Dystrophy GDLD, and TTD2. In any of the above embodiments, an anti-Trop2 antibody of an Ab-CIDE is humanized. In one embodiments, the anti-Trop2 antibodies are described in US-2014/0377287 and US-2015/0366988, each of which is incorporated by reference in its entirety.

vi. Anti-MSLN Antibodies

In certain embodiments, an Ab-CIDE can comprise anti-MSLN antibodies. MSLN (mesothelin) is a glycosylphosphatidylinositol-anchored cell-surface protein that may function as a cell adhesion protein. MSLN is also known as CAKI and MPF. This protein is overexpressed in epithelial mesotheliomas, ovarian cancers and in specific squamous cell carcinomas. In any of the above embodiments, an anti-MSLN antibody of an Ab-CIDE is humanized. In one embodiment, the anti-MSLN antibody is h7D9.v3 described in Scales, S. J. et al., Mol. Cancer Ther. 2014, 13(11), 2630-2640, which is incorporated by reference in its entirety.

vii. Anti-EpCAM Antibodies

In certain embodiments, an Ab-CIDE can comprise anti-EpCAM antibodies. In an aspect, the antibody of the Ab-CIDE may be an antibody that is directed to a protein that is found on numerous cells or tissue types. Examples of such antibodies include EpCAM. Epithelial cell adhesion molecule (EpCAM) is a transmembrane glycoprotein mediating Ca2+-independent homotypic cell-cell adhesion in epithelia (Litvinov, S. et al. (1994) Journal of Cell Biology 125(2):437-46). Also known as DIAR5, EGP-2, EGP314, EGP40, ESA, HNPCC8, KS1/4, KSA, M4S1, MIC18, MK-1, TACSTD1, TROP1, EpCAM is also involved in cell signaling, (Maetzel, D. et al. (2009) Nature Cell Biology 11(2):162-71), migration (Osta, W A; et al. (2004) Cancer Res. 64(16):5818-24), proliferation, and differentiation (Litvinov, S. et al. (1996) Am J Pathol. 148(3):865-75). Additionally, EpCAM has oncogenic potential via its capacity to upregulate c-myc, e-fabp, and cyclins A & E (Munz, M. et al. (2004) Oncogene 23(34):5748-58). Since EpCAM is expressed exclusively in epithelia and epithelial-derived neoplasms, EpCAM can be used as a diagnostic marker for various cancers. In other words, an Ab-CIDE can be used to deliver a CIDE to many cells or tissues rather than specific cell types or tissue types as when using a using a targeted antibody.

1. Antibody Affinity

In certain embodiments, an antibody provided herein has a dissociation constant (Kd) of ≤1 M, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER© multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

2. Linkers (L1)

As described herein, a “linker” (L1, Linker-1) is a bifunctional or multifunctional moiety that can be used to link one or more CIDE moieties (D) to an antibody (Ab) to form an Ab-CIDE. In some embodiments, Ab-CIDEs can be prepared using a L1 having reactive functionalities for covalently attaching to the CIDE and to the antibody. For example, in some embodiments, a cysteine thiol of an antibody (Ab) can form a bond with a reactive functional group of a linker or a linker L1-CIDE group to make an Ab-CIDE. Particularly, the chemical structure of the linker can have significant impact on both the efficacy and the safety of an Ab-CIDE (Ducry & Stump, Bioconjugate Chem, 2010, 21, 5-13). Choosing the right linker influences proper drug delivery to the intended cellular compartment of target cells.

In certain embodiments, the L1 linker can be self-immolative.

In certains embodiments, the L1 linker is selected from the group consisting of L1a, L1b and L1:

### Examples of L1a

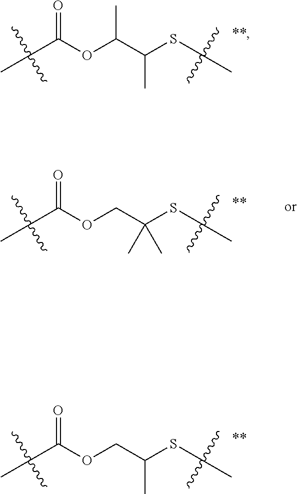

### Examples of L1b

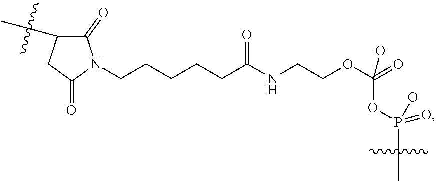

### Examples of L1e

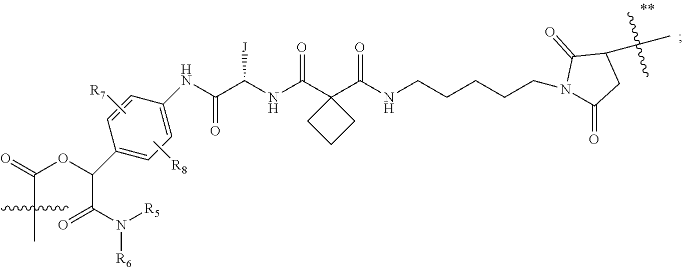 [0247] wherein,
   J is —CH₂—CH₂—CH₂—NH—C(O)—NH₂; —CH₂—CH₂—CH₂—CH₂—NH₂; —CH₂—CH₂—CH₂—CH₂—NH—CH₃; or —CH₂—CH₂—CH₂—CH₂—N(CH₃)₂;
   R₅ and R₆ are independently hydrogen or C₁₋₅ alkyl; or R₅ and R₆ together with the nitrogen to which each is attached form an optionally substituted 5- to 7-member heterocyclyl;
   R₇ and R₈ are each independently hydrogen, halo, C₁₋₅ alkyl, C₁₋₅ alkoxy or hydroxy;
and wherein

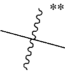

is the point of attachment to Ab.

In certain embodiments, the L1 linker is a hydrophilic self-immolative linker. Examples of these types of L1 linkers are those described in WO2014/100762, herein incorporated by reference in its entirety. L1 linkers include, but are not limited to, Formulae I-XII.

The present disclosure provides an L1 linker of Formula (I):

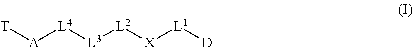

or a salt or solvate or stereoisomer thereof;
wherein:
D is drug moiety or CIDE;
T is a targeting moiety such as an antibody;
X is a hydrophilic self-immolative linker;
L¹ is distinct from L1, and is a bond, a second self-immolative linker, or a cyclization self-elimination linker;
L² is a bond or a second self-immolative linker;
   wherein if L¹ is a second self-immolative linker or a cyclization self-elimination linker, then L is a bond;
   wherein if L² is a second self-immolative linker, then L¹ is a bond;
L³ is a peptide linker;
L⁴ is bond or a spacer; and
A is an acyl unit.

In some embodiments, provided is a L1 linker of Formula (Ia):

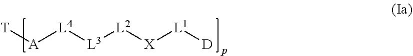

or a salt or solvate or stereoisomer thereof; wherein D, T, X, L¹, L², L³, L4 and A are as defined for Formula (I), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4.

The present disclosure also provides a L1 linker of Formula (II):

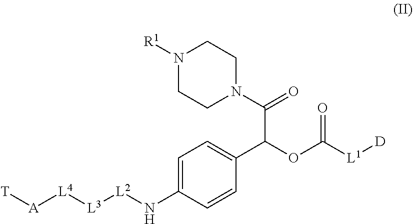

or a salt or solvate or stereoisomer thereof;
wherein:
D is drug moiety or CIDE;
T is a targeting moiety or antibody;
R¹ is hydrogen, unsubstituted or substituted C₁₋₃ alkyl, or unsubstituted or substituted heterocyclyl;
L¹ is a bond, a second self-immolative linker, or a cyclization self-elimination linker; 2
L² is a bond, a second self-immolative linker;
   wherein if L¹ is a second self-immolative linker or a cyclization self-elimination linker, then L² is a bond;
   wherein if L¹ is a second self-immolative linker, then L² is a bond;
L³ is a peptide linker;
L⁴ is bond or a spacer; and
A is an acyl unit.

In some embodiments, provided is a L1 linker of Formula (IIa):

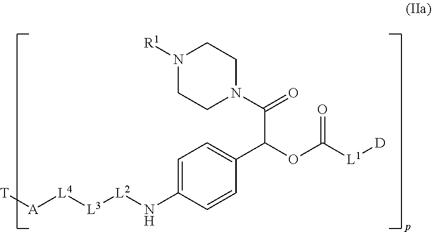

or a salt or solvate or stereoisomer thereof; wherein D, T, L¹, L², L³, L⁴ and A are as defined for Formula (II), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4.

The present disclosure also provides a L1 linker of Formula (III):

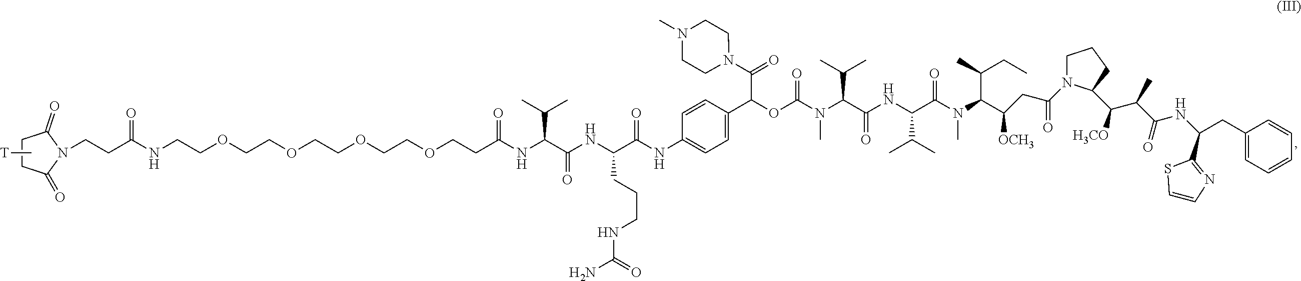

or a salt or solvate or stereoisomer thereof;
wherein T is a targeting moiety.

In some embodiments, provided is a L1 linker of Formula (IIIa):

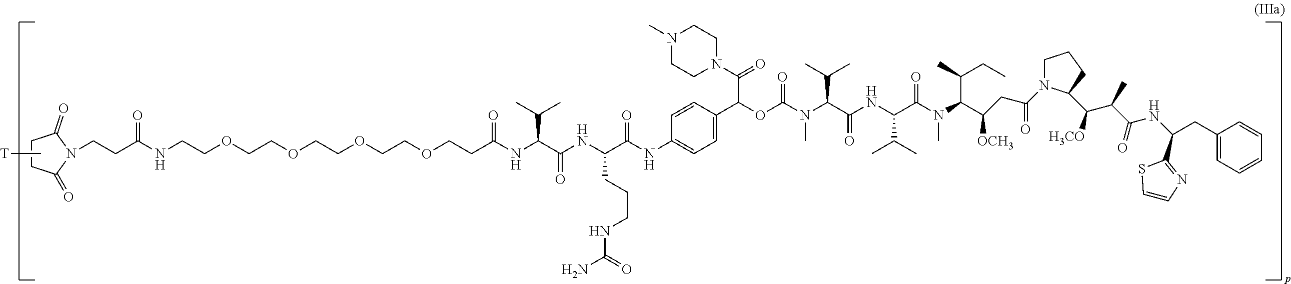

or a salt or solvate or stereoisomer thereof; wherein T is a targeting moiety and p is 1 to 20.

In some embodiments, p is 1 to 8. In some embodiments, p is 1 to 6. In some embodiments, p is 1 to 4. In some embodiments, p is 2 to 4. In some embodiments, p is 1, 2, 3 or 4.

The present disclosure provides a L1 linker of Formula (IV):

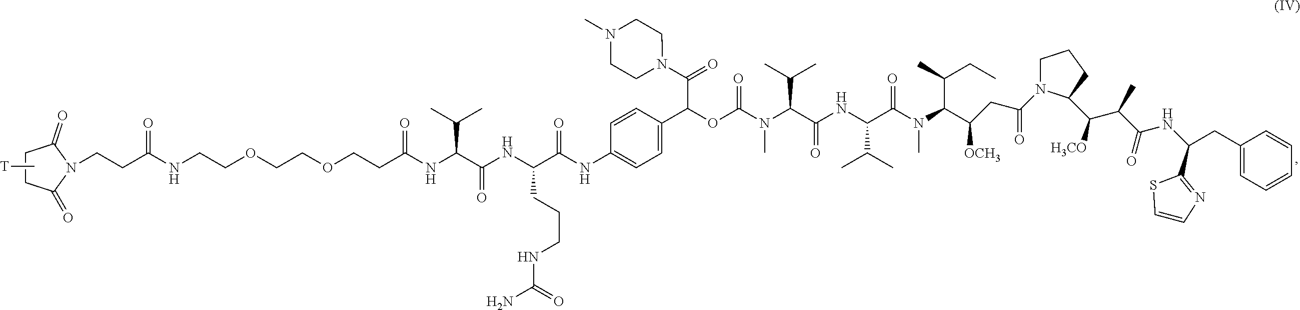

or a salt or solvate or stereoisomer thereof;
wherein T is a targeting moiety.

In some embodiments, provided is a L1 linker of Formula (IVa):

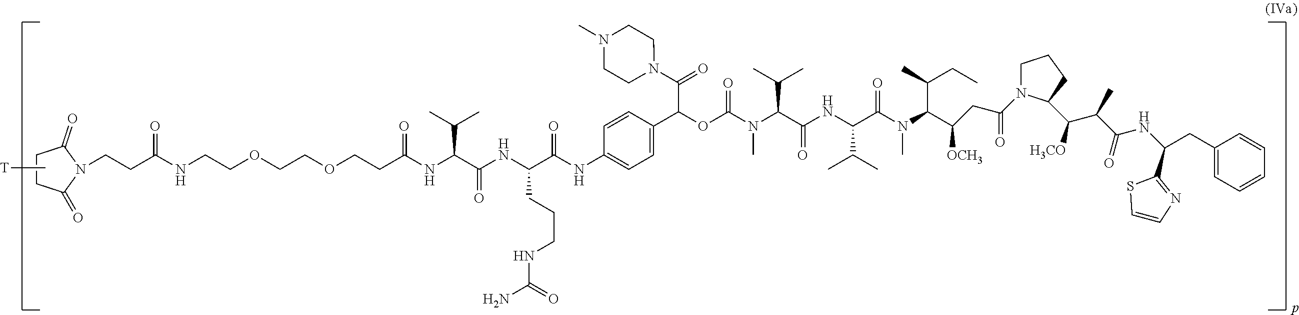

or a salt or solvate or stereoisomer thereof; wherein T is a targeting moiety and p is 1 to 20.

In some embodiments, p is 1 to 8. In some embodiments, p is 1 to 6. In some embodiments, p is 1 to 4. In some embodiments, p is 2 to 4. In some embodiments, p is 1, 2, 3 or 4.

The present disclosure provides a L1 linker of Formula (V):

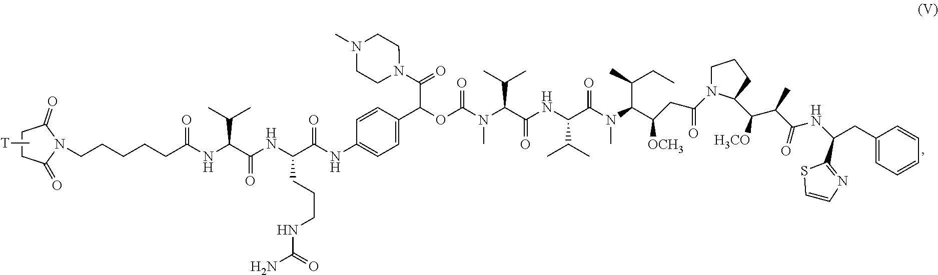

or a salt or solvate or stereoisomer thereof;
wherein T is a targeting moiety.

In some embodiments, provided is a L1 linker of Formula (Va):

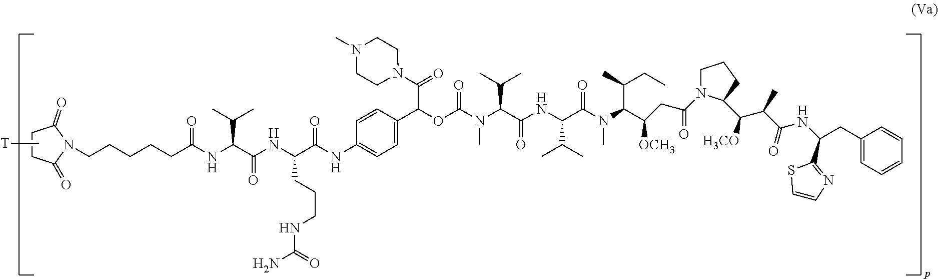

or a salt or solvate or stereoisomer thereof; wherein T is a targeting moiety and p is 1 to 20.

In some embodiments, p is 1 to 8. In some embodiments, p is 1 to 6. In some embodiments, p is 1 to 4. In some embodiments, p is 2 to 4. In some embodiments, p is 1, 2, 3 or 4.

The present disclosure provides a L1 linker of Formula (VI):

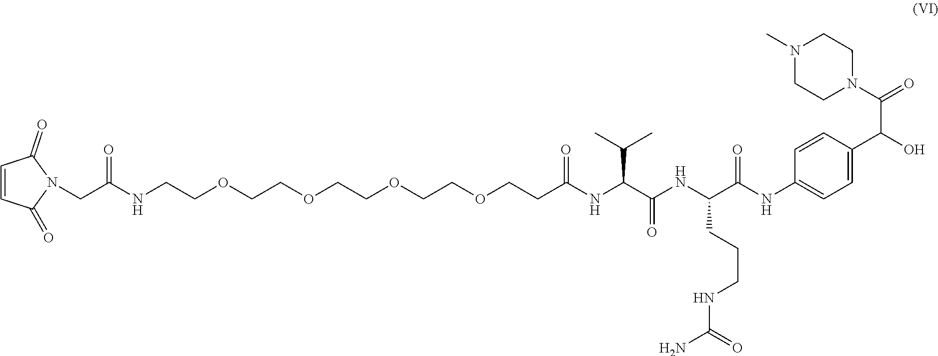 [SMILES: CC(C)[C@H](NC(=O)CCOCCOCCOCCOCCNC(=O)CN1C(=O)C=CC1=O)C(=O)N[C@@H](CCCNC(N)=O)C(=O)Nc1ccc(C(O)C(=O)N2CCN(C)CC2)cc1]

or a salt or solvate thereof.

The present disclosure provides a L1 linker of Formula (VII):

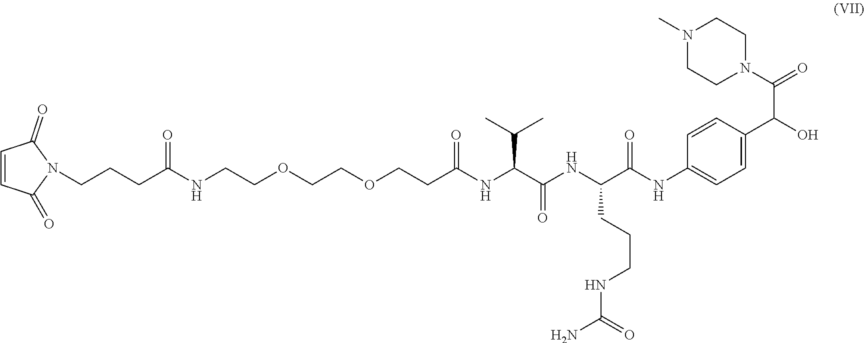 [SMILES: CC(C)[C@H](NC(=O)CCOCCOCCNC(=O)CCCN1C(=O)C=CC1=O)C(=O)N[C@@H](CCCNC(N)=O)C(=O)Nc1ccc(C(O)C(=O)N2CCN(C)CC2)cc1]

or a salt or solvate thereof.

The present disclosure provides a L1 linker of Formula (VIII):

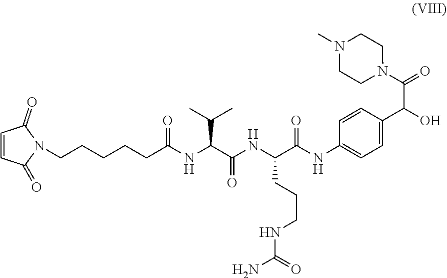 [SMILES: CC(C)[C@H](NC(=O)CCCCCN1C(=O)C=CC1=O)C(=O)N[C@@H](CCCNC(N)=O)C(=O)Nc1ccc(C(O)C(=O)N2CCN(C)CC2)cc1]

The present disclosure provides a L1 linker of Formula (XII):

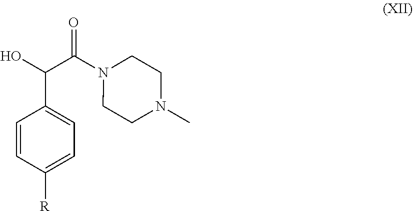

or a salt or solvate or stereoisomer thereof; wherein R is NO₂ or NH₂.

In certain embodiments, L1 linkers can also be generally divided into two categories: cleavable (such as peptide, hydrzone, or disulfide) or non-cleavable (such as thioether). If a linker is a non-cleavable linker, then its position on the E3LB portion is such that it does not interfere with VHL binding. Specifically, the non-cleavable linker is not to be covalently linked at the hydroxyl position on the proline of the VHL-binding domain. Peptide linkers, such as Valine-Citrulline (Val-Cit), that can be hydrolyzed by lysosomal enzymes (such as Cathepsin B) have been used to connect the drug with the antibody (U.S. Pat. No. 6,214,345). They have been particularly useful, due in part to their relative stability in systemic circulation and the ability to efficiently release the drug in tumor. However, the chemical space represented by natural peptides is limited; therefore, it is desirable to have a variety of non-peptide linkers which act like peptides and can be effectively cleaved by lysosomal proteases. The greater diversity of non-peptide structures may yield novel, beneficial properties that are not afforded by the peptide linkers. Provided herein are different types of non-peptide linkers for linker L1 that can be cleaved by lysosomal enzymes.

a. Peptidomimetic Linkers

Provided herein are different types of non-peptide, peptidomimetic linkers for Ab-CIDE that are cleavable by lysosomal enzymes. For example, the amide bond in the middle of a dipeptide (e.g. Val-Cit) was replaced with an amide mimic; and/or entire amino acid (e.g., valine amino acid in Val-Cit dipeptide) was replaced with a non-amino acid moiety (e.g., cycloalkyl dicarbonyl structures (for example, ring size=4 or 5)).

When L1 is a peptidomimetic linker, it is represented by the following formula

-Str-(PM)-Sp-,
wherein:
Str is a stretcher unit covalently attached to Ab;
Sp is a bond or spacer unit covalently attached to a CIDE moiety; and
PM is a non-peptide chemical moiety selected from the group consisting of:

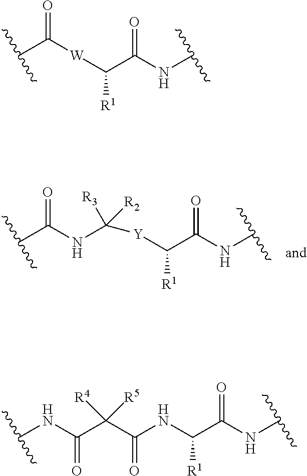

W is —NH-heterocycloalkyl- or heterocycloalkyl;
Y is heteroaryl, aryl, —C(O)C₁-C₆alkylene, C₁-C₆alkylene-NH₂, C₁-C₆alkylene-NH—CH₃, C₁-C₆alkylene-N—(CH₃)₂, C₁-C₆alkenyl or C₁-C₆alkylenyl;
each R¹ is independently C₁-C₁₀alkyl, C₁-C₁₀alkenyl, (C₁-C₁₀alkyl)NHC(NH)NH₂ or (C₁-C₁₀alkyl)NHC(O)NH₂;
R³ and R² are each independently H, C₁-C₁₀alkyl, C₁-C₁₀alkenyl, arylalkyl or heteroarylalkyl, or R³ and R² together may form a C₃-C₇cycloalkyl; and
R⁴ and R⁵ are each independently C₁-C₁₀alkyl, C₁-C₁₀alkenyl, arylalkyl, heteroarylalkyl, (C₁-C₁₀alkyl)OCH₂—, or R⁴ and R⁵ may form a C₃-C₇cycloalkyl ring.

It is noted that L1 may be connected to the CIDE through any of the E3LB, L2, or PB groups.

In embodiments, Y is heteroaryl; R⁴ and R⁵ together form a cyclobutyl ring.

In embodiments, Y is a moiety selected from the group consisting of:

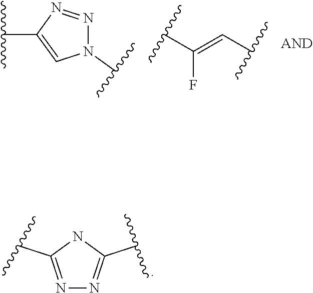 [SMILES: *c1cn(*)nn1 ; */C=C(/*)F ; *c1nnc(*)[nH]1]

In embodiments, Str is a chemical moiety represented by the following formula:

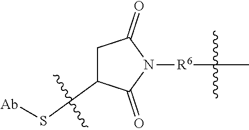

wherein R⁶ is selected from the group consisting of C₁-C₁₀alkylene, C₁-C₁₀alkenyl, C₃-C₈cycloalkyl, (C₁-C₈alkylene)O—, and C₁-C₁₀alkylene-C(O)N(R^{a})_C₂-C₆alkylene, where each alkylene may be substituted by one to five substituents selected from the group consisting of halo, trifluoromethyl, difluoromethyl, amino, alkylamino, cyano, sulfonyl, sulfonamide, sulfoxide, hydroxy, alkoxy, ester, carboxylic acid, alkylthio, C₃-C₅cycloalkyl, C₄-C₇heterocycloalkyl, aryl, arylalkyl, heteroarylalkyl and heteroaryl each R^{a} is independently H or C₁-C₆alkyl; Sp is —Ar—R^{b}—, wherein Ar is aryl or heteroaryl, R^{b} is (C₁-C₁₀alkylene)O—. Conjugation to the antibody can occur as the maleimide reacts via Michael addition with an exposed Cys residue on the antibody. The exposed Cys residue can either be artificially introduced by molecular engineering and/or produced by reduction of the interchain disulfide bonds)

In embodiments, Str has the formula:

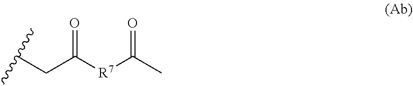

wherein R⁷ is selected from C₁-C₁₀alkylene, C₁-C₁₀alkenyl, (C₁-C₁₀alkylene)O—, N(R^{c})—(C₂-C₆ alkylene)-N(R^{c}) and N(R^{c})—(C₂-C₆alkylene); where each R^{c} is independently H or C₁-C₆ alkyl; Sp is —Ar—R^{b}—, wherein Ar is aryl or heteroaryl, R^{b} is (C₁-C₁₀alkylene)O— or Sp-C₁-C₆alkylene-C(O)NH—.

In embodiments, L1 is a non-peptide chemical moiety represented by the following formula

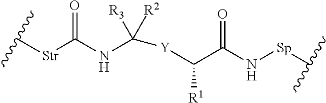

R¹ is C₁-C₆alkyl, C₁-C₆alkenyl, (C₁-C₆alkyl)NHC(NH)NH₂ or (C₁-C₆alkyl)NHC(O)NH₂;
R³ and R² are each independently H or C₁-C₁₀alkyl.

In embodiments, L1 is a non-peptide chemical moiety represented by the following formula

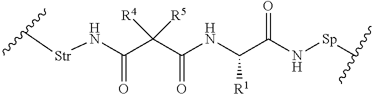

R¹ is C₁-C₆ alkyl, (C₁-C₆alkyl)NHC(NH)NH₂ or (C₁-C₆alkyl)NHC(O)NH₂;
   R⁴ and R⁵ together form a C₃-C₇cycloalkyl ring.

In embodiments, L1 is a non-peptide chemical moiety represented by the following formula

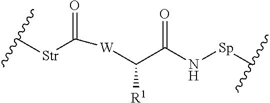

R¹ is C₁-C₆alkyl, (C₁-C₆alkyl)NHC(NH)NH₂ or (C₁-C₆alkyl)NHC(O)NH₂ and W is as defined above.

In some embodiments, the linker may be a peptidomimetic linker such as those described in WO2015/095227, WO2015/095124 or WO2015/095223, each of which is hereby incorporated by reference in its entirety.

In certain embodiments, the linker is selected from the group consisting of:

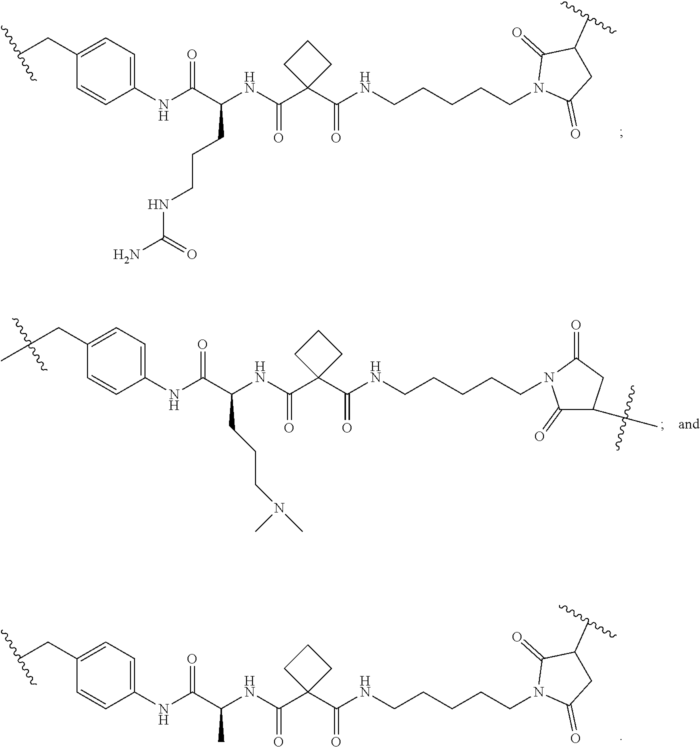 [SMILES: *Cc1ccc(NC(=O)[C@H](CCCNC(N)=O)NC(=O)C2(C(=O)NCCCCCN3C(=O)CC(*)C3=O)CCC2)cc1 ; *Cc1ccc(NC(=O)[C@H](CCCN(C)C)NC(=O)C2(C(=O)NCCCCCN3C(=O)CC(*)C3=O)CCC2)cc1 ; *Cc1ccc(NC(=O)[C@H](C)NC(=O)C2(C(=O)NCCCCCN3C(=O)CC(*)C3=O)CCC2)cc1]

b. Non-Peptidomimetic Linkers

In an aspect, a Linker L1 may be covalently bound to an antibody and a CIDE as follows:

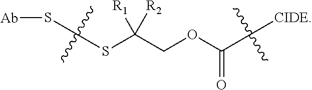

In an aspect, a Linker L1 forms a disulfide bond with the antibody, and the linker has the structure:

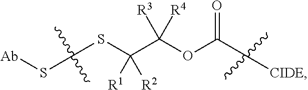

wherein R¹, R², R³, and R⁴ are independently selected from the group consisting of H, optionally substituted branched or linear C₁-C₅ alkyl, and optionally substituted C₃-C₆ cycloalkyl, or R¹ and R² taken together or R³ and R⁴ taken together with the carbon atom to which they are bound form an optionally substituted C₃-C₆ cycloalkyl ring or a 3 to 6-membered heterocycloalkyl ring.

In one aspect the carbonyl group of the linker is connected to an amine group in the CIDE. It is also noted that the sulfur atom connected to Ab is a sulfur group from a cysteine in the antibody. In another aspect, a linker L1 has a functionality that is capable of reacting with a free cysteine present on an antibody to form a covalent bond. Nonlimiting examples of such reactive functionalities include maleimide, haloacetamides, a-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a L1 linker has a functionality that is capable of reacting with an electrophilic group present on an antibody. Examples of such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting examples of such reactive functionalities include, but are not limited to, hydrazide, oxime, amino, hydrazine, thiosemicarbazone, hydrazine carboxylate, and arylhydrazide.

A L1 linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

A L1 linker may be a “cleavable linker,” facilitating release of a CIDE. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker has the following Formula:

-Aₐ-W_{w}—Y_{y}—
wherein A is a “stretcher unit”, and a is an integer from 0 to 1; W is an “amino acid unit”, and w is an integer from 0 to 12; Y is a “spacer unit”, and y is 0, 1, or 2. Exemplary embodiments of such linkers are described in U.S. Pat. No. 7,498,298.

In some embodiments, a L1 linker component comprises a “stretcher unit” that links an antibody to another linker component or to a CIDE moiety. Nonlimiting exemplary stretcher units are shown below (wherein the wavy line indicates sites of covalent attachment to an antibody, CIDE, or additional linker components):

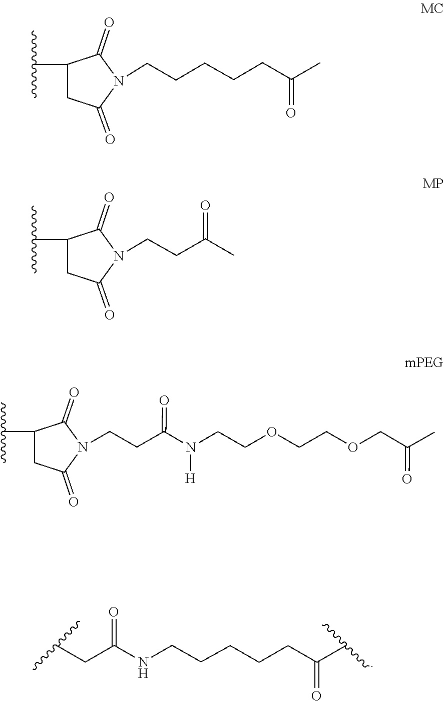 [SMILES: *C1CC(=O)N(CCCCCC(C)=O)C1=O ; *C1CC(=O)N(CCC(C)=O)C1=O ; *C1CC(=O)N(CCC(=O)NCCOCCOCC(C)=O)C1=O ; *CC(=O)NCCCCCC(*)=O]

In certain embodiments, the linker is:

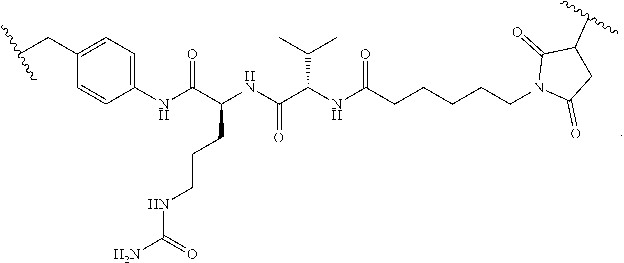 [SMILES: *Cc1ccc(NC(=O)[C@H](CCCNC(N)=O)NC(=O)[C@@H](NC(=O)CCCCCN2C(=O)CC(*)C2=O)C(C)C)cc1]

In certain embodiments, a linker has the following Formula:

-Aₐ-Y_{y}—
wherein A and Y are defined as above. In certain embodiments, the spacer unit Y may be a phosphate, such as a monophosphate or a bisphosphate. In certain embodiments, the stretcher component A comprises:

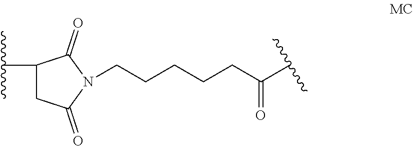 [SMILES: *C(=O)CCCCCN1C(=O)CC(*)C1=O]

In certain embodiments, the linker is:

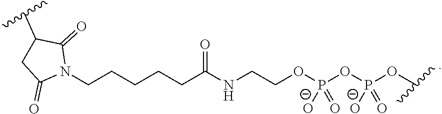 [SMILES: *OP(=O)([O-])OP(=O)([O-])OCCNC(=O)CCCCCN1C(=O)CC(*)C1=O]

3. CIDE (“D”)

Useful CIDEs have the general formula described above.

Useful Ab-L1-CIDEs and unconjugated degraders exhibit desirable properties such as cell targeting, and protein targeting and degradation. In certain embodiments, the Ab-L1-CIDEs exhibit a DC50 (μg/mL) from 0.0001 to less than about 2.0, or less than about 1.0, or less than about 0.8, or less than about 0.7, or less than about 0.6, or less than about 0.5, or less than about 0.4, or less than about 0.3, or less than about 0.2. In certain embodiments, the Ab-L1-CIDEs exhibit a DCmax of at least 70, 75, 80, 85, 90, 95, 96, 97, 98 or 99.

CIDEs include those having the following components.

a. E3 Ubiquitin Ligases Binding Groups (E3LB)

E3 ubiquitin ligases (of which over 600 are known in humans) confer substrate specificity for ubiquitination. There are known ligands which bind to these ligases. As described herein, an E3 ubiquitin ligase binding group is a peptide or small molecule that can bind an E3 ubiquitin ligase that is von Hippel-Lindau (VHL).

A particular E3 ubiquitin ligase is von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels.

In one aspect, the subject matter herein is directed to an E3LB portion of a CIDE having the chemical structure:

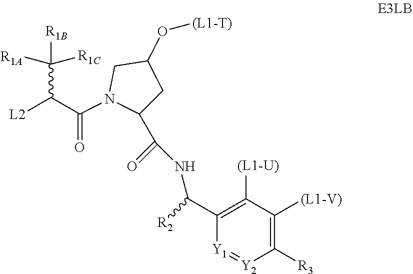

wherein, R_{1A}, R_{1B} and R_{1C} are each independently hydrogen, or C₁₋₅ alkyl; or two of R_{1A}, R_{1B} and R_{1C} together with the carbon to which each is attached form a C₁₋₅ cycloalkyl;
R₂ is a C₁₋₅ alkyl;
R₃ is selected from the group consisting of cyano,

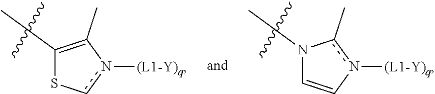

wherein, [symbol] is a single or double bond; and q is 1 or zero; [symbol]
one of Y₁ and Y₂ is —CH, the other of Y₁ and Y₂ is —CH or N;
wherein, L1-T, L1-U, L1-V and L1-Y are each independently as described elsewhere herein; and L2 is as described elsewhere herein.

In certain embodiments, E3LB has the structure wherein R₃ is cyano.

In certain embodiments, E3LB has the structure wherein R₃ is

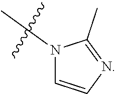 [SMILES: *n1ccnc1C]

In certain embodiments, E3LB has the structure wherein R₃ is

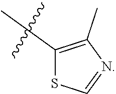 [SMILES: *c1scnc1C]

In certain embodiments, E3LB has the structure wherein R_{1A}, R_{1B} and R_{1C} are each independently hydrogen or methyl.

In certain embodiments, E3LB has the structure wherein R_{1A} and R_{iB} are each methyl.

In certain embodiments, E3LB has one of the following formulae:

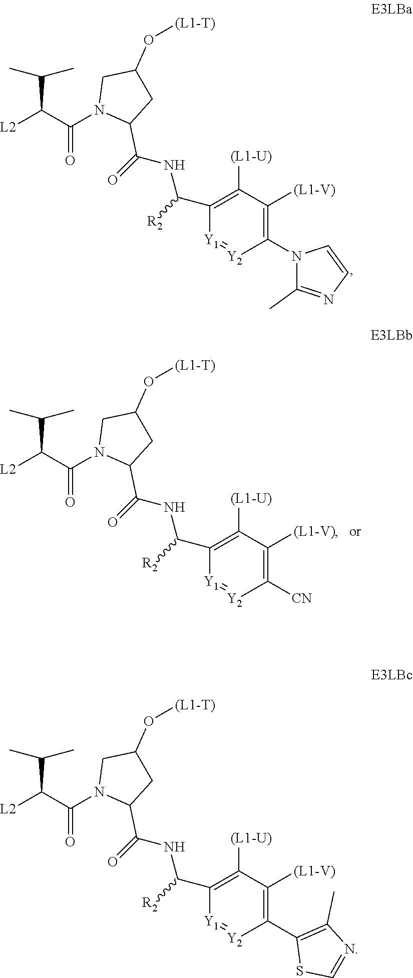

In certain embodiments, E3LB has the structure wherein R₂ is hydrogen, methyl, ethyl or propyl.

In certain embodiments, E3LB has the structure wherein R₂ is methyl.

In certain embodiments, E3LB has the structure wherein R₂ is

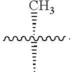

In certain embodiments, E3LB has the structure wherein Y₁ and Y₂ are each —CH.

In certain embodiments, E3LB has the structure wherein Y₁ is N and Y₂ is —CH.

In certain embodiments, E3LB has the structure wherein Y₁ is —CH and Y₂ is N.

In certain embodiments, the proline portion of E3LB has the structure:

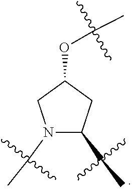 [SMILES: *O[C@@H]1C[C@@H](*)N(*)C1]

The E3LB portion has at least one terminus with a moeity that is or can be covalently linked to the L2 portion, and at least one terminus with a moeity that is or can be covalently linked to the L1 portion. For example, the E3LB portion terminates in a —NHCOOH moeity that can be covalently linked to the L2 portion through an amide bond.

In any of the aspects or embodiments described herein, the E3LB as described herein may be a pharmaceutically acceptable salt, enantiomer, diastereomer, solvate or polymorph thereof. In addition, in any of the aspects or embodiments described herein, the E3LB as described herein may be coupled to a PB directly via a bond or by a chemical linker.

b. BRM Protein Binding Group (PB)

The PB portion of the CIDE is a small molecule moeity that binds to BRM, including all variants, mutations, splice variants, indels and fusions of BRM. BRM is also known as Subfamily A, Member 2, SMARCA2 and BRAHMA. Such small molecule target protein binding moieties also include pharmaceutically acceptable salts, enantiomers, solvates and polymorphs of these compositions, as well as other small molecules that may target a protein of interest.

The CIDEs or DACs described herein can comprise any residue of a known BRM binding compound, binding compounds including those disclosed in WO2019/195201, herein incorporated by reference in its entirety.

In certain embodiments, the BRM binding compound is a compound of Formula I:

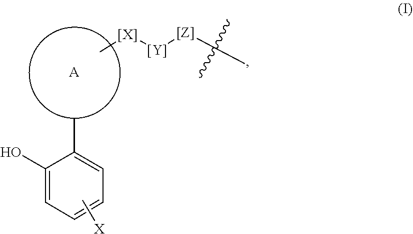

or a stereoisomer or tautomer thereof, or a pharmaceutically acceptable salt of any of the foregoing, wherein:
wherein X is hydrogen or halogen;

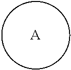

is selected from the group consisting of:

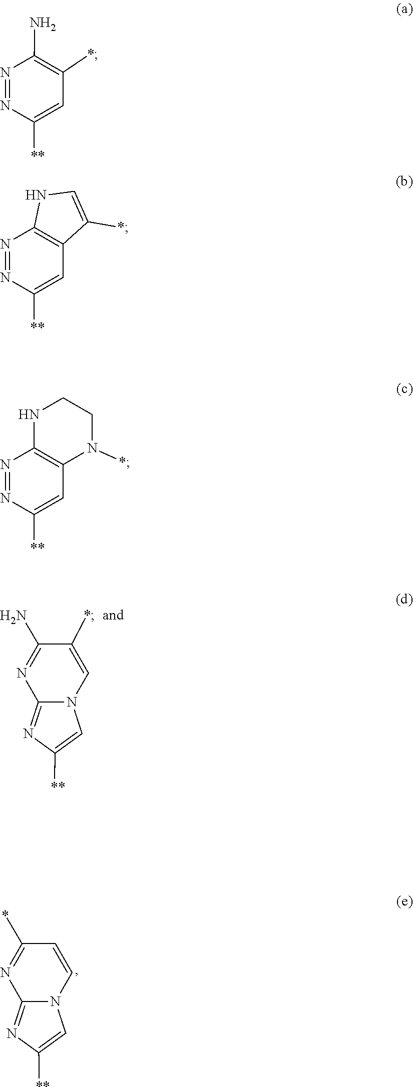

wherein, for (a)-(e), * denotes the point of attachment to [X], or, if [X] is absent, * denotes the point of attachment to [Y], and ** denotes the point of attachment to the phenyl ring; and wherein:
   (i) [X] is 3-15 membered heterocyclyl or 5-20 membered heteroaryl,
provided that, when

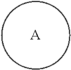

is (a), then [X] is not

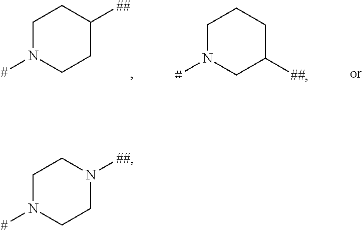

wherein #denotes the point of attachment to

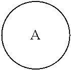

and ##denotes the point of attachment to L2,
   [Y] is absent, and
   [Z] is absent; or
(ii) [X] is 3-15 membered heterocyclyl or 5-20 membered heteroaryl, wherein the 3-15 membered heterocyclyl of [X] is optionally substituted with one or more —OH or C₁₋₆alkyl,
   [Y] is absent, and
   [Z] is 3-15 membered heterocyclyl or 5-20 membered heteroaryl, provided that, when

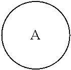

is (a) and [X] is

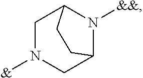

wherein & denotes the point of attachment to

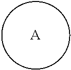

and && denotes the point of attachment to [Z], then [Z] is not

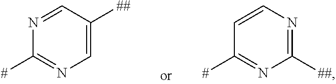

wherein #denotes the point of attachment to [X] and ##denotes the point of attachment to L2; or
(iii) [X] is 3-15 membered heterocyclyl or 5-20 membered heteroaryl,
   [Y] is methylene, wherein the methylene of [Y] is optionally substituted with one or more methyl group, and
   [Z] is 3-15 membered heterocyclyl; or
(iv) [X] is absent,
   [Y] is ethenylene, wherein the ethenylene of [Y] is optionally substituted with one or more halo, and
   [Z] is 5-20 membered heteroaryl,
provided that

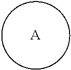

is (a), (b), (d), or (e); or
   (v) [X] is absent,
      [Y] is ethynylene, and
      [Z] is 5-20 membered heteroaryl,
provided that

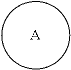

is (a), (b), (d), or (e); or
   (vi) [X] is absent,
      [Y] is cyclopropyl or cyclobutyl, and
      [Z] is 5-20 membered heteroaryl,
provided that

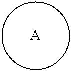

is (a), (b), (d), or (e).

In certain embodiments, the BRM binding compound is a compound of formula (I-A):

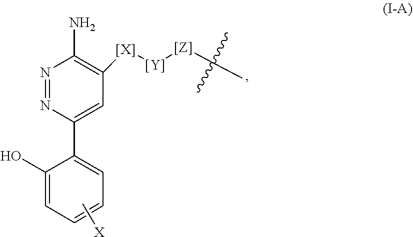

or a stereoisomer or tautomer thereof, or a pharmaceutically acceptable salt of any of the foregoing, wherein X is hydrogen or halogen, and wherein [X], [Y] and [Z] are as defined above for a compound of formula (I).

In certain embodiments, the BRM binding compound is a compound of formula (I-B):

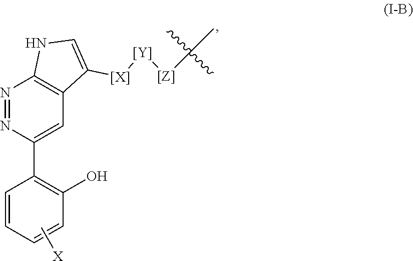

or a stereoisomer or tautomer thereof, or a pharmaceutically acceptable salt of any of the foregoing, wherein X is hydrogen or halogen, and wherein [X], [Y] and [Z] are as defined above for a compound of formula (I).

In certain embodiments, the BRM binding compound is a compound of formula (I-C):

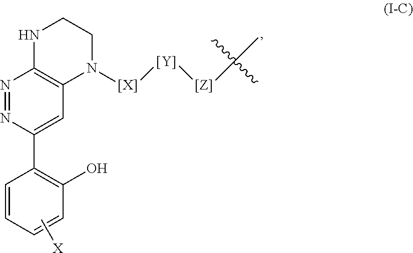

or a stereoisomer or tautomer thereof, or a pharmaceutically acceptable salt of any of the foregoing, wherein X is hydrogen or halogen, and wherein [X], [Y] and [Z] are as defined above for a compound of formula (I).

In certain embodiments, the BRM binding compound is a compound of formula (I-D):

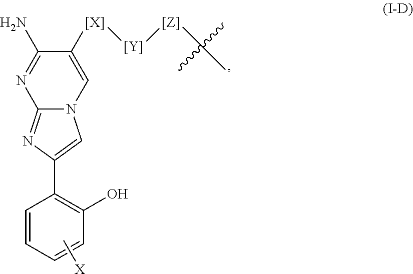

or a stereoisomer or tautomer thereof, or a pharmaceutically acceptable salt of any of the foregoing, wherein X is hydrogen or halogen, and wherein [X], [Y] and [Z] are as defined above for a compound of formula (I).

In certain embodiments, the BRM binding compound is a compound of formula (I-E):

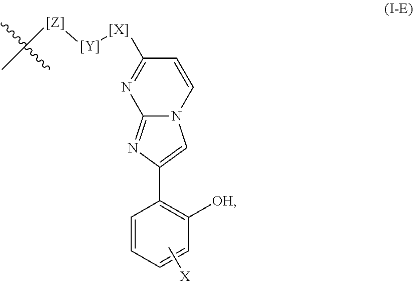

or a stereoisomer or tautomer thereof, or a pharmaceutically acceptable salt of any of the foregoing, wherein X is hydrogen or halogen, and wherein [X], [Y] and [Z] are as defined above for a compound of formula (I).

In certain embodiments, the PB (BRM) portion of the CIDE has the structure:

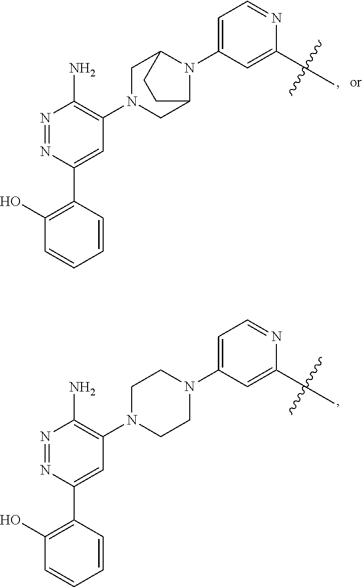

wherein,

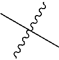

is the point of covalent attachment to L2.

c. Linker L2

The E3LB and PB portions of CIDEs as described herein can be connected with linker (L2, Linker L2, Linker-2). In certain embodiments, the Linker L2 is covalently bound to the E3LB portion and covalently bound to the PB portion, thus making up the CIDE.

In certain embodiments, the L2 portion can be selected from linkers disclosed in WO2019/195201, herein incorporated by reference in its entirety.

Although the E3LB group and PB group may be covalently linked to the linker group through any group which is appropriate and stable to the chemistry of the linker, in certain aspects, the L2 is independently covalently bonded to the E3LB group and the PB group through an amide, ester, thioester, keto group, carbamate (urethane) or ether, each of which groups may be inserted anywhere on the E3LB group and PB group to allow binding of the E3LB group to the ubiquitin ligase and the PB group to the BRM target protein to be degraded. In other words, as shown herein, the linker can be designed and connected to E3LB and PB to modulate the binding of E3LB and PB to their respective binding partners.

In certain embodiments, L2 is a linker covalently bound to E3LB and PB, the L2 having the formula:

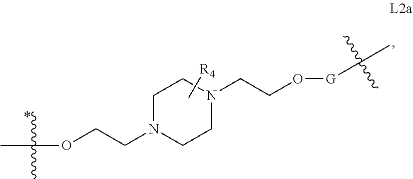

wherein,
   R₄ is hydrogen or methyl,

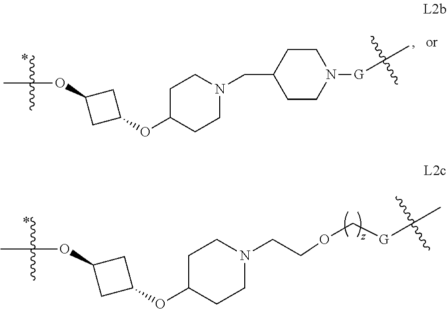

wherein,
   z is one or zero,
   G is

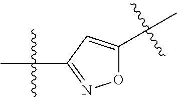 [SMILES: *c1cc(*)on1]

or —C(O)NH—; and,

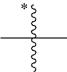

is the point of attachment to PB.

In certain embodiments of L2a, R₄ is hydrogen.

In certain embodiments of L2a, R₄ is methyl.

In certain embodiments of L2a, R₄ is a methyl, such that the methyl is oriented relative to the piperazine to which it is attached as follows:

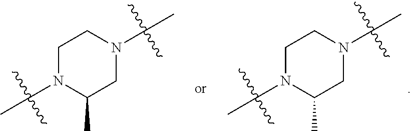

In certain embodiments of L2c, z is zero.

In certain embodiments of L2c, z is one.

Referring now to an Ab-CIDE, an Ab-CIDE can comprise a single antibody where the single antibody can have more than one CIDE, each CIDE covalently linked to the antibody through a linker L1. The “CIDE loading” is the average number of CIDE moieties per antibody. CIDE loading may range from 1 to 20 CIDE (D) per antibody (Ab). That is, in the Ab-CIDE formula, Ab-(L1-D)ₚ, p has a value from about 1 to about 20, from about 1 to about 8, from about 1 to about 5, from about 1 to about 4, or from about 1 to about 3. Each CIDE covalently linked to the antibody through linker L1 can be the same or different CIDE and can have a linker of the same type or different type as any other L1 covalently linked to the antibody. In certain embodiments, Ab is a cysteine engineered antibody and p is about 2.

The average number of CIDEs per antibody in preparations of Ab-CIDEs from conjugation reactions may be characterized by conventional means such as mass spectrometry, ELISA assay, electrophoresis, and HPLC. The quantitative distribution of Ab-CIDEs in terms of p may also be determined. By ELISA, the averaged value of p in a particular preparation of Ab-CIDE may be determined (Hamblett et al (2004) Clin. Cancer Res. 10:7063-7070; Sanderson et al (2005) Clin. Cancer Res. 11:843-852). However, the distribution of the value of p is not discernible by the antibody-antigen binding and detection limitation of ELISA. Also, ELISA assay for detection of Ab-CIDEs does not determine where the CIDE moieties are attached to the antibody, such as the heavy chain or light chain fragments, or the particular amino acid residues. In some instances, separation, purification, and characterization of homogeneous Ab-CIDEs where p is a certain value from Ab-CIDEs with other CIDE loadings may be achieved by means such as reverse phase HPLC or electrophoresis.

For some Ab-CIDEs, p may be limited by the number of attachment sites on the antibody. For example, an antibody may have only one or several cysteine thiol groups, or may have only one or several sufficiently reactive thiol groups through which a linker may be attached. Another reactive site on an Ab to connect L1-Ds are the amine functional group of lysine residues. Values of p include values from about 1 to about 20, from about 1 to about 8, from about 1 to about 5, from about 1 about 4, from about 1 to about 3, and where p is equal to 2. In some embodiments, the subject matter described herein is directed to any the Ab-CIDEs, wherein p is about 1, 2, 3, 4, 5, 6, 7, or 8.

Generally, fewer than the theoretical maximum of CIDE moieties is conjugated to an antibody during a conjugation reaction. An antibody may contain, for example, many lysine residues that do not react with the linker L1-CIDE group (L1-D) or linker reagent. Only the most reactive lysine groups may react with an amine-reactive linker reagent. Also, only the most reactive cysteine thiol groups may react with a thiol-reactive linker reagent or linker L1-CIDE group. Generally, antibodies do not contain many, if any, free and reactive cysteine thiol groups which may be linked to a CIDE moiety. Most cysteine thiol residues in the antibodies of the compounds exist as disulfide bridges and must be reduced with a reducing agent such as dithiothreitol (DTT) or TCEP, under partial or total reducing conditions. However, the CIDE loading (CIDE/antibody ratio, “CAR”) of a CAR may be controlled in several different manners, including: (i) limiting the molar excess of linker L1-CIDE group or linker reagent relative to antibody, (ii) limiting the conjugation reaction time or temperature, and (iii) partial or limiting reductive conditions for cysteine thiol modification.

### III. L1-CIDE Compounds

The CIDEs described herein can be covalently linked to a linker L1 to prepare L1-CIDE groups. These compounds have the following general formula:

(L1-D),
wherein, D is a CIDE having the structure E3LB-L2-PB; wherein, E3LB is an E3 ligase binding group covalently bound to L2; L2 is a linker covalently bound to E3LB and PB; PB is a BRM protein binding group covalently bound to L2; and L1 is a linker, covalently bound to D. Useful groups for each of these components is as described above.

In particular embodiments, L1 is as described elsewhere herein, including a peptidomimetic linker. In these embodiments, the L1-CIDE has the following formula:

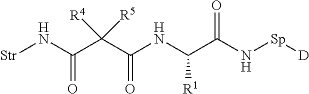

wherein
Str is a stretcher unit;
Sp is a bond or a spacer unit covalently attached to D, i.e., a CIDE moiety;
R¹ is C₁-C₁₀alkyl, (C₁-C₁₀alkyl)NHC(NH)NH₂ or (C₁-C₁₀alkyl)NHC(O)NH₂;
R⁴ and R⁵ are each independently C₁-C₁₀alkyl, arylalkyl, heteroarylalkyl, (C₁-C₁₀alkyl)OCH₂—, or R⁴ and R⁵ may form a C₃-C₇cycloalkyl ring;
D is a CIDE moiety.

An L1-CIDE compound can be represented by the following formula:

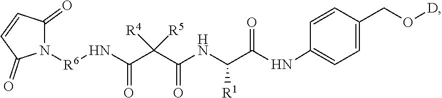

wherein R₆ is C₁-C₁₀alkylene; R⁴ and R⁵ together form a C₃-C₇cycloalkyl ring, and D is a CIDE moeity.

An L1-CIDE compound can be represented by the following formula:

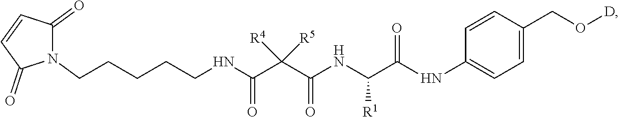

wherein R¹, R⁴ and R⁵ are as described elsewhere herein, and D is a CIDE moiety.

An L1-CIDE compound can be represented by the following formula:

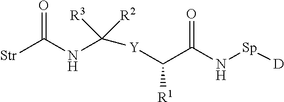

wherein
Str is a stretcher unit;
Sp is an optional spacer unit covalently attached to D, i.e., a CIDE moiety;
Y is heteroaryl, aryl, —C(O)C₁-C₆alkylene, C₁-C₆alkylene-NH₂, C₁-C₆alkylene-NH—CH₃, C₁-C₆alkylene-N—(CH₃)₂, C₁-C₆alkenyl or C₁-C₆alkylenyl;
R¹ is C₁-C₁₀alkyl, (C₁-C₁₀alkyl)NHC(NH)NH₂ or (C₁-C₁₀alkyl)NHC(O)NH₂;
R³ and R² are each independently H, C₁-C₁₀alkyl, arylalkyl or heteroarylalkyl, or R³ and R² together may form a C₃-C₇cycloalkyl; and
D is a CIDE moiety.

An L1-CIDE compound can be represented by the following formula:

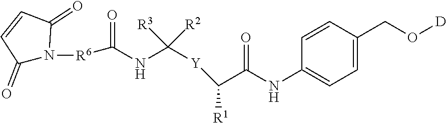

wherein, R⁶ is C₁-C₁₀alkylene, and R¹, R² and R³ are as described elsewhere herein, and D is a CIDE moiety

An L1-CIDE compound can be represented by the following formula:

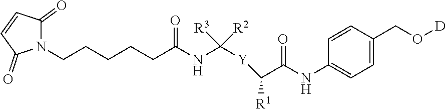

wherein R¹, R² and R³ are as described elsewhere herein, and D is a CIDE moiety.

In any of the above L1-CIDE compounds, Str can have the following formula:

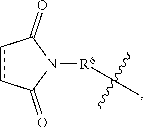

wherein R⁶ is selected from the group consisting of C₁-C₁₀alkylene, C₃-C₅cycloalkyl, O—(C₁-C₈alkylene), and C₁-C₁₀alkylene-C(O)N(R^{a})—C₂-C₆alkylene, where each alkylene may be substituted by one to five substituents selected from the group consisting of halo, trifluoromethyl, difluoromethyl, amino, alkylamino, cyano, sulfonyl, sulfonamide, sulfoxide, hydroxy, alkoxy, ester, carboxylic acid, alkylthio, C₃-C₅cycloalkyl, C₄-C₇heterocycloalkyl aryl, arylalkyl, heteroarylalkyl and heteroaryl; each R^{a} is independently H or C₁-C₆alkyl; Sp is —Ar—R^{b}—, wherein Ar is aryl or heteroaryl, Rb is (C₁-C₁₀alkylene)O—.

In certain L1-CIDE compounds, R⁶ is C₁-C₁₀alkylene, Sp is —Ar—R^{b}—, wherein Ar is aryl R^{b} is (C₁-C₆alkylene)O—; or R₆ is —(CH₂)_{q} is 1-10;

In any of the above L1-CIDE compounds, Str can have the following formula:

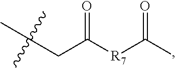

wherein, [symbol] indicates a moiety capable of conjugating to an antibody, R⁷ is selected from C₁-C₁₀alkylene, C₁-C₁₀alkylene-O, N(R^{c})—(C₂-C₆ alkylene)-N(R^{c}) and N(R^{c})—(C₂-C₆alkylene); where each R^{c} is independently H or C₁-C₆ alkyl;
Sp is —Ar—R^{b}—, wherein Ar is aryl or heteroaryl, R^{b} is (C₁-C₁₀ alkylene)O—; or wherein R⁶ is C₁-C₁₀ alkylene, Sp is —Ar—R^{b}—, wherein Ar is aryl R^{b} is (C₁-C₆ alkylene)O—.

An L1-CIDE can have the following formulae, wherein in each instance, D is a CIDE moiety:

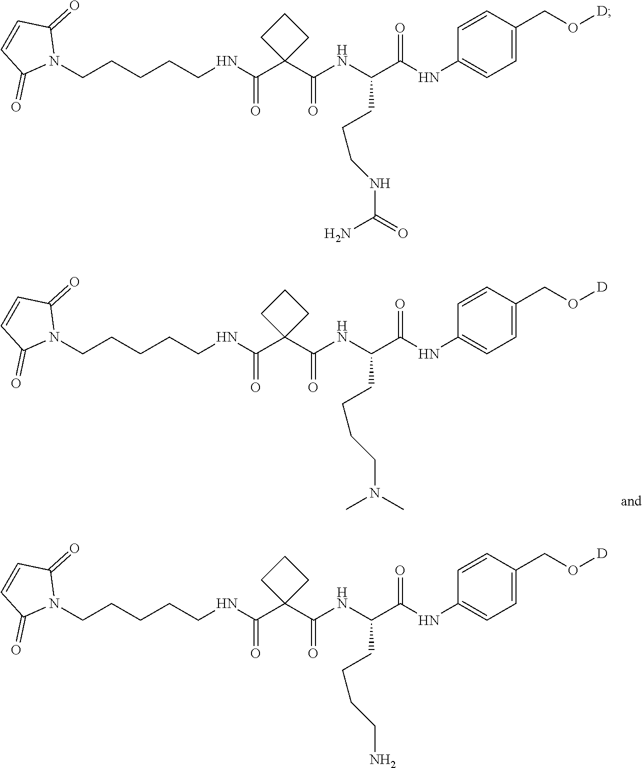

Referring now to the PB group of the CIDE, in particular embodiments, PB is as described elsewhere herein. Referring now to the E3LB group of the CIDE, E3LB is as described elsewhere herein. Ab-CIDEs can include any combination of PB, E3LB, Ab, L1 and L2.

In view of the subject matter disclosed herein, those of skill in the art would understand that the L1 and L2 points of attachment can vary. Further, portions of the linkers, such as -Str-(PM)-Sp- can be interchanged. Additionally, portions of linkers L1 can be interchanged. Non-limiting examples of L1 linker attachments to the CIDE, to the antibody and to other linkers that can be interchanged include, but are not limited to, those depicted in Table 1-L1.

| CIDE Portion to | CIDE Attachment | | Antibody Attachment |
|---|---|---|---|
| which L1 Attached | Portion of L1 | Linker Portion of L1 | Portion of L1 |
| 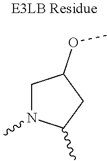 | NA | 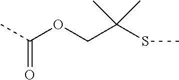 | NA |
| 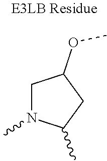 | NA | 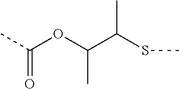 | NA |
| 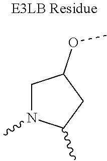 | NA | 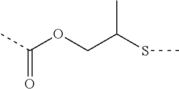 | NA |
| 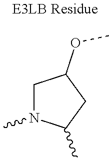 | NA | 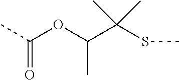 | NA |
| 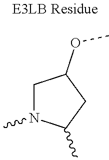 | 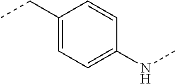 | 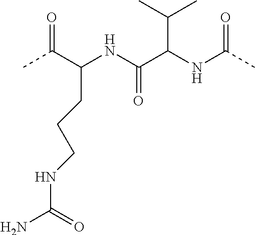 | 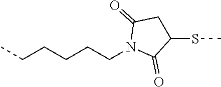 |
| 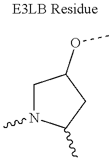 | 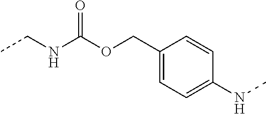 | 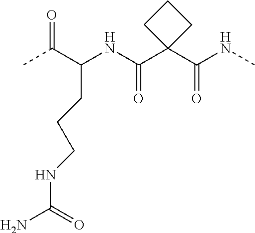 | 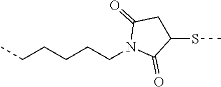 |
| 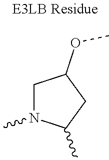 | 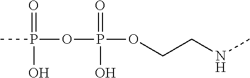 | 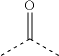 | 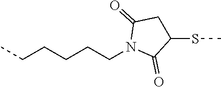 |
| 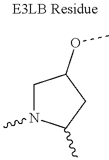 | 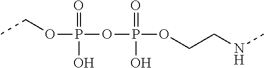 | 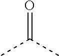 | 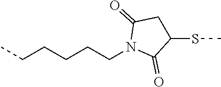 |
| 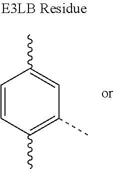 | 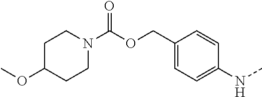 | 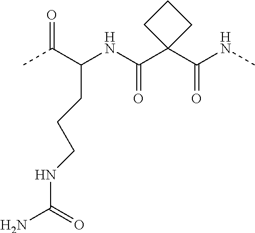 | 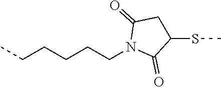 |
| 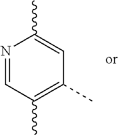 | | | |
| 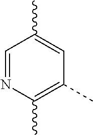 | | | |
| 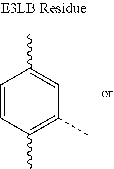 | 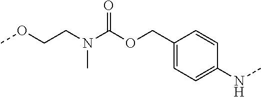 | 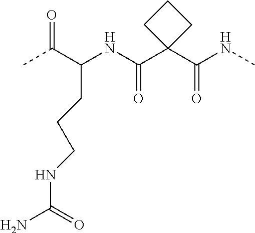 | 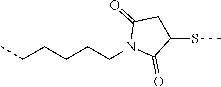 |
| 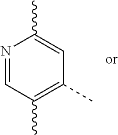 | | | |
| 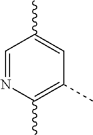 | | | |
| 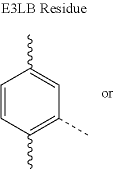 | 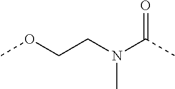 | NA | 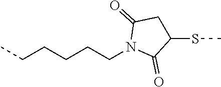 |
| 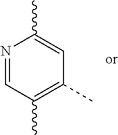 | | | |
| 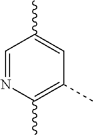 | | | |
| 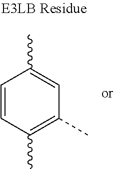 | 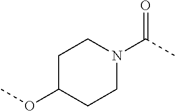 | NA | 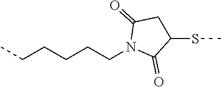 |
| 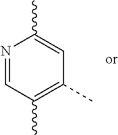 | | | |
| 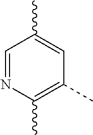 | | | |
| 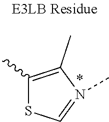 | 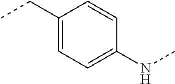 | 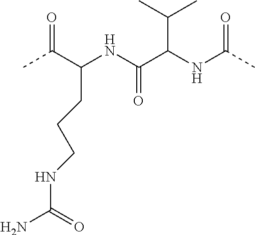 | 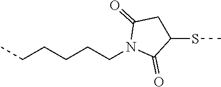 |
| L2 to Protein | | | |
| Binding group-any | | | |
| available position | | | |

In certain embodiments, the linker L1 can be covalently linked to the E3LB residue in different positions, L1-T, L1-U, L1-V and L1-Y (from the R₃ group):

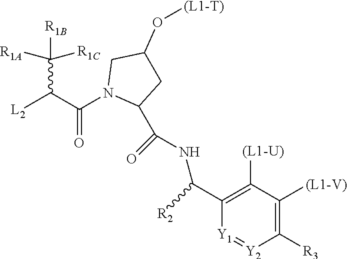

R₃ is selected from the group consisting of cyano,

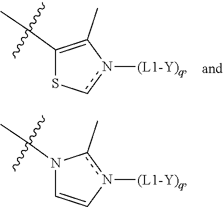

wherein [symbol] is a single or double bond; [symbol]
Ab is an antibody covalently bound to at least one L1 that is a linker;
L1-T, L1-U, and L1-V are each independently hydrogen or a L1 linker covalently bound to Ab and D;
L1-Y is hydrogen or a L1 linker covalently bound to Ab and D; and
q is 1 or zero.

The Linker-L1 can be attached to any position of an antibody so long as the covalent bond between Linker L1 and the antibody is a disulphide bond.

In embodiments, an antibody, Ab, is conjugated to one to eight Chemical Inducers of Degradation (CIDEs), D, each via a linker, L1.

Ab-(L1-D)ₚ, wherein p is 1 to 8
D comprises an E3 ligase binding (E3LB) ligand linked to a target protein binding (PB) ligand via a linker, L2 as follows:

E3LB-L2-PB

In embodiments, L1 forms a disulfide bond with the sulfur of an engineered Cys residue of the antibody to link the CIDE to the Ab.

In embodiments, the antibody is linked via L1 to the E3LB ligand of the CIDE.

In embodiments, L1 is linked to an E3LB ligand residue of the E3LB ligand of the CIDE.

For example, in embodiments, L1 is covalently bound to a portion of BRM at attachment point (L1-Q) as illustrated below:

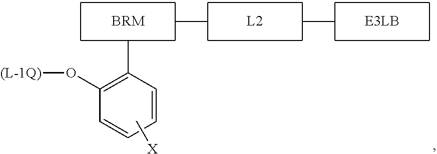

wherein X is hydrogen or halogen, and L1 is selected from L1b and L1c.

In embodiments, L1 is covalently bound to a portion of BRM at attachment point (L1-Q′) as illustrated below:

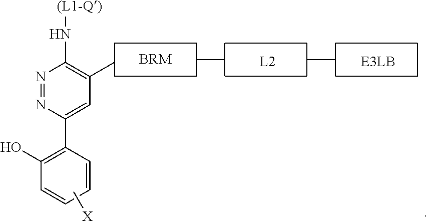

wherein X is hydrogen or halogen, and L1 is selected from L1b and L1c.

In embodiments, L1 is covalently bound to a portion of E3LB at attachment point (L1-Q′) as illustrated below:

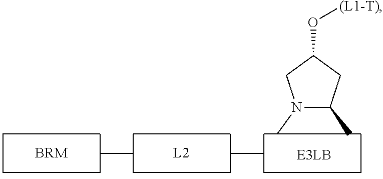

wherein L1 is selected from L1a, L1b and L1c.

Referring now to an Ab-CIDE and a L1-CIDE compound, as described herein, these can exist in solid or liquid form. In the solid state, it may exist in crystalline or noncrystalline form, or as a mixture thereof. The skilled artisan will appreciate that pharmaceutically acceptable solvates may be formed for crystalline or non-crystalline compounds. In crystalline solvates, solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The subject matter described herein includes all such solvates.

The skilled artisan will further appreciate that certain compounds and Ab-CIDEs described herein that exist in crystalline form, including the various solvates thereof, may exhibit polymorphism (i.e. the capacity to occur in different crystalline structures). These different crystalline forms are typically known as “polymorphs.” The subject matter disclosed herein includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

Compounds and Ab-CIDEs described herein or a salt thereof may exist in stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt of Formula (I) may exist in tautomeric forms other than that shown in the formula and these are also included within the scope of the subject matter disclosed herein. It is to be understood that the subject matter disclosed herein includes all combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes all combinations and subsets of the particular groups defined hereinabove.

The subject matter disclosed herein also includes isotopically-labelled forms of the compounds described herein, but for the fact that one or more atoms are replaced by an atom having an atomic mass or mass number different from the atomic mass or mass number usually found in nature. Examples of isotopes that can be incorporated into compounds described herein and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds and Ab-CIDEs as disclosed herein and pharmaceutically acceptable salts thereof that contain the aforementioned isotopes and/or other isotopes of other atoms are within the scope of the subject matter disclosed herein. Isotopically-labelled compounds are disclosed herein, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are commonly used for their ease of preparation and detectability. ¹¹C and ¹⁸F isotopes are useful in PET (positron emission tomography), and ¹²⁵I isotopes are useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds can generally be prepared by carrying out the procedures disclosed in the Schemes and/or in the Examples below, by substituting a readily available isotopically labelled reagent for a non-isotopically labelled reagent.

In embodiments, D is
or

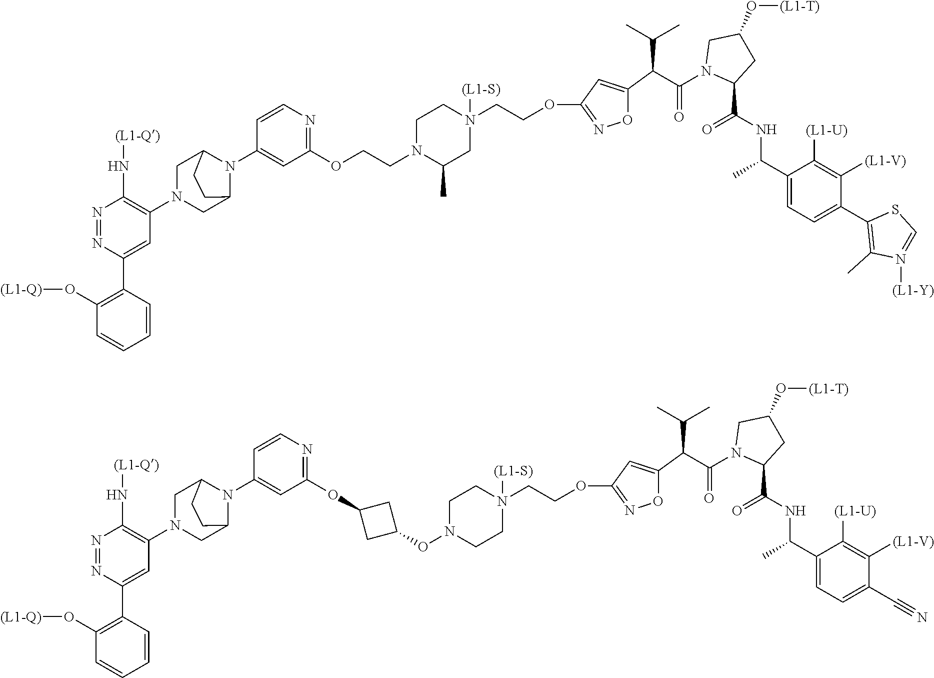

wherein L1 is covalently linked to D at one attachment point selected from the group consisting of L1-Q, L1-Q′, L1-S, L1-T, L1-U, L1-V and L1-Y. It should be understood that each of L1-Q, L1-Q′, L1-S, L1-T, L1-U, L1-V and L1-Y that is not an attachment point for L1 retains its original valence. For example, if L1 is attached at L1-Q′, it is not attached at L1-Q, L1-S, L1-T, L1-U, L1-V or L1-Y, and D has the structure:

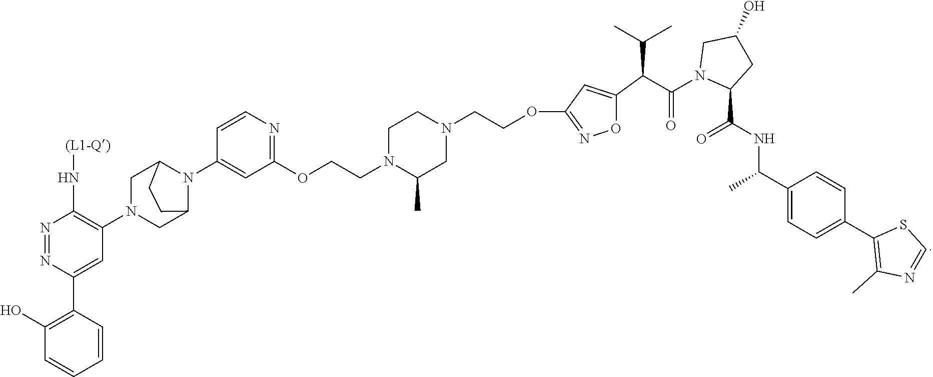

In embodiments, L1 is L1a having the structure

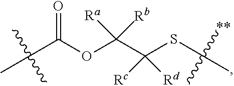

wherein
R^{a}, R^{b}, R^{c}, and R^{d} are each independently selected from the group consisting of H, optionally substituted branched or linear C₁-C₅ alkyl, and optionally substituted C₃-C₆ cycloalkyl, or R^{a} and R^{b} taken together or R^{c} and R^{d} taken together with the carbon atom to which they are bound form an optionally substituted C₃-C₆ cycloalkyl ring or a 3 to 6-membered heterocycloalkyl ring, and wherein

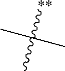

is the point of attachment to Ab.

In embodiments, L1a is attached at L1-T, and at least one of R^{a}, R^{b}, R^{c}, and R^{d} is methyl.

In embodiments, L1a is attached at L1-T, and at least two of R^{a}, R^{b}, R^{c}, and R^{d} are methyl.

In embodiments, L1a is attached at L1-T, and R^{a} and R^{c} are each methyl, and Rh and R^{d} are each hydrogen.

In embodiments, L1a is attached at L1-T, and R^{a}, R^{c} and R^{d} are each methyl, and R^{b} is hydrogen.

In embodiments, L1a is attached at L1-T, and R^{a} and R^{b} are each hydrogen and R^{c} and R^{d} combine together with the carbon atom to which they are bound to form an optionally substituted 3 to 6-membered heterocycloalkyl ring. In embodiments, the 3 to 6-membered heterocycloalkyl ring is an optionally substituted piperidine ring. In embodiments, the piperidine ring is substituted with a methyl.

In embodiments, L1a is attached at L1-T, wherein at least two of R^{a}, R^{b}, R^{c}, and R^{d} are methyl; and a phosphate moiety is attached at L1-Q, wherein the phosphate moiety has the structure

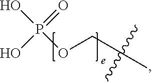

wherein e is 1.

In embodiments, L1 is Lib having the structure

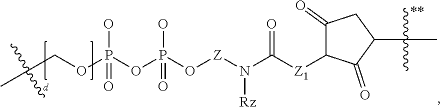

wherein, Z and Z₁ are each independently a C₁₋₁₂ alkylene or —[CH₂]_{g}—[—O—CH₂]ₕ—, wherein g is 0, 1 or 2, and h is 1-5; R_{z} is H or C₁₋₃alkyl; d is 0, 1 or 2; and wherein

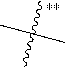

is the point of attachment to Ab.

In embodiments, Z and Z₁ are each independently a C₁₋₁₂ alkylene, R_{z} is hydrogen, and d is 0 or 1.

In embodiments, Z is C₂ alkylene, and Z₁ is C₅ alkylene, R_{z} is hydrogen, and d is 0 or 1.

In embodiments, L1b is attached at L1-Q, and d is 1.

In embodiments, L1b is attached at L1-T, and d is 0.

In embodiments, L1 is L1c having the structure

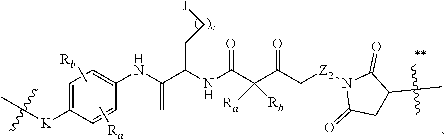

wherein
Z₂ is a C₁₋₁₂ alkylene or —[CH₂]g-[—O—CH₂]ₕ—, wherein g is 0, 1 or 2, and h is 1-5;
w is 0, 1, 2, 3, 4 or 5, and wherein

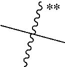

is the point of attachment to Ab;
J is hydrogen, —N(Rₓ)(R_{y}), —C(O)NH₂, —NH—C(O)—NH₂, —NH—C(═NH)—NH₂, wherein, Rₓ and R_{y} are each independently selected from hydrogen and C₁₋₃alkyl, wherein Rₓ and R_{y} are each independently selected from hydrogen and C₁₋₃alkyl;
K is selected from —CH₂—, —CH(R)—, —CH(R)—O-\hat{ }, —C(O)—, \hat{ }—C(O)—O—CH(R)—, —CH₂—O—C(O)-\hat{ }, —CH₂—O—C(O)—NH-\hat{ }, \hat{ }—O—C(L1c)-C(O)—NRₓR_{y}—, \hat{ }—C(L1c)-C(O)—NRₓR_{y}—, CH₂—O—C(O)—NH—CH₂—, —CH₂—O—C(O)—R—[CH₂]_{q}—O-\hat{ }, —CH₂—O—C(O)—R—[CH₂]_{q}-\hat{ }, wherein \hat{ } indicates the attachment to CIDE, wherein R is hydrogen, C₁₋₃alkyl, N(Rₓ)(R_{y}), —O—N(Rₓ)(R_{y}) or C(O)—N(Rₓ)(R_{y}), wherein q is 0, 1, 2, or 3, and Rₓ and R_{y} are each independently selected from hydrogen and C₁₋₃alkyl, or Rₓ and R_{y} together with the nitrogen to which each is attached form an optionally substituted 5- to 7-member heterocyclyl;
Ra and Rb are each independently selected from hydrogen and C₁₋₃alkyl, or Ra and Rb together with the carbon to which each is attached form an optionally substituted C₃₋₆cycloalkyl; and
R₇ and R₈ are each independently hydrogen, halo, C₁₋₅ alkyl, C₁₋₅ alkoxy or hydroxyl.

In embodiments, Z₂ is a C₁₋₁₂ alkylene, w is 2, J is —NH—C(O)—NH₂, Ra and Rb together with the carbon to which each is attached form an optionally substituted C₃₋₆cycloalkyl, and R₇ and R₈ are each independently hydrogen.

In embodiments, Z₂ is a C₅ alkylene, w is 2, J is —NH—C(O)—NH₂, Ra and Rb together with the carbon to which each is attached form an optionally substituted C₄ cycloalkyl, and R₇ and R₈ are each independently hydrogen.

In embodiments, Z₂ is a C₁₋₁₂ alkylene, w is 2, J is —NH—C(O)—NH₂, K is —CH₂—O—C(O)—, Ra and Rb together with the carbon to which each is attached form an optionally substituted C₃₋₆cycloalkyl, and R₇ and R₈ are each independently hydrogen.

In embodiments, Z₂ is a C₅ alkylene, w is 2, J is —NH—C(O)—NH₂, K is —CH₂—O—C(O)—, Ra and Rb together with the carbon to which each is attached form an optionally substituted C₄ cycloalkyl, and R₇ and R₈ are each independently hydrogen.

In embodiments, Z₂ is a C₁₋₁₂ alkylene, w is 3, J is —N(Rₓ)(R_{y}) wherein Rₓ and R_{y} are each independently selected from hydrogen and C₁₋₃alkyl, Ra and Rb together with the carbon to which each is attached form an optionally substituted C₃₋₆cycloalkyl, and R₇ and R₈ are each independently hydrogen.

In embodiments, Z₂ is a C₅ alkylene, w is 3, J is —N(Rₓ)(R_{y}) wherein Rₓ and R_{y} are each methyl, Ra and Rb together with the carbon to which each is attached form C₄ cycloalkyl, and R₇ and R₈ are each independently hydrogen.

In embodiments, Z₂ is a C₁₋₁₂ alkylene, w is 3, J is —N(Rₓ)(R_{y}) wherein Rₓ and R_{y} are each independently selected from hydrogen and C₁₋₃alkyl, K is —CH₂—, Ra and Rb together with the carbon to which each is attached form an optionally substituted C₃₋₆cycloalkyl, and R₇ and R₈ are each independently hydrogen.

In embodiments, Z₂ is a C₅ alkylene, w is 3, J is —N(Rₓ)(R_{y}) wherein Rₓ and R_{y} are each methyl, K is —CH₂—, Ra and Rb together with the carbon to which each is attached form C₄ cycloalkyl, and R₇ and R₈ are each independently hydrogen.

In embodiments, Z₂ is a C₁₋₁₂ alkylene, w is 0, J is hydrogen, Ra and Rb together with the carbon to which each is attached form an optionally substituted C₃₋₆cycloalkyl, and R₇ and R₈ are each independently hydrogen.

In embodiments, Z₂ is a C₅ alkylene, w is 0, J is hydrogen, Ra and Rb together with the carbon to which each is attached form C₄ cycloalkyl, and R₇ and R₈ are each independently hydrogen.

In embodiments, Z₂ is a C₁₋₁₂ alkylene, w is 0, J is hydrogen, K is —CH₂—, Ra and Rb together with the carbon to which each is attached form an optionally substituted C₃₋₆cycloalkyl, and R₇ and R₈ are each independently hydrogen.

In embodiments, Z₂ is a C₅ alkylene, w is 0, J is hydrogen, K is —CH₂—, Ra and Rb together with the carbon to which each is attached form C₄ cycloalkyl, and R₇ and R₈ are each independently hydrogen.

In embodiments, Z₂ is a C₁₋₁₂ alkylene, w is 2, J is —NH—C(O)—NH₂, Ra and Rb together with the carbon to which each is attached form an optionally substituted C₃₋₆cycloalkyl, and R₇ and R₈ are each independently hydrogen.

In embodiments, Z₂ is a C₅ alkylene, w is 2, J is —NH—C(O)—NH₂, Ra and Rb together with the carbon to which each is attached form an optionally substituted C₄ cycloalkyl, and R₇ and R₈ are each independently hydrogen.

In embodiments, Z₂ is a C₁₋₁₂ alkylene, w is 2, J is —NH—C(O)—NH₂, K is —CH(R)—O—C(O)—, wherein R is C(O)—N(Rₓ)(R_{y}), wherein Rₓ and R_{y} together with the nitrogen to which each is attached form an optionally substituted 5- to 7-member heterocyclyl, Ra and Rb together with the carbon to which each is attached form an optionally substituted C₃₋₆cycloalkyl, and R₇ and R₈ are each independently hydrogen.

In embodiments, Z₂ is a C₅ alkylene, w is 2, J is —NH—C(O)—NH₂, K is —CH(R)—O—C(O)—, wherein R is C(O)—N(Rₓ)(R_{y}), wherein Rₓ and R_{y} together with the nitrogen to which each is attached form an optionally substituted piperazine, Ra and Rb together with the carbon to which each is attached form an optionally substituted C₄ cycloalkyl, and R₇ and R₈ are each independently hydrogen.

In embodiments, Z₂ is a C₁₋₁₂ alkylene, w is 3, J is —N(Rₓ)(R_{y}) wherein Rₓ and R_{y} are each independently selected from hydrogen and C₁₋₃alkyl, K is —CH₂—O—C(O)—, Ra and Rb together with the carbon to which each is attached form an optionally substituted C₃₋₆cycloalkyl, and R₇ and R₈ are each independently hydrogen.

In embodiments, Z₂ is a C₅ alkylene, w is 3, J is —N(Rₓ)(R_{y}) wherein Rₓ and R_{y} are each methyl, K is —CH₂—O—C(O)—, Ra and Rb together with the carbon to which each is attached form C₄ cycloalkyl, and R₇ and R₈ are each independently hydrogen.

In embodiments, L1c is attached at L1-Q, and K is —CH₂—.

In embodiments, L1c is attached at L1-Q′, and K is —CH₂—O—C(O)—.

In embodiments, L1c is attached at L1-S, and K is —CH₂—.

In embodiments, L1c is attached at L1-T, and K is —CH(R)—O—C(O)—, wherein R is C(O)—N(Rₓ)(R_{y}), wherein Rₓ and R_{y} together with the nitrogen to which each is attached form an optionally substituted 5- to 7-member heterocyclyl.

In embodiments, L1c is attached at L1-U.

In embodiments, L1c is attached at L1-V.

In embodiments, L1c is attached at L1-Y, and K is —CH₂—.

In embodiments, L1c is attached at L1-Q, wherein K is —CH₂—; and a phosphate moiety is attached at L1-T, wherein the phosphate moiety has the structure

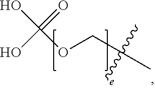

wherein e is 0.

In certain embodiments, the subject matter described herein includes the following L1-CIDEs.

| | |
|---|---|
| 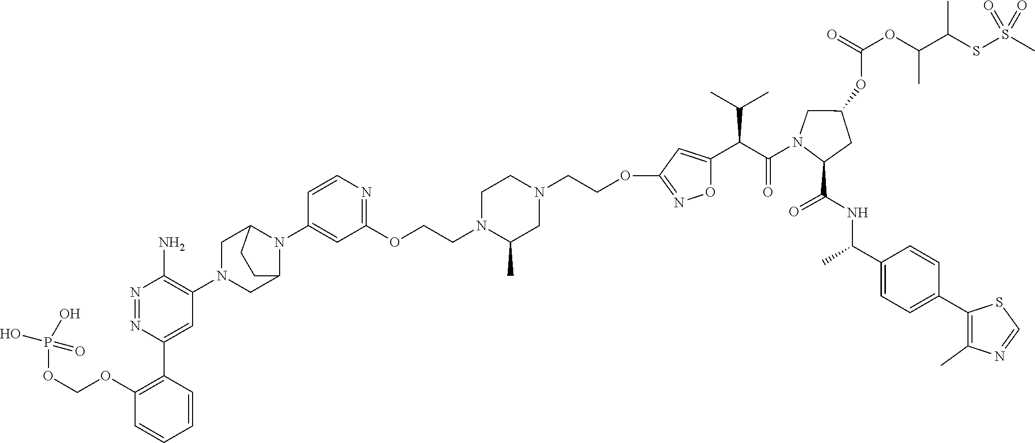 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1] | L1- CIDE- BRM1- 10 |
| 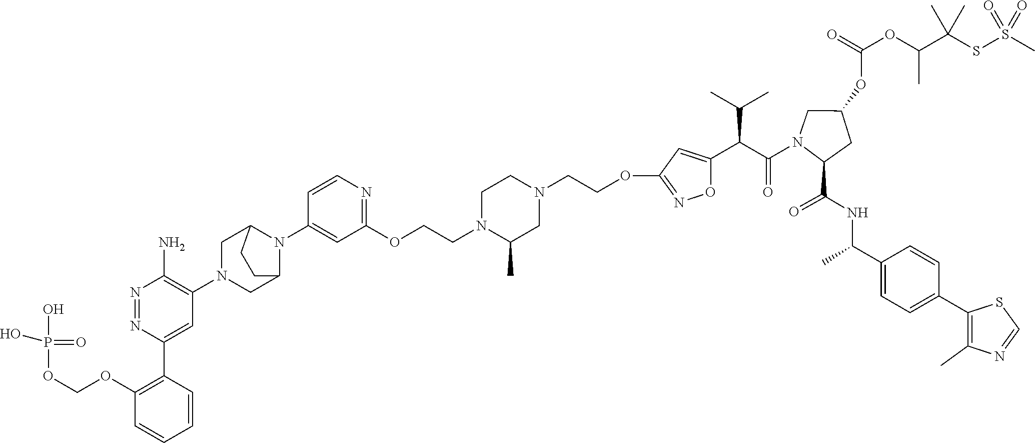 [SMILES: Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1] | L1- CIDE- BRM1- 9 |
| 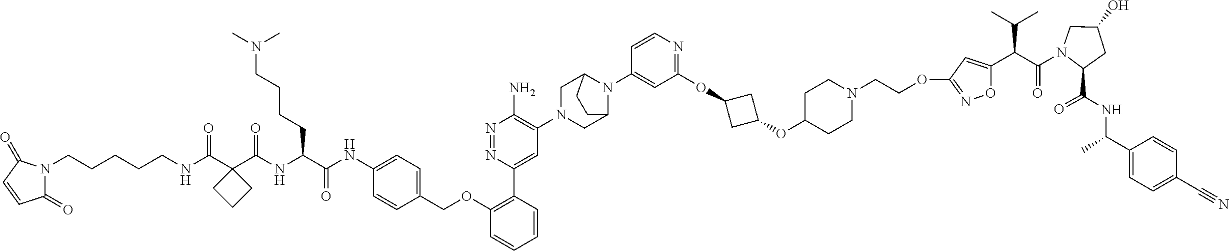 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)C3)CC2)no1] | L1- CIDE- BRM1- 19 |
| 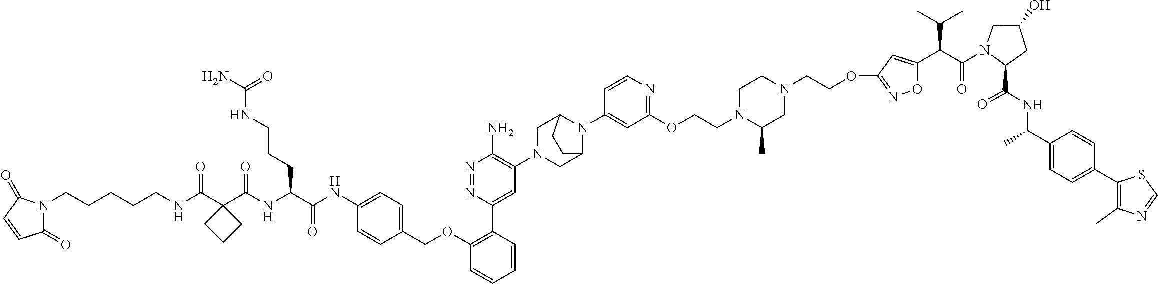 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1] | L1- CIDE- BRM1- 13 |
| 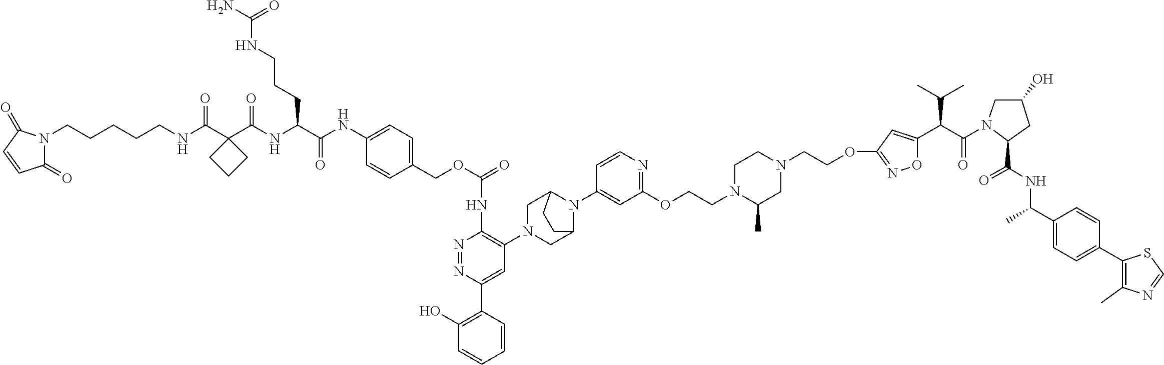 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCNC(N)=O)NC(=O)C7(C(=O)NCCCCCN8C(=O)C=CC8=O)CCC7)cc5)C6)ccn4)[C@H](C)C3)no2)C(C)C)cc1] | L1- CIDE- BRM1- 20 |
| 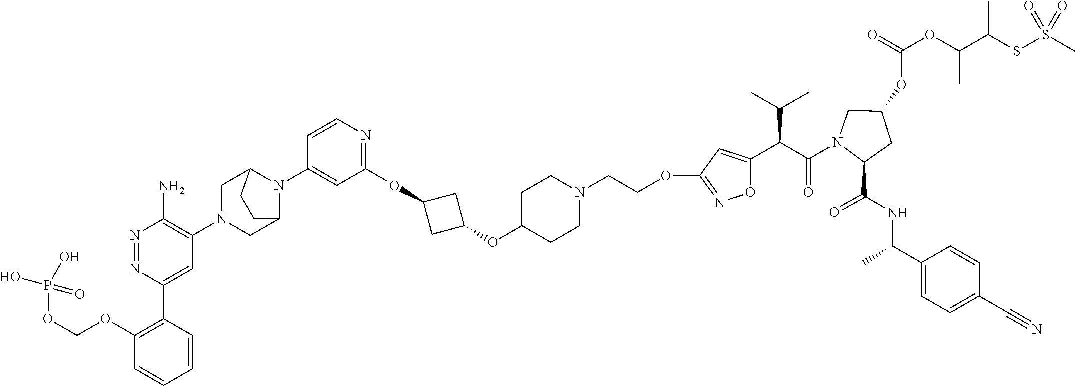 [SMILES: CC(OC(=O)O[C@@H]1C[C@@H](C(=O)N[C@@H](C)c2ccc(C#N)cc2)N(C(=O)[C@@H](c2cc(OCCN3CCC(O[C@H]4C[C@H](Oc5cc(N6C7CCC6CN(c6cc(-c8ccccc8OCOP(=O)(O)O)nnc6N)C7)ccn5)C4)CC3)no2)C(C)C)C1)C(C)SS(C)(=O)=O] | L1- CIDE- BRM1- 11 |
| 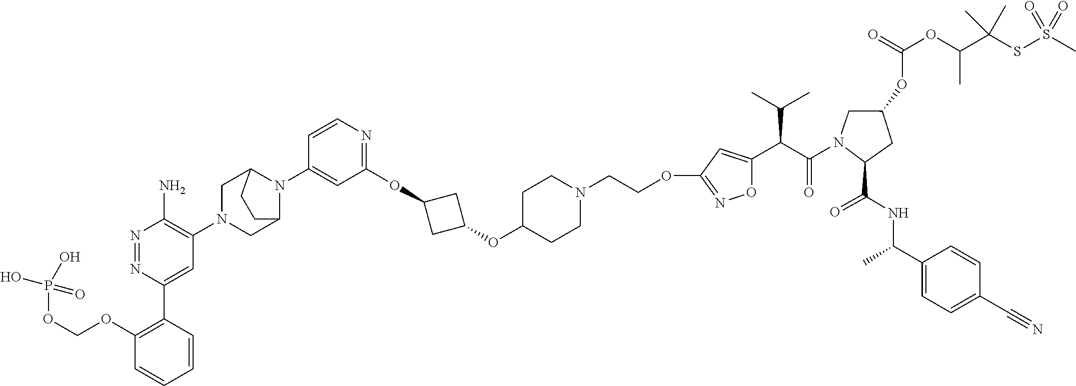 [SMILES: CC(C)[C@@H](C(=O)N1C[C@H](OC(=O)OC(C)C(C)(C)SS(C)(=O)=O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)O)nnc5N)C6)ccn4)C3)CC2)no1] | L1- CIDE- BRM1- 12 |
| 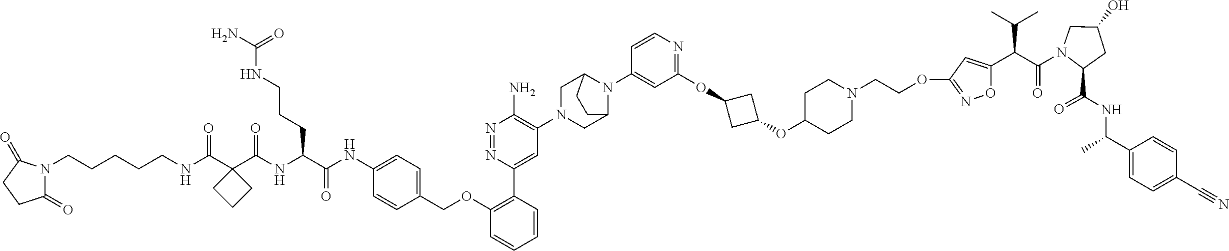 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCNC(N)=O)NC(=O)C8(C(=O)NCCCCCN9C(=O)CCC9=O)CCC8)cc7)nnc5N)C6)ccn4)C3)CC2)no1] | L1- CIDE- BRM1- 14 |
| 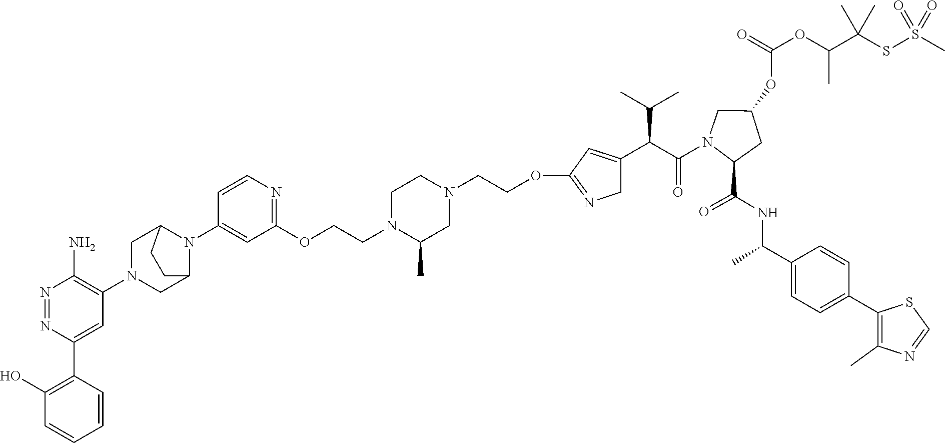 [SMILES: Cc1ncsc1-c1ccc([C@H](C)NC(=O)[C@@H]2C[C@@H](OC(=O)OC(C)C(C)(C)SS(C)(=O)=O)CN2C(=O)[C@@H](C2=CC(OCCN3CCN(CCOc4cc(N5C6CCC5CN(c5cc(-c7ccccc7O)nnc5N)C6)ccn4)[C@H](C)C3)=NC2)C(C)C)cc1] | L1- CIDE- BRM1- 7 |
| 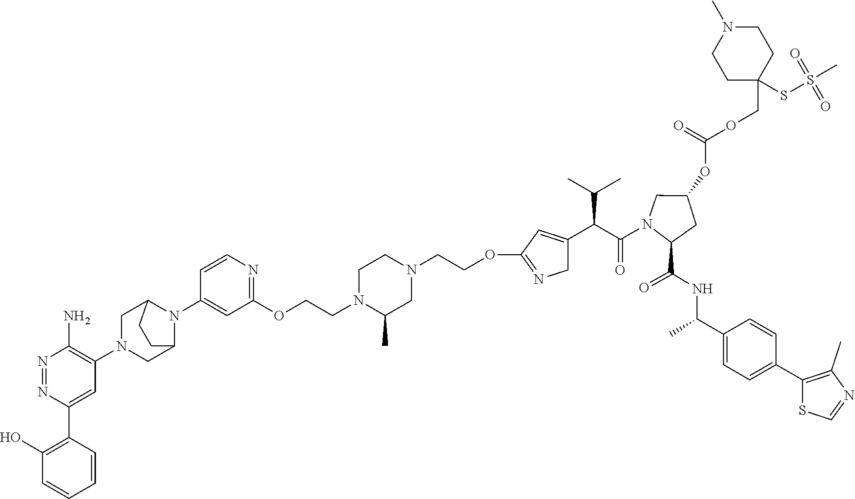 [SMILES: Cc1ncsc1-c1ccc([C@H](C)NC(=O)[C@@H]2C[C@@H](OC(=O)OCC3(SS(C)(=O)=O)CCN(C)CC3)CN2C(=O)[C@@H](C2=CC(OCCN3CCN(CCOc4cc(N5C6CCC5CN(c5cc(-c7ccccc7O)nnc5N)C6)ccn4)[C@H](C)C3)=NC2)C(C)C)cc1] | L1- CIDE- BRM1- 8 |
| 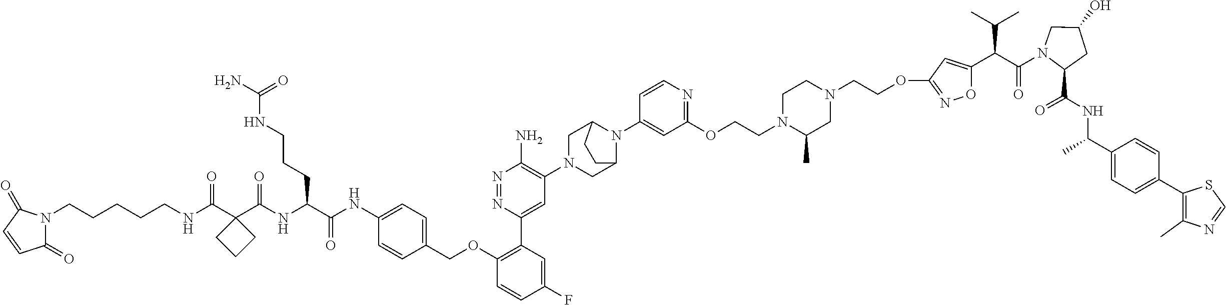 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7cc(F)ccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1] | L1- CIDE- BRM1- 16 |
| 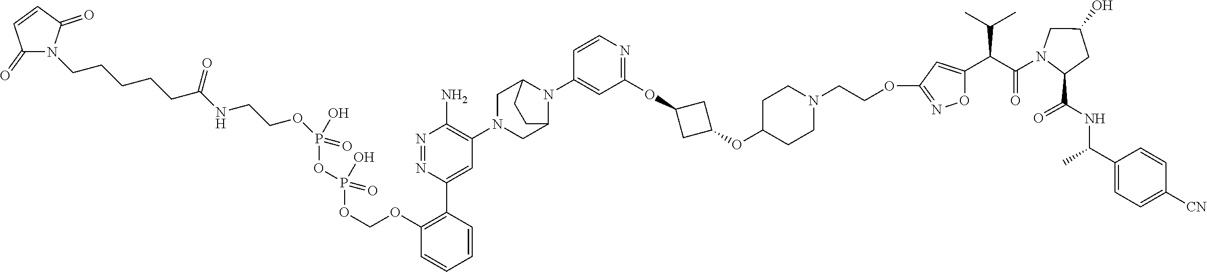 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)OP(=O)(O)OCCNC(=O)CCCCCN7C(=O)C=CC7=O)nnc5N)C6)ccn4)C3)CC2)no1] | L1- CIDE- BRM1- 17 |
| 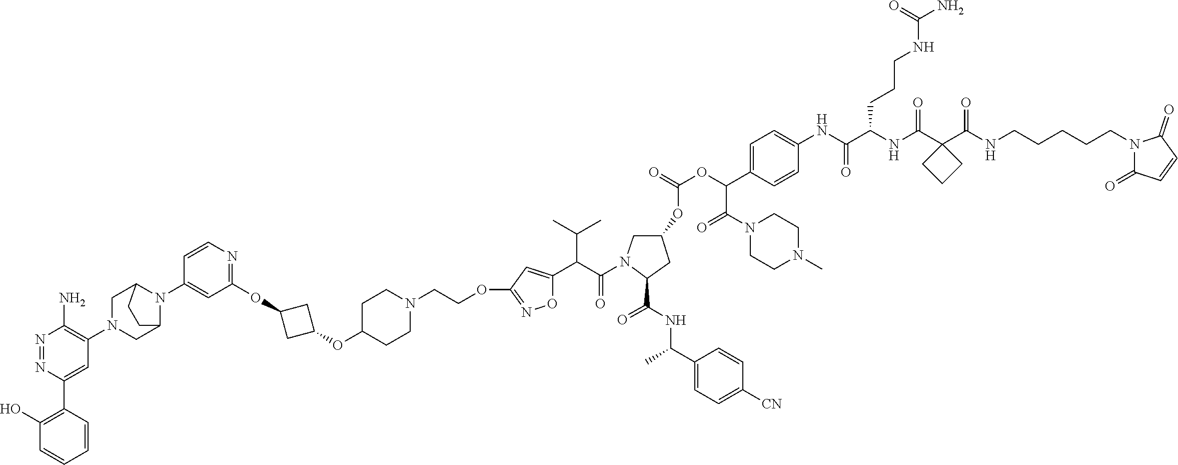 [SMILES: CC(C)C(C(=O)N1C[C@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7O)nnc5N)C6)ccn4)C3)CC2)no1] | L1- CIDE- BRM1- 18 |
| 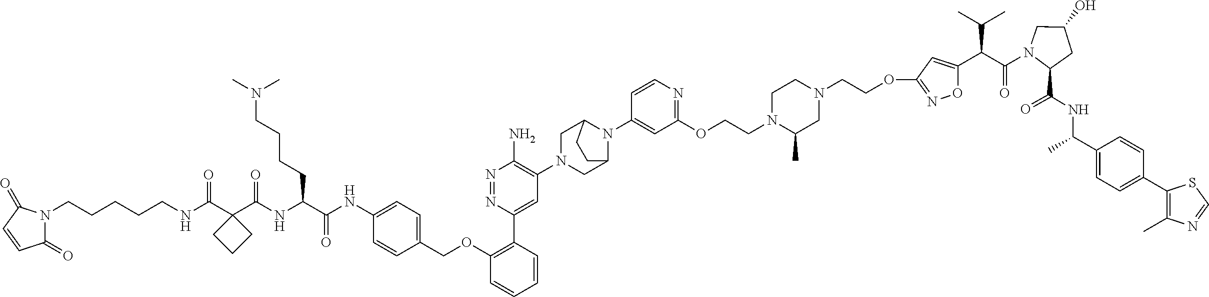 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)[C@H](C)C3)no2)C(C)C)cc1] | L1- CIDE- BRM1- 21 |
| 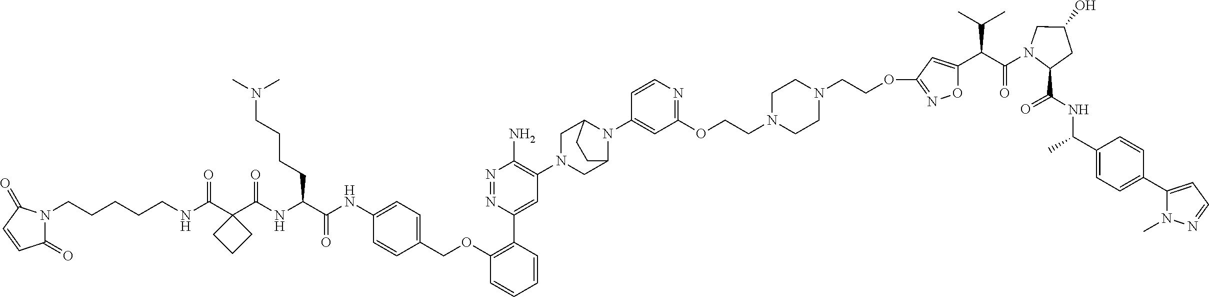 [SMILES: CC(C)[C@@H](C(=O)N1C[C@H](O)C[C@H]1C(=O)N[C@@H](C)c1ccc(-c2ccnn2C)cc1)c1cc(OCCN2CCN(CCOc3cc(N4C5CCC4CN(c4cc(-c6ccccc6OCc6ccc(NC(=O)[C@H](CCCCN(C)C)NC(=O)C7(C(=O)NCCCCCN8C(=O)C=CC8=O)CCC7)cc6)nnc4N)C5)ccn3)CC2)no1] | L1- CIDE- BRM1- 22 |

The subject matter disclosed herein include the following non-limiting embodiments:

1. A conjugate having the chemical structure

Ab-(L1-D)ₚ,
wherein,
   D isₐ CIDE having the structure E3LB-L2-PB;
   E3LB is covalently bound to L2, said E3LB having the formula:

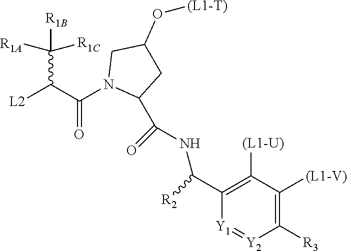

wherein,
   R_{1A}, R_{1B} and R_{1C} are each independently hydrogen, or C₁₋₅ alkyl; or two of R_{1A}, R_{1B} and R_{1C} together with the carbon to which each is attached form a C₁₋₅ cycloalkyl;
   R₂ is a C₁₋₅ alkyl;
   R₃ is selected from the group consisting of cyano,

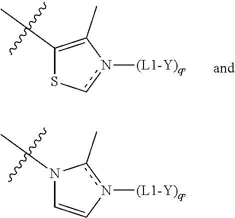

wherein, [symbol] is a single or double bond;
one of Y₁ and Y₂ is —CH, the other of Y₁ and Y₂ is —CH or N;
L2 is a linker covalently bound to E3LB and PB, said L2 having the formula:

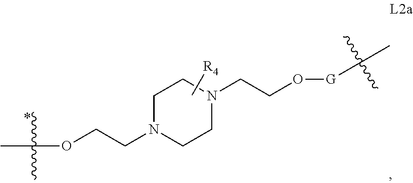

wherein,
   R₄ is hydrogen or methyl,

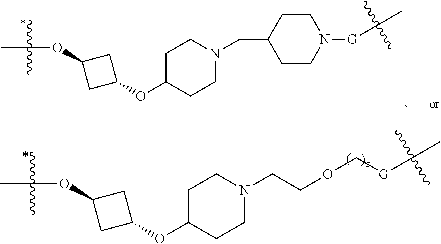

wherein,
   z is one or zero,
   G is

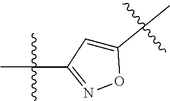 [SMILES: *c1cc(*)on1]

or —C(O)NH—; and,

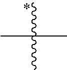

is the point of attachment to PB;
PB is a protein binding group covalently bound to L2, having the structure:

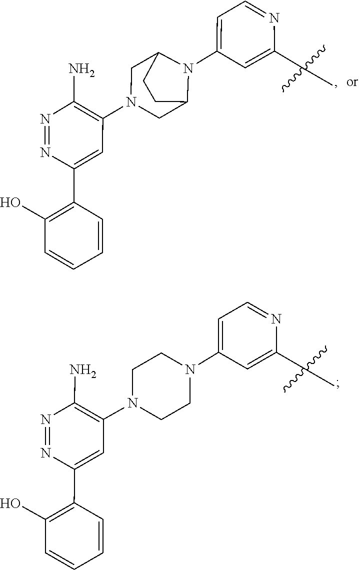 [SMILES: *c1cc(N2C3CCC2CN(c2cc(-c4ccccc4O)nnc2N)C3)ccn1 ; *c1cc(N2CCN(c3cc(-c4ccccc4O)nnc3N)CC2)ccn1]

Ab is an antibody covalently bound to at least one L1 that is a linker;
L1-T, L1-U, and L1-V are each independently hydrogen or a L1 linker covalently bound to Ab and D;
L1-Y is hydrogen or a L1 linker covalently bound to Ab and D;
q is 1 or zero;
and,
   p has a value from about 1 to about 8.

2. The conjugate of embodiment 1, wherein R₃ is cyano.

3. The conjugate of embodiment 1, wherein R₃ is

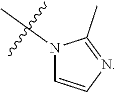 [SMILES: *n1ccnc1C]

4. The conjugate of embodiment 1, wherein R₃ is

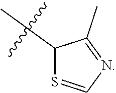

5. The conjugate of embodiment 1, wherein R_{1A}, R_{iB} and R_{1C} are each independently hydrogen or methyl.

6. The conjugate of embodiment 5, wherein R_{1A} and R_{iB} are each methyl.

7. The conjugate of embodiment 6, wherein E3LB has the formula:

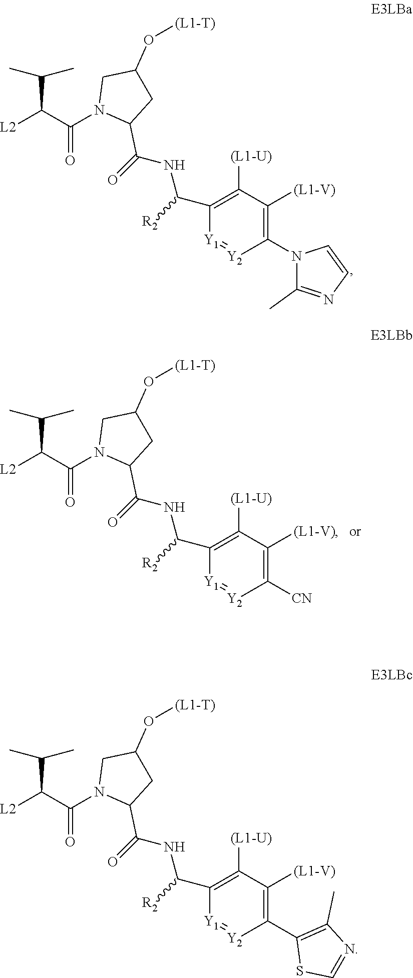

8. The conjugate of embodiment 1, wherein L1 in each instance is independently a linker selected from the group consisting of:

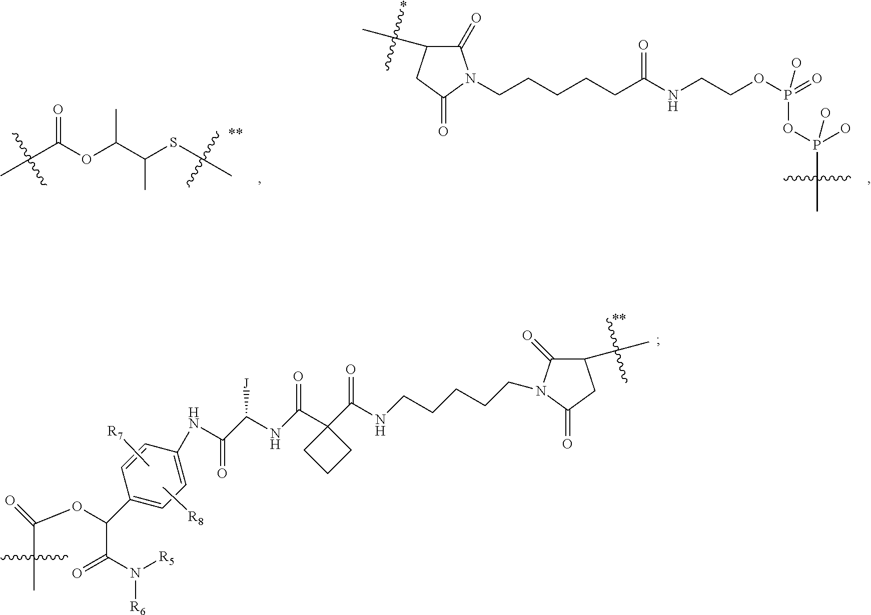

wherein,
   J is —CH₂—CH₂—CH₂—NH—C(O)—NH₂; —CH₂—CH₂—CH₂—CH₂—NH₂; —CH₂—CH₂—CH₂—CH₂—NH—CH₃; or —CH₂—CH₂—CH₂—CH₂—N(CH₃)₂;
   R₅ and R₆ are independently hydrogen or C₁₋₅ alkyl; or R₅ and R₆ together with the nitrogen to which each is attached form an optionally substituted 5- to 7-member heterocyclyl;
   R₇ and R₈ are each independently hydrogen, halo, C₁₋₅ alkyl, C₁₋₅ alkoxy or hydroxy;

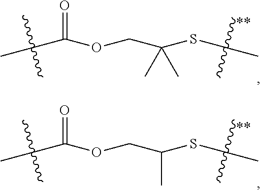

and wherein

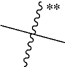

is the point of attachment to Ab.

9. The conjugate of embodiment 8, having the structure:

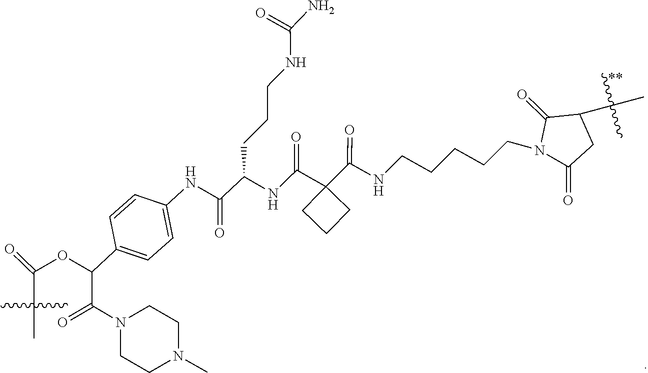 [SMILES: *C(=O)OC(C(=O)N1CCN(C)CC1)c1ccc(NC(=O)[C@H](CCCNC(N)=O)NC(=O)C2(C(=O)NCCCCCN3C(=O)CC(*)C3=O)CCC2)cc1]

10. The conjugate of embodiment 1, wherein L1-T is a linker.

11. The conjugate of embodiment 1, wherein L1-U or L1-V is a linker.

12. The conjugate of embodiment 1, wherein L1-Y is a linker, and q is 1,

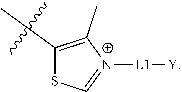

13. The conjugate of embodiment 12, wherein L1-Y has the structure,

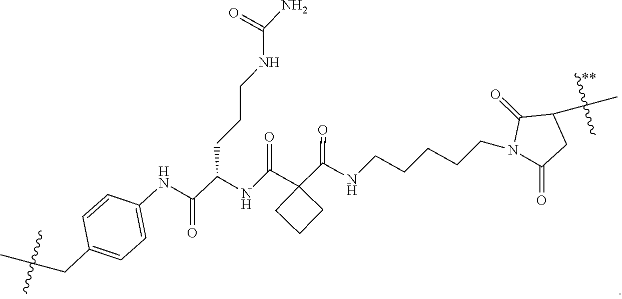 [SMILES: *Cc1ccc(NC(=O)[C@H](CCCNC(N)=O)NC(=O)C2(C(=O)NCCCCCN3C(=O)CC(*)C3=O)CCC2)cc1]

14. The conjugate of embodiment 1, wherein
L1-T is a linker;
L1-U and L1-T are each hydrogen; and
q is zero.

15. The conjugate of embodiment 1, wherein z is zero.

16. The conjugate of embodiment 1, wherein z is one.

17. The conjugate of embodiment 1, wherein R₂ is hydrogen, methyl, ethyl or propyl.

18. The conjugate of embodiment 17, wherein R₂ is methyl.

19. The conjugate of embodiment 18, wherein R₂ is bound to E3LB as

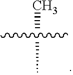

20. The conjugate of embodiment 1, wherein Y₁ and Y₂ are each —CH.

21. The conjugate of embodiment 1, wherein Y₁ is N and Y₂ is —CH.

22. The conjugate of embodiment 1, wherein Y₁ is —CH and Y₂ is N.

23. The conjugate of embodiment 1, wherein R₄ is hydrogen.

24. The conjugate of embodiment 1, wherein R₄ is methyl.

25. The conjugate of embodiment 24, wherein R₄ is a methyl as follows:

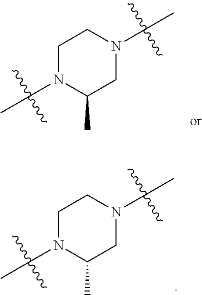

26. The conjugate of embodiment 1, wherein Ab is an antibody that binds to one or more of polypeptides selected from the group consisting of CD71, Trop2, NaPi2b, Ly6E, EpCAM, MSLN, and CD22.

27. The conjugate of embodiment 26, wherein Ab is an antibody that binds to one or more polypeptides selected from the group consisting of CD71 and Trop2.

28. The conjugate of embodiment 1, wherein PB is a protein binding group covalently bound to L2, having the structure:

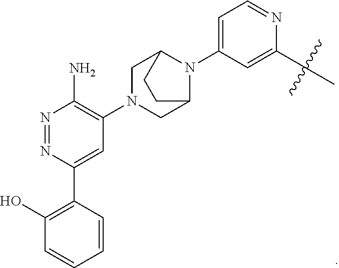 [SMILES: *c1cc(N2C3CCC2CN(c2cc(-c4ccccc4O)nnc2N)C3)ccn1]

29. The conjugate ofembodiment 1 having the Formula Ia

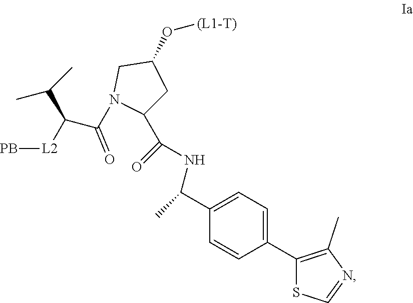

wherein,
   L1-T is a linker covalently bound to Ab; Ab is an antibody that binds to one or more polypeptides selected from the group consisting of CD71, Trop2, NaPi2b, Ly6E, EpCAM, MSLN, and CD22;
   PB is a protein binding group covalently bound to L2, having the structure:

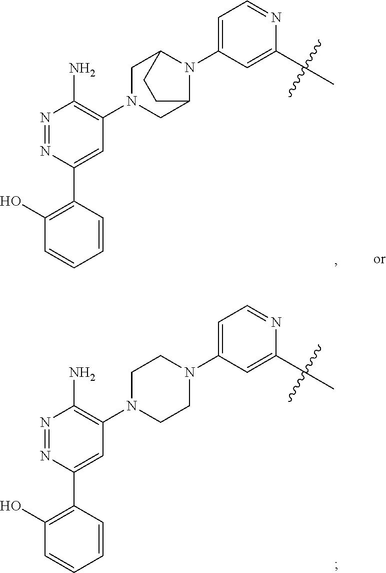 [SMILES: *c1cc(N2C3CCC2CN(c2cc(-c4ccccc4O)nnc2N)C3)ccn1 ; *c1cc(N2CCN(c3cc(-c4ccccc4O)nnc3N)CC2)ccn1]

L2 is selected from the group consisting of L2a, L2b and L2c; and,
p has a value from about 4 to about 8.

30. The conjugate of embodiment 29, wherein L1-T is a linker selected from the group consisting of:

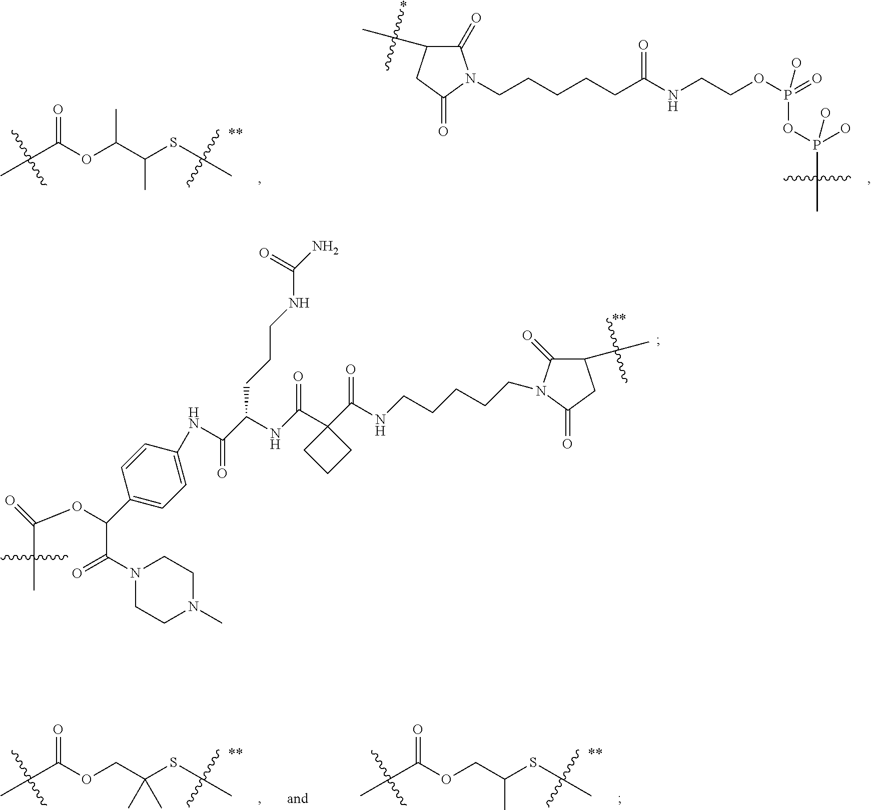

wherein,

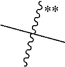

is the point of attachment to Ab.

31. The conjugate of embodiment 29, wherein L2 is L2a.

32. The conjugate of embodiment 31, wherein G is

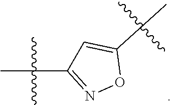 [SMILES: *c1cc(*)on1]

33. The conjugate of embodiment 31, wherein R₄ is methyl.

34. The conjugate of embodiment 29, wherein PB is:

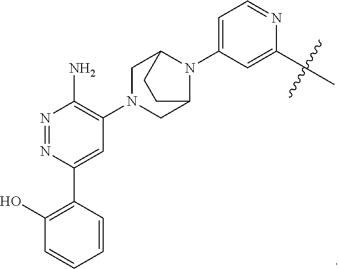 [SMILES: *c1cc(N2C3CCC2CN(c2cc(-c4ccccc4O)nnc2N)C3)ccn1]

35. The conjugate of embodiment 29, wherein p has a value from about 5 to about 7.

36. The conjugate of embodiment 1, having the structure:

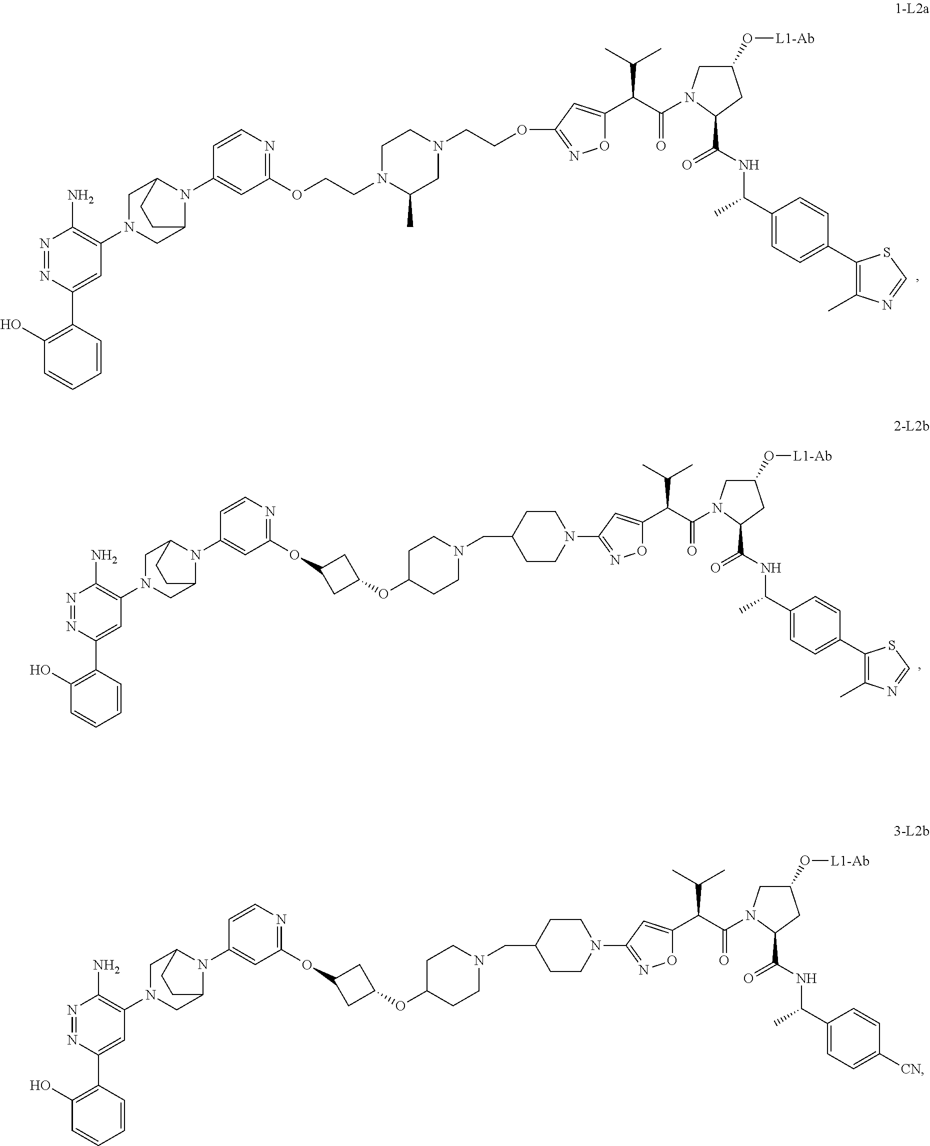

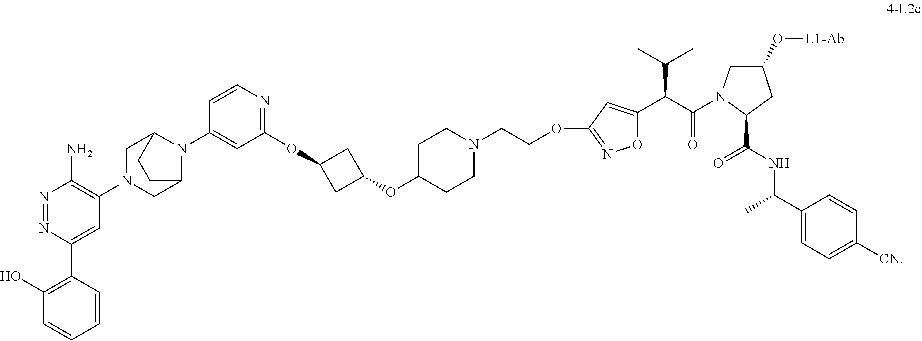

37. The conjugate of embodiment 1 having the structure:

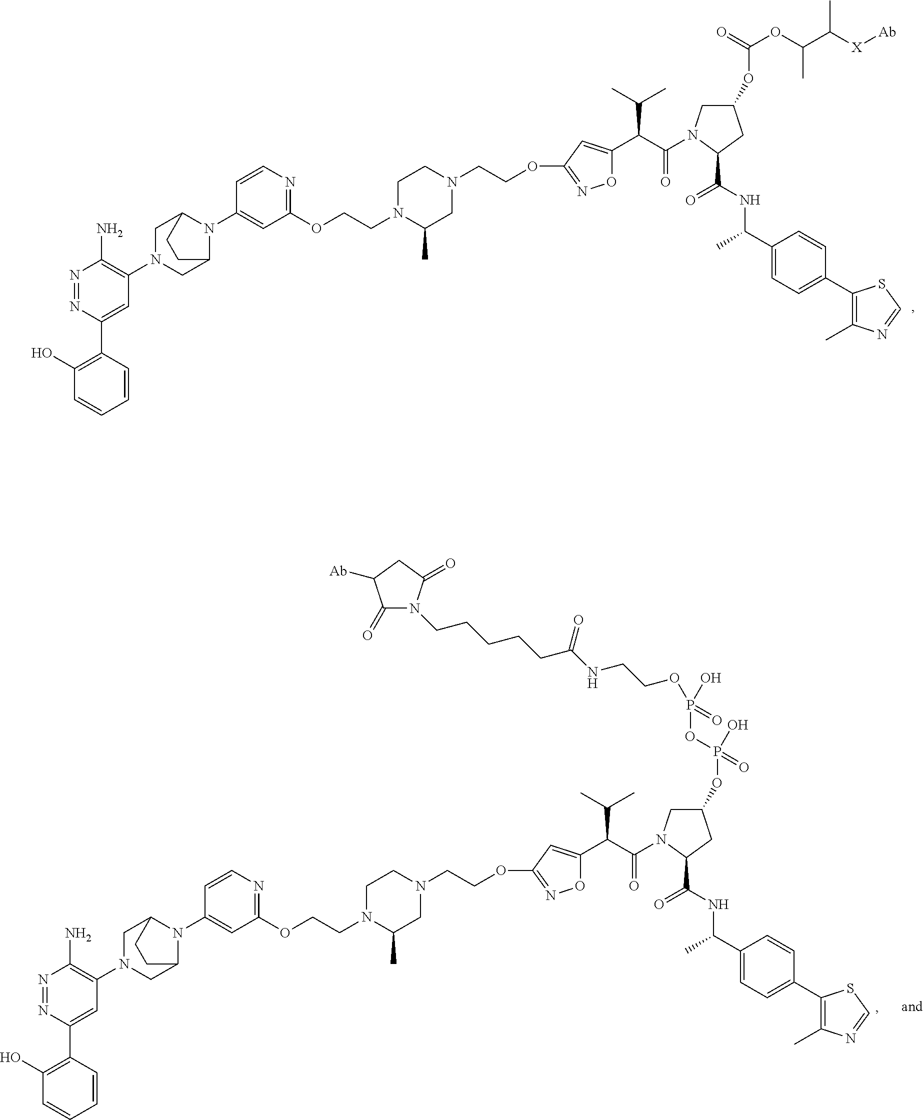

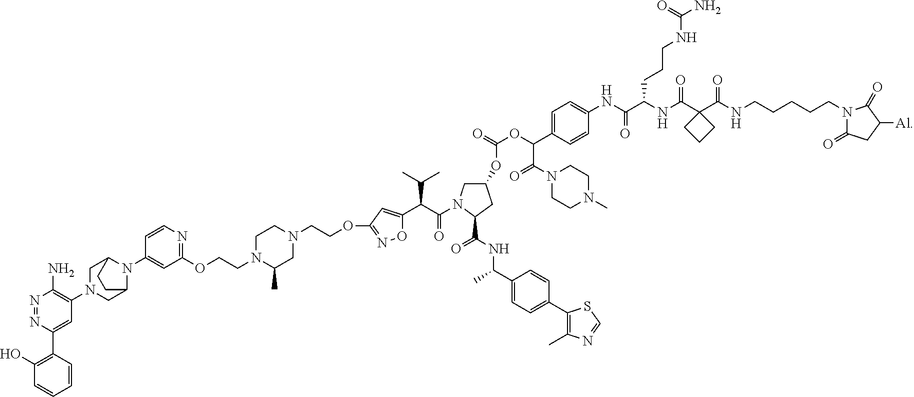

38. A pharmaceutical composition comprising a conjugate of embodiment 1 and one or more pharmaceutically acceptable excipients.

39. A method of treating a disease in a human in need thereof, comprising administering to said human an effective amount of a conjugate of embodiment 1 or a composition of embodiment 38.

40. The method of embodiment 39, wherein said disease is cancer.

41. The method of embodiment 40, wherein said cancer is BRM-dependent.

42. The method of embodiment 40, wherein said cancer is non-small cell lung cancer.

43. A method of reducing the level of a target BRM protein in a subject comprising,
administering a conjugate of embodiment 1 or composition of embodiment 38 to said subject, wherein said PB portion binds said target BRM protein, wherein ubiquitin ligase effects degradation of said bound target BRM protein, wherein the level of said BRM target protein is reduced.

### IV. Formulations

Pharmaceutical formulations of therapeutic Ab-CIDEs as described herein can be prepared for parenteral administration, e.g., bolus, intravenous, intratumor injection with a pharmaceutically acceptable parenteral vehicle and in a unit dosage injectable form. An Ab-CIDE having the desired degree of purity is optionally mixed with one or more pharmaceutically acceptable excipients (Remington's Pharmaceutical Sciences (1980) 16th edition, Osol, A. Ed.), in the form of a lyophilized formulation for reconstitution or an aqueous solution.

An Ab-CIDE can be formulated in accordance with standard pharmaceutical practice as a pharmaceutical composition. According to this aspect, there is provided a pharmaceutical composition comprising an Ab-CIDE in association with one or more pharmaceutically acceptable excipients.

A typical formulation is prepared by mixing an Ab-CIDE with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the Ab-CIDE is being applied. Solvents are generally selected based on solvents recognized by persons skilled in the art as safe (GRAS) to be administered to a mammal.

In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the Ab-CIDE or aid in the manufacturing of the pharmaceutical product. The formulations may be prepared using conventional dissolution and mixing procedures.

Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The Ab-CIDE formulations can be sterile. In particular, formulations to be used for in vivo administration must be sterile. Such sterilization is readily accomplished by filtration through sterile filtration membranes.

The Ab-CIDE ordinarily can be stored as a solid composition, a lyophilized formulation or as an aqueous solution.

The pharmaceutical compositions comprising an Ab-CIDE can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The Ab-CIDE can be formulated into pharmaceutical dosage forms to provide an easily controllable dosage of the drug and to enable patient compliance with the prescribed regimen. The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of Ab-CIDE that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally or by any other desired route.

### V. Indications and Methods of Treatment

It is contemplated that the Ab-CIDEs disclosed herein may be used to treat various diseases or disorders that are related to BRM. Also provided herein is an Ab-CIDE or a composition comprising an Ab-CIDE for use in therapy. In some embodiments, provided herein is an Ab-CIDE or a composition comprising an Ab-CIDE for the treatment or prevention of diseases and disorders as disclosed herein. Also provided herein is the use of an Ab-CIDE or a composition comprising an Ab-CIDE in therapy. In some embodiments, provided herein is the use of an Ab-CIDE for the treatment or prevention of diseases and disorders as disclosed herein. Also provided herein is the use of an Ab-CIDE or a composition comprising an Ab-CIDE in the manufacture of a medicament for the treatment or prevention of diseases and disorders as disclosed herein.

Generally, the disease or disorder to be treated is BRM-dependent disease or disorder, for example, a hyperproliferative disease such as cancer. Examples of cancer to be treated herein include BRM-dependent cancers. In certain embodiments, the cancer is non-small cell lung cancer.

In certain embodiments, the subject matter described herein is directed to a method of reducing the level of a target BRM protein in a subject comprising,
administering an Ab-CIDE as described herein or composition comprising an Ab-CIDE as described herein to a subject, wherein the PB portion binds a target BRM protein, wherein ubiquitin ligase effects degradation of a bound target BRM protein, wherein the level of a BRM target protein is reduced.

In certain embodiments, an Ab-CIDE comprising an anti-NaPi2b antibody, such as those described above, is used in a method of treating solid tumor, e.g., ovarian. In certain embodiments, an Ab-CIDE comprising an anti-CD71, Trop2, NaPi2b, Ly6E, EpCAM, MSLN, or CD22 antibody is used in a method of treating a tumor or cancer.

An Ab-CIDE may be administered by any route appropriate to the condition to be treated. The Ab-CIDE will typically be administered parenterally, i.e. infusion, subcutaneous, intramuscular, intravenous, intradermal, intrathecal and epidural.

An Ab-CIDE can be used either alone or in combination with other agents in a therapy. For instance, an Ab-CIDE may be co-administered with at least one additional therapeutic agent. Such combination therapies noted above encompass combined administration (where two or more therapeutic agents are included in the same or separate formulations), and separate administration, in which case, administration of the Ab-CIDE can occur prior to, simultaneously, and/or following, administration of the additional therapeutic agent and/or adjuvant. An Ab-CIDE can also be used in combination with radiation therapy.

An Ab-CIDE (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an Ab-CIDE (when used alone or in combination with one or more other additional therapeutic agents) will depend on the type of disease to be treated, the type of Ab-CIDE, the severity and course of the disease, whether the Ab-CIDE is administered for preventive or therapeutic purposes, previous therapy, the patient's clinical history and response to the Ab-CIDE, and the discretion of the attending physician. The Ab-CIDE is suitably administered to the patient at one time or over a series of treatments. Depending on the type and severity of the disease, about 1 μg/kg to 15 mg/kg (e.g. 0.1 mg/kg-10 mg/kg) of an Ab-CIDE can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an Ab-CIDE would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses). An initial higher loading dose, followed by one or more lower doses may be administered. However, other dosage regimens may be useful. The progress of this therapy is easily monitored by conventional techniques and assays.

The methods described herein include methods of degrading target proteins. In certain embodiments, the methods comprise administering an Ab-CIDE to a subject, wherein the target protein is degraded. The level of degradation of the protein can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

The methods described herein include methods of reducing proliferation of a neoplastic tissue, such as non-small cell lung cancer. In certain embodiments, the methods comprise administering an Ab-CIDE to a subject, wherein the proliferation of a neoplastic tissue is reduced. The level of reduction can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

### VI. Articles of Manufacture

In another aspect, described herein are articles of manufacture, for example, a “kit,” containing materials useful for the treatment of the diseases and disorders described above is provided. The kit comprises a container comprising an Ab-CIDE. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products.

Suitable containers include, for example, bottles, vials, syringes, blister pack, etc. A “vial” is a container suitable for holding a liquid or lyophilized preparation. In one embodiment, the vial is a single-use vial, e.g. a 20-cc single-use vial with a stopper. The container may be formed from a variety of materials such as glass or plastic. The container may hold an Ab-CIDE or a formulation thereof which is effective for treating the condition and may have a sterile access port (for example, the container may be an intravenous solution bag or a vial having a stopper pierceable by a hypodermic injection needle).

At least one active agent in the composition is an Ab-CIDE. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising an Ab-CIDE can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the Ab-CIDE and, if present, the second pharmaceutical formulation. For example, if the kit comprises a first composition comprising an Ab-CIDE, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of an Ab-CIDE, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with an Ab-CIDE contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises an Ab-CIDE and a second therapeutic agent, the kit may comprise a container for containing the separate compositions such as a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### VII. Methods of Making Conjugates

Synthesis Routes

The subject matter described herein is also directed to methods of preparing a CIDE, a L1-CIDE, and an Ab-CIDE from a L1-CIDE. Generally, the method comprises contacting an antibody, or variants, mutations, splice variants, indels and fusions thereof, with a L1-CIDE under conditions where the antibody is covalently bound to any available point of attachment on a L1-CIDE, wherein an Ab-CIDE is prepared. The subject matter described herein is also directed to methods of preparing an Ab-CIDE from an Ab-L1 portion, i.e., an antibody, or variants, mutations, splice variants, indels and fusions thereof, covalently attached to a L1, the methods comprising contacting a CIDE with an Ab-L1 under conditions where the CIDE is covalently bound to any available point of attachment on the Ab-L1, wherein an Ab-CIDE is prepared. The methods can further comprise routine isolation and purification of the Ab-CIDEs.

CIDEs, L1-CIDEs and Ab-CIDEs and other compounds described herein can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990). Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing the CIDEs, L1-CIDEs and Ab-CIDEs and other compounds as described herein and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof. In preparing CIDEs, L1-CIDEs and Ab-CIDEs and other compounds, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz or CBZ) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

The General Procedures and Examples provide exemplary methods for preparing CIDEs, L1-CIDEs and Ab-CIDEs and other compounds described herein. Those skilled in the art will appreciate that other synthetic routes may be used to synthesize the Ab-CIDEs and compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

Generally, an Ab-CIDE can be prepared by connecting a CIDE with a L1 linker reagent according to the procedures of WO 2013/055987; WO 2015/023355; WO 2010/009124; WO 2015/095227, to prepare a L1-CIDE, and conjugating the L1-CIDE with any of the antibodies or variants, mutations, splice variants, indels and fusions thereof, including cysteine engineered antibodies, described herein. Alternatively, an Ab-CIDE can be prepared by first connecting an antibody or variant, mutation, splice variant, indel and fusion thereof, including a cysteine engineered antibody, described herein with a L1 linker reagent, and conjugating it with any CIDE.

The following synthetic routes describe exemplary methods of preparing CIDEs, L1-CIDEs and Ab-CIDEs and other compounds and components thereof. Other synthetic routes for preparing CIDEs, L1-CIDEs and Ab-CIDEs and other compounds and components thereof are disclosed elsewhere herein.

1. Linker L1

With respect to Linker L1, Schemes 1-4 depict synthesis routes to exemplary linkers L1 for disulfide attachment to antibody Ab. The Ab is connected to L1 through a disulfide bond and the CIDE is connected to L1 through any available attachment on the CIDE.

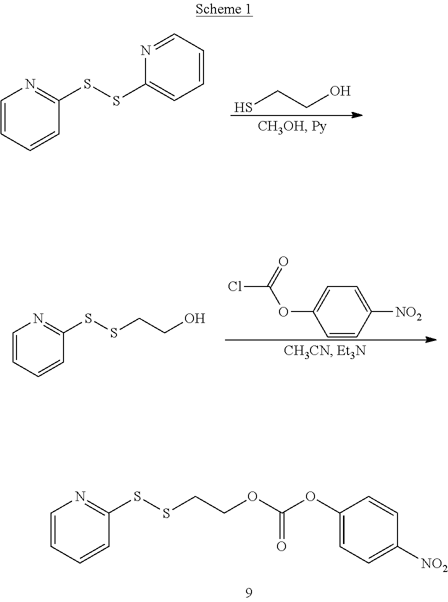

Referring to Scheme 1, 1,2-Di(pyridin-2-yl)disulfane and 2-mercaptoethanol were reacted in pyridine and methanol at room temperature to give 2-(pyridin-2-yldisulfanyl)ethanol. Acylation with 4-nitrophenyl carbonochloridate in triethylamine and acetonitrile gave 4-nitrophenyl 2-(pyridin-2-yldisulfanyl)ethyl carbonate 9.

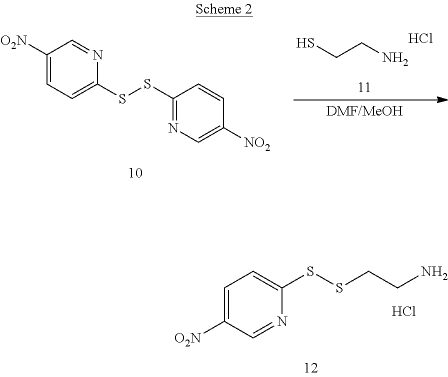

Referring to Scheme 2, to a mixture of 1,2-bis(5-nitropyridin-2-yl)disulfane 10 (1.0 g, 3.22 mmol) in anhydrous DMF/MeOH (25 mL/25 mL) was added HOAc (0.1 mL), followed by 2-aminoethanethiol hydrochloride 11 (183 mg, 1.61 mmol). After the reaction mixture was stirred at r.t. overnight, it was concentrated under vacuum to remove the solvent, and the residue was washed with DCM (30 mL×4) to afford 2-((5-nitropyridin-2-yl)disulfanyl)ethanamine hydrochloride 12 as pale yellow solid (300 mg, 69.6%). ¹H NMR (400 MHz, DMSO-d₆) δ 9.28 (d, J=2.4 Hz, 1H), 8.56 (dd, J=8.8, 2.4 Hz, 1H), 8.24 (s, 4H), 8.03 (d, J=8.8 Hz, 1H), 3.15-3.13 (m, 2H), 3.08-3.06 (m, 2H).

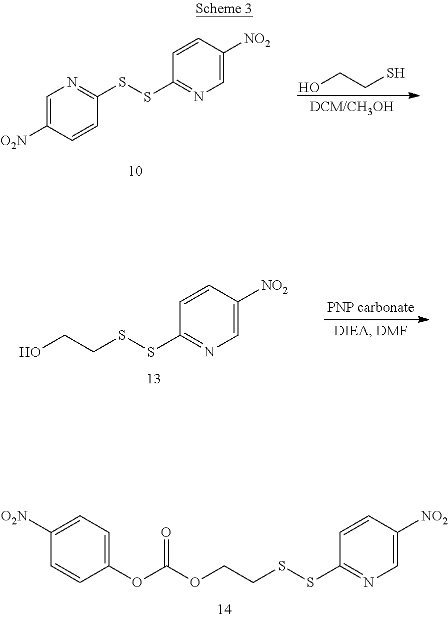

Referring to Scheme 3, a solution of 1,2-bis(5-nitropyridin-2-yl)disulfane 10 (9.6 g, 30.97 mmol) and 2-mercaptoethanol (1.21 g, 15.49 mmol) in anhydrous DCM/CH₃OH (250 mL/250 mL) was stirred at r.t. under N₂ for 24 h. After the mixture was concentrated under vacuum, and the residue was diluted with DCM (300 mL). MnO₂ (10 g) was added and the mixture was stirred at r.t. for another 0.5 h. The mixture was purified by column chromatography on silica gel (DCM/MeOH=100/1 to 100/1) to afford 2-((5-nitropyridin-2-yl)disulfanyl)ethanol 13 (2.2 g, 61.1%) as brown oil. ¹H NMR (400 MHz, CDCl₃) δ 9.33 (d, J=2.8 Hz, 1H), 8.38-8.35 (dd, J=9.2, 2.8 Hz, 1H), 7.67 (d, J=9.2 Hz, 1H), 4.10 (t, J=7.2 Hz, 1H), 3.81-3.76 (q, 2H), 3.01 (t, J=5.2 Hz, 2H).

To a solution of 13 (500 mg, 2.15 mmol) in anhydrous DMF (10 mL) was added DIEA (834 mg, 6.45 mmol), followed by PNP carbonate (bis(4-nitrophenyl) carbonate, 1.31 g, 4.31 mmol). The reaction solution was stirred at r.t for 4 h and the mixture was purified by prep-HPLC (FA) to afford 4-nitrophenyl 2-((5-nitropyridin-2-yl)disulfanyl)ethyl carbonate 14 (270 mg, 33.1%) as light brown oil. ¹H NMR (400 MHz, CDCl₃) δ 9.30 (d, J=2.4 Hz, 1H), 8.43-8.40 (dd, J=8.8, 2.4 Hz, 1H), 8.30-8.28 (m, 2H), 7.87 (d, J=8.8 Hz, 1H), 7.39-7.37 (m, 2H), 4.56 (t, J=6.4 Hz, 2H), 3.21 (t, J=6.4 Hz, 2H).

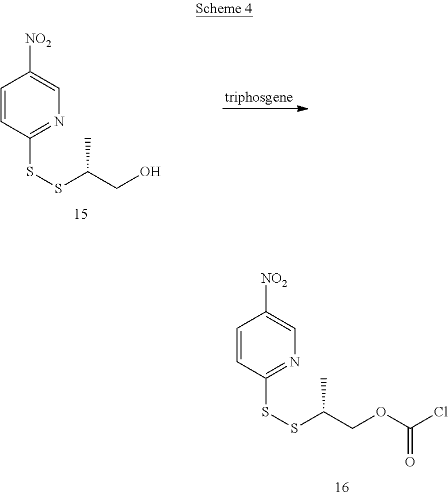 [SMILES: C[C@H](CO)SSc1ccc([N+](=O)[O-])cn1 ; C[C@H](COC(=O)Cl)SSc1ccc([N+](=O)[O-])cn1]

Referring to Scheme 4, sulfuryl chloride (2.35 mL of a 1.0M solution in DCM, 2.35 mmol) was added drop-wise to a stirred suspension of 5-nitropyridine-2-thiol (334 mg, 2.14 mmol) in dry DCM (7.5 mL) at 0° C. (ice/acetone) under an argon atmosphere. The reaction mixture turned from a yellow suspension to a yellow solution and was allowed to warm to room temperature then stirred for 2 hours after which time the solvent was removed by evaporation in vacuo to provide a yellow solid. The solid was re-dissolved in DCM (15 mL) and treated drop-wise with a solution of (R)-2-mercaptopropan-1-ol (213 mg, 2.31 mmol) in dry DCM (7.5 mL) at 0° C. under an argon atmosphere. The reaction mixture was allowed to warm to room temperature and stirred for 20 hours at which point analysis by LC/MS revealed substantial product formation at retention time 1.41 minutes (ES+) m/z 247 ([M+H]⁺, ˜100% relative intensity). The precipitate was removed by filtration and the filtrate evaporated in vacuo to give an orange solid which was treated with H₂O (20 mL) and basified with ammonium hydroxide solution. The mixture was extracted with DCM (3×25 mL) and the combined extracts washed with H₂O (20 mL), brine (20 mL), dried (MgSO₄), filtered and evaporated in vacuo to give the crude product. Purification by flash chromatography (gradient elution in 1% increments: 100% DCM to 98:2 v/v DCM/MeOH) gave (R)-2-((5-nitropyridin-2-yl)disulfanyl)propan-1-ol 15 as an oil (111 mg, 21% yield).

To a solution of triphosgene, Cl₃COCOOCCl₃, Sigma Aldrich, CAS Reg. No. 32315-10-9 (241 mg, 0.812 mmol) in DCM (10 mL) was added a solution of (R)-2-((5-nitropyridin-2-yl)disulfanyl)propan-1-ol 15 (500 mg, 2.03 mmol) and pyridine (153 mg, 1.93 mmol) in DCM (10 mL) dropwise at 20° C. After the reaction mixture was stirred at 20° C. for 30 min, it was concentrated and (R)-2-((5-nitropyridin-2-yl)disulfanyl)propyl carbonochloridate 16 can be used directly without further purification to covalently link through the carbonochloridate group any available group on the CIDE.

2. Cysteine Engineered Antibodies

With regard to cysteine engineered antibodies for conjugation by reduction and reoxidation, they can be prepared generally as follows. Light chain amino acids are numbered according to Kabat (Kabat et al., Sequences of proteins of immunological interest, (1991) 5th Ed., US Dept of Health and Human Service, National Institutes of Health, Bethesda, MD). Heavy chain amino acids are numbered according to the EU numbering system (Edelman et al (1969) Proc. Natl. Acad. of Sci. 63(1):78-85), except where noted as the Kabat system. Single letter amino acid abbreviations are used.

Full length, cysteine engineered monoclonal antibodies (THIOMAB™ antibodies) expressed in CHO cells bear cysteine adducts (cystines) or are glutathionylated on the engineered cysteines due to cell culture conditions. As is, THIOMAB™ antibodies purified from CHO cells cannot be conjugated to Cys-reactive linker L1-CIDE intermediates. Cysteine engineered antibodies may be made reactive for conjugation with L1-CIDE intermediates described herein, by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, MA) followed by re-formation of the inter-chain disulfide bonds (re-oxidation) with a mild oxidant such as dehydroascorbic acid. Full length, cysteine engineered monoclonal antibodies (THIOMAB™ antibodies) expressed in CHO cells (Gomez et al (2010) Biotechnology and Bioeng. 105(4):748-760; Gomez et al (2010) Biotechnol. Prog. 26:1438-1445) were reduced, for example, with about a 50 fold excess of DTT overnight in 50 mM Tris, pH 8.0 with 2 mM EDTA at room temperature, which removes Cys and glutathione adducts as well as reduces interchain disulfide bonds in the antibody. Removal of the adducts was monitored by reverse-phase LCMS using a PLRP-S column. The reduced THIOMAB™ antibody was diluted and acidified by addition to at least four volumes of 10 mM sodium succinate, pH 5 buffer.

Alternatively, the antibody was diluted and acidified by adding to at least four volumes of 10 mM succinate, pH 5 and titration with 10% acetic acid until pH was approximately five. The pH-lowered and diluted THIOMAB™ antibody was subsequently loaded onto a HiTrap S cation exchange column, washed with several column volumes of 10 mM sodium acetate, pH 5 and eluted with 50 mM Tris, pH 8.0, 150 mM sodium chloride. Disulfide bonds were reestablished between cysteine residues present in the parent Mab by carrying out reoxidation. The eluted reduced THIOMAB™ antibody described above is treated with 15× dehydroascorbic acid (DHAA) for about 3 hours or, alternatively, with 200 nM to 2 mM aqueous copper sulfate (CuSO₄) at room temperature overnight. Other oxidants, i.e. oxidizing agents, and oxidizing conditions, which are known in the art may be used. Ambient air oxidation may also be effective. This mild, partial reoxidation step forms intrachain disulfides efficiently with high fidelity. Reoxidation was monitored by reverse-phase LCMS using a PLRP-S column. The reoxidized THIOMAB™ antibody was diluted with succinate buffer as described above to reach pH approximately 5 and purification on an S column was carried out as described above with the exception that elution was performed with a gradient of 10 mM succinate, pH 5, 300 mM sodium chloride (buffer B) in 10 mM succinate, pH 5 (buffer A). To the eluted THIOMAB™ antibody, EDTA was added to a final concentration of 2 mM and concentrated, if necessary, to reach a final concentration of more than 5 mg/mL. The resulting THIOMAB™ antibody, ready for conjugation, was stored at −20° C. or −80° C. in aliquots. Liquid chromatography/Mass Spectrometric Analysis was performed on a 6200 series TOF or QTOF Agilent LC/MS. Samples were chromatographed on a PRLP-S®, 1000 A, microbore column (50 mm×2.1 mm, Polymer Laboratories, Shropshire, UK) heated to 80° C. A linear gradient from 30-40% B (solvent A: 0.05% TFA in water, solvent B: 0.04% TFA in acetonitrile) was used and the eluent was directly ionized using the electrospray source. Data were collected and deconvoluted by the MassHunter software (Agilent). Prior to LC/MS analysis, antibodies or conjugates (50 micrograms) were treated with PNGase F (2 units/ml; PROzyme, San Leandro, CA) for 2 hours at 37° C. to remove N-linked carbohydrates.

Alternatively, antibodies or conjugates were partially digested with LysC (0.25 μg per 50 μg (microgram) antibody or conjugate) for 15 minutes at 37° C. to give a Fab and Fc fragment for analysis by LCMS. Peaks in the deconvoluted LCMS spectra were assigned and quantitated. CIDE-to-antibody ratios (CAR) were calculated by calculating the ratio of intensities of the peak or peaks corresponding to CIDE-conjugated antibody relative to all peaks observed.

3. Conjugation of Linker L1-CIDE group to antibodies

In one method of conjugating Linker L1-CIDE compounds to antibodies, after the reduction and reoxidation procedures above, the cysteine-engineered antibody (THIOMAB™ antibody), in 10 mM succinate, pH 5, 150 mM NaCl, 2 mM EDTA, is pH-adjusted to pH 7.5-8.5 with 1M Tris. An excess, from about 3 molar to 20 equivalents of a linker-CIDE intermediate with a thiol-reactive group (e.g., maleimide or 4-nitropyridy disulfide, or methanethiosulfonyl (MTS) disulfide), is dissolved in DMF, DMA or propylene glycol and added to the reduced, reoxidized, and pH-adjusted antibody. The reaction is incubated at room temperature or 37 C and monitored until completion (1 to about 24 hours), as determined by LC-MS analysis of the reaction mixture. When the reaction is complete, the conjugate is purified by one or any combination of several methods, the goal being to remove remaining unreacted L1-CIDE intermediate and aggregated protein (if present at significant levels). For example, the conjugate may be diluted with 10 mM histidine-acetate, pH 5.5 until final pH is approximately 5.5 and purified by S cation exchange chromatography using either HiTrap S columns connected to an Akta purification system (GE Healthcare) or S maxi spin columns (Pierce). Alternatively, the conjugate may be purified by gel filtration chromatography using an S200 column connected to an Akta purification system or Zeba spin columns. Alternatively, dialysis may be used. The THIOMAB™ antibody CIDE conjugates were formulated into 20 mM His/acetate, pH 5, with 240 mM sucrose using either gel filtration or dialysis. The purified conjugate is concentrated by centrifugal ultrafiltration and filtered through a 0.2-μm filter under sterile conditions and frozen for storage. The Ab-CIDEs were characterized by BCA assay to determine protein concentration, analytical SEC (size-exclusion chromatography) for aggregation analysis and LC-MS after treatment with Lysine C endopeptidase (LysC) to calculate CAR.

Size exclusion chromatography is performed on conjugates using a Shodex KW802.5 column in 0.2M potassium phosphate pH 6.2 with 0.25 mM potassium chloride and 15% IPA at a flow rate of 0.75 ml/min. Aggregation state of the conjugate was determined by integration of eluted peak are an absorbance at 280 nm.

LC-MS analysis may be performed on Ab-CIDE using an Agilent QTOF 6520 ESI instrument. As an example, the CAR is treated with 1:500 w/w Endoproteinase Lys C (Promega) in Tris, pH 7.5, for 30 min at 37° C. The resulting cleavage fragments are loaded onto a 1000 Å (Angstrom), 8 μm (micron) PLRP-S (highly cross-linked polystyrene) column heated to 80° C. and eluted with a gradient of 30% B to 40% B in 5 minutes. Mobile phase A was H₂O with 0.05% TFA and mobile phase B was acetonitrile with 0.04% TFA. The flow rate was 0.5 ml/min. Protein elution was monitored by UV absorbance detection at 280 nm prior to electrospray ionization and MS analysis. Chromatographic resolution of the unconjugated Fc fragment, residual unconjugated Fab and drugged Fab was usually achieved. The obtained m/z spectra were deconvoluted using Mass Hunter™ software (Agilent Technologies) to calculate the mass of the antibody fragments.

General Synthetic Methods

General methods for preparing a conjugate having the chemical structure Ab-(L1-D)ₚ are described below.

1.1 General Synthetic Method for Coupling of L2 to E3LB to Prepare a E3LB-L2 Intermediate

In certain embodiments, L2 is first contacted with a first suitable solvent, a first base and a first coupling reagent to prepare a first solution. In certain embodiments, the contacting of L2 with a first suitable solvent, a first base, and a first coupling reagent proceeds for about 15 minutes at room temperature (about 25° C.). The E3LB is then contacted with said first solution.

In certain embodiments, the contacting of E3LB with the first solution proceeds for about one hour at room temperature (about 25° C.). The solution is then concentrated and optionally purified.

In certain embodiments, the molar ratio of L2 to first base to first coupling reagent is about 1:4:1.19. In certain embodiments, the molar ratio of L2 to first base to first coupling reagent is about 1:2:0.5, about 1:3:1, about 1:4:2, about 1:5:3, or about 1:6:4.

In certain embodiments, the molar ratio of L2 to E3LB is about 1:1. In certain embodiments, the molar ratio of L2 to E3LB is about 1:0.5, about 1:0.75, about 1:2, or about 0.5:1.

1.2 General Synthetic Method for Coupling E3LB-L2 Intermediate to PB to Prepare a CIDE

In certain embodiments, the E3LB-L2 intermediate is coupled to a PB to prepare a CIDE. In certain embodiments, the PB is first contacted with a second suitable solvent, a second base, and second coupling reagent. In certain embodiments, the contacting proceeds for about 10 minutes at room temperature (about 25° C.). The solution is then contacted with the E3LB-L2 intermediate. In certain embodiments, the contacting of the second solution with the E3LB-L2 intermediate proceeds for about 1 hour at room temperature (about 25° C.). The solution is then concentrated and optionally purified to prepare a CIDE.

In certain embodiments, the molar ratio of PB to second base to second coupling reagent is about 1:4:1.2. In certain embodiments, the molar ratio of PB to second base to second coupling reagent is about 1:3:0.75, about 1:5:1, about 1:3:2, or about 1:5:3.

In certain embodiments, the molar ratio of PB to E3LB-L2 intermediate is about 1:1. In certain embodiments, the molar ratio of PB to E3LB-L2 intermediate is about 1:0.5, about 1:0.75, about 1:2, or about 0.5:1.

1.3 General Synthetic Method for Coupling CIDE to L1 to Prepare L1-CIDE

In certain embodiments, the CIDE is contacted with L1 and a third base in a third suitable solvent to prepare a solution. In certain embodiments, the contacting proceeds for about 2 hours at about (about 25° C.). The solution can then be optionally purified to prepare L1-CIDE.

In certain embodiments, the molar ratio of CIDE to L1 is about 1:4. In certain embodiments, the molar ratio of CIDE to L1 is about 1:1, 1:2, 1:3, 1:5, 1:6, 1:7, or about 1:8.

1.4 General Synthetic Method for Coupling L1-CIDE to Antibody

In certain embodiments, the L1-CIDE is contacted with a thiol and a fourth suitable solvent to form a fourth solution. This solution is then contacted with an antibody to prepare the conjugate. In certain embodiments, the

In certain embodiments, the thiol is maleimide or 4-nitropyridy disulfide. In certain embodiments, the suitable solvent is selected from the group consisting of dimethylformamide, dimethylacetamide, and propylene glycol.

In certain embodiments, the molar ratio of L1-CIDE to thiol-reactive group is about 3:1 to about 20:1.

In certain embodiments, contacting the solution comprising the L1-CIDE, the thiol-reactive group and the suitable solvent with the antibody proceeds for about 1 to about 24 hours. In certain embodiments, contacting the solution comprising the L1-CIDE, the thiol-reactive group and the suitable solvent with the antibody proceeds at about room temperature (about 25° C.) to about 37° C.

In certain embodiments of the general methods above, the suitable solvent is a polar aprotic solvent, selected from the group consisting of dimethylformamide, tetrahydrofuran, ethyl acetate, acetone, acetonitrile, dimethyl sulfoxide, and propylene carbonate.

In certain embodiments of the general methods above, the base is selected from the group consisting of N,N-Diisopropylethylamine (DIEA), triethylamine, and 2,2,2,6,6-tetramethylpiperidine. In certain embodiments, the coupling reagent is selected from the group consisting of 1-[Bis(dimethylamino)methylene]-1H-1,2,3-triazolo[4,5-b]pyridinium 3-oxide hexafluorophosphate (HATU), (Benzotriazol-1-yloxy)tris(dimethylamino)phosphonium hexafluorophosphate (BOP), (7-Azabenzotriazol-1-yloxy)tripyrrolidinophosphonium hexafluorophosphate (PyAOP), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(6-Chlorobenzotriazol-1-yl)-N,N,N′,N′-tetramethyluronium hexafluorophosphate (HCTU), O—(N-Suc-cinimidyl)-1,1,3,3-tetramethyl-uronium tetrafluoroborate (TSTU), O-(5-Norbornene-2,3-dicarboximido)-N,N,N′,N′-tetramethyluronium tetrafluoroborate (TNTU), O-(1,2-Dihydro-2-oxo-1-pyridyl-N,N,N′,N′-tetramethyluronium tetrafluoroborate (TPTU), and Carbonyldiimidazolc (CDI).

In a preferred embodiment, the solvent is dimethylformamide, the base is N,N-Diisopropylethylamine, and the coupling reagent is HATU.

In certain embodiments of the general methods above, contacting proceeds for about 30 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 30 minutes, 60 minutes, 90 minutes, 120 minutes, 180 minutes, 4 hours, 5 hours, 6 hours, 7 hours, 8 hours, 9 hours, 10 hours, 20 hours, 40 hours, 60 hours, or 72 hours.

In certain embodiments of the general methods above, contacting proceeds at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 60° C., 70° C., 80° C., 90° C., or 100° C.

The following examples are offered by way of illustration and not by way of limitation.

### EXAMPLES

All compounds are mixtures of olefin isomers (approximately 1:1) unless otherwise specified. ¹³C resonances listed in parentheses represent olefin isomers and/or alternate N-Me amide bond rotamers of the major isomer of a particular compound.

### Synthesis Example 1

### Syntheses of L1-CIDE-BRM1-1

L1-CIDE-BRM1-1 was synthesized by the following scheme, Scheme 1:

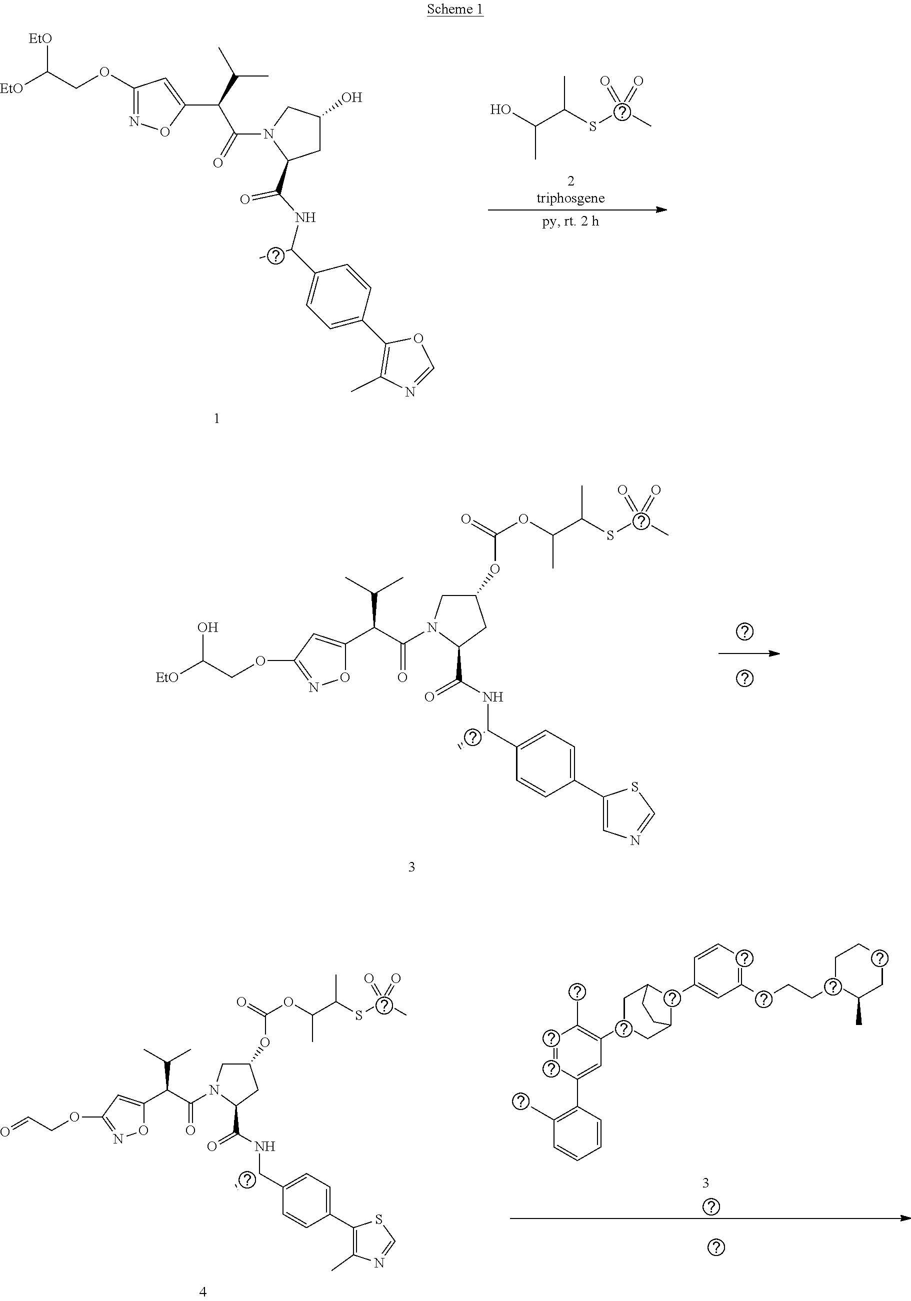

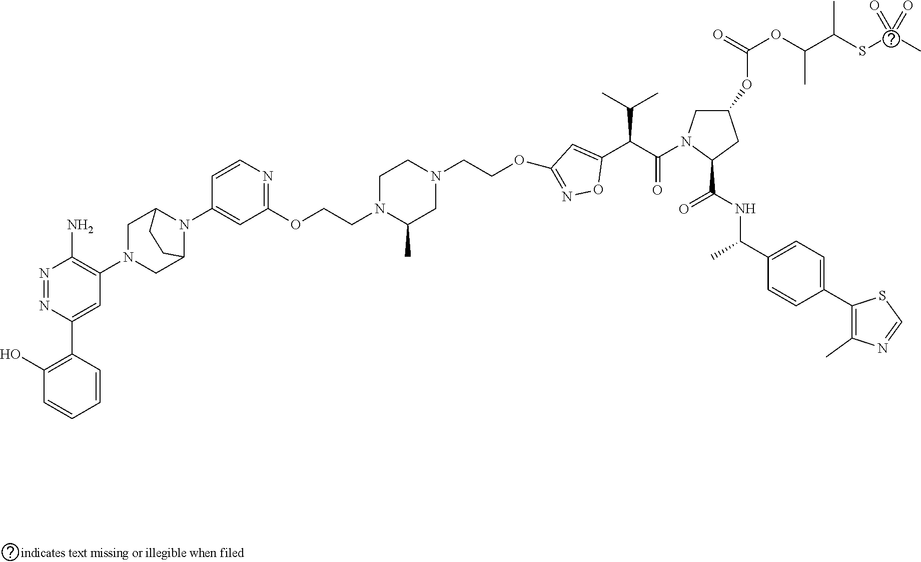

3,3′-disulfanediylbis(butan-2-ol) was synthesized in 2 steps as follows:

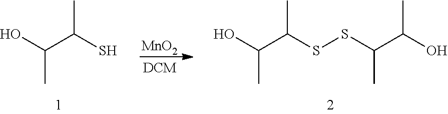

To a 23° C. solution of 3-mercaptobutan-2-ol (2.0 g, 19 mmol) in anhydrous dichloromethane (40 mL) was added MnO₂ (2.46 g, 28 mmol). The reaction mixture was stirred at 23° C. for 1 h then was filtered. The filtrate was concentrated in vacuo to afford 3,3′-disulfanediylbis(butan-2-ol) (1.97 g, 99%) as colorless oil. ¹H NMR (400 MHz, CDCl₃) δ 4.13-4.08 and 3.83-3.78 (m, 2H), 2.94-2.91 and 2.82-2.78 (m, 2H), 2.38-2.26 and 2.14-2.02 (m, 2H), 1.35-1.22 (m, 12H).

S-(3-hydroxybutan-2-yl) methanesulfonothioate, compound 3 below (which is compound 2 in Scheme 1 above) was synthesized as follows:

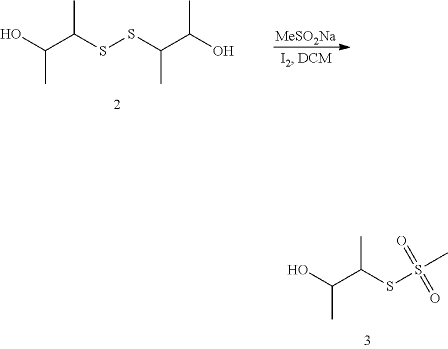

To a 23° C. solution of 3,3′-disulfanediylbis(butan-2-ol) (1.97 g, 9.36 mmol) in anhydrous dichloromethane (50 mL) was added sodium methanesulfinate (1.91 g, 18.7 mmol) and iodine (2.38 g, 9.36 mmol). The reaction mixture was stirred in dark at 23° C. for 1 day then was filtered. The filtrate was concentrated, and the residue was purified by chromatography on silica eluting with 0 to 5% MeOH in DCM to afford S-(3-hydroxybutan-2-yl) methanesulfonothioate (1.20 g, 70%) as pale yellow oil. ¹H NMR (400 MHz, CDCl₃) δ 4.13-4.11 and 3.96-3.93 (m, 1H), 3.77-3.73 and 3.51-3.47 (m, 1H), 3.42 and 3.39 (s, 3H), 2.04 and 1.96 (brs, 1H), 1.53 and 1.42 (d, J=7.2 Hz, 3H), 1.33 and 1.23 (d, J=6.0 Hz, 3H).

### Synthesis of S-(3-((Chlorocarbonyl)oxy)butan-2-yl) methanesulfonothioate

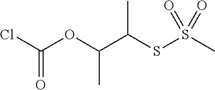

To a solution of S-(3-hydroxybutan-2-yl) methanesulfonothioate (300 mg, 1.63 mmol) and pyridine (516 mg, 6.51 mmol) in dichloromethane (2 mL) at 23° C. was added a solution of triphosgene (242 mg, 0.81 mmol) in dichloromethane (2 mL). The reaction was stirred at 23° C. for 30 min. The reaction mixture was concentrated to dryness to afford the title compound (380 mg, 95%) as yellow oil which was used directly in next step.

### S-(3-(((((3R,5S)-1-((R)-2-(3-(2,2-Diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

To a mixture of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide [Compound 1 in the Example 1 Scheme above; see p 293-294 (top of page numbering) of US 2020/0038378 for preparation.] (300 mg, 0.49 mmol) and 4 Å MS (100 mg) in anhydrous dichloromethane (5 mL) at 23° C. was added triethylamine (198 mg, 1.95 mmol) and a solution of S-(3-((chlorocarbonyl)oxy)butan-2-yl) methanesulfonothioate (362 mg, 1.46 mmol) in anhydrous dichloromethane (2 mL) slowly at 23° C. The mixture was stirred at 23° C. for 16 hours then was concentrated under reduced pressure. The residue was purified by flash chromatography on silica gel (0-70% ethyl acetate in petroleum ether) to afford the title compound (120 mg, 30%) as a white solid. LCMS (ESI) m/z: 825.3 [M+H]⁺.

### Synthesis of S-(3-(((((3R,5S)-1-((R)-3-Methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

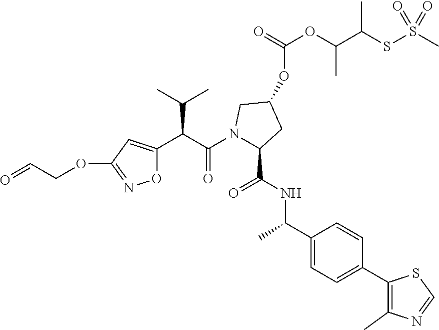 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCC=O)no2)C(C)C)cc1]

To a solution of S-(3-(((((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (120 mg, 0.15 mmol) was added formic acid (2 mL, 2 mmol) in water (1 mL) at 23° C. The mixture was stirred at 50° C. for 1 h then was concentrated to afford the title compound (105 mg, 96%) as yellow oil. LCMS (ESI) m/z: 751.2 [M+H]⁺.

### Synthesis of S-(3-(((((3R,5S)-1-((2R)-2-(3-(2-((3R)-4-(2-((4-(3-(3-Amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

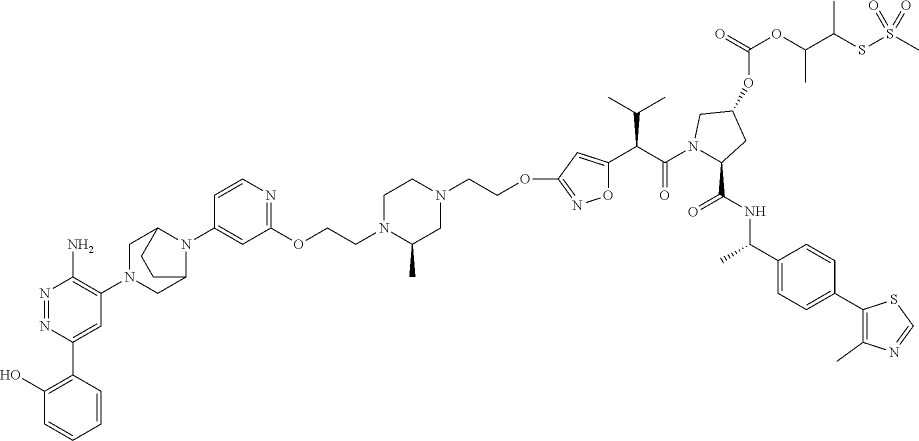 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a 23° C. solution of S-(3-(((((3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (105 mg, 0.14 mmol) and 2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenol [Compound 3 in the above Example 1 Scheme. This is compound 4 in the Example 4 Scheme below.] (73 mg, 0.14 mmol) and HOAc (0.2-0.3 mL) in dichloromethane (2 mL) and methanol (2 mL) was added NaBH(OAc)₃ (593 mg, 2.80 mmol). The reaction mixture was stirred at 23° C. for 3 hours then was concentrated. The residue was purified by prep-TLC (8% methanol in dichloromethane) to afford the title compound (48 mg, 27%) as a white solid. ¹H NMR (400 MHz, DMSO-d₆): δ 14.21-14.08 (m, 1H), 9.01-8.97 (m, 1H), 8.59-8.46 (m, 1H), 7.92 (d, J=4.8 Hz, 1H), 7.79 (d, J=6.0 Hz, 1H), 7.55-7.33 (m, 5H), 7.28-7.19 (m, 1H), 6.96-6.79 (m, 2H), 6.60-6.48 (m, 1H), 6.18-6.08 (m, 2H), 6.05-5.91 (m, 2H), 5.24-5.11 (m, 1H), 4.99-4.86 (m, 2H), 4.57-4.44 (m, 2H), 4.43-4.35 (m, 1H), 4.31-4.17 (m, 4H), 3.94-3.79 (m, 2H), 3.77-3.67 (m, 2H), 3.61-3.51 (m, 2H), 3.29-3.23 (m, 4H), 3.21-3.14 (m, 3H), 3.04-2.93 (m, 3H), 2.87-2.78 (m, 1H), 2.64-2.58 (m, 3H), 2.48-2.41 (m, 3H), 2.38-2.31 (m, 2H), 2.20-2.16 (m, 2H), 2.07-1.82 (m, 2H), 1.49-1.21 (m, 10H), 1.06-0.90 (m, 6H), 0.88-0.76 (m, 3H); LCMS (ESI) m/z: 1251.0 [M+H]⁺.

### Synthesis Example 2

### Syntheses of L1-CIDE-BRM1-2

L1-CIDE-BRM1-2 was synthesized by the following scheme, Scheme 2:

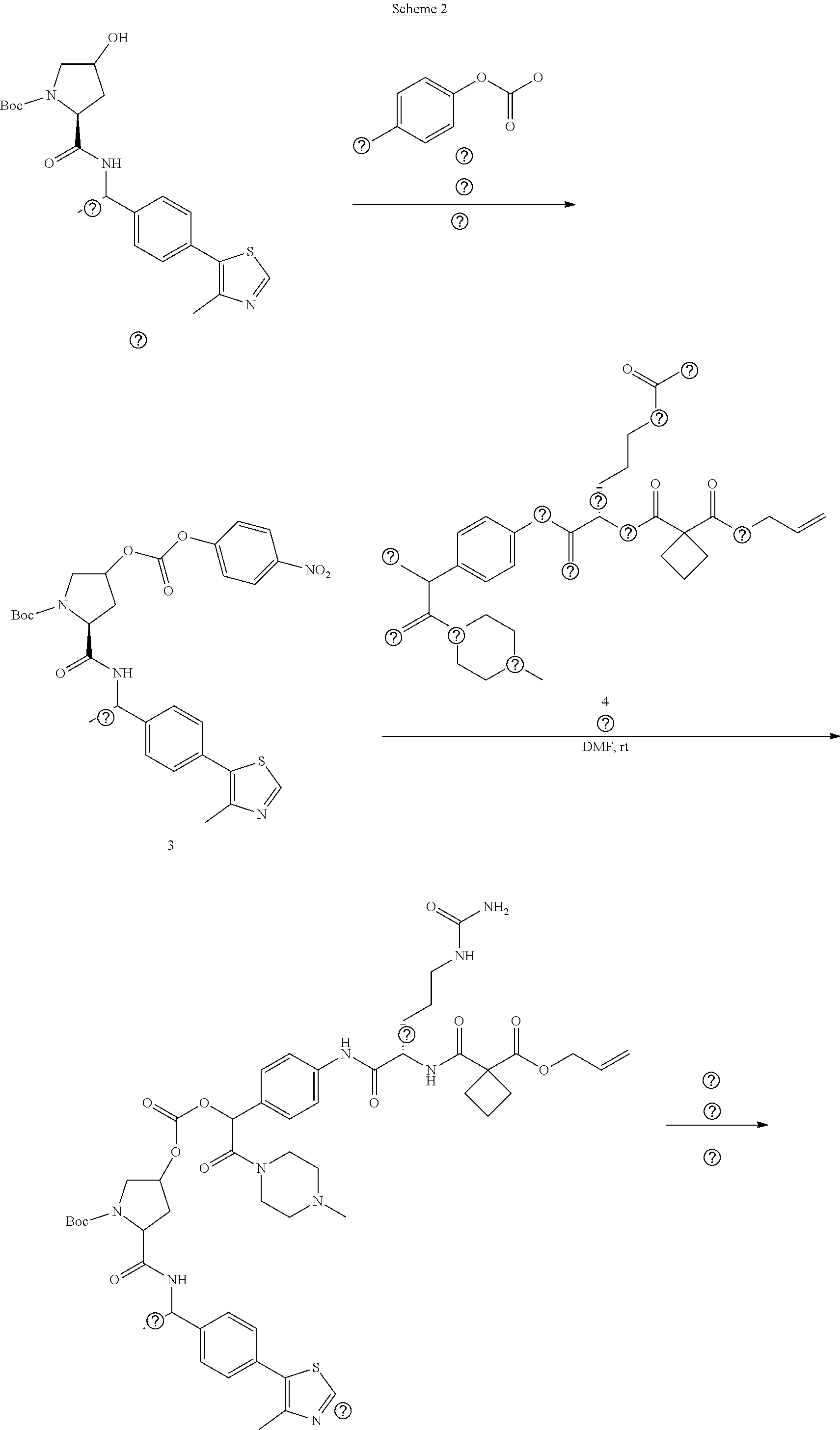 [SMILES: Cc1ncsc1-c1ccc(C(C)NC(=O)[C@@H]2CC(O)CN2C(=O)OC(C)(C)C)cc1 ; Cc1ccc(OC(=O)O)cc1 ; Cc1ncsc1-c1ccc(C(C)NC(=O)[C@@H]2CC(OC(=O)Oc3ccc([N+](=O)[O-])cc3)CN2C(=O)OC(C)(C)C)cc1 ; C=CCCC(=O)C1(C(=O)C[C@@H](CCCCC(C)=O)C(=C)Cc2ccc(C(C)C(=C)C3CCC(C)CC3)cc2)CCC1 ; C=CCOC(=O)C1(C(=O)N[C@@H](CCCNC(N)=O)C(=O)Nc2ccc(C(OC(=O)OC3CC(C(=O)NC(C)c4ccc(-c5scnc5C)cc4)N(C(=O)OC(C)(C)C)C3)C(=O)N3CCN(C)CC3)cc2)CCC1]

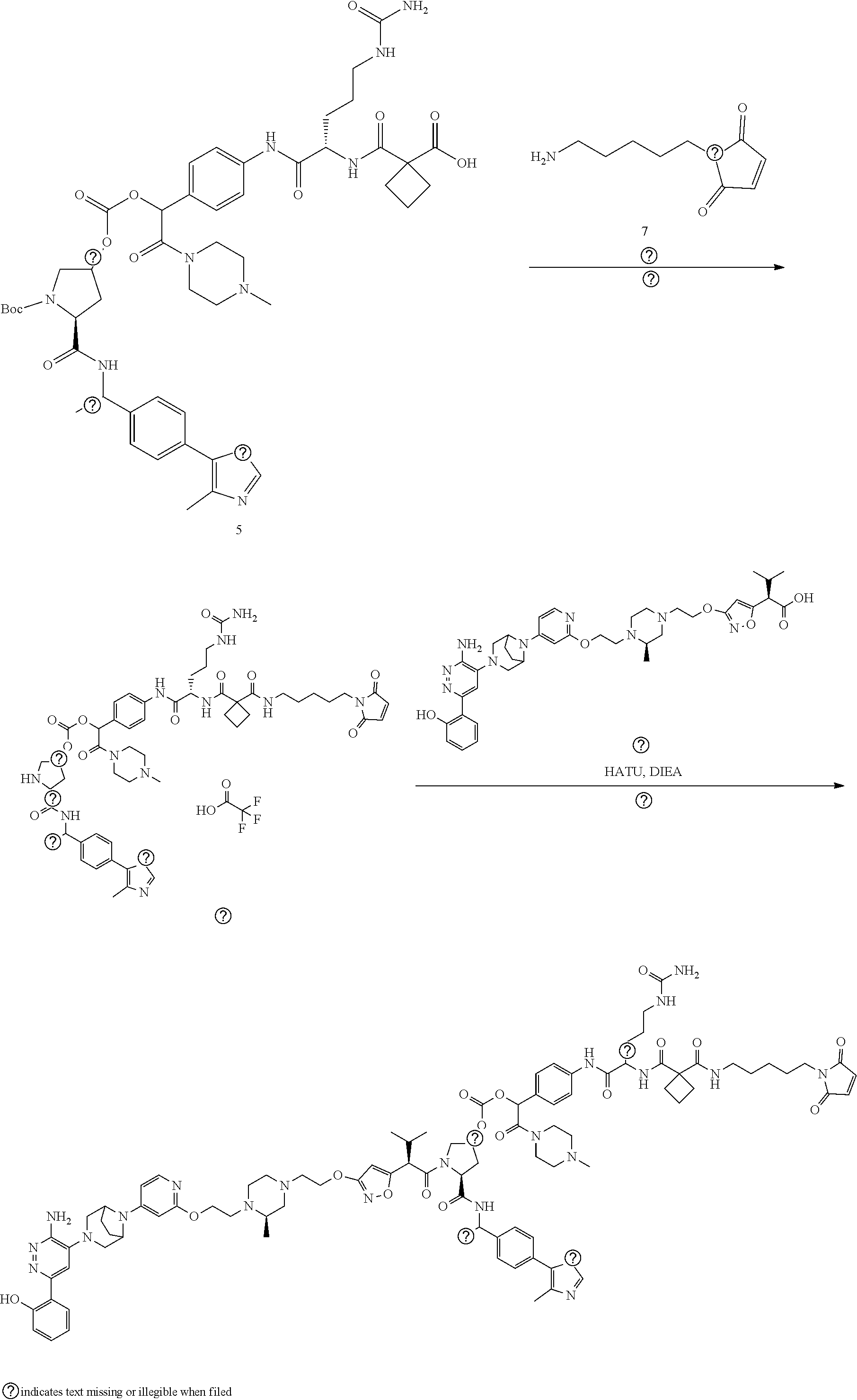

### Synthesis of (2S,4R)-tert-Butyl 2-(((S)-1-(4-(4-methylthiazol-5-yl)phenyl)ethyl)carbamoyl)-4-(((4-nitrophenoxy)carbonyl)oxy)pyrrolidine-1-carboxylate

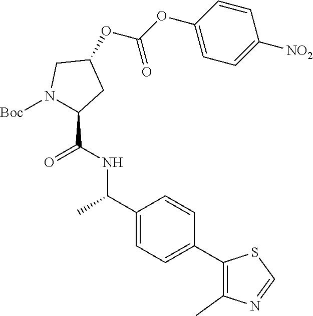 [SMILES: Cc1ncsc1-c1ccc([C@H](C)NC(=O)[C@@H]2C[C@@H](OC(=O)Oc3ccc([N+](=O)[O-])cc3)CN2C(=O)OC(C)(C)C)cc1]

To a mixture of 4-nitrophenyl carbonochloridate (1.68 g, 8.34 mmol) and (2S,4R)-tert-butyl-4-hydroxy-2-(((S)-1-(4-(4-methylthiazol-5-yl)phenyl)ethyl)carbamoyl)pyrrolidine-1-carboxylate (See Compound 1 in the Example Scheme 2 above; see J. Med. Chem. 2019, 62, 941 or J. Med. Chem. 2014, 57, 8657 for preparation) (3.0 g, 6.95 mmol) in anhydrous dichloromethane (80 mL) at 23° C. was added 2,6-lutidine (1.12 g, 10.4 mmol). The reaction mixture was stirred at 23° C. for 18 hours then was concentrated to afford the title compound (4.0 g, 99%) as a yellow solid. This material was used directly in next step. LCMS (ESI) m/z: 597.2 [M+H]⁺.

### Compound 4, Scheme 2: Synthesis of allyl 1-(((2S)-1-((4-(1-hydroxy-2-(4-methylpiperazin-1-yl)-2-oxoethyl)phenyl)amino)-1-oxo-5-ureidopentan-2-yl)carbamoyl)cyclobutanecarboxylate via Scheme 2a

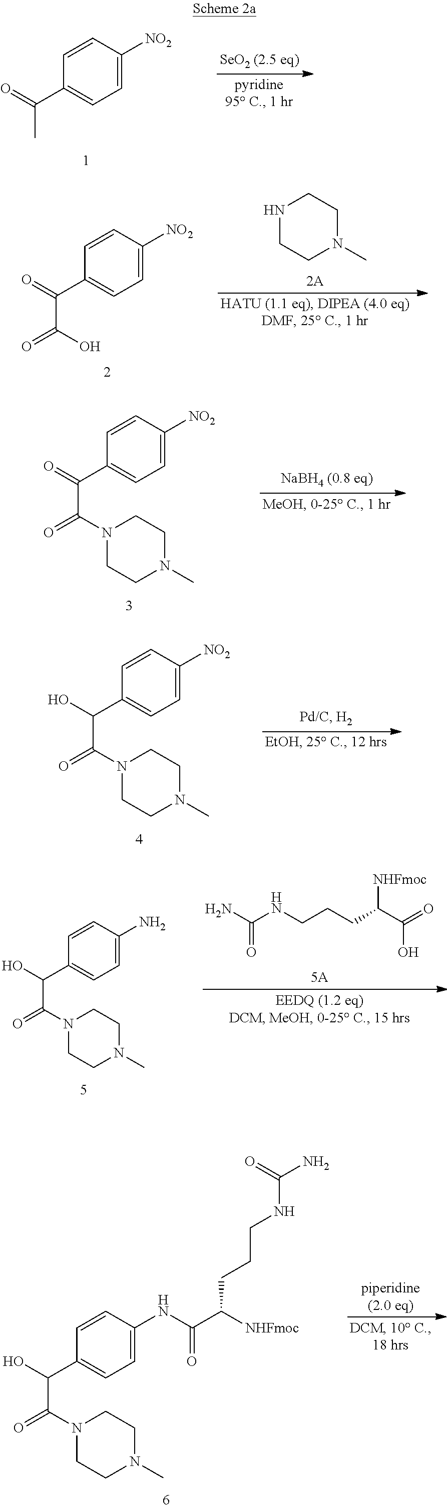

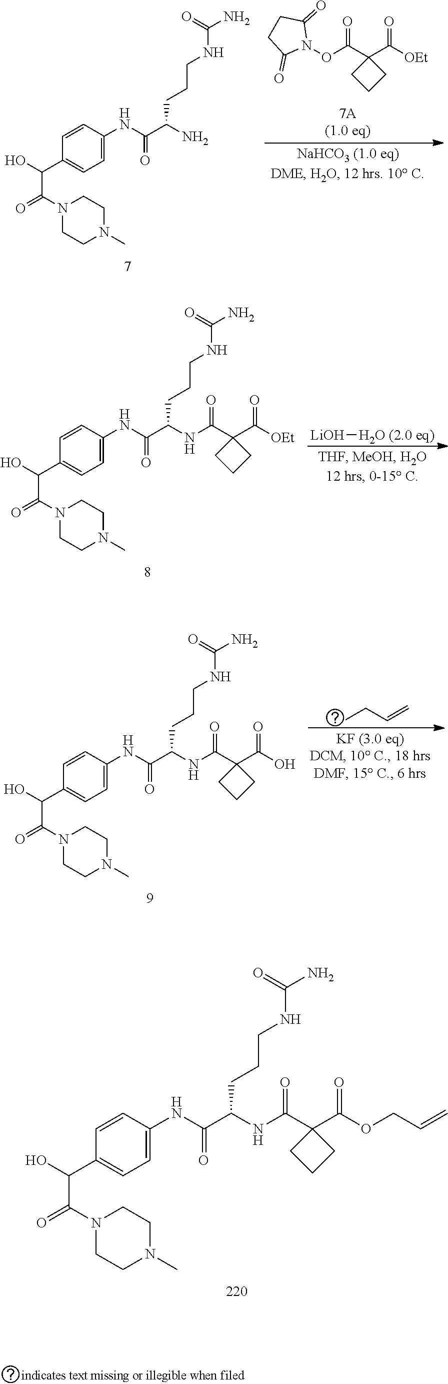

### i. General Procedure for Preparation of Compound 2 of Scheme 2a

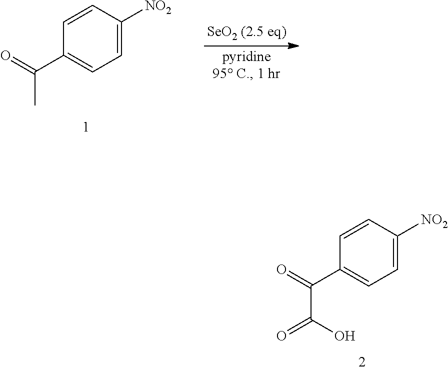

Four separate reactions were carried out in parallel. To a solution of compound 1 (200 g, 1.21 mol) in pyridine (3.00 L) was added SeO₂ (336 g, 3.03 mol) at 23° C. The mixture was then heated in an oil bath at 95° C. for 1 hour. The four reactions were combined for workup. The combined reactions were filtered at 45-50° C., and the filtrate was then cooled to 23° C. and maintained at that temperature for 1.5 hours. The mixture was filtered and the filter cake was dried in vacuum to give compound 2. The filtrate was concentrated under reduced pressure to 5.00 L, and was the stirred at 23° C. for 12 hours. The mixture was filtered and the filter cake dried in vacuum to give additional compound 2 (both lots combined=830 g, 88% yield) as a yellow solid. ¹H NMR (400 MHz, DMSO-d₆): δ 8.63-8.64 (m, 2H), 8.37-8.40 (m, 2H), 8.17-8.19 (m, 2H), 7.90-7.94 (m, 1H), 7.48-7.52 (m, 2H).

### ii. General Procedure for Preparation of Compound 3 of Scheme 2a

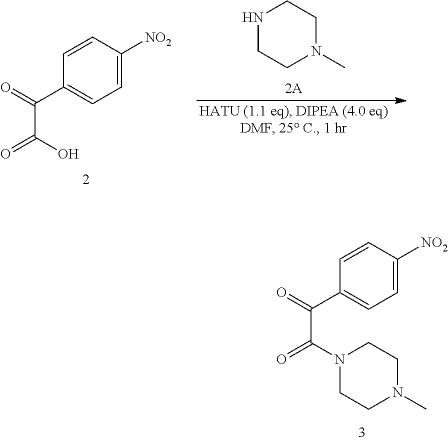

Two reactions were carried out in parallel. To a 23° C. solution of compound 2 (140 g, 538 mmol) in DMF (700 mL) was added compound 2A (54 g, 538 mmol), HATU (225 g, 592 mmol) and DIPEA (278 g, 2.15 mol). The mixture was stirred at 23° C. for 1 hour. The two reactions were then combined for workup. The combined reaction mixtures were diluted with DCM (3.00 L) and were washed with brine (1.00 L×3). The organic layer was dried over Na₂SO₄, filtered, and was concentrated under reduced pressure. The residue was purified by column chromatography (SiO₂, petroleum ether/ethyl acetate=50/1 to 0/1) to give compound 3 (200 g, 67% yield) as a yellow solid.

### iii. General Procedure for Preparation of Compound 4 of Scheme 2a

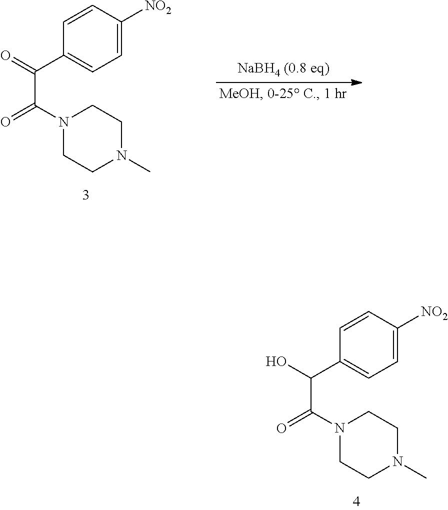 [SMILES: CN1CCN(C(=O)C(=O)c2ccc([N+](=O)[O-])cc2)CC1 ; CN1CCN(C(=O)C(O)c2ccc([N+](=O)[O-])cc2)CC1]

To a solution of compound 3 (184 g, 531 mmol) in MeOH (1.30 L) was added NaBH₄ (16.1 g, 425 mmol) at 0° C. The mixture was warmed to 23° C. and was stirred at that temperature for 1 hour. The reaction mixture was concentrated under reduced pressure, and the residue was diluted with water, adjusted to pH=7 with HCl (1 M), and extracted with EtOAc (1.00 L×3). The combined organic layers were dried over Na₂SO₄, filtered, and were concentrated under reduced pressure. The residue was purified by column chromatography (SiO₂, dichloromethane/methanol=100/1 to 10/1). The crude product was triturated with EtOH (500 mL) at 23° C. for 10 mins to give compound 4 (161 g, 54% yield) as a yellow solid. ¹H NMR: (400 MHz, DMSO-d₆): δ 8.24 (d, J=8.4 Hz, 2H), 7.64 (d, J=8.4 Hz, 2H), 6.14 (d, J=6.4 Hz, 1H), 5.60 (d, J=6.0 Hz, 1H), 3.57-3.47 (m, 2H), 3.43 (s, 2H), 2.23 (s, 2H), 2.12 (s, 5H).

### iv. General Procedure for Preparation of Compound 5 of Scheme 2a

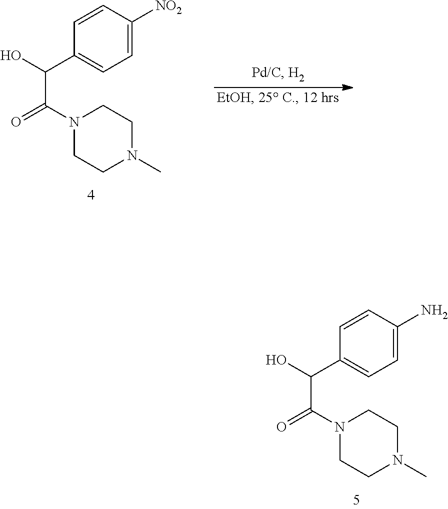 [SMILES: CN1CCN(C(=O)C(O)c2ccc([N+](=O)[O-])cc2)CC1 ; CN1CCN(C(=O)C(O)c2ccc(N)cc2)CC1]

Four reactions were carried out in parallel. To a solution of compound 4 (40 g, 143 mmol) in EtOH (600 mL) was added Pd/C (8.50 g, 10%) under an N₂ atmosphere. The suspension was degassed and purged with H₂ 3 times. The mixture was stirred under H₂ (15 psi) at 23° C. for 12 hours. The four reactions were then combined for workup. The combined reaction mixtures were filtered and the filter cake was washed with MeOH (1.00 L). The combined filtrate and washings were concentrated under reduced pressure to give compound 5 (140 g, 98% yield) as a yellow solid. ¹H NMR: (400 MHz, CD₃OD): δ 7.11 (d, J=8.4 Hz, 2H), 6.72 (d, J=8.4 Hz, 2H), 5.29 (s, 1H), 3.74 (s, 1H), 3.62-3.49 (m, 1H), 3.47-3.35 (m, 2H), 2.48 (s, 1H), 2.35-2.25 (m, 2H), 2.22 (s, 3H), 1.90 (s, 1H).

### v. General Procedure for Preparation of Compound 6 of Scheme 2a

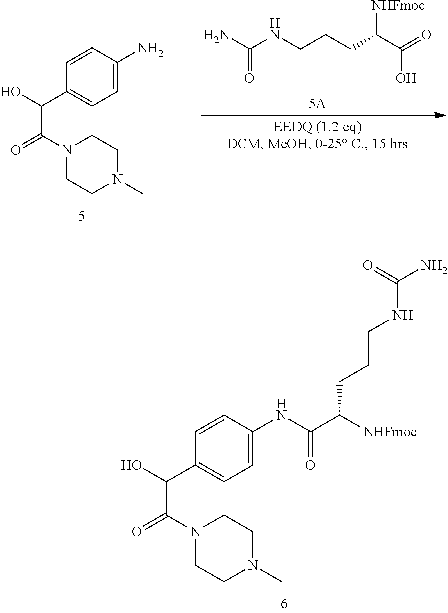

To the solution of Fmoc-L-citrulline (compound 5A) (95 g, 239 mmol) and compound 5 (72 g, 287 mmol) in MeOH (350 mL) and DCM (700 mL) was added EEDQ (71 g, 287 mmol) in one portion at 0° C. The mixture was warmed to 23° C. and was stirred at that temperature for 15 hours under N₂. The reaction mixture was concentrated under reduced pressure. The crude product was triturated with MTBE (1.00 L) at 15° C. for 2 hours to give compound 6 (185 g, crude) as an orange solid.

### vi. General Procedure for Preparation of Compound 7 of Scheme 2a

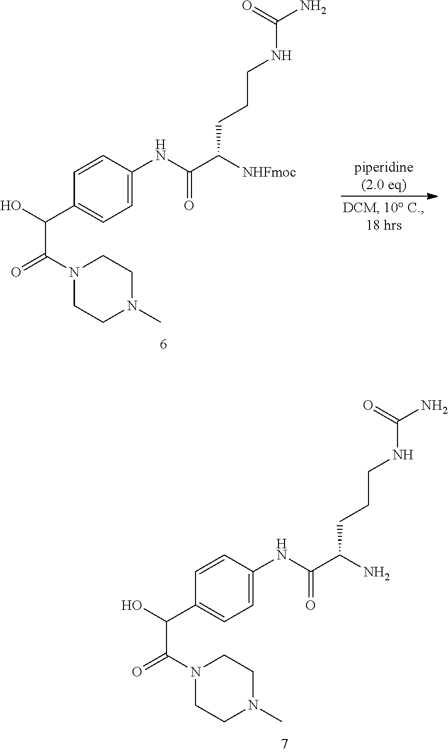

To a stirred solution of compound 6 (190 g, 302 mmol) in DCM (1.40 L) was added piperidine (52 g, 604 mmol) at 10° C. The mixture was stirred at 10° C. for 18 hours then was concentrated under reduced pressure. The residue was purified by column chromatography (SiO₂, dichloromethane/methanol=100/1 to 3/1) to give compound 7 (85 g, 68% yield) as a yellow oil. ¹H NMR (400 MHz, CD₃OD): δ 7.65 (d, J=8.4 Hz, 2H), 7.37 (d, J=8.4 Hz, 2H), 5.43 (s, 1H), 3.68 (s, 1H), 3.62 (d, J=4.0 Hz, 1H), 3.53-3.44 (m, 2H), 3.24-3.06 (m, 2H), 2.81 (d, J=5.2 Hz, 2H), 2.45 (s, 1H), 2.36-2.25 (m, 2H), 2.22 (s, 3H), 1.97 (s, 1H), 1.86-1.75 (m, 1H), 1.68-1.54 (m, 6H).

### vii. General Procedure for Preparation of Compound 8 of Scheme 2a

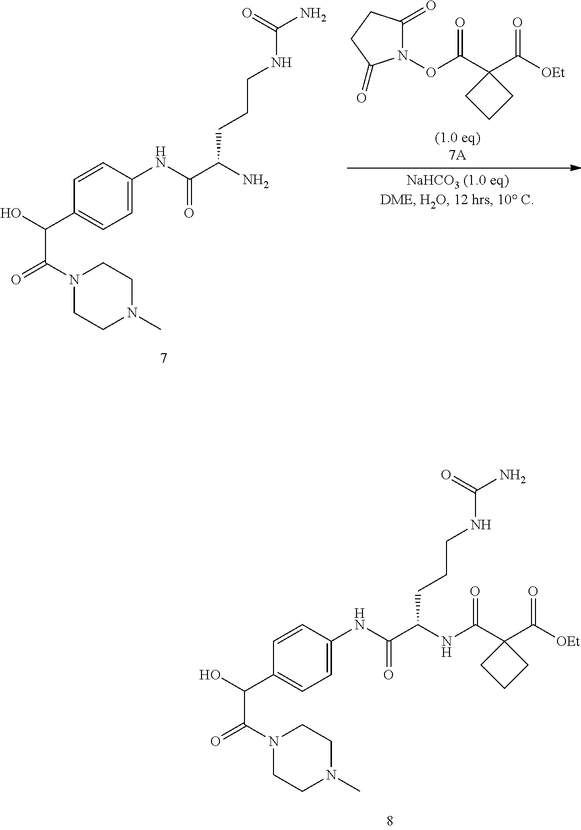 [SMILES: CN1CCN(C(=O)C(O)c2ccc(NC(=O)[C@@H](N)CCCNC(N)=O)cc2)CC1 ; CCOC(=O)C1(C(=O)ON2C(=O)CCC2=O)CCC1 ; CCOC(=O)C1(C(=O)N[C@@H](CCCNC(N)=O)C(=O)Nc2ccc(C(O)C(=O)N3CCN(C)CC3)cc2)CCC1]

To a 10° C. solution of compound 7 (76 g, 187 mmol) in DME (470 mL) and H₂O (290 mL) was added compound 7A (63 g, 234 mmol) and NaHCO₃ (20 g, 234 mmol). The mixture was stirred at 10° C. for 12 hours then was concentrated under reduced pressure. The residue was purified by column chromatography (SiO₂, dichloromethane/methanol=10/1 to 3/1) to give compound 8 (92 g, 70% yield) as a yellow solid.

### viii. General Procedure for Preparation of Compound 9 of Scheme 2a

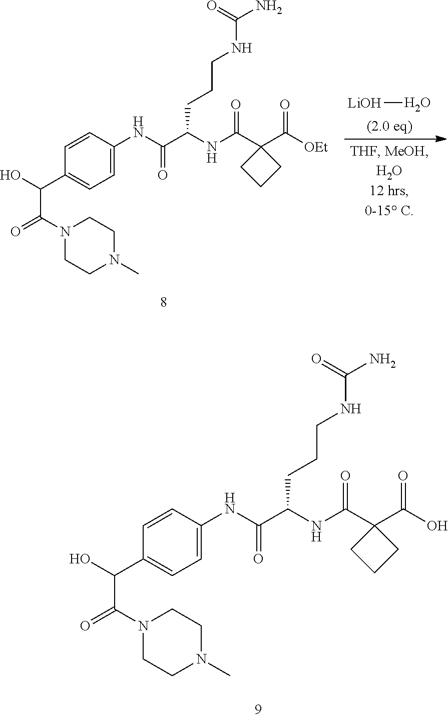

To a stirred solution of compound 8 (63 g, 112 mmol) in THF (190 mL) and MeOH (95 mL) was added a solution of LiOH·H₂O (9.43 g, 225 mmol) in H₂O (190 mL) at 0° C. The reaction mixture was warmed to 15° C. and was stirred at that temperature for 12 hours. The reaction mixture was then concentrated under reduced pressure. Purification of the residue by reversed-phase HPLC (0.1% TFA condition) gave compound 9 (50. g, 81% yield) as a white solid. ¹H NMR (400 MHz, CD₃OD): δ 7.68 (d, J=7.6 Hz, 2H), 7.37 (d, J=8.4 Hz, 2H), 5.48 (s, 1H), 4.50-4.53 (m, 1H), 3.78 (s, 2H), 3.69-3.56 (m, 1H), 3.27-3.10 (m, 5H), 2.79 (s, 3H), 2.71-2.62 (m, 2H), 2.60-2.50 (m, 2H), 2.17-2.07 (m, 1H), 2.06-2.03 (m, 4H), 2.03-1.97 (m, 1H), 1.97-1.86 (m, 1H), 1.74-1.78 (m, 1H), 1.69-1.53 (m, 2H).

### ix. General Procedure for Preparation of Compound 4 of Scheme 2 (220)

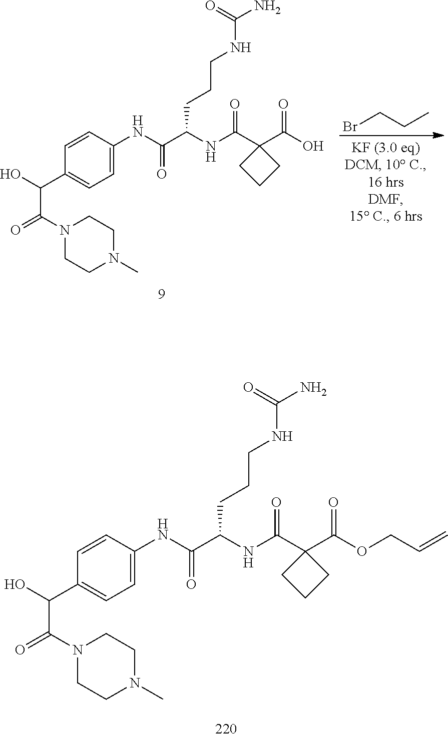

To a 15° C. solution of compound 9 (70 g, 131 mmol) in DMF (350 mL) was added KF (23 g, 394 mmol) and Bu₄NHSO₄ (12.5 g, 36.8 mmol). 3-Bromoprop-1-ene (398 g, 3.29 mol) was then added dropwise at 15° C., and the mixture was stirred at that temperature for an additional 6 hours. The reaction mixture was filtered, and the filtrated was concentrated under reduced pressure. The residue was purified by prep-HPLC (column: Phenomenex luna c18 250 mm*100 mm*10 um; mobile phase: [water (0.1% TFA)-ACN]; B %: 0%-20%, 30 min) to give 220 (26 g, 34% yield) as a white solid. ¹H NMR (400 MHz, CD₃OD): δ 7.69 (d, J=8.4 Hz, 2H), 7.39 (d, J=8.4 Hz, 2H), 6.02 (s, 1H), 5.75 (d, J=10.0 Hz, 2H), 5.49 (s, 1H), 4.50-4.54 (m, 1H), 4.34-4.09 (m, 1H), 4.03 (s, 2H), 3.96-3.51 (m, 3H), 3.50-3.33 (m, 3H), 3.26-3.06 (m, 6H), 2.75-2.49 (m, 4H), 2.22-2.08 (m, 1H), 2.06-1.87 (m, 2H), 1.81-1.72 (m, 1H), 1.70-1.52 (m, 2H). LCMS: (M+H⁺=573.3)

### Compound 6, Scheme 2: Synthesis of 1-(((2S)-1-((4-(1-(((((3R,5S)-1-(tert-Butoxycarbonyl)-5-(((S)-1-(4-(4-methylthiazol-5-yl)phenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid

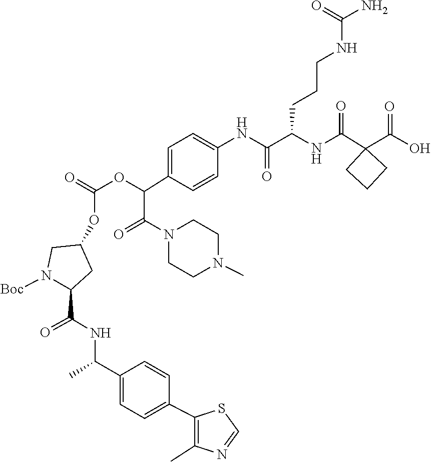 [SMILES: Cc1ncsc1-c1ccc([C@H](C)NC(=O)[C@@H]2C[C@@H](OC(=O)OC(C(=O)N3CCN(C)CC3)c3ccc(NC(=O)[C@H](CCCNC(N)=O)NC(=O)C4(C(=O)O)CCC4)cc3)CN2C(=O)OC(C)(C)C)cc1]

To a solution of (2S,4R)-tert-butyl 4-(((1-(4-((S)-2-(1-((allyloxy)carbonyl)cyclobutanecarboxamido)-5-ureidopentanamido)phenyl)-2-(4-methylpiperazin-1-yl)-2-oxoethoxy)carbonyl)oxy)-2-(((S)-1-(4-(4-methylthiazol-5-yl)phenyl)ethyl)carbamoyl)pyrrolidine-1-carboxylate (76 mg, 0.07 mmol) and 1,3-dimethylpyrimidine-2,4,6(1H,3H,5H)-trione (58 mg, 0.37 mmol) in dichloromethane (5 mL) and methyl alcohol (5 mL) at 23° C. was added Pd(PPh₃)₄ (17 mg, 0.01 mmol). The reaction mixture was stirred under nitrogen atmosphere at 23° C. for 10 hours then was concentrated. The residue was purified by prep-HPLC with the following conditions: Column: Phenomenex Gemini-NX 80*30 mm*3 um, mobile phase: (25-45%) water (10 mM NH₄HCO₃)-ACN to afford the title compound (50 mg, 69%) as a yellow solid. LCMS (ESI) m/z: 990.6 [M+H]⁺.

### Synthesis of (2S,4R)-tert-Butyl 4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)-2-(((S)-1-(4-(4-methylthiazol-5-yl)phenyl)ethyl)carbamoyl)pyrrolidine-1-carboxylate

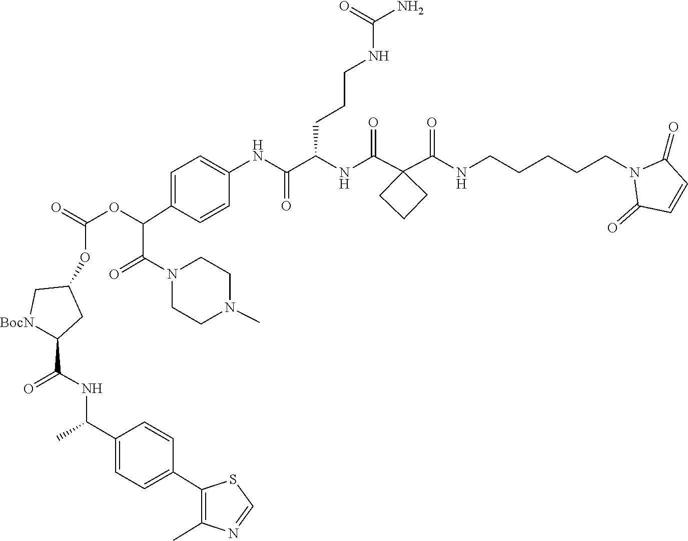 [SMILES: Cc1ncsc1-c1ccc([C@H](C)NC(=O)[C@@H]2C[C@@H](OC(=O)OC(C(=O)N3CCN(C)CC3)c3ccc(NC(=O)[C@H](CCCNC(N)=O)NC(=O)C4(C(=O)NCCCCCN5C(=O)C=CC5=O)CCC4)cc3)CN2C(=O)OC(C)(C)C)cc1]

To a mixture of 1-(((2S)-1-((4-(1-(((((3R,5S)-1-(tert-butoxycarbonyl)-5-(((S)-1-(4-(4-methylthiazol-5-yl)phenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid (69 mg, 0.07 mmol) and 1-(5-aminopentyl)-1H-pyrrole-2,5-dione (16 mg, 0.08 mmol) in DMF (8 mL) at 23° C. was added N,N-diisopropylethylamine (0.03 mL, 0.21 mmol) and HATU (32 mg, 0.08 mmol). The reaction mixture was stirred at 23° C. for 16 hours then was concentrated. The residue was purified by prep-HPLC (Boston Green ODS 150*30 mm*5 um, (25-45%) water (0.075% TFA)-ACN) to afford the title compound (67 mg, 84%) as a white solid. LCMS (ESI) m/z: 1155.6 [M+H]⁺.

### Compound 7, Scheme 2: Synthesis of 1-(4-((S)-2-(1-((5-(2,5-Dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl ((3R,5S)-5-(((S)-1-(4-(4-methylthiazol-5-yl)phenyl)ethyl)carbamoyl)pyrrolidin-3-yl) carbonate 2,2,2-trifluoroacetate

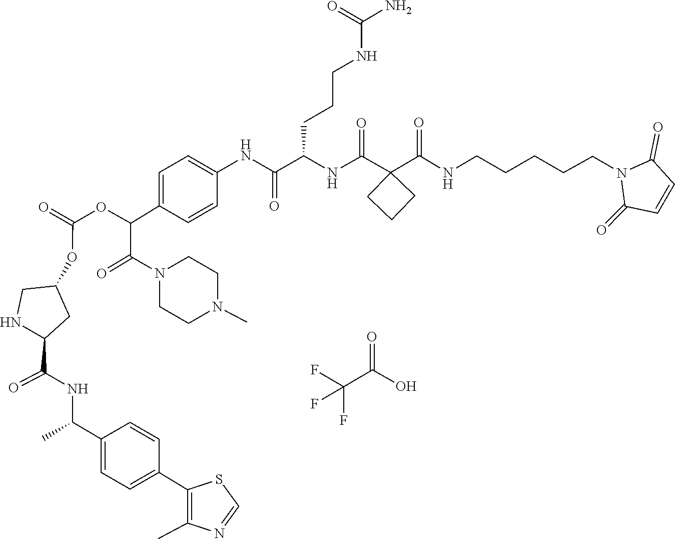 [SMILES: Cc1ncsc1-c1ccc([C@H](C)NC(=O)[C@@H]2C[C@@H](OC(=O)OC(C(=O)N3CCN(C)CC3)c3ccc(NC(=O)[C@H](CCCNC(N)=O)NC(=O)C4(C(=O)NCCCCCN5C(=O)C=CC5=O)CCC4)cc3)CN2)cc1 ; O=C(O)C(F)(F)F]

A solution of (2S,4R)-tert-butyl 4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)-2-(((S)-1-(4-(4-methylthiazol-5-yl)phenyl)ethyl)carbamoyl)pyrrolidine-1-carboxylate (67.4 mg, 0.06 mmol) in 5% TFA in HFIP (2 mL, 0.06 mmol) was stirred at 23° C. for 1.5 hours. The reaction mixture was then concentrated to afford the title compound (62 mg, 99.9%) as a yellow oil. LCMS (ESI) m/z: 1054.7 [M+H]⁺.

### L1-CIDE-BRM1-2: Synthesis of (3R,5S)-1-((2R)-2-(3-(2-((3R)-4-(2-((4-(3-(3-Amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate

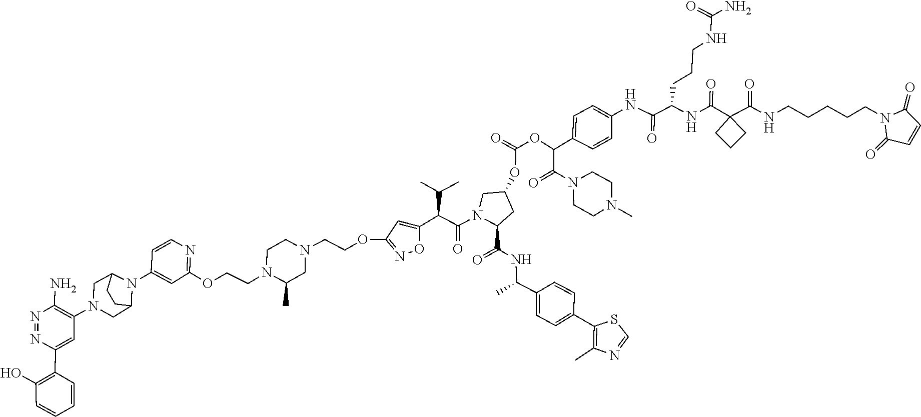 [SMILES: Cc1ncsc1-c1ccc([C@H](C)NC(=O)[C@@H]2C[C@@H](OC(=O)OC(C(=O)N3CCN(C)CC3)c3ccc(NC(=O)[C@H](CCCNC(N)=O)NC(=O)C4(C(=O)NCCCCCN5C(=O)C=CC5=O)CCC4)cc3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a mixture of 1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl ((3R,5S)-5-(((S)-1-(4-(4-methylthiazol-5-yl)phenyl)ethyl)carbamoyl)pyrrolidin-3-yl) carbonate 2,2,2-trifluoroacetate (62 mg, 0.06 mmol) and (2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid (50 mg, 0.07 mmol) in DMF (2.5 mL) at 23° C. was added N,N-diisopropylethylamine (0.03 mL, 0.18 mmol) and HATU (27 mg, 0.07 mmol). The reaction mixture was stirred at 23° C. for 16 hours then was concentrated. The residue was purified by prep-HPLC with the following conditions: Column: Phenomenex Gemini-NX 80*30 mm*3 um; mobile phase: (26-46%) water (10 mM NH₄HCO₃)-ACN to afford the title compound (44 mg, 42%) as a white solid. ¹H NMR (400 MHz, CD₃OD): δ 8.88-8.83 (m, 1H), 7.80-7.75 (m, 1H), 7.75-7.65 (m, 3H), 7.49-7.32 (m, 7H), 7.25-7.19 (m, 1H), 6.93-6.84 (m, 2H), 6.76 (s, 1H), 6.75-6.71 (m, 1H), 6.57-6.54 (m, 1H), 6.29-6.19 (m, 2H), 5.25-5.21 (m, 1H), 4.98-4.93 (m, 2H), 4.64-4.62 (m, 2H), 4.51 (s, 3H), 4.41-4.27 (m, 3H), 4.23-4.08 (m, 1H), 4.01-3.89 (m, 1H), 3.79-3.54 (m, 4H), 3.48-3.40 (m, 2H), 3.27-3.16 (m, 4H), 3.15-3.03 (m, 4H), 2.97-2.85 (m, 2H), 2.83-2.69 (m, 4H), 2.61-2.51 (m, 3H), 2.50-2.33 (m, 8H), 2.29-2.17 (m, 6H), 2.14-2.11 (m, 3H), 1.93 (s, 4H), 1.75 (s, 1H), 1.62-1.45 (m, 9H), 1.35-1.23 (m, 4H), 1.16-1.10 (m, 3H), 1.09-0.92 (m, 3H), 0.92-0.80 (m, 3H); LCMS (ESI) m/z: 1764.8 [M+H]⁺.

### Synthesis Example 3

### Syntheses of L1-CIDE-BRM1-3

L1-CIDE-BRM1-3 was synthesized by the following scheme, Scheme 3:

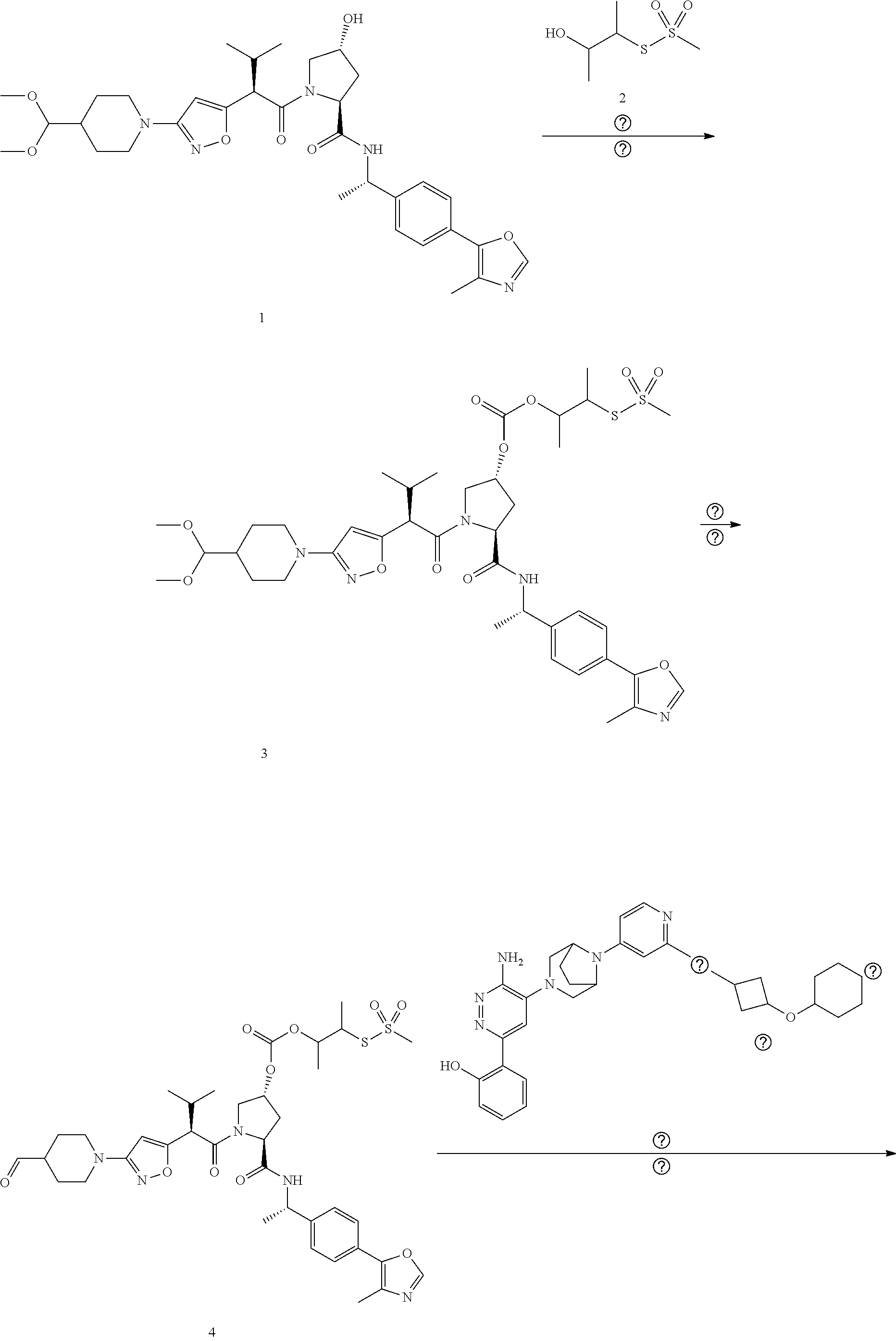

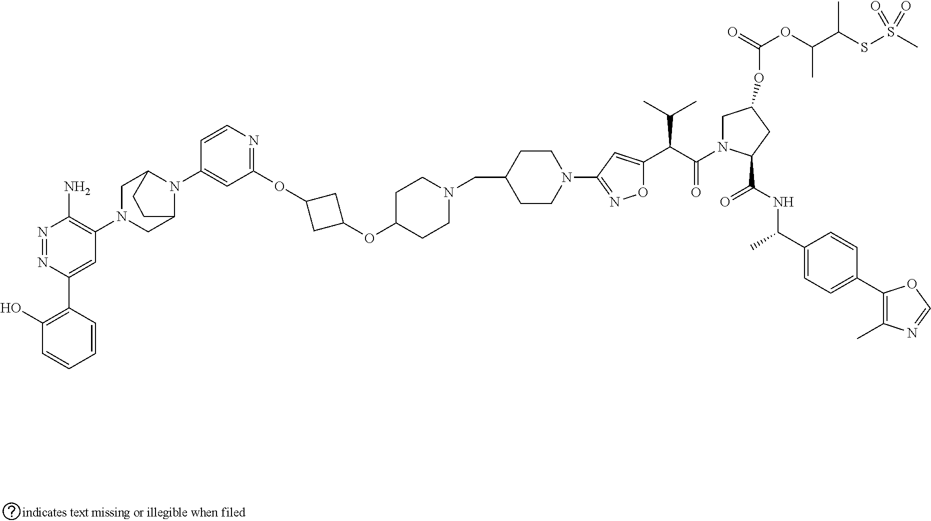 [SMILES: Cc1ncoc1-c1ccc([C@H](C)NC(=O)[C@@H]2C[C@@H](OC(=O)OC(C)C(C)SS(C)(=O)=O)CN2C(=O)[C@@H](c2cc(N3CCC(CN4CCC(OC5CC(Oc6cc(N7C8CCC7CN(c7cc(-c9ccccc9O)nnc7N)C8)ccn6)C5)CC4)CC3)no2)C(C)C)cc1]

### Synthesis of S-(3-((Chlorocarbonyl)oxy)butan-2-yl) methanesulfonothioate

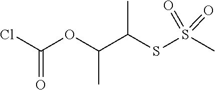

To a solution of S-(3-hydroxybutan-2-yl) methanesulfonothioate (300 mg, 1.63 mmol) in dichloromethane (2 mL) and pyridine (516 mg, 6.51 mmol) was added a solution of triphosgene (242 mg, 0.81 mmol) in dichloromethane (2 mL) at 23° C. The reaction was stirred at 23° C. for 30 min then was concentrated to dryness to afford the title compound (380 mg, 95%) as a yellow oil. This material was used directly in next step.

### Synthesis of S-(3-(((((3R,5S)-1-((R)-2-(3-(4-(Dimethoxymethyl)piperidin-1-yl)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

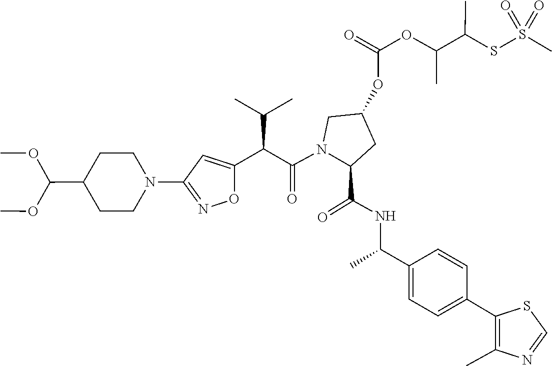

To a 23° C. mixture of (2S,4R)-1-((R)-2-(3-(4-(dimethoxymethyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (250 mg, 0.39 mmol) (See Compound 1 in Scheme 3 above; the preparation is described on pages 451-452 of US 2020/0038378, herein incorporated by reference in its entirety) and 4 Å MS (50 mg) in dichloromethane (2 mL) was added pyridine (0.09 mL, 1.17 mmol) and a solution of S-(3-((chlorocarbonyl)oxy)butan-2-yl) methanesulfonothioate (220 mg, 0.89 mmol) in dichloromethane (1 mL). The reaction was stirred at 23° C. for 30 min then was concentrated. The residue was purified by flash chromatography column on silica gel (0-60% dichloromethane in ethyl acetate) to afford the title compound 3 (130 mg, 39%) as a white solid. LCMS (ESI) m/z: 850.3 [M+H]⁺.

### Synthesis S-(3-(((((3R,5S)-1-((R)-2-(3-(4-Formylpiperidin-1-yl)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

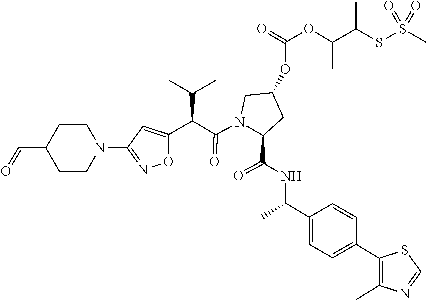 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(N3CCC(C=O)CC3)no2)C(C)C)cc1]

A solution of S-(3-(((((3R,5S)-1-((R)-2-(3-(4-(dimethoxymethyl)piperidin-1-yl)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (130 mg, 0.15 mmol) in water (1 mL) and formic acid (3 mL) was stirred at 50° C. for 2 hours. The reaction mixture was then concentrated to afford the title compound (120 mg, 98%) as a yellow oil. LCMS (ESI) m/z: 804.3 [M+H]⁺.

### L1-CIDE-BRM1-3: Synthesis of S-(3-(((((3R,5S)-1-((2R)-2-(3-(4-((4-(trans-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidin-1-yl)methyl)piperidin-1-yl)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

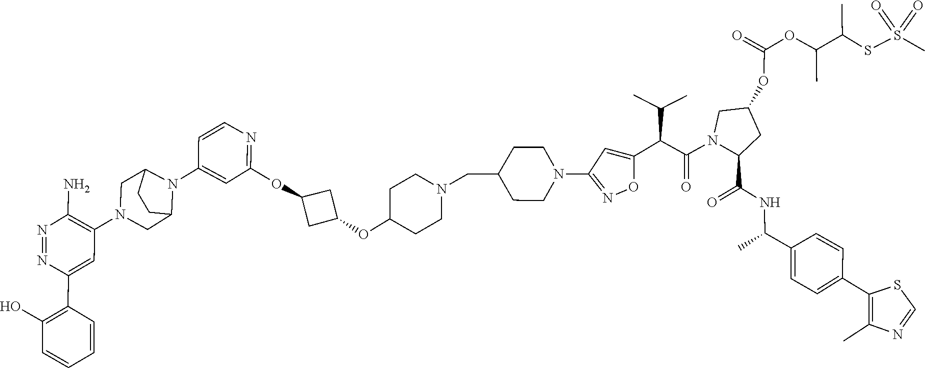 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(N3CCC(CN4CCC(O[C@H]5C[C@H](Oc6cc(N7C8CCC7CN(c7cc(-c9ccccc9O)nnc7N)C8)ccn6)C5)CC4)CC3)no2)C(C)C)cc1]

To a 23° C. solution of S-(3-(((((3R,5S)-1-((R)-2-(3-(4-formylpiperidin-1-yl)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (120 mg, 0.15 mmol) in dichloromethane (1 mL) and methanol (1 mL) was added 2-(6-amino-5-(8-(2-(trans-3-(piperidin-4-yloxy)cyclobutoxy)pyridin-4-yl)-3,8-diazabicyclo[3.2.1]octan-3-yl)pyridazin-3-yl)phenol hydrochloride (See Compound 5 in Scheme 3 above; see p 306-307 (top of page numbering) of US 2020/0038378 for preparation.) (82 mg, 0.15 mmol), HOAc (0.2 ml) and sodium triacetoxyborohydride (317 mg, 1.49 mmol). The reaction mixture was stirred at 23° C. for 3 hours then was concentrated. The crude residue was purified by prep-TLC (methanol:dichloromethane=1:10) to afford the title compound (31 mg, 14%) as a white solid. ¹H NMR (400 MHz, DMSO-d₆): δ 14.13 (s, 1H), 8.98 (s, 1H), 8.49 (d, J=7.2 Hz, 1H), 7.91 (d, J=7.2 Hz, 1H), 7.76 (d, J=6.4 Hz, 1H), 7.52-7.41 (m, 3H), 7.40-7.33 (m, 2H), 7.24-7.21 (m, 1H), 6.90-6.80 (m, 2H), 6.54-6.51 (m, 1H), 6.14-6.12 (m, 2H), 6.00-5.91 (m, 2H), 5.18-5.15 (m, 2H), 4.95-4.90 (m, 2H), 4.50-4.46 (m, 2H), 4.39-4.35 (m, 1H), 4.32-4.22 (m, 1H), 3.94-3.66 (m, 3H), 3.64-3.55 (m, 4H), 3.54-3.52 (m, 2H), 3.28-3.21 (m, 4H), 3.02-3.01 (m, 2H), 2.77-2.69 (m, 2H), 2.45 (s, 3H), 2.30-2.22 (m, 4H), 2.19-2.15 (m, 2H), 2.10-2.05 (m, 2H), 2.04-1.89 (m, 5H), 1.81-1.57 (m, 5H), 1.47-1.34 (m, 8H), 1.33-1.28 (m, 2H), 1.27-1.20 (m, 2H), 1.16-1.02 (m, 2H), 0.95-0.91 (m, 3H), 0.85-0.75 (m, 3H); LCMS (ESI) m/z: 1331.9 [M+H]⁺.

### Synthesis Example 4

### Syntheses of L1-CIDE-BRM1-4

L1-CIDE-BRM1-4 was synthesized by the following scheme, Scheme 4:

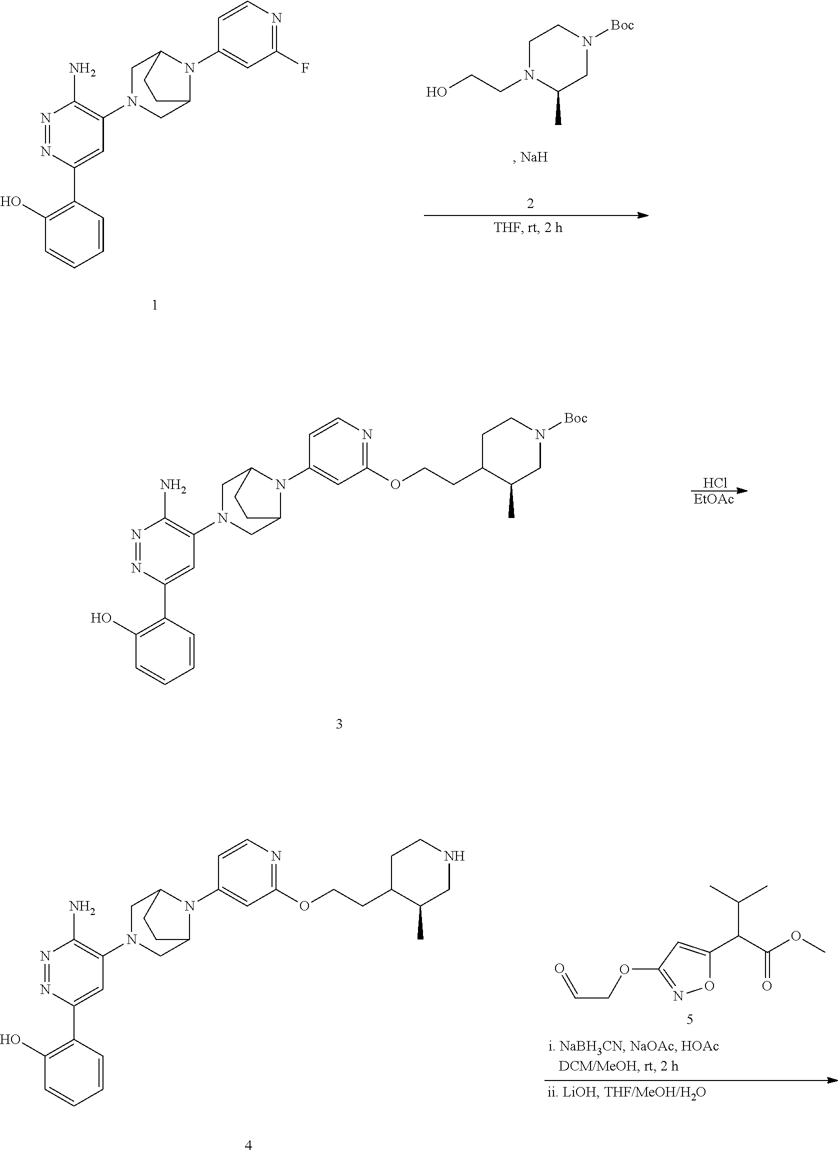

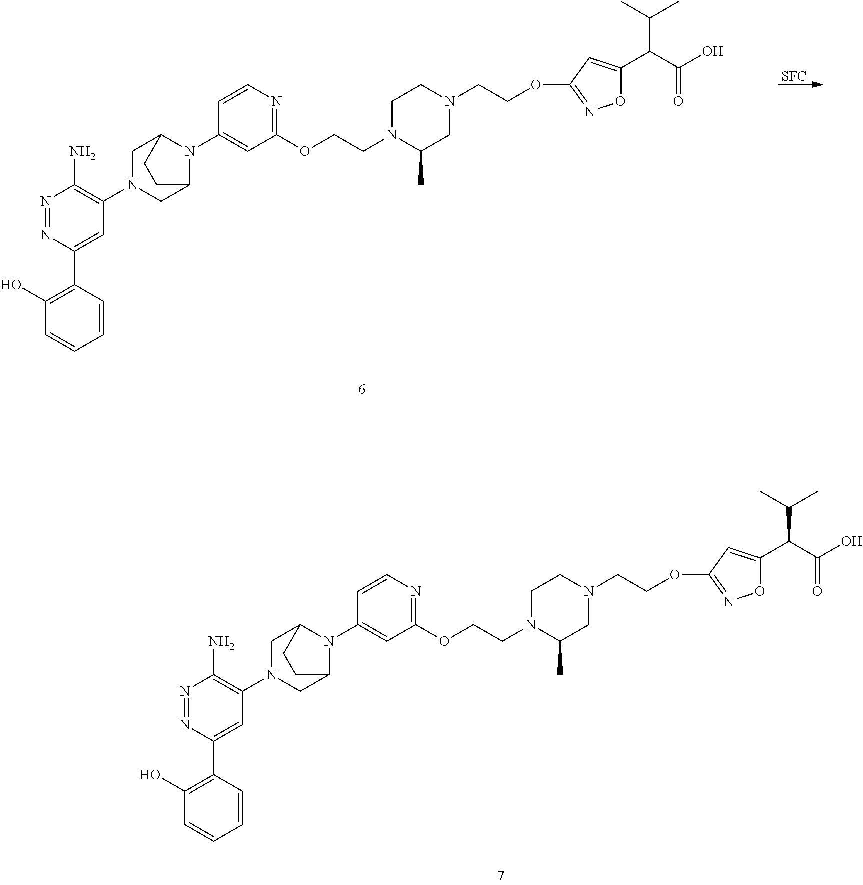 [SMILES: CC(C)C(C(=O)O)c1cc(OCCN2CCN(CCOc3cc(N4C5CCC4CN(c4cc(-c6ccccc6O)nnc4N)C5)ccn3)[C@H](C)C2)no1 ; "SFC" ; CC(C)[C@@H](C(=O)O)c1cc(OCCN2CCN(CCOc3cc(N4C5CCC4CN(c4cc(-c6ccccc6O)nnc4N)C5)ccn3)[C@H](C)C2)no1]

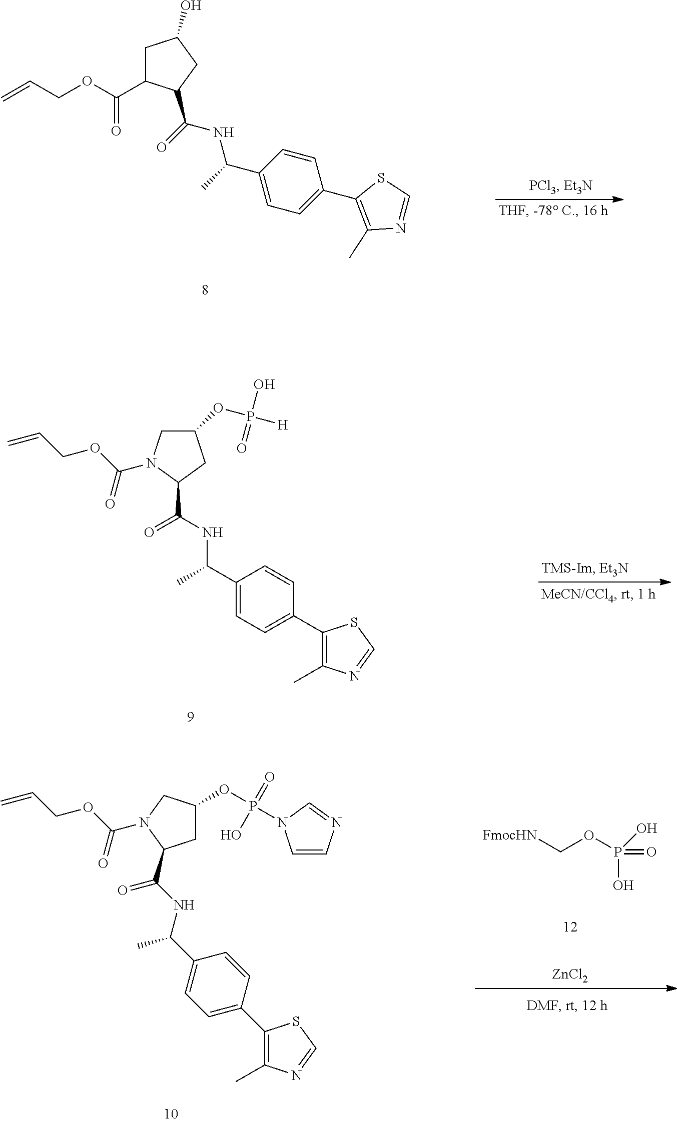

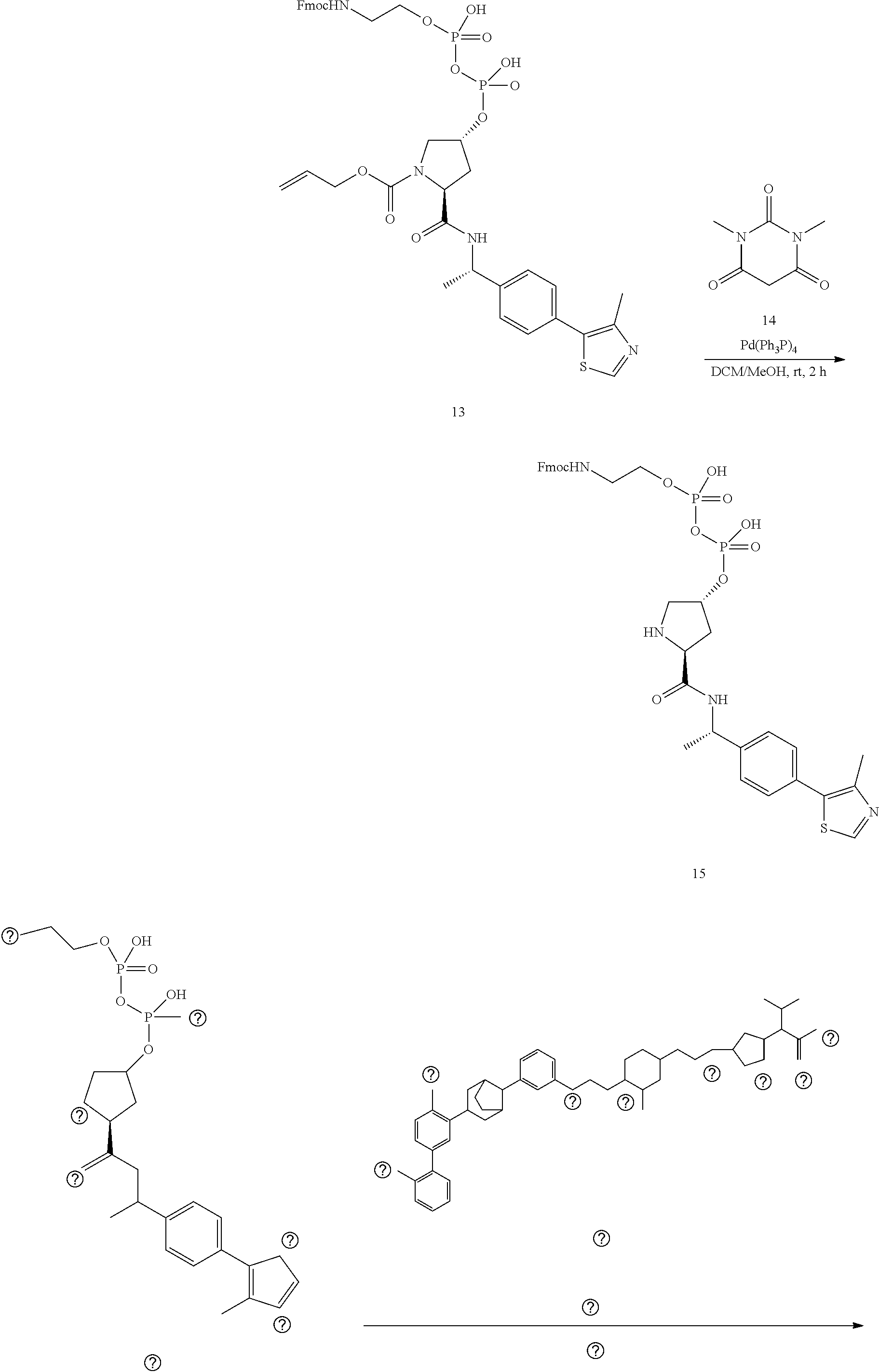

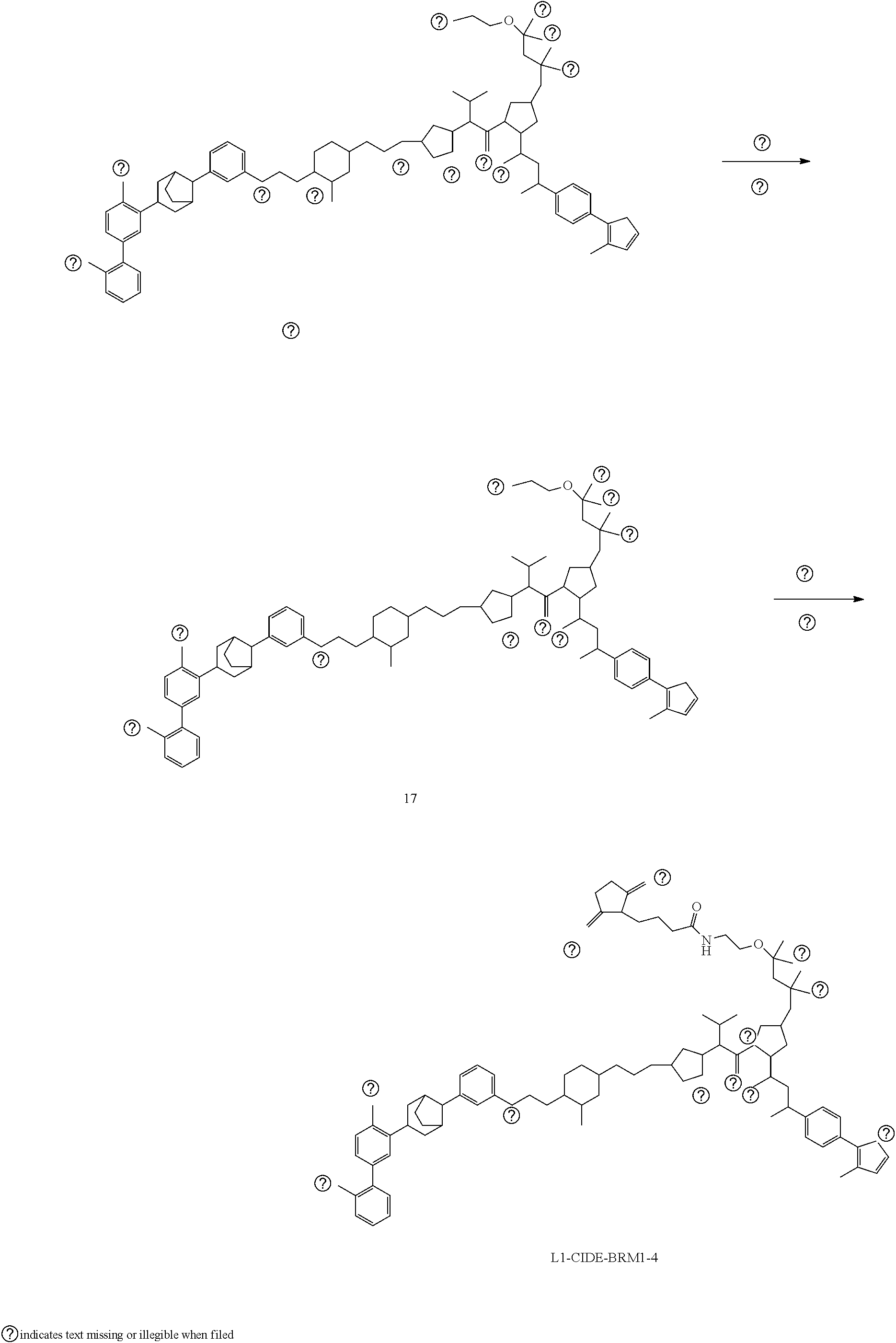 [SMILES: C=C(C1CC(CC(C)(C)CC(C)(C)OCCC)CC1C(C)CC(C)c1ccc(C2=C(C)C=CC2)cc1)C(C(C)C)C1CCC(CCCC2CCC(CCCc3cccc(C4C5CCC4CC(c4cc(-c6ccccc6C)ccc4C)C5)c3)C(C)C2)C1 ; C=C(C1CC(CC(C)(C)CC(C)(C)OCCC)CC1C(C)CC(C)c1ccc(C2=C(C)C=CC2)cc1)C(C(C)C)C1CCC(CCCC2CCC(CCCc3cccc(C4C5CCC4CC(c4cc(-c6ccccc6C)ccc4C)C5)c3)C(C)C2)C1 ; C=C1CCC(=C)C1CCCC(=O)NCCOC(C)(C)CC(C)(C)CC1CC(C(=C)C(C(C)C)C2CCC(CCCC3CCC(CCCc4cccc(C5C6CCC5CC(c5cc(-c7ccccc7C)ccc5C)C6)c4)C(C)C3)C2)C(C(C)CC(C)c2ccc(C3=C(C)C=CC3)cc2)C1]

### Compound 1, Scheme 4

### Step 1: Preparation of 182

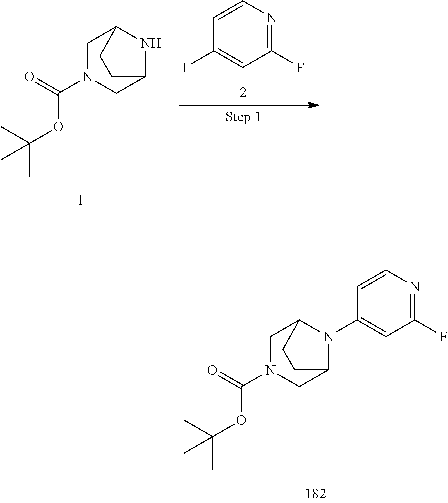

2-Fluoro-4-iodopyridine (2) (52 g, 230 mmol) was added to a 1-neck 2 L round bottom flask containing a magnetically stirred mixture of tert-butyl 3,8-diazabicyclo[3.2.1]octane-3-carboxylate (1) (37 g, 175 mmol), sodium tert-butoxide (26 g, 265 mmol), potassium fluoride (17 g, 284 mmol), and xantphos (4.8 g, 8.2 mmol) in 1,4-dioxane (750 mL). The mixture was purged with nitrogen gas for 15 minutes, then tris(dibenzylideneacetone)di-palladium(0) (3.7 g, 4.0 mmol) was added. The reaction flask was fitted with a condenser capped with a nitrogen inlet and was placed in a pre-heated oil bath set to 110° C. After stirring under a nitrogen atmosphere at 110° C. for 1.25 hours the resultant brown/red suspension was cooled to 23° C. and was filtered through Celite. The filter cake was washed with Et₂O, and the combined filtrate and washings were concentrated under reduced pressure to a red oil (127 g). The crude material was purified by flash chromatography using 0-40% EtOAc/DCM to provide 182 as a yellow foamy solid (56 g, ˜100%).

### Step 2: Preparation of 187

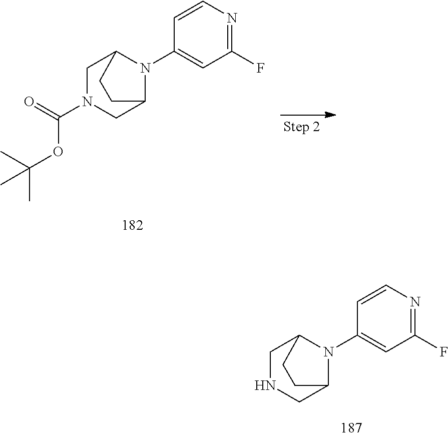 [SMILES: CC(C)(C)OC(=O)N1CC2CCC(C1)N2c1ccnc(F)c1 ; Fc1cc(N2C3CCC2CNC3)ccn1]

A solution of 4 M HCl in 1,4-dioxane (220 mL, 880 mmol) was added over 50 minutes to a magnetically stirred solution of 182 (56 g, ˜175 mmol) in MeCN (650 mL) within a 1-neck 2 L round-bottom flask at 23° C. The mixture was allowed to stir at 23° C. for 1 hour during which time it became a yellow/orange suspension. The reaction mixture was concentrated to a yellow solid, and this material was triturated with Et₂O (1000 mL) at 23° C. for 1 hour. The mixture was filtered, and the collected material was dried under reduced pressure to provide the tri-HCl salt of 187 as a yellow powder (61 g). The salt was suspended in DCM (1000 mL), and slowly neutralized with saturated aqueous NaHCO₃ (500 mL). The layers were separated, and the aqueous phase was further extracted with DCM (2×500 mL). The combined organic phases were washed with saturated NaCl (250 mL), dried over anhydrous Na₂SO₄, filtered, and concentrated to provide the free base of 187 as a yellow solid (32 g, 86%). Note: ensure that the NaHCO₃ solution is sufficiently saturated to prevent loss of product within the aqueous phase.

### Step 3: Preparation of 208

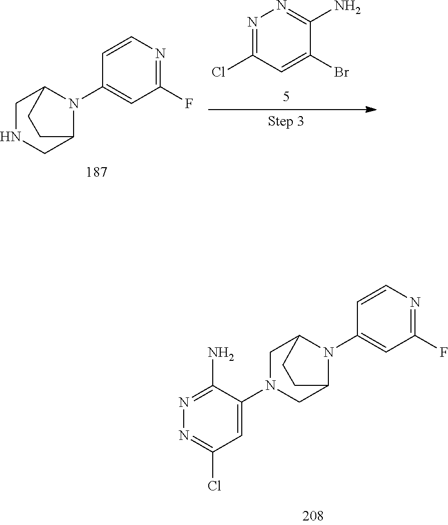 [SMILES: Fc1cc(N2C3CCC2CNC3)ccn1 ; Nc1nnc(Cl)cc1Br ; Nc1nnc(Cl)cc1N1CC2CCC(C1)N2c1ccnc(F)c1]

1,8-Diazabicyclo[5.4.0]undec-7-ene (5) (3.0 mL, 20 mmol) was added to a magnetically stirred solution of 187 (30 g, 145 mmol) 3-amino-4-bromo-6-chloropyridazine (44 g, 209 mmol), and N,N-diisopropylethylamine (80 mL, 460 mmol) in anhydrous DMF (300 mL) within a 1 L Erlenmeyer flask at 23° C. The solution was split evenly between two 450 mL sealable round bottom flasks, and then magnetically stirred in oil baths set at 100° C. for 23 hours. The clear red reaction mixtures were combined and were concentrated under high vacuum to a brown residue (107 g). The residue was purified twice by flash chromatography using 0-5% MeOH/DCM to provide a yellow solid of 208 complexed to one equivalent of N,N-diisopropylethylamine (20 g, 30%). The complex contained approximately 72 wt. % of 208 (˜15 g of 208).

### Step 4: Preparation of 04-1

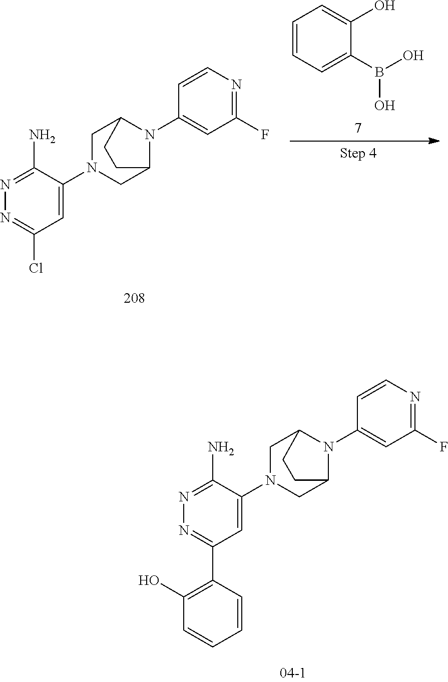 [SMILES: Nc1nnc(Cl)cc1N1CC2CCC(C1)N2c1ccnc(F)c1 ; OB(O)c1ccccc1O ; Nc1nnc(-c2ccccc2O)cc1N1CC2CCC(C1)N2c1ccnc(F)c1]

2-Hydroxyphenylboronic acid (9.0 g, 65 mmol) was added to a 1-neck 2 L round bottom flask containing a magnetically stirred mixture of the 208 amine complex (17 g, 37 mmol), and potassium carbonate (16 g, 113 mmol) in a mixture of 1,4-dioxane (600 mL) and deionized water (120 mL) at 23° C. The mixture was purged with nitrogen gas for 30 minutes then RuPhos-Pd-G3 (1.8 g, 2.1 mmol) was added. The flask was fitted with a condenser capped with a nitrogen inlet and was placed in a pre-heated oil bath set to 100° C. After stirring under a nitrogen atmosphere at 100° C. for 23 hours, the resultant deep red solution was cooled to 23° C. and was concentrated under reduced pressure to a brown solid (40 g). ¹H NMR analysis of the crude material showed 04-1 as the major product. The material from above was combined with 6.8 g of crude 04-1 from an earlier batch with a similar purity. The combined batches were purified by flash chromatography using 0-100% EtOAc/DCM followed by further chromatography eluting with 0-10% MeOH/DCM to provide 98% pure 04-1 by HPLC as a yellow solid. The solid was triturated with Et₂O, collected by filtration, and dried under reduced pressure to provide 04-1 as a yellow powder (8.0 g, 47% combined yield).

### Compound 3, Scheme 4: Synthesis of (3R)-tert-Butyl 4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

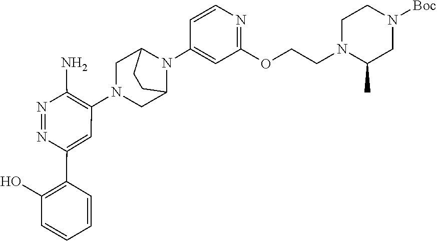 [SMILES: C[C@@H]1CN(C(=O)OC(C)(C)C)CCN1CCOc1cc(N2C3CCC2CN(c2cc(-c4ccccc4O)nnc2N)C3)ccn1]

To a solution of 2-(6-amino-5-(8-(2-fluoropyridin-4-yl)-3,8-diazabicyclo[3.2.1]octan-3-yl)pyridazin-3-yl)phenol (1.5 g, 3.82 mmol) (See Compound 1 in Scheme 4 above) and sodium hydride (60% in mineral oil; 0.46 g, 11.47 mmol) in THF (20 mL) was added (R)-tert-butyl 4-(2-hydroxyethyl)-3-methylpiperazine-1-carboxylate (See compound 2 in above Scheme 4; also see synthetic route at page 88, column 89 of WO2011/28685, herein incorporated by reference in its entirety) (1.87 g, 7.64 mmol) dropwise at 23° C. The mixture was then stirred at 60° C. for 12 hours. After cooling to 23° C., the reaction was diluted with water (100 mL) and the resulting mixture was extracted with ethyl acetate (150 mL×3). The combined organic layers were washed with brine (100 mL), dried over anhydrous sodium sulfate and filtered. The filtrate was concentrated, and the residue was purified by flash chromatography column on silica gel (0-5% of methanol in DCM) to afford the title compound (1.2 g, 64%) as a gray solid. LCMS (ESI) m/z: 617.6 [M+H]⁺.

### Compound 4, Scheme 4: Synthesis of 2-(6-Amino-5-(8-(2-(2-((R)-2-methylpiperazin-1-yl)ethoxy)pyridin-4-yl)-3,8-diazabicyclo[3.2.1]octan-3-yl)pyridazin-3-yl)phenol

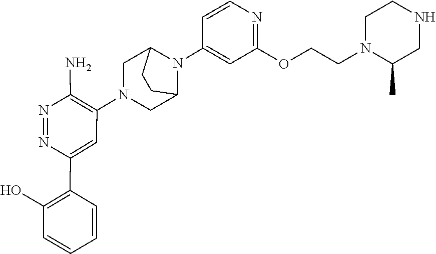 [SMILES: C[C@@H]1CNCCN1CCOc1cc(N2C3CCC2CN(c2cc(-c4ccccc4O)nnc2N)C3)ccn1]

To a 23° C. solution of (3R)-tert-butyl 4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (1.2 g, 1.95 mmol) in ethyl acetate (10 mL) was added 4 M HCl/EtOAc (20 mL, 1.95 mmol). The mixture was stirred at 23° C. for 16 hours then was concentrated. The residue was purified by flash chromatography column on silica gel (0-10% MeOH (1% NH₃·H₂O) in DCM) to afford the title compound (900 mg, 90%) as a yellow solid.

### Synthesis of Methyl 2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoate

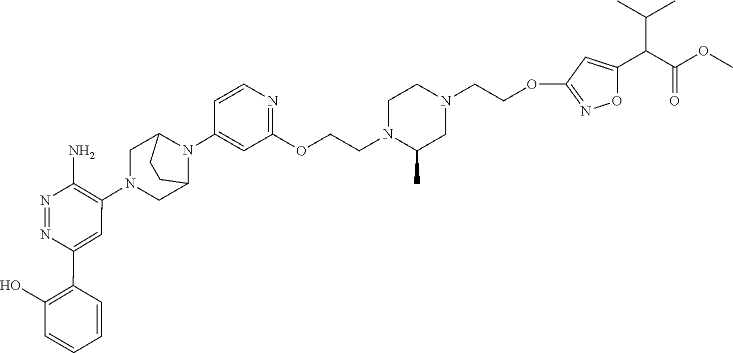 [SMILES: COC(=O)C(c1cc(OCCN2CCN(CCOc3cc(N4C5CCC4CN(c4cc(-c6ccccc6O)nnc4N)C5)ccn3)[C@H](C)C2)no1)C(C)C]

To a solution of NaBH(OAc)₃ (746 mg, 3.48 mmol), 2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenol (900 mg, 1.74 mmol) and methyl 3-methyl-2-(3-(2-oxoethoxy)isoxazol-5-yl)butanoate (See Compound 5 in Scheme 4 above; see synthetic route at page 428 of US 2020/0038378, herein incorporated by reference in its entirety) (463 mg, 1.92 mmol) in methyl alcohol (10 mL) and dichloromethane (10 mL) at 23° C. was added sodium acetate (712 mg, 8.71 mmol). The reaction mixture was stirred at 23° C. for 16 hours then was concentrated. The residue was purified by flash chromatography column on silica gel (0-10% MeOH in DCM) to afford the title compound (1.0 g, 77%) as a yellow solid. LCMS (ESI) m/z: 742.6 [M+H]⁺.

### Compound 6, Scheme 4: Synthesis of 2-(3-(2-((3R)-4-(2-((4-(3-(3-Amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid

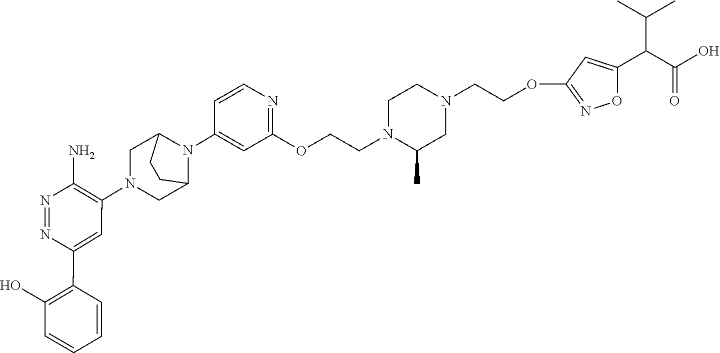 [SMILES: CC(C)C(C(=O)O)c1cc(OCCN2CCN(CCOc3cc(N4C5CCC4CN(c4cc(-c6ccccc6O)nnc4N)C5)ccn3)[C@H](C)C2)no1]

To a 23° C. solution of methyl 2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoate (1.0 g, 1.35 mmol) in water (6 mL) and methyl alcohol (6 mL) was added lithium hydroxide monohydrate (3 mg, 6.74 mmol). The mixture was stirred at 23° C. for 16 hours then was concentrated to afford the title compound (980 mg) as a yellow solid. LCMS (ESI) m/z: 728.3 [M+H]⁺.

### Compound 7, Scheme 4: Synthesis of (2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid

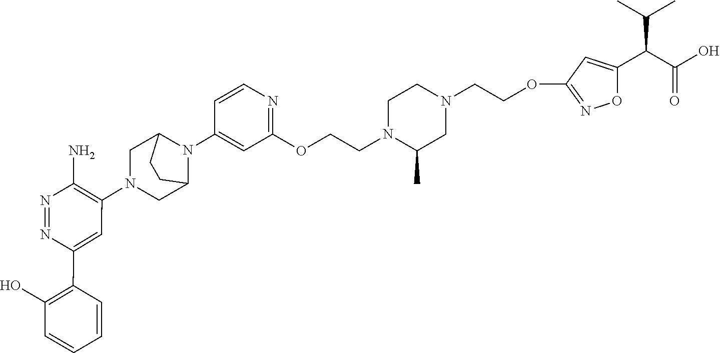 [SMILES: CC(C)[C@@H](C(=O)O)c1cc(OCCN2CCN(CCOc3cc(N4C5CCC4CN(c4cc(-c6ccccc6O)nnc4N)C5)ccn3)[C@H](C)C2)no1]

2-(3-(2-((3R)-4-(2-((4-(3-(3-Amino-6-(2-hydroxyphenyl)isoxazole-4-yl)-3,8-diazabicyclo[3.2.1]octan-8-yl)isoxazol-2-yl)oxy)ethyl)-3-methylpiperazin-1-yl)ethoxy) isoxazole-5-yl)-3-methylbutanoic acid (900 mg, 1.24 mmol) was separated by chiral SFC (DAICEL CHIRALPAK AD (250 mm*50 mm, 10 um) 0.1% NH₃H₂O, IPA, 18%) to afford the first peak (2S)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid (400 mg, 44%) and the second peak (2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid (450 mg, 50%) both as white solids.

### Synthesis of Compound 15, Scheme 4

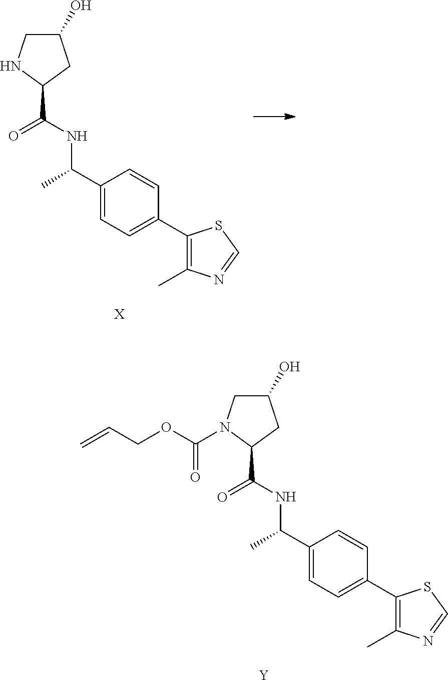

### Preparation of Compound 8 (referred to as Compound Y below) of Scheme 4. Synthesis of Allyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamate

Allyl chloroformate (485 mg, 4.02 mmol) was added to a 0° C. mixture of (2S,4R)-4-hydroxy-N—((S)-1-(4-(4-methylthiazol-5-yl)phenyl)ethyl)pyrrolidine-2-carboxamide (See, e.g., Compound 1 above; see preparation described in: J. Med. Chem. 2019, 62, 941) (1.65 g, 3.83 mmol) and NaHCO₃ (1.61 g, 19.2 mmol) in 1:1 THF:H₂O (34 mL). The resulting mixture was allowed to warm to 25° C. and stirred at that temperature for 12 h. After diluting with water (50 mL), the reaction mixture was extracted with EtOAc (3×50 mL), and the combined organic layers were washed with brine (50 mL), dried over Na₂SO₄, filtered, and concentrated. The residue was purified by flash column chromatography on silica gel eluting with 0-3% MeOH in DCM to give (2S,4R)-allyl-4-hydroxy-2-(((S)-1-(4-(4-methylthiazol-5-yl)phenyl)ethyl)carbamoyl)pyrrolidine-1-carboxylate (See compound Y above) (1.60 g, 81%) as a gray solid. LCMS (10-80, AB, 7.0 min): RT=2.57 min, m/z=537.1 [M+Na]⁺.

### Preparation of Compound 9 of Scheme 4. (2S,4R)-Allyl 4-((hydroxyhydrophosphoryl)oxy)-2-(((S)-1-(4-(4-methylthiazol-5-yl)phenyl)ethyl)carbamoyl)pyrrolidine-1-carboxylate

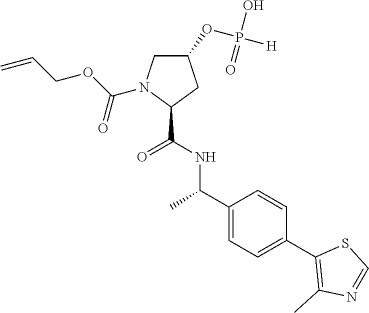 [SMILES: C=CCOC(=O)N1C[C@H](O[PH](=O)O)C[C@H]1C(=O)N[C@@H](C)c1ccc(-c2scnc2C)cc1]

To a solution of (2S,4R)-allyl 4-hydroxy-2-(((S)-1-(4-(4-methylthiazol-5-yl)phenyl)ethyl)carbamoyl)pyrrolidine-1-carboxylate (2.0 g, 4.81 mmol) in THF (30 mL) was added PCl₃ (1.67 mL, 19.3 mmol) in THF (5 mL) and Et₃N (4.03 mL, 29 mmol) in THF (3 mL) at −78° C. The reaction mixture was stirred at −78° C. for 20 min then was allowed warm to 23° C. The resulting mixture was stirred at 23° C. for 12 hours then was quenched with water (20 mL) and aq NaHCO₃ (5 mL). After stirring at 23° C. for 10 min, the mixture was acidified with 1 N HCl to pH=3, and was subsequently concentrated under reduced pressure. The residue was purified by flash chromatography column on silica gel (0-10% methanol in DCM) to afford the title compound (1.5 g, 65%) as a colorless solid. LCMS (ESI) m/z: 480.2 [M+H]⁺.

### Preparation of Compound 10 of Scheme 4. Synthesis of (2S,4R)-Allyl 4-((hydroxy(1H-imidazol-1-yl)phosphoryl)oxy)-2-(((S)-1-(4-(4-methylthiazol-5-yl)phenyl)ethyl)carbamoyl)pyrrolidine-1-carboxylate

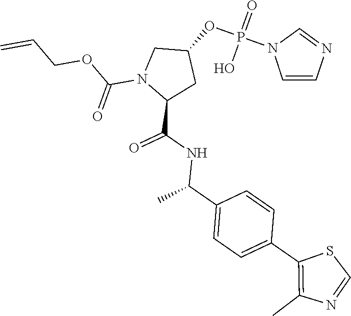 [SMILES: C=CCOC(=O)N1C[C@H](OP(=O)(O)n2ccnc2)C[C@H]1C(=O)N[C@@H](C)c1ccc(-c2scnc2C)cc1]

To a 23° C. solution of (2S,4R)-allyl 4-((hydroxyhydrophosphoryl)oxy)-2-(((S)-1-(4-(4-methylthiazol-5-yl)phenyl)ethyl)carbamoyl)pyrrolidine-1-carboxylate (600 mg, 1.25 mmol) and Et₃N (0.52 mL, 3.75 mmol) in CCl₄ (8 mL) and acetonitrile (8 mL) was added 1-(trimethylsilyl)-1H-imidazole (0.53 g, 3.75 mmol) at 23° C. The reaction mixture was stirred at 23° C. for 40 min then was concentrated. The residue was triturated with MTBE/EtOAc=5/1 (3 mL), and the resulting precipitate was collected by filtration, washed with MTBE (3 mL), and air-dried to afford the title compound (680 mg, 99%). LCMS (ESI) m/z: 546.3 [M+H]⁺.

### Preparation of Compound 10 of Scheme 4. Synthesis of (2S,4R)-Allyl 4-(((((2-((((9H-fluoren-9-yl)methoxy)carbonyl)amino)ethoxy)(hydroxy)phosphoryl)oxy)(hydroxy)phosphoryl)oxy)-2-(((S)-1-(4-(4-methylthiazol-5-yl)phenyl)ethyl)carbamoyl)pyrrolidine-1-carboxylate

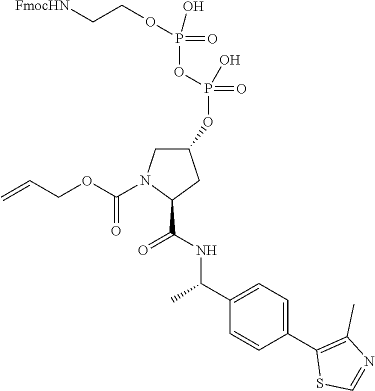

To a 23° C. solution of (2S,4R)-allyl 4-((hydroxy(1H-imidazol-1-yl)phosphoryl)oxy)-2-(((S)-1-(4-(4-methylthiazol-5-yl)phenyl)ethyl)carbamoyl)pyrrolidine-1-carboxylate (600 mg, 1.1 mmol) and (9H-fluoren-9-yl)methyl (2-(phosphonooxy)ethyl)carbamate (Compound 12 in Scheme 4 above); prepared as described in J. Org. Chem. 2007, 72, 3116.) (400 mg, 1.1 mmol) in N,N-Dimethylformamide (13 mL) was added 1 M zinc chloride in Et₂O (5.5 mL, 5.5 mmol). The reaction mixture was stirred at 23° C. for 12 hours then was concentrated. The residue was purified by flash chromatography column on silica gel (0-30% methanol (3% NH₃·H₂O) in DCM) to afford the title compound (340 mg, 37%) as a yellow oil. LCMS (ESI) m/z: 814.3 [M+H]⁺.

### Compound 15, Scheme 4: Synthesis of (9H-fluoren-9-yl)methyl (2-((hydroxy((hydroxy(((3R,5S)-5-(((S)-1-(4-(4-methylthiazol-5-yl)phenyl)ethyl)carbamoyl)pyrrolidin-3-yl)oxy)phosphoryl)oxy)phosphoryl)oxy)ethyl)carbamate

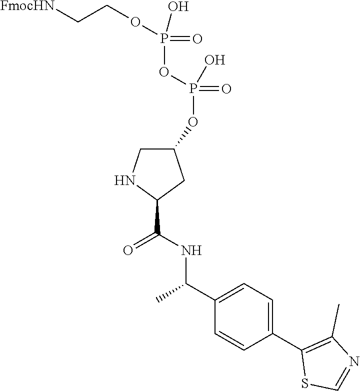

To a 23° C. solution of (2S,4R)-allyl 4-(((((2-((((9H-fluoren-9-yl)methoxy)carbonyl)amino)ethoxy)(hydroxy)phosphoryl)oxy)(hydroxy)phosphoryl)oxy)-2-(((S)-1-(4-(4-methylthiazol-5-yl)phenyl)ethyl)carbamoyl)pyrrolidine-1-carboxylate (340 mg, 0.40 mmol) and 1,3-dimethylpyrimidine-2,4,6(1H,3H,5H)-trione (316 mg, 2.0 mmol) in dichloromethane (5 mL) and methyl alcohol (5 mL) was added Pd(PPh₃)₄ (94 mg, 0.08 mmol). The reaction mixture was stirred under nitrogen atmosphere at 23° C. for 2 hours then was concentrated. The residue was purified by prep-HPLC with the following conditions: Column: YMC Triart C18 150*25 mm*5 um; mobile phase: 20-50% water (10 mM NH₄HCO₃)-ACN; Detector, UV 254 nm to afford the title compound (202 mg, 66%) as a white solid. LCMS (ESI) m/z: 757.4 [M+H]⁺.

### Compound 16, Scheme 4: Synthesis of (9H-Fluoren-9-yl)methyl (2-(((((((3R,5S)-1-((2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)(hydroxy)phosphoryl)oxy)(hydroxy)phosphoryl)oxy)ethyl)carbamate

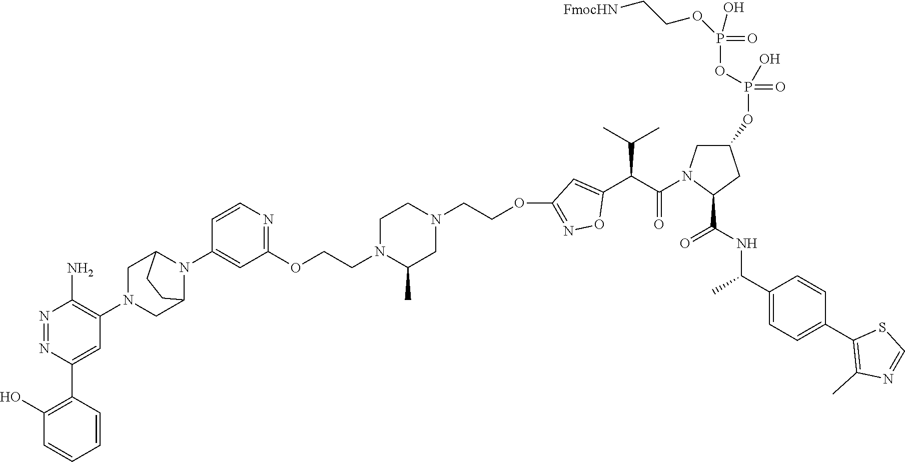

A solution of (2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoic acid (308 mg, 0.42 mmol), HATU (201 mg, 0.53 mmol) and (9H-fluoren-9-yl)methyl (2-((hydroxy((hydroxy(((3R,5S)-5-(((S)-1-(4-(4-methylthiazol-5-yl)phenyl)ethyl)carbamoyl)pyrrolidin-3-yl)oxy)phosphoryl)oxy)phosphoryl)oxy)ethyl)carbamate (80 mg, 0.11 mmol) in anhydrous N,N-dimethylformamide (2 mL) was stirred at 23° C. for 20 min. N,N-diisopropylethylamine (1 mL, 0.63 mmol) was then added, and the resulting mixture was stirred at 23° C. for 2 days. The mixture was the directly purified by prep-HPLC with the following conditions: Column: Phenomenex Gemini-NX 150*30 mm*5 um; mobile phase: 24-51% water (0.05% NH₃·H₂O)-ACN to afford the title compound (60 mg, 39%) as white solid. LCMS (ESI) m/z: 1467.7 [M+H]⁺.

### Compound 17, Scheme 4: Synthesis of [2-Aminoethoxy(hydroxy)phosphoryl] [(3R,5S)-1-[(2R)-2-[3-[2-[(3R)-4-[2-[[4-[3-[3-amino-6-(2-hydroxyphenyl)pyridazin-4-yl]-3,8-diazabicyclo[3.2.1]octan-8-yl]-2-pyridyl]oxy]ethyl]-3-methyl-piperazin-1-yl]ethoxy]isoxazol-5-yl]-3-methyl-butanoyl]-5-[[(1S)-1-[4-(4-methylthiazol-5-yl)phenyl]ethyl]carbamoyl]pyrrolidin-3-yl] hydrogen phosphate

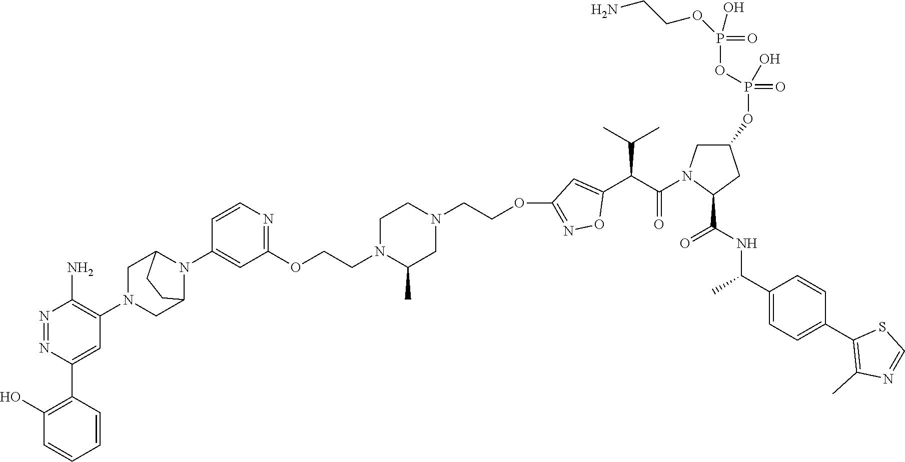 [SMILES: Cc1ncsc1-c1ccc([C@H](C)NC(=O)[C@@H]2C[C@@H](OP(=O)(O)OP(=O)(O)OCCN)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

A solution of (9H-fluoren-9-yl)methyl (2-(((((((3R,5S)-1-((2R)-2-(3-(2-((3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)(hydroxy)phosphoryl)oxy)(hydroxy)phosphoryl)oxy)ethyl)carbamate (20 mg, 0.01 mmol) and piperidine (2 mg, 0.01 mmol) in anhydrous N,N-dimethylformamide (0.5 mL) was stirred at 23° C. for 2 hours. The mixture was then directly purified by prep-HPLC (Phenomenex Gemini-NX 150*30 mm*5 um, 20-50% water (0.05% NH₃9H₂O)-ACN) to afford the title compound (18 mg, 94%) as a white solid. ¹H NMR (400 MHz, DMSO-d₆): (9.01-8.91 (m, 1H), 7.93-7.89 (m, 1H), 7.79-7.75 (m, 1H), 7.48 (s, 1H), 7.43-7.28 (m, 5H), 7.23-7.19 (m, 2H), 6.88-6.80 (m, 3H), 6.54-6.52 (m, 1H), 6.14-6.10 (m, 2H), 5.97 (s, 2H), 4.89-4.86 (m, 2H), 4.52-4.35 (m, 3H), 4.26-4.21 (m, 5H), 4.08-3.73 (m, 5H), 3.06-2.98 (m, 4H), 2.95-2.91 (m, 3H), 2.89-2.84 (m, 6H), 2.69-2.66 (m, 4H), 2.46-2.41 (m, 6H), 2.36-2.31 (m, 2H), 2.17-2.15 (m, 3H), 1.98-1.95 (m, 3H), 1.89-1.84 (m, 2H), 1.55-1.51 (m, 9H), 1.39-1.31 (m, 3H), 1.26-1.21 (m, 3H), 1.03-0.92 (m, 8H), 0.80-0.71 (m, 5H); LCMS (ESI) m/z: 1245.6 [M+H]⁺.

### L1-CIDE-BRM1-4: Synthesis of [(3R,5S)-1-[2-[3-[2-[(3R)-4-[2-[[4-[3-[3-Amino-6-(2-hydroxyphenyl)pyridazin-4-yl]-3,8-diazabicyclo[3.2.1]octan-8-yl]-2-pyridyl]oxy]ethyl]-3-methyl-piperazin-1-yl]ethoxy]isoxazol-5-yl]-3-methyl-butanoyl]-5-[[(1S)-1-[4-(4-methylthiazol-5-yl)phenyl]ethyl]carbamoyl]pyrrolidin-3-yl][2-[6-(2,5-dioxopyrrol-1-yl)hexanoylamino]ethoxy-hydroxy-phosphoryl] hydrogen phosphate

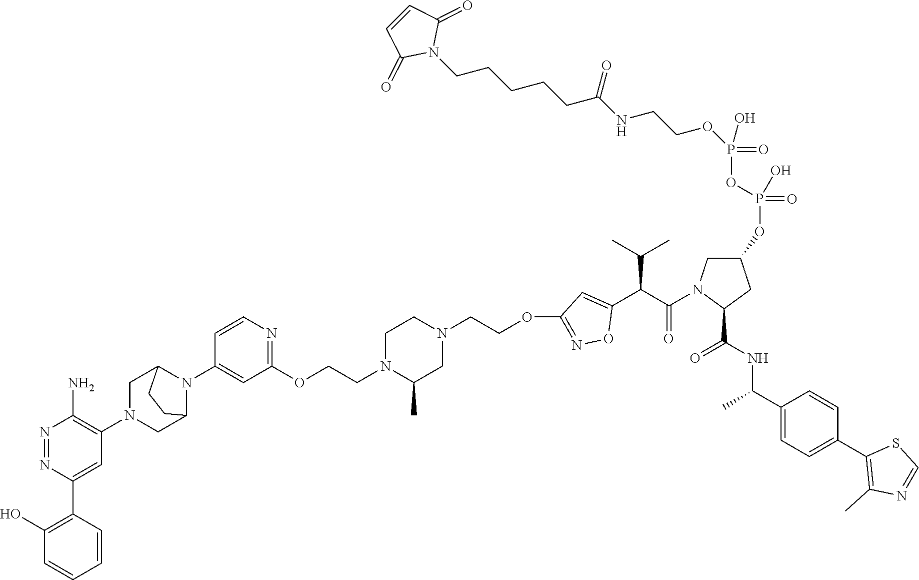 [SMILES: Cc1ncsc1-c1ccc([C@H](C)NC(=O)[C@@H]2C[C@@H](OP(=O)(O)OP(=O)(O)OCCNC(=O)CCCCCN3C(=O)C=CC3=O)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a 23° C. solution of [2-aminoethoxy(hydroxy)phosphoryl] [(3R,5S)-1-[2-[3-[2-[(3R)-4-[2-[[4-[3-[3-amino-6-(2-hydroxyphenyl)pyridazin-4-yl]-3,8-diazabicyclo[3.2.1]octan-8-yl]-2-pyridyl]oxy]ethyl]-3-methyl-piperazin-1-yl]ethoxy]isoxazol-5-yl]-3-methyl-butanoyl]-5-[[(1S)-1-[4-(4-methylthiazol-5-yl)phenyl]ethyl]carbamoyl]pyrrolidin-3-yl] hydrogen phosphate (50 mg, 0.04 mmol) in N,N-dimethylformamide (2 mL) was added 1-(6-(2,5-dioxopyrrolidin-1-yl)-6-oxohexyl)-1H-pyrrole-2,5-dione (24 mg, 0.08 mmol) and N,N-diisopropylethylamine (0.01 mL, 0.08 mmol). The mixture was stirred at 23° C. for 1 h then was directly purified by prep-HPLC (Phenomenex Gemini-NX 80*30 mm*3 um to, 17-47% water (10 mM NH₄HCO₃)-ACN) afford the title compound (7.9 mg, 14%) as a white solid. ¹H NMR (400 MHz, DMSO-d₆): (8.98-8.95 (m, 1H), 8.60-8.41 (m, 1H), 7.94-7.90 (m, 1H), 7.79-7.75 (m, 1H), 7.48 (s, 1H), 7.45-7.30 (m, 4H), 7.23-7.20 (m, 1H), 6.99-6.94 (m, 2H), 6.88-6.80 (m, 2H), 6.54-6.50 (m, 1H), 6.14-6.10 (m, 1H), 5.98-5.96 (m, 2H), 4.89-4.86 (m, 2H), 4.54-4.35 (m, 3H), 4.26-4.21 (m, 4H), 3.95-3.71 (m, 5H), 3.24-3.21 (m, 1H), 3.04-2.98 (m, 3H), 2.82-2.75 (m, 1H), 2.69-2.64 (m, 1H), 2.45-2.42 (m, 5H), 2.36-2.31 (m, 1H), 2.18-2.15 (m, 2H), 2.07-1.92 (m, 5H), 1.79 (s, 12H), 1.46-1.44 (m, 5H), 1.38-1.35 (m, 3H), 1.26-1.22 (m, 3H), 1.16-1.14 (m, 2H), 0.99-0.95 (m, 6H), 0.89-0.76 (m, 4H); LCMS (ESI) m/z: 1438.8 [M+H]⁺.

### Synthesis Example 5

### Syntheses of L1-CIDE-BRM1-5

L1-CIDE-BRM1-2 was synthesized by the following scheme, Scheme 5:

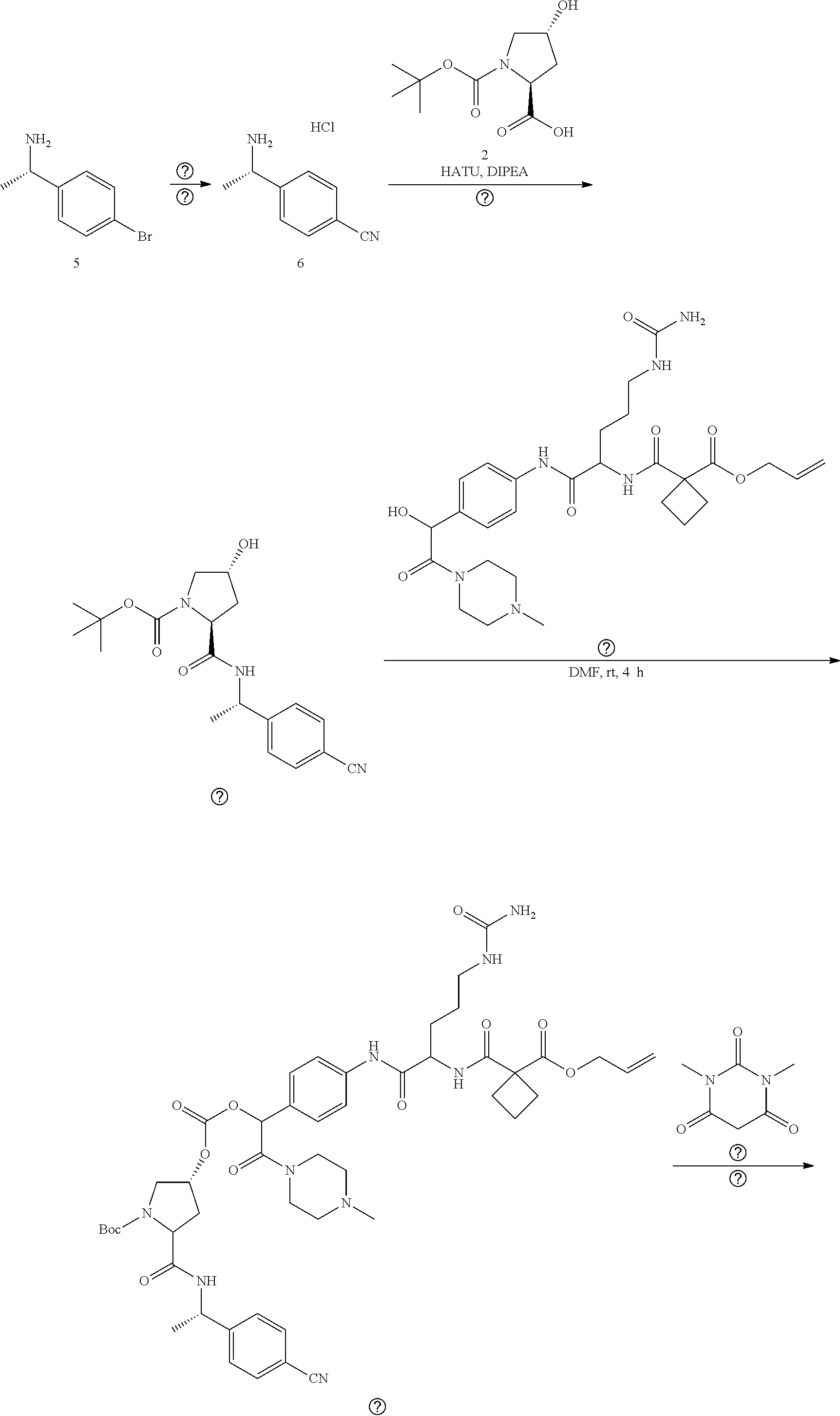

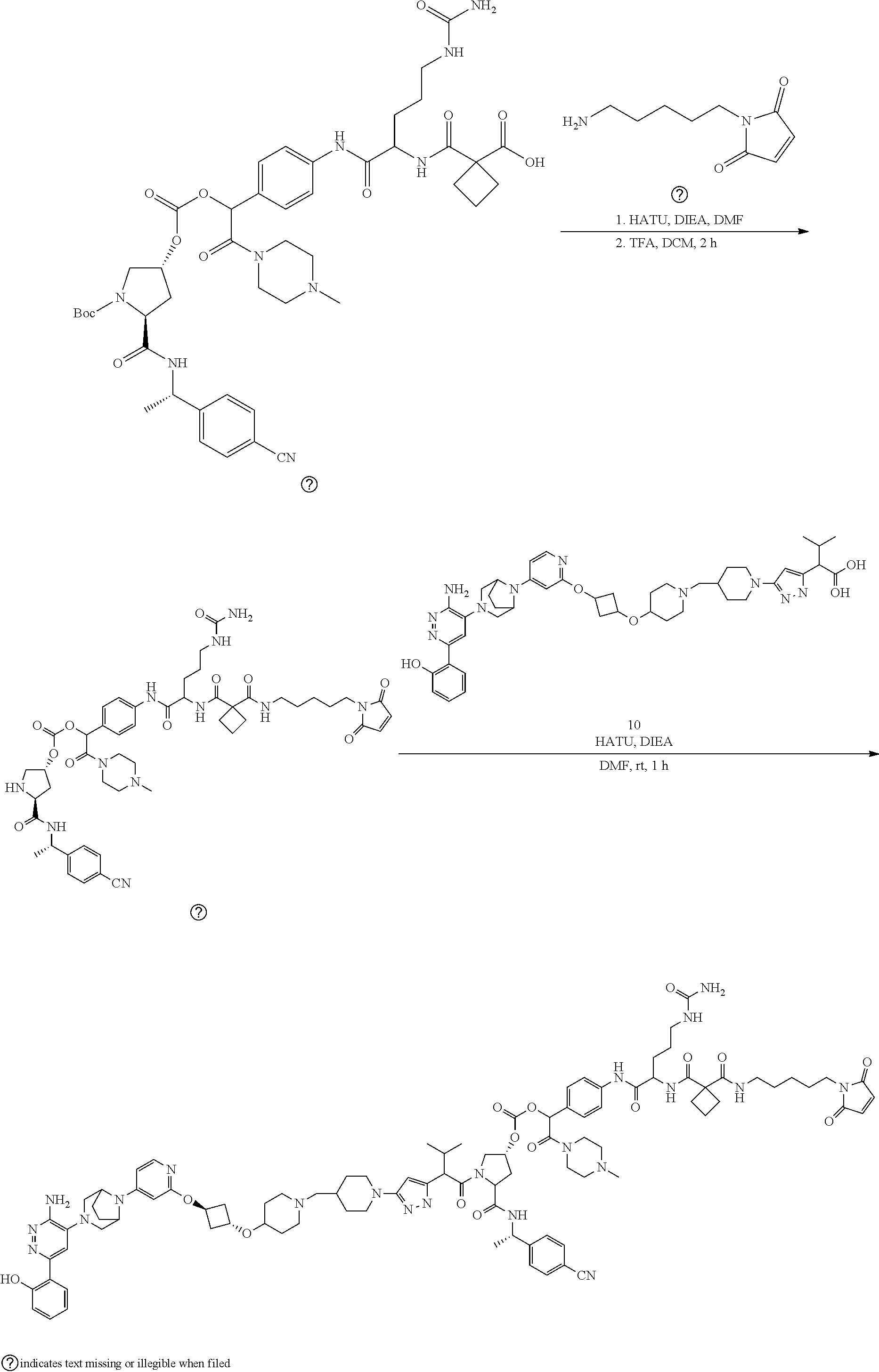

### Synthesis of (S)N-(1-(4-Bromophenyl)ethyl)acetamide

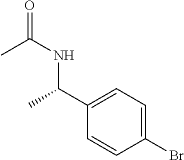

Acetyl chloride (2.35 g, 30 mmol) was added dropwise to a solution of (S)-1-(4-bromophenyl)ethanamine (5.0 g, 25 mmol) and Et₃N (3.8 g, 37.49 mmol) in THF (50 mL) at 0° C. The reaction was stirred at that temperature for 2.5 hours then was partitioned between EtOAc (50 mL×3) and saturated NaCl solution (50 mL). The combined organic layers were dried over sodium sulfate, filtered, and concentrated. The residue was purified by flash chromatography on silica gel (0-50% EtOAc in petroleum ether) to afford the title compound (4.0 g, 66%) as a white solid. LCMS (ESI) m/z: 241.8 [M+H]⁺.

### Synthesis of (S)—N-(1-(4-Cyanophenyl)ethyl)acetamide

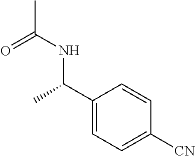

A mixture of copper(I) cyanide (1.78 g, 20 mmol) and (S)—N-(1-(4-bromophenyl)ethyl)acetamide (4.0 g, 16.5 mmol) in N,N-dimethylformamide (40 mL) was refluxed for 24 hours. After cooling to 23° C., the mixture was filtered and the filtrate was concentrated. The residue was added to a saturated aqueous NaHCO₃ solution (80 mL) at 23° C. and was stirred for 10 min. Saturated aqueous sodium hypochlorite solution was then added, and stirring was continued at 23° C. for 24 h. The mixture was extracted with EtOAc (70 mL×3), and the combined organic layers were washed with water (70 mL×3), dried over sodium sulfate, filtered, and concentrated to afford the title compound (2.7 g, 87%) as a yellow solid. LCMS (ESI) m/z: 189.2 [M+H]⁺.

### Synthesis of (S)-4-(1-Aminoethyl)benzonitrile hydrochloride

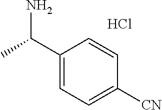

A solution of (S)—N-(1-(4-cyanophenyl)ethyl)acetamide (2.4 g, 12.8 mmol) and aqueous 2 M HCl (20 mL, 12.8 mmol) was stirred at 100° C. for 24 hours. The reaction solution was cooled to 23° C. and was then concentrated to afford the title compound (1.8 g, 97%) as a yellow solid. LCMS (ESI) m/z: 147.1 [M+H]⁺.

### Compound 4, Scheme 5: Synthesis of (2S,4R)-tert-butyl 2-(((S)-1-(4-Cyanophenyl)ethyl)carbamoyl)-4-hydroxypyrrolidine-1-carboxylate

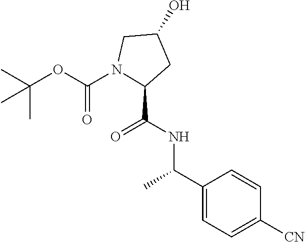 [SMILES: C[C@H](NC(=O)[C@@H]1C[C@@H](O)CN1C(=O)OC(C)(C)C)c1ccc(C#N)cc1]

To a 23° C. mixture of (S)-4-(1-aminoethyl)benzonitrile hydrochloride (1.6 g, 11 mmol) and (2S,4R)-1-(tert-butoxycarbonyl)-4-hydroxypyrrolidine-2-carboxylic acid (2.8 g, 12 mmol) in N,N-dimethylformamide (50 mL) was added N,N-diisopropylethylamine (4.3 g, 33 mmol) and HATU (1.63 g, 12 mmol). The reaction was stirred at 23° C. for 16 hours then was concentrated. The residue was purified by flash chromatography on silica gel (50-100% ethyl acetate in dichloromethane) to afford the title compound (1.7 g, 43%) as yellow solid. LCMS (ESI) m/z: 360.1 [M+H]⁺.

### Synthesis of (2S,4R)-tert-Butyl 2-(((S)-1-(4-cyanophenyl)ethyl)carbamoyl)-4-((4-nitrobenzoyl)oxy)pyrrolidine-1-carboxylate

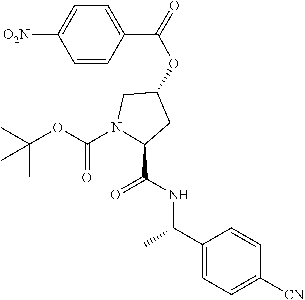 [SMILES: C[C@H](NC(=O)[C@@H]1C[C@@H](OC(=O)c2ccc([N+](=O)[O-])cc2)CN1C(=O)OC(C)(C)C)c1ccc(C#N)cc1]

To a mixture of 4-nitrophenylchloroformate (1.68 g, 8.3 mmol) and (2S,4R)-tert-butyl 2-(((S)-1-(4-cyanophenyl)ethyl)carbamoyl)-4-hydroxypyrrolidine-1-carboxylate (3.0 g, 7.0 mmol) in anhydrous dichloromethane (80 mL) at 23° C. was added 2,6-lutidine (1.1 g, 10.43 mmol). The reaction mixture was stirred at 23° C. for 18 hours then was concentrated to afford the title compound (437 mg, 99.8%) as a yellow solid. LCMS (ESI) m/z: 597.2 [M+H]⁺. Compound 6, Scheme 5: Synthesis of (2S,4R)-tert-Butyl 4-(((1-(4-((S)-2-(1-((allyloxy)carbonyl)cyclobutanecarboxamido)-5-ureidopentanamido)phenyl)-2-(4-methylpiperazin-1-yl)-2-oxoethoxy)carbonyl)oxy)-2-(((S)-1-(4-cyanophenyl)ethyl)carbamoyl)pyrrolidine-1-carboxylate

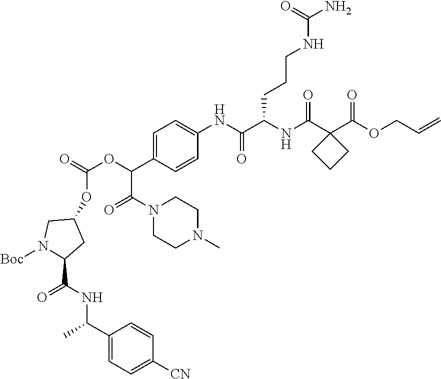 [SMILES: C=CCOC(=O)C1(C(=O)N[C@@H](CCCNC(N)=O)C(=O)Nc2ccc(C(OC(=O)O[C@@H]3C[C@@H](C(=O)N[C@@H](C)c4ccc(C#N)cc4)N(C(=O)OC(C)(C)C)C3)C(=O)N3CCN(C)CC3)cc2)CCC1]

To a 23° C. mixture of tert-butyl (2S,4R)-tert-butyl 2-(((S)-1-(4-cyanophenyl)ethyl)carbamoyl)-4-((4-nitrobenzoyl)oxy)pyrrolidine-1-carboxylate (437 mg, 0.83 mmol) and allyl 1-(((2S)-1-((4-(1-hydroxy-2-(4-methylpiperazin-1-yl)-2-oxoethyl)phenyl)amino)-1-oxo-5-ureidopentan-2-yl)carbamoyl)cyclobutanecarboxylate (334 mg, 0.58 mmol) in anhydrous N,N-dimethylformamide (10 mL) was added DMAP (203 mg, 1.67 mmol). The reaction mixture was stirred at 40° C. for 18 hours then was cooled to 23° C. and was filtered. The filtrate was directly purified by prep-HPLC (Xtimate C18 150*40 mm*5 um/water (0.225% FA)-ACN, 18-48%) to afford the title compound (65 mg, 8%) as a yellow solid. LCMS (ESI) m/z: 958.6 [M+H]⁺.

### Compound 7, Scheme 5: Synthesis of 1-(((2S)-1-((4-(1-(((((3R,5S)-1-(tert-Butoxycarbonyl)-5-(((S)-1-(4-cyanophenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid

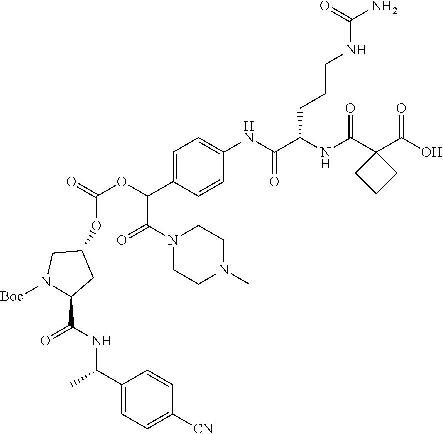 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)O)CCC3)cc2)CN1C(=O)OC(C)(C)C)c1ccc(C#N)cc1]

To a solution of (2S,4R)-tert-butyl 4-(((1-(4-((S)-2-(1-((allyloxy)carbonyl)cyclobutanecarboxamido)-5-ureidopentanamido)phenyl)-2-(4-methylpiperazin-1-yl)-2-oxoethoxy)carbonyl)oxy)-2-(((S)-1-(4-cyanophenyl)ethyl)carbamoyl)pyrrolidine-1-carboxylate (65 mg, 0.07 mmol) and 1,3-dimethylpyrimidine-2,4,6(1H,3H,5H)-trione (53 mg, 0.34 mmol) in dichloromethane (3 mL) and methyl alcohol (3 mL) was added Pd(PPh₃)₄ (16 mg, 0.01 mmol) at 23° C. The reaction mixture was stirred under a nitrogen atmosphere at 23° C. for 10 hours then was concentrated. The residue was purified by prep-HPLC with the following conditions: Column: Phenomenex Gemini-NX 80*30 mm*3 um, mobile phase: (24-50%) water (10 mM NH₄HCO₃)-ACN to afford the title compound (40 mg, 64%) as a red solid. LCMS (ESI) m/z: 918.6 [M+H]⁺.

### Compound 9, Scheme 5: Synthesis of (2S,4R)-tert-Butyl 2-(((S)-1-(4-cyanophenyl)ethyl)carbamoyl)-4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)pyrrolidine-1-carboxylate

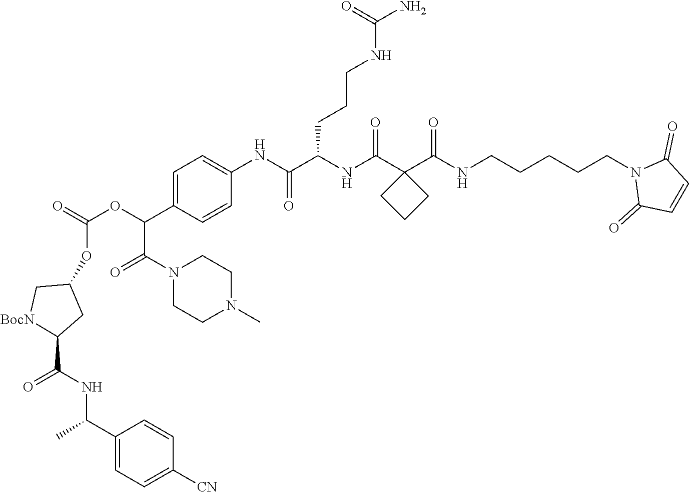 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)CN1C(=O)OC(C)(C)C)c1ccc(C#N)cc1]

To a 23° C. mixture of 1-(((2S)-1-((4-(1-(((((3R,5S)-1-(tert-butoxycarbonyl)-5-(((S)-1-(4-cyanophenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid (40 mg, 0.04 mmol) and 1-(5-aminopentyl)-1H-pyrrole-2,5-dione (10 mg, 0.05 mmol) in N,N-dimethylformamide (4 mL) was added N,N-diisopropylethylamine (0.02 mL, 0.13 mmol) and HATU (20 mg, 0.05 mmol). The reaction mixture was stirred at 23° C. for 16 hours then was concentrated. The residue was purified by prep-HPLC (Boston Green ODS 150*30 mm*5 um, water (0.075% TFA)-ACN, 20%-50%) to afford the title compound (31 mg, 65%) as a blue solid. LCMS (ESI) m/z: 1082.7 [M+H]⁺.

### Synthesis of (3R,5S)-5-(((S)-1-(4-Cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate 2,2,2-trifluoroacetate

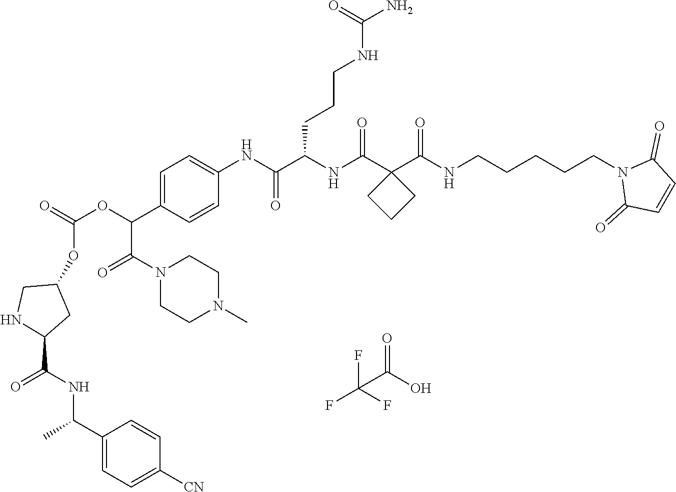 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)CN1)c1ccc(C#N)cc1 ; O=C(O)C(F)(F)F]

A solution of (2S,4R)-tert-butyl 2-(((S)-1-(4-cyanophenyl)ethyl)carbamoyl)-4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)pyrrolidine-1-carboxylate (30.5 mg, 0.03 mmol) in 5% TFA in HFIP (3 mL) was stirred at 23° C. for 1.5 hours. The reaction mixture was then concentrated to afford the title compound (28 mg, 99%) as a pink oil. LCMS (ESI) m/z: 982.7 [M+H]⁺.

### L1-CIDE-BRM-1-5: Synthesis of (3R,5S)-1-(2-(3-(4-(trans-3-((4-(3-(3-Amino-6-(2-hydroxyphenyl)pyridazin-4-yl)-3,8-diazabicyclo[3.2.1]octan-8-yl)pyridin-2-yl)oxy)cyclobutoxy)piperidin-1-yl)methyl)piperidin-1-yl)isoxazol-5-yl)-3-methylbutanoyl)-5-(((S)-1-(4-cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate

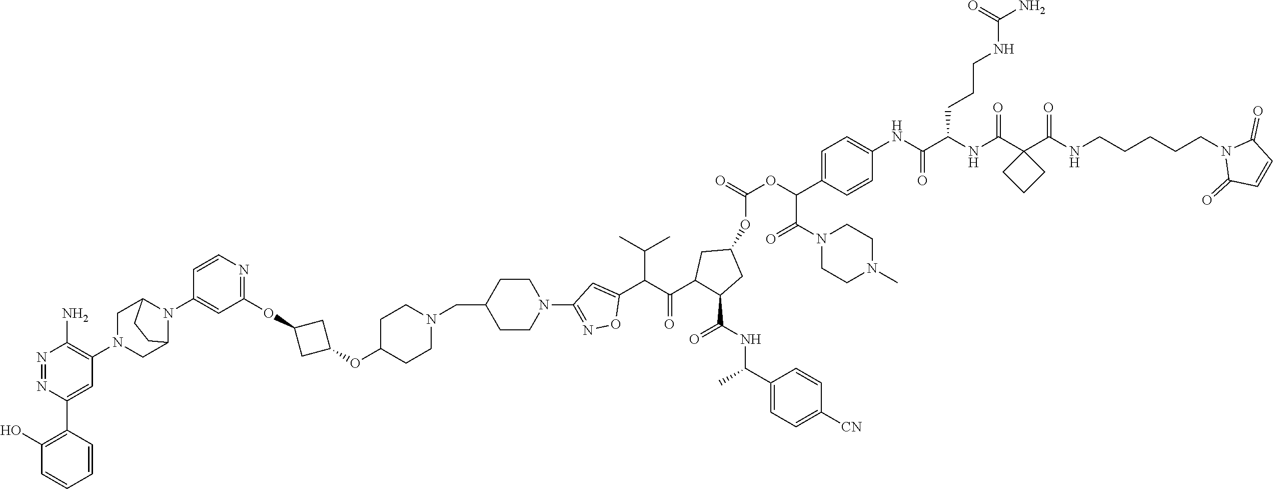 [SMILES: CC(C)C(C(=O)C1C[C@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)C[C@H]1C(=O)N[C@@H](C)c1ccc(C#N)cc1)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1]

To a mixture of (3R,5S)-5-(((S)-1-(4-cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate 2,2,2-trifluoroacetate (28 mg, 0.03 mmol) and 2-(3-(4-((4-(trans-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidin-1-yl)methyl)piperidin-1-yl)isoxazol-5-yl)-3-methylbutanoic acid (27 mg, 0.03 mmol) in DMF (3 mL) at 23° C. was added N,N-diisopropylethylamine (0.01 mL, 0.08 mmol) and HATU (13 mg, 0.03 mmol). The mixture was stirred at 23° C. for 4 hours then was concentrated. The residue was purified by prep-HPLC with the following conditions: Column: Phenomenex Gemini-NX 80*30 mm*3 um; mobile phase: (18-36%) water (10 mM NH₄HCO₃)-ACN to afford the title compound (23 mg, 31%) as a white solid. ¹H NMR (400 MHz, CD₃OD): δ 7.80-7.58 (m, 6H), 7.57-7.34 (m, 5H), 7.24-7.21 (m, 1H), 6.96-6.84 (m, 2H), 6.80-6.73 (m, 1H), 6.56-6.54 (m, 1H), 6.33-6.20 (m, 1H), 6.17-6.03 (m, 1H), 5.27-5.19 (m, 1H), 5.14-5.12 (m, 1H), 4.62-4.60 (m, 5H), 4.54-4.51 (m, 3H), 4.42-4.39 (m, 1H), 4.17-3.86 (m, 2H), 3.84-3.76 (m, 1H), 3.74-3.64 (m, 3H), 3.63-3.50 (m, 4H), 3.48-3.43 (m, 3H), 3.38-3.35 (m, 2H), 3.25-3.22 (m, 2H), 3.26-3.18 (m, 1H), 3.17-3.00 (m, 4H), 2.86-2.83 (m, 2H), 2.59-2.55 (m, 6H), 2.48-2.35 (m, 7H), 2.29-2.12 (m, 8H), 2.09-1.86 (m, 8H), 1.85-1.68 (m, 1H), 1.79-1.75 (m, 5H), 1.67-1.52 (m, 7H), 1.51-1.43 (m, 2H), 1.41-1.23 (m, 9H), 1.10-0.94 (m, 3H), 0.93-0.81 (m, 3H); LCMS (ESI) m/z: 1772.9 [M+H]⁺.

### Synthesis Example 6

### Syntheses of L1-CIDE-BRM1-6

L1-CIDE-BRM1-6 was synthesized by the following scheme, Scheme 6:

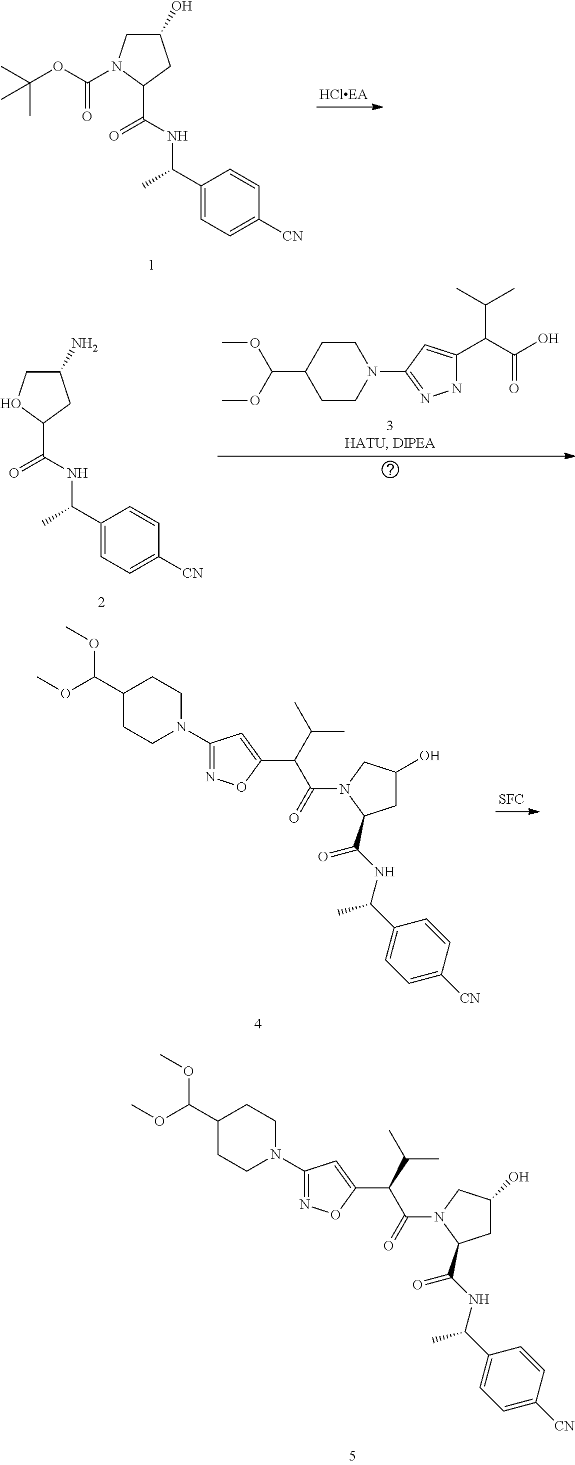 [SMILES: C[C@H](NC(=O)C1C[C@@H](O)CN1C(=O)OC(C)(C)C)c1ccc(C#N)cc1 ; C[C@H](NC(=O)C1C[C@@H](N)CO1)c1ccc(C#N)cc1 ; COC(OC)C1CCN(c2cc(C(C(=O)O)C(C)C)[nH]n2)CC1 ; COC(OC)C1CCN(c2cc(C(C(=O)N3CC(O)C[C@H]3C(=O)N[C@@H](C)c3ccc(C#N)cc3)C(C)C)on2)CC1 ; "SFC" ; COC(OC)C1CCN(c2cc([C@H](C(=O)N3C[C@H](O)C[C@H]3C(=O)N[C@@H](C)c3ccc(C#N)cc3)C(C)C)on2)CC1]

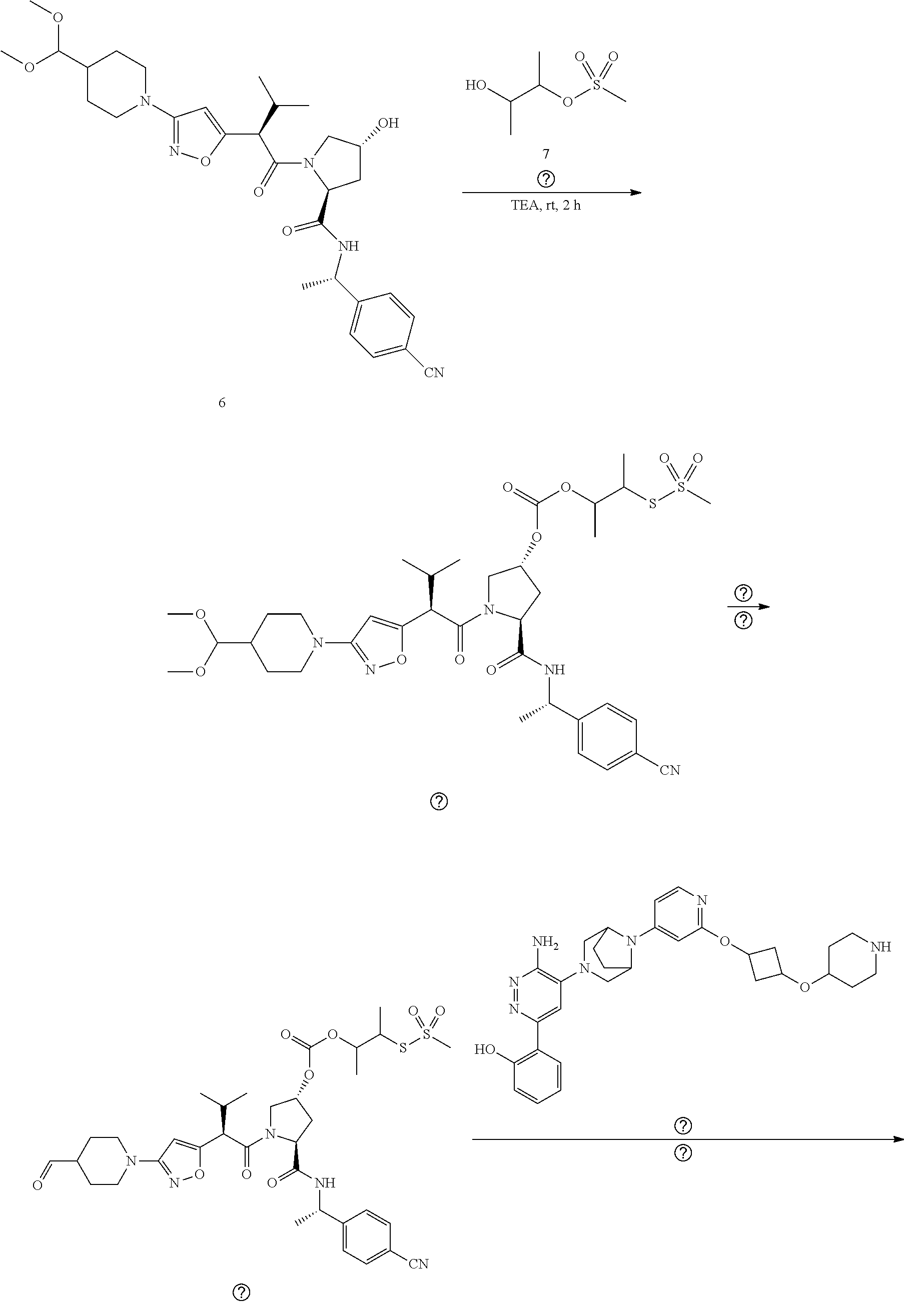

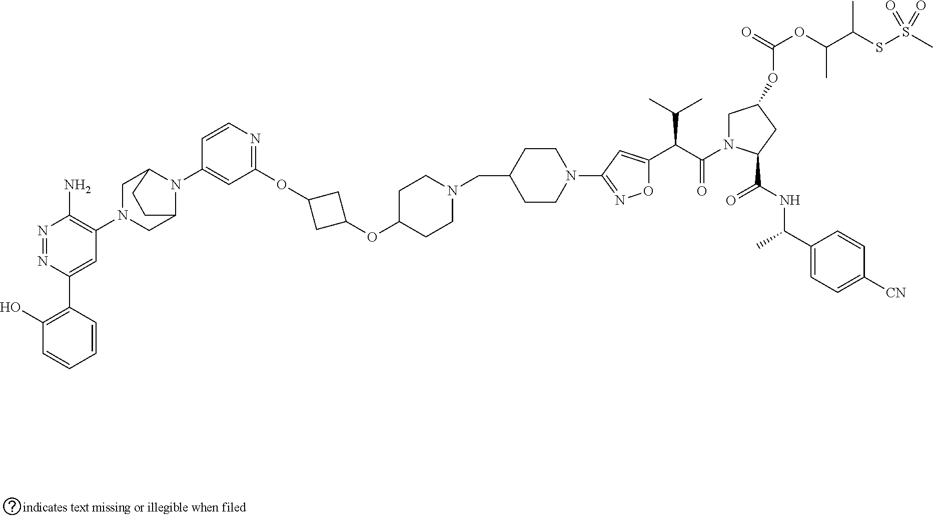 [SMILES: CC(OC(=O)O[C@@H]1C[C@@H](C(=O)N[C@@H](C)c2ccc(C#N)cc2)N(C(=O)[C@@H](c2cc(N3CCC(CN4CCC(OC5CC(Oc6cc(N7C8CCC7CN(c7cc(-c9ccccc9O)nnc7N)C8)ccn6)C5)CC4)CC3)no2)C(C)C)C1)C(C)SS(C)(=O)=O]

### Synthesis of (2S,4R)—N—((S)-1-(4-Cyanophenyl)ethyl)-4-hydroxypyrrolidine-2-carboxamide hydrochloride

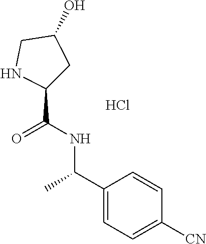 [SMILES: C[C@H](NC(=O)[C@@H]1C[C@@H](O)CN1)c1ccc(C#N)cc1 ; Cl]

To a 23° C. solution of (2S,4R)-tert-butyl 2-(((S)-1-(4-cyanophenyl)ethyl)carbamoyl)-4-hydroxypyrrolidine-1-carboxylate (See Compound 4 from Scheme 5 above) (2.0 g, 5.56 mmol) in ethyl acetate (5 mL) was added 4 M HCl (10 mL; prepared by bubbling dry HCl gas into dry EtOAc). The reaction mixture was stirred at 23° C. for 16 hours then was concentrated to afford the title compound (1.4 g, 97%) as a white solid.

### Compound 4, Scheme 6: Synthesis of (2S,4R)—N—((S)-1-(4-Cyanophenyl)ethyl)-1-(2-(3-(4-(dimethoxymethyl)piperidin-1-yl)isoxazol-5-yl)-3-methylbutanoyl)-4-hydroxypyrrolidine-2-carboxamide

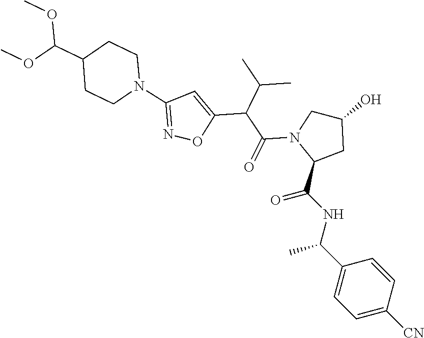 [SMILES: COC(OC)C1CCN(c2cc(C(C(=O)N3C[C@H](O)C[C@H]3C(=O)N[C@@H](C)c3ccc(C#N)cc3)C(C)C)on2)CC1]

To a 23° C. solution of (2S,4R)—N—((S)-1-(4-cyanophenyl)ethyl)-4-hydroxypyrrolidine-2-carboxamide hydrochloride (900 mg, 3.47 mmol) and 2-(3-(4-(dimethoxymethyl)piperidin-1-yl)isoxazol-5-yl)-3-methylbutanoic acid (See Compound 3 in Scheme 6 above; see the synthetic route disclosed at page 450 of US 2020/0038378, herein incorporated by reference in its entirety) (1.4 g, 3.43 mmol) in anhydrous N,N-dimethylformamide (20 mL) was added DIEA (1.71 mL, 10.29 mmol) and HATU (2.0 g, 5.15 mmol). The mixture was stirred at 23° C. for 2 hours then was partitioned between water (100 mL) and ethyl acetate (100 mL×3). The combined organic layers were washed with brine (50 mL×2), dried over sodium sulfate, filtered and concentrated. The residue was purified by flash chromatography on silica gel (0-100% ethyl acetate in petroleum ether) to afford the title compound (2.0 g, 98%) as a colorless oil. LCMS (ESI) m/z: 568.3 [M+H]⁺.

### Compound 5, Scheme 6: Synthesis of (2S,4R)—N—((S)-1-(4-Cyanophenyl)ethyl)-1-((R)-2-(3-(4-(dimethoxymethyl)piperidin-1-yl)isoxazol-5-yl)-3-methylbutanoyl)-4-hydroxypyrrolidine-2-carboxamide

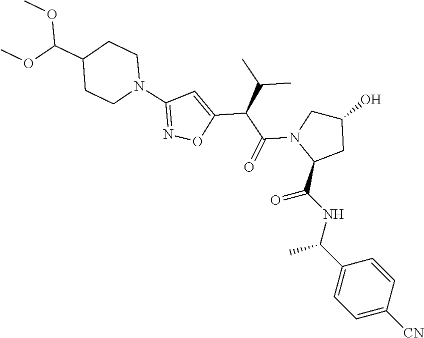 [SMILES: COC(OC)C1CCN(c2cc([C@H](C(=O)N3C[C@H](O)C[C@H]3C(=O)N[C@@H](C)c3ccc(C#N)cc3)C(C)C)on2)CC1]

(2S,4R)—N—((S)-1-(4-Cyanophenyl)ethyl)-1-(2-(3-(4-(dimethoxymethyl)piperidin-1-yl)isoxazol-5-yl)-3-methylbutanoyl)-4-hydroxypyrrolidine-2-carboxamide (2 g, 3.52 mmol) was separated by chiral SFC (DAICEL CHIRALPAK OD (250 mm*30 mm, 10 um); (20%) 0.1% NH₃H₂O, EtOH) to afford the first peak (2S,4R)—N—((S)-1-(4-cyanophenyl)ethyl)-1-((S)-2-(3-(4-(dimethoxymethyl)piperidin-1-yl)isoxazol-5-yl)-3-methylbutanoyl)-4-hydroxypyrrolidine-2-carboxamide (400 mg, 20%) and the second peak (2S,4R)—N—((S)-1-(4-cyanophenyl)ethyl)-1-((R)-2-(3-(4-(dimethoxymethyl)piperidin-1-yl)isoxazol-5-yl)-3-methylbutanoyl)-4-hydroxypyrrolidine-2-carboxamide (700 mg, 35%) both as white solid. ¹H NMR (400 MHz, DMSO-d₆,): δ 8.49 (d, J=7.2 Hz, 1H), 7.82-7.76 (m, 2H), 7.50-7.44 (m, 2H), 6.11 (s, 1H), 5.13 (d, J=3.6 Hz, 1H), 4.93-4.89 (m, 1H), 4.36-4.31 (m, 1H), 4.27-4.25 (s, 1H), 4.07 (d, J=7.6 Hz, 1H), 3.72-3.53 (m, 4H), 3.45-3.40 (m, 1H), 3.26 (s, 6H), 2.76-2.67 (m, 2H), 2.25-2.16 (m, 1H), 2.08-1.96 (m, 1H), 1.79-1.61 (m, 4H), 1.44-1.33 (m, 3H), 1.30-1.19 (m, 2H), 0.98-0.89 (m, 3H), 0.84-0.74 (m, 3H).

### Compound 8, Scheme 6: Synthesis of S-(3-(((((3R,5S)-5-(((S)-1-(4-Cyanophenyl)ethyl)carbamoyl)-1-((R)-2-(3-(4-(dimethoxymethyl)piperidin-1-yl)isoxazol-5-yl)-3-methylbutanoyl)pyrrolidin-3-yl)oxy)carbonyl)oxy)butan-2-yl) methanesulfonothioate

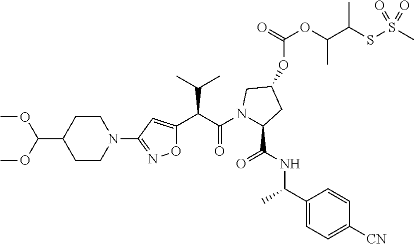

To a 23° C. mixture of (2S,4R)—N—((S)-1-(4-cyanophenyl)ethyl)-1-((R)-2-(3-(4-(dimethoxymethyl)piperidin-1-yl)isoxazol-5-yl)-3-methylbutanoyl)-4-hydroxypyrrolidine-2-carboxamide (300 mg, 0.53 mmol) and 4 Å MS (100 mg) in anhydrous dichloromethane (3 mL) was slowly added a solution of S-(3-((chlorocarbonyl)oxy)butan-2-yl) methanesulfonothioate (See Compound 7 in Scheme 6 above or Compound 2 in Scheme 1 above) (391 mg, 1.59 mmol) in dichloromethane (2 mL) and Et₃N (214 mg, 2.11 mmol) in anhydrous dichloromethane (3 mL). The reaction was stirred at 23° C. for 16 hours then was filtered. The filtrate was concentrated, and the residue was purified by flash chromatography on silica gel (0-70% ethyl acetate in petroleum ether) to afford the title compound (200 mg, 49%) as a white solid. LCMS (ESI) m/z: 778.1 [M+H]⁺.

### Compound 9, Scheme 6: Synthesis of S-(3-(((((3R,5S)-5-(((S)-1-(4-Cyanophenyl)ethyl)carbamoyl)-1-((R)-2-(3-(4-formylpiperidin-1-yl)isoxazol-5-yl)-3-methylbutanoyl)pyrrolidin-3-yl)oxy)carbonyl)oxy)butan-2-yl) methanesulfonothioate

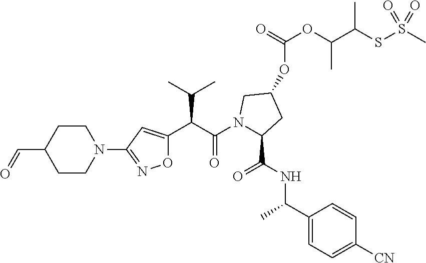 [SMILES: CC(OC(=O)O[C@@H]1C[C@@H](C(=O)N[C@@H](C)c2ccc(C#N)cc2)N(C(=O)[C@@H](c2cc(N3CCC(C=O)CC3)no2)C(C)C)C1)C(C)SS(C)(=O)=O]

To a solution of S-(3-(((((3R,5S)-5-(((S)-1-(4-cyanophenyl)ethyl)carbamoyl)-1-((R)-2-(3-(4-(dimethoxymethyl)piperidin-1-yl)isoxazol-5-yl)-3-methylbutanoyl)pyrrolidin-3-yl)oxy)carbonyl)oxy)butan-2-yl) methanesulfonothioate (100 mg, 0.13 mmol) in THF (1 mL) at 23° C. was added formic acid (1 mL) and water (3 mL). The mixture was stirred at 50° C. for 16 hours then was cooled to 23° C. and was concentrated to afford the title compound (80 mg, 85%) as a yellow oil. LCMS (ESI) m/z: 732.0 [M+H]⁺.

### L1-CIDE-BRM1-6: Synthesis of S-(3-(((((3R,5S)-1-((2R)-2-(3-(4-((4-(trans-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidin-1-yl)methyl)piperidin-1-yl)isoxazol-5-yl)-3-methylbutanoyl)-5-(((S)-1-(4-cyanophenyl)ethyl)carbamoyl)pyrrolidin-3-yl)oxy)carbonyl)oxy)butan-2-yl) methanesulfonothioate

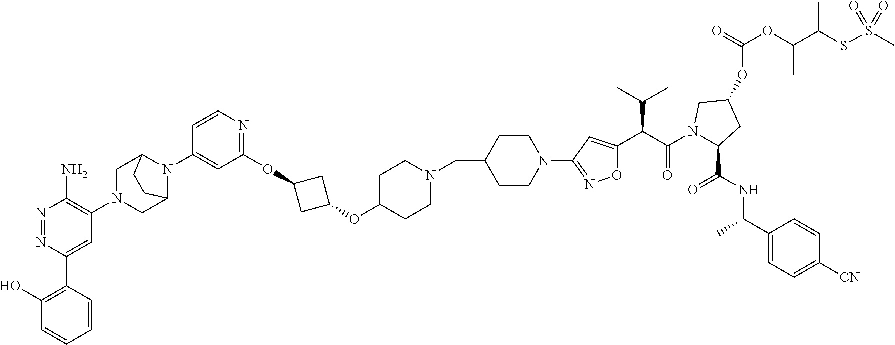 [SMILES: CC(OC(=O)O[C@@H]1C[C@@H](C(=O)N[C@@H](C)c2ccc(C#N)cc2)N(C(=O)[C@@H](c2cc(N3CCC(CN4CCC(O[C@H]5C[C@H](Oc6cc(N7C8CCC7CN(c7cc(-c9ccccc9O)nnc7N)C8)ccn6)C5)CC4)CC3)no2)C(C)C)C1)C(C)SS(C)(=O)=O]

To a 23° C. solution of S-(3-(((((3R,5S)-5-(((S)-1-(4-cyanophenyl)ethyl)carbamoyl)-1-((R)-2-(3-(4-formylpiperidin-1-yl)isoxazol-5-yl)-3-methylbutanoyl)pyrrolidin-3-yl)oxy)carbonyl)oxy)butan-2-yl) methanesulfonothioate (80 mg, 0.11 mmol) and 2-(6-amino-5-(8-(2-(3-(piperidin-4-yloxy)cyclobutoxy)pyridin-4-yl)-3,8-diazabicyclo[3.2.1]octan-3-yl)pyridazin-3-yl)phenol hydrochloride (See Compound 5 in Scheme 6 above; see synthetic route at pages 306-307 of US 2020/0038378, herein incorporated by reference in its entirety) (60 mg, 0.11 mmol) and HOAc (0.2 ml) in dichloromethane (1 mL) and methanol (1 mL) was added NaBH(OAc)₃ (232 mg, 1.09 mmol). The reaction mixture was stirred at 23° C. for 3 hours then was concentrated. The residue was purified by prep-HPLC (Boston Green ODS 150*30 mm*5 um (water (0.225% FA)-ACN, 15-45%)) to afford the title compound (35 mg, 26%) as a white solid. After pre-HPLC (FA), the desired product was a FA salt. ¹H NMR (400 MHz, DMSO-d₆): δ 8.55-8.53 (m, 1H), 8.17-8.15 (m, 1H), 7.91 (d, J=7.6 Hz, 1H), 7.81-7.75 (m, 3H), 7.51-7.44 (m, 3H), 7.23-7.20 (m, 1H), 6.89-6.82 (m, 2H), 6.54-6.51 (m, 1H), 6.12 (d, J=8.8 Hz, 2H), 5.97 (s, 2H), 5.26-5.07 (m, 2H), 5.01-4.85 (m, 2H), 4.51-4.46 (m, 2H), 4.38-4.27 (m, 2H), 3.87-3.80 (m, 2H), 3.59-3.56 (m, 3H), 3.54-3.52 (m, 3H), 3.28-3.24 (m, 1H), 3.03-2.99 (m, 2H), 2.76-2.70 (m, 2H), 2.36-2.31 (m, 2H), 2.19-2.09 (m, 4H), 2.01-1.94 (m, 4H), 1.80-1.62 (m, 5H), 1.45-1.32 (m, 9H), 1.27-1.25 (m, 1H), 1.14-1.02 (m, 2H), 0.95-0.89 (m, 3H), 0.88-0.70 (m, 3H); LCMS (ESI) m/z: 1259.1 [M+H]⁺.

### Synthesis Example 7

### Synthesis of L1-CIDE-BRM1-7

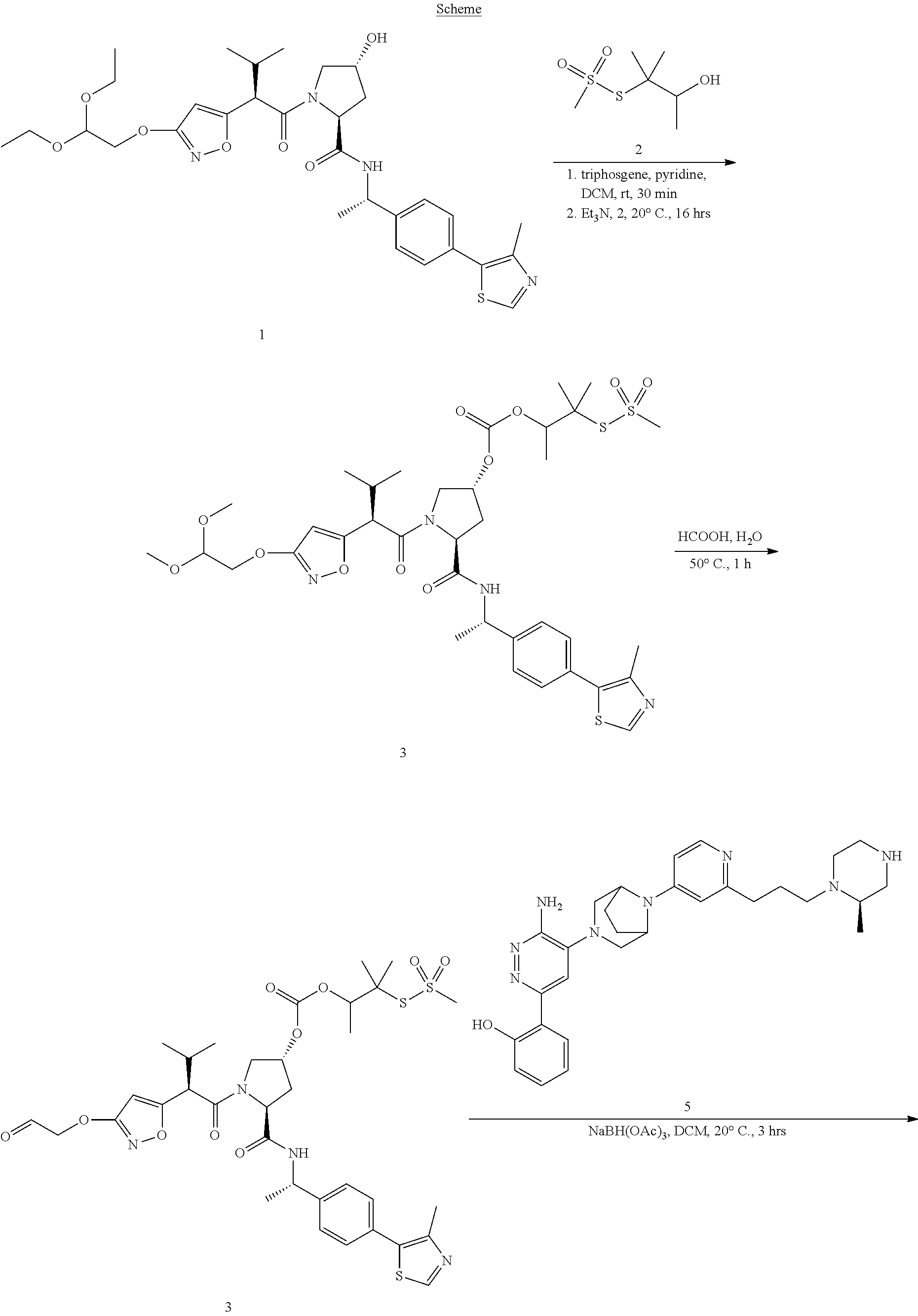

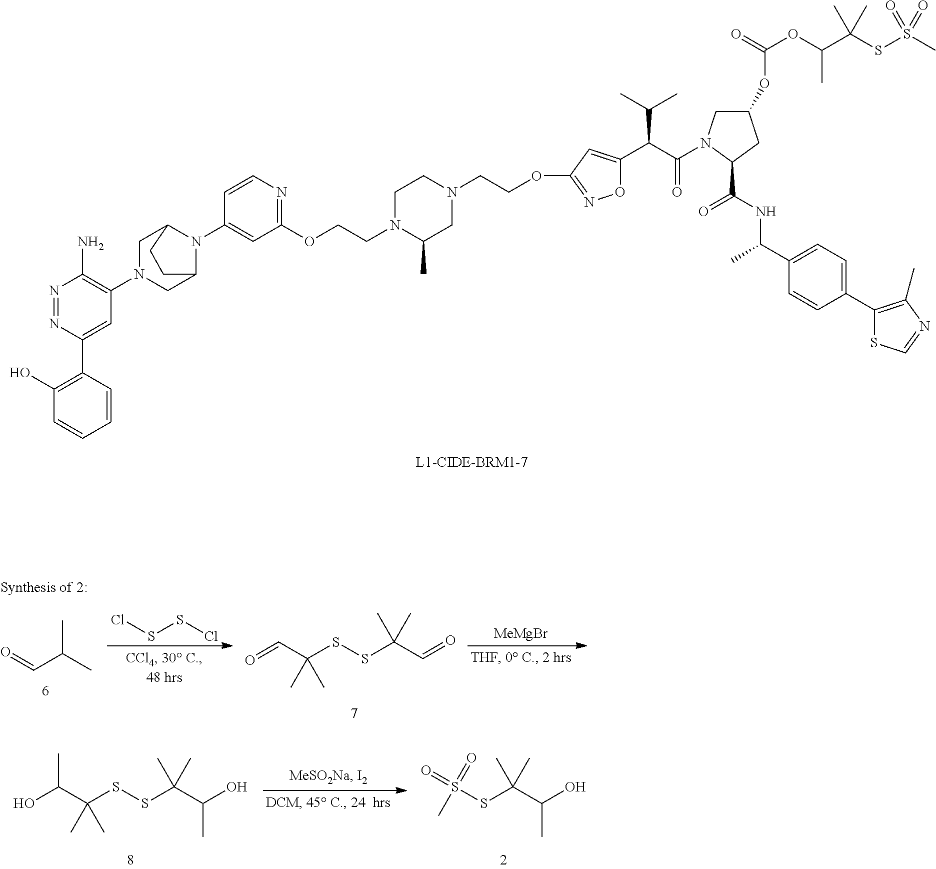

### Experimentals

General Procedure for Preparation of Compound 7:

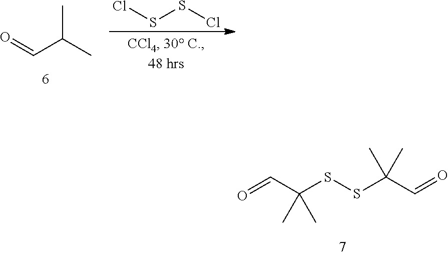

To a stirred solution isobutyraldehyde (2.7 mL, 29.6 mmol) in carbon tetrachloride (10 mL) was added dropwise disulfurdichloride (1.2 mL, 14.8 mmol) at 50° C. under a nitrogen atmosphere. The reaction was stirred for an additional 48 hours at 30° C. under a current of nitrogen to remove the hydrogen chloride liberated. The TLC (25% ethyl acetate in petroleum ether, Rf=0.5) indicated the reaction was completed. The solution is distilled under vacuum and purified by flash chromatography eluting with 25% ethyl acetate in petroleum ether to afford 2-[(1,1-dimethyl-2-oxo-ethyl)disulfanyl]-2-methyl-propanal (3000 mg, 98.2%) as a colorless oil. ¹H NMR (400 MHz, chloroform-d₆): δ=9.09 (s, 2H), 1.37 (s, 12H).

### General Procedure for Preparation of Compound 8

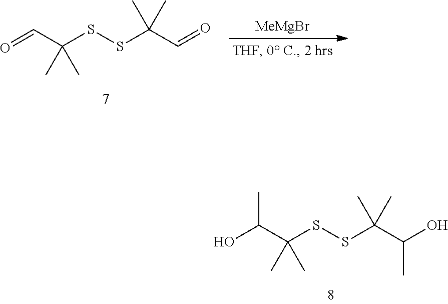

To a solution of 2,2′-disulfanediylbis(2-methylpropanal) (1500.0 mg, 7.3 mmol) in tetrahydrofuran (30 mL), methylmagnesiumbromide (9.7 mL, 29.1 mmol) was added dropwise over 5 min at 0° C. The mixture was stirred for 2 h at 0° C.

The TLC (20% ethyl acetate in petroleum ether, Rf=0.5) indicated the reaction was completed. The mixture was quenched with saturated aqueous NH₄Cl solution (10 mL) and extracted with EtOAc (20 ml×3). The combined organic phase was washed with water (20 mL) and brine (10 mL), dried over Na2SO4, filtered and concentrated to give 3-[(2-hydroxy-1,1-dimethyl-propyl)disulfanyl]-3-methyl-butan-2-ol (1370 mg, 79%) as yellow oil. ¹H NMR (400 MHz, chloroform-d₆): δ=3.77-3.74 (m, 1H), 1.31 (s, 6H), 1.26 (d, J=2.4 Hz, 3H), 1.19 (d, J=6.4 Hz, 3H).

### General Procedure for Preparation of Compound 2

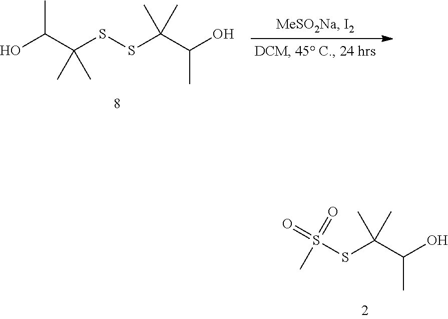

A solution of 2-methyl-2-[(5-nitro-2-pyridyl)disulfanyl]propan-1-ol (5983.8 mg, 22.99 mmol) in dichloromethane (50 mL) was added 3-[(2-hydroxy-1,1-dimethyl-propyl)disulfanyl]-3-methyl-butan-2-ol (1370.0 mg, 5.75 mmol) and iodine (2917 mg, 11.49 mmol) at 25° C., The reaction mixture was stirred at 45° C. for 24 h. TLC (33% EtOAc in petroleum ether Rf=0.4) showed the reaction had gone to completion. The mixture was filtered and the filtrate was concentrated in vacuo and purified by flash chromatography eluting with 0-50% EtOAc in petroleume ether to give 3-methyl-3-methylsulfonylsulfanyl-butan-2-ol (460 mg, 40.4% yield) as a yellow oil. ¹H NMR (400 MHz, chloroform-d₆): δ=4.14-4.09 (m, 1H), 3.42 (s, 3H), 1.68 (s, 3H), 1.45 (s, 3H), 1.29 (d, J=6.4 Hz, 3H).

### General Procedure for Preparation of Compound 3

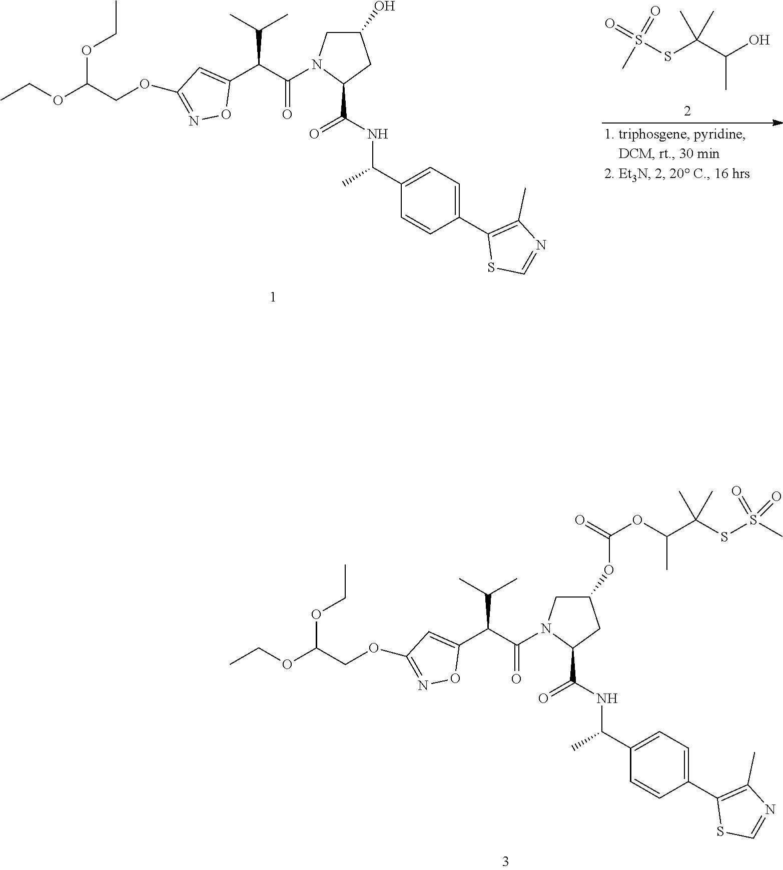 [SMILES: CCOC(COc1cc([C@H](C(=O)N2C[C@H](O)C[C@H]2C(=O)N[C@@H](C)c2ccc(-c3scnc3C)cc2)C(C)C)on1)OCC ; CC(O)C(C)(C)SS(C)(=O)=O ; CCOC(COc1cc([C@H](C(=O)N2C[C@H](OC(=O)OC(C)C(C)(C)SS(C)(=O)=O)C[C@H]2C(=O)N[C@@H](C)c2ccc(-c3scnc3C)cc2)C(C)C)on1)OCC]

To a solution of triphosgene (112.3 mg, 0.38 mmol) in dichloromethane (2 mL) was added a solution of 3-methyl-3-methylsulfonylsulfanyl-butan-2-ol (150.0 mg, 0.76 mmol) and pyridine (239.3 mg, 3.03 mmol) in dichloromethane (2 mL), the reaction was stirred at 25° C. for 30 min. The reaction mixture was concentrated to dryness to give the crude product which was used directly in next step.

To above crude product in anhydrous dichloromethane (2 mL) was added 4 Å MS (100 mg), followed by addition of a solution of triethylamine (32.9 mg, 0.33 mmol) and compound 1 (50.0 mg, 0.08 mmol) in anhydrous dichloromethane (2 mL) slowly at 20° C. and stirred for additional 16 hrs. The residue was concentrated and purified by flash chromatography on silica gel eluting with 0-70% ethyl acetate in petroleum ether to afford compound 3 (64 mg, 93.8%) as white solid. LCMS (5-95, AB, 1.5 min): RT=0.974 min, m/z=839.3 [M+H]⁺.

### General Procedure for Preparation of Compound 4

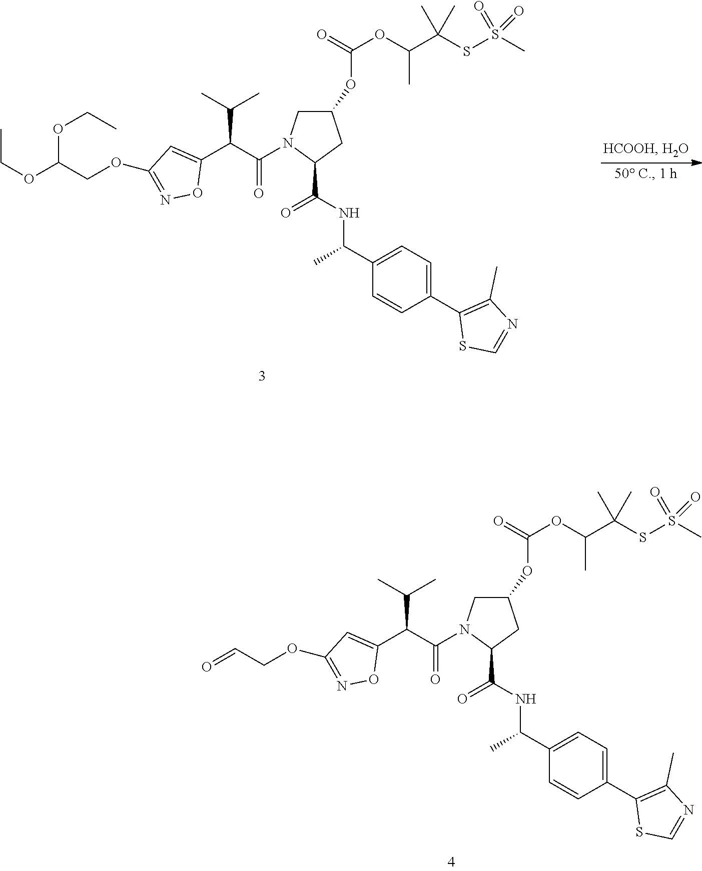 [SMILES: CCOC(COc1cc([C@H](C(=O)N2C[C@H](OC(=O)OC(C)C(C)(C)SS(C)(=O)=O)C[C@H]2C(=O)N[C@@H](C)c2ccc(-c3scnc3C)cc2)C(C)C)on1)OCC ; Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCC=O)no2)C(C)C)cc1]

A solution of was added compound 3 (50.0 mg, 0.06 mmol) in formic acid (2 mL) and water (2 mL) was stirred at 50° C. for 1 hours. The reaction mixture was concentrated to afford compound 4 (45 mg, 98.7%) as a yellow solid, which was used for the next step directly.

LCMS (5-95, AB, 1.5 min): R_{T}=0.848 min, m/z=765.2 [M+H]⁺.

General Procedure for Preparation of L1-CIDE-BRM1-7:

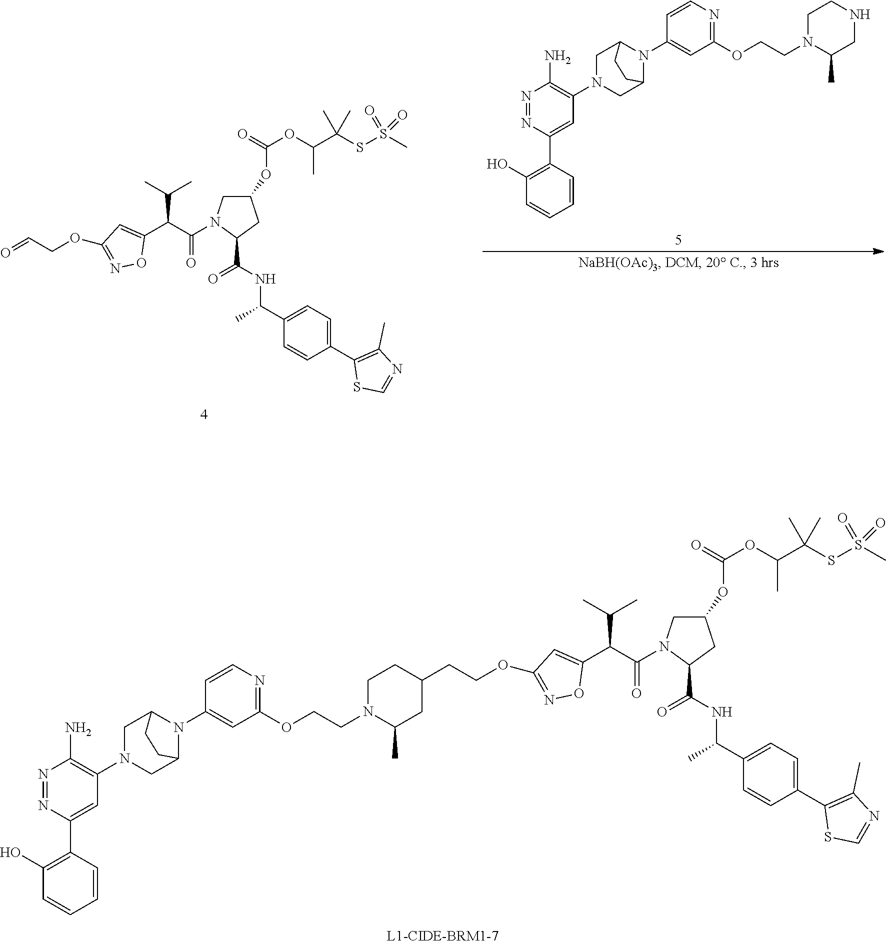 [SMILES: Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCC=O)no2)C(C)C)cc1 ; C[C@@H]1CNCCN1CCOc1cc(N2C3CCC2CN(c2cc(-c4ccccc4O)nnc2N)C3)ccn1 ; Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCCC3CCN(CCOc4cc(N5C6CCC5CN(c5cc(-c7ccccc7O)nnc5N)C6)ccn4)[C@H](C)C3)no2)C(C)C)cc1]

To a solution of compound 4 (45.0 mg, 0.06 mmol) in dichloromethane (2 mL) was added compound 5 (33.1 mg, 0.06 mmol) and sodiumtriacetoxyborohydride (249.4 mg, 1.18 mmol). The reaction mixture was stirred at 20° C. for 3 h. The mixture was concentrated and purified by TLC (8% MeOH in DCM) to give L1-CIDE-BRM1-7 (15.0 mg, 19.4%) as a white solid.

¹H NMR (400 MHz, methanol-d4): δ=8.88 (s, 1H), 7.81-7.75 (m, 2H), 7.49-7.41 (m, 5H), 7.24-7.20 (m, 1H), 6.92-6.87 (m, 2H), 6.57 (d, J=4.0 Hz, 1H), 6.22 (d, J=2.0 Hz, 1H), 6.01 (d, J=3.2 Hz, 1H), 5.28-5.24 (m, 1H), 5.05-5.02 (m, 1H), 4.61 (s, 2H), 4.53-4.49 (m, 3H), 4.36-4.31 (m, 4H), 4.04-3.90 (m, 2H), 3.67-3.65 (m, 1H), 3.46-3.43 (m, 1H), 3.39 (s, 3H), 3.16-3.03 (m, 4H), 2.95-2.91 (m, 2H), 2.78-2.72 (m, 3H), 2.68-2.63 (m, 2H), 2.49 (s, 3H), 2.39-2.33 (m, 2H), 2.26-2.23 (m, 2H), 2.19-2.06 (m, 4H), 1.59-1.52 (m, 7H), 1.46 (d, J=4.0 Hz, 2H), 1.40-1.29 (m, 6H), 1.14 (d, J=6.4 Hz, 3H), 1.05 (d, J=6.4 Hz, 3H), 0.88 (d, J=6.4 Hz, 3H).

LCMS (5-95, AB, 1.5 min): RT=0.829 min, m/z=633.5 [M/2+H]⁺. HRMS (5-95AB): m/z=1265.5126 [M+H]⁺.

### Synthesis Example 8

### Synthesis of L1-CIDE-BRM1-8

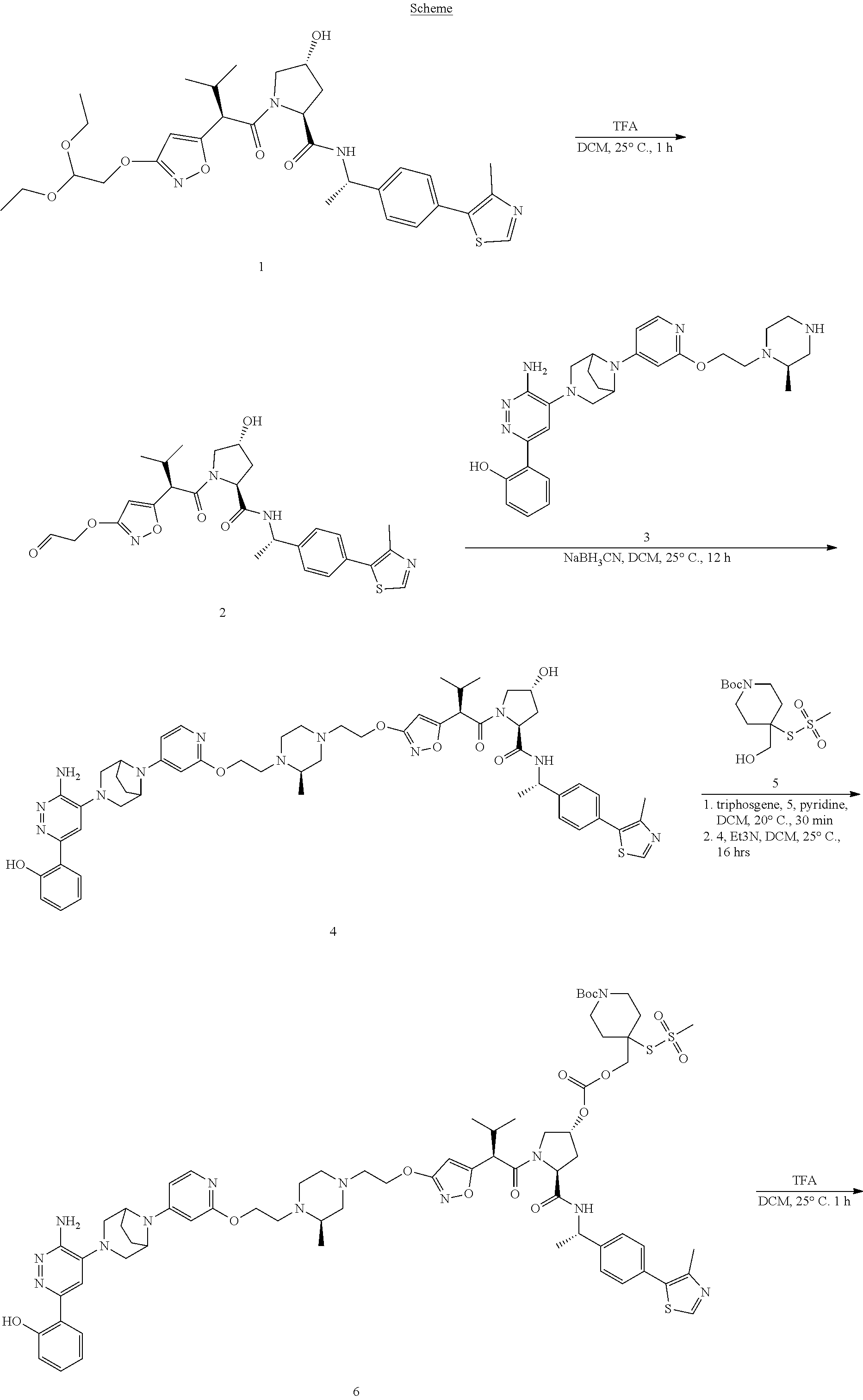

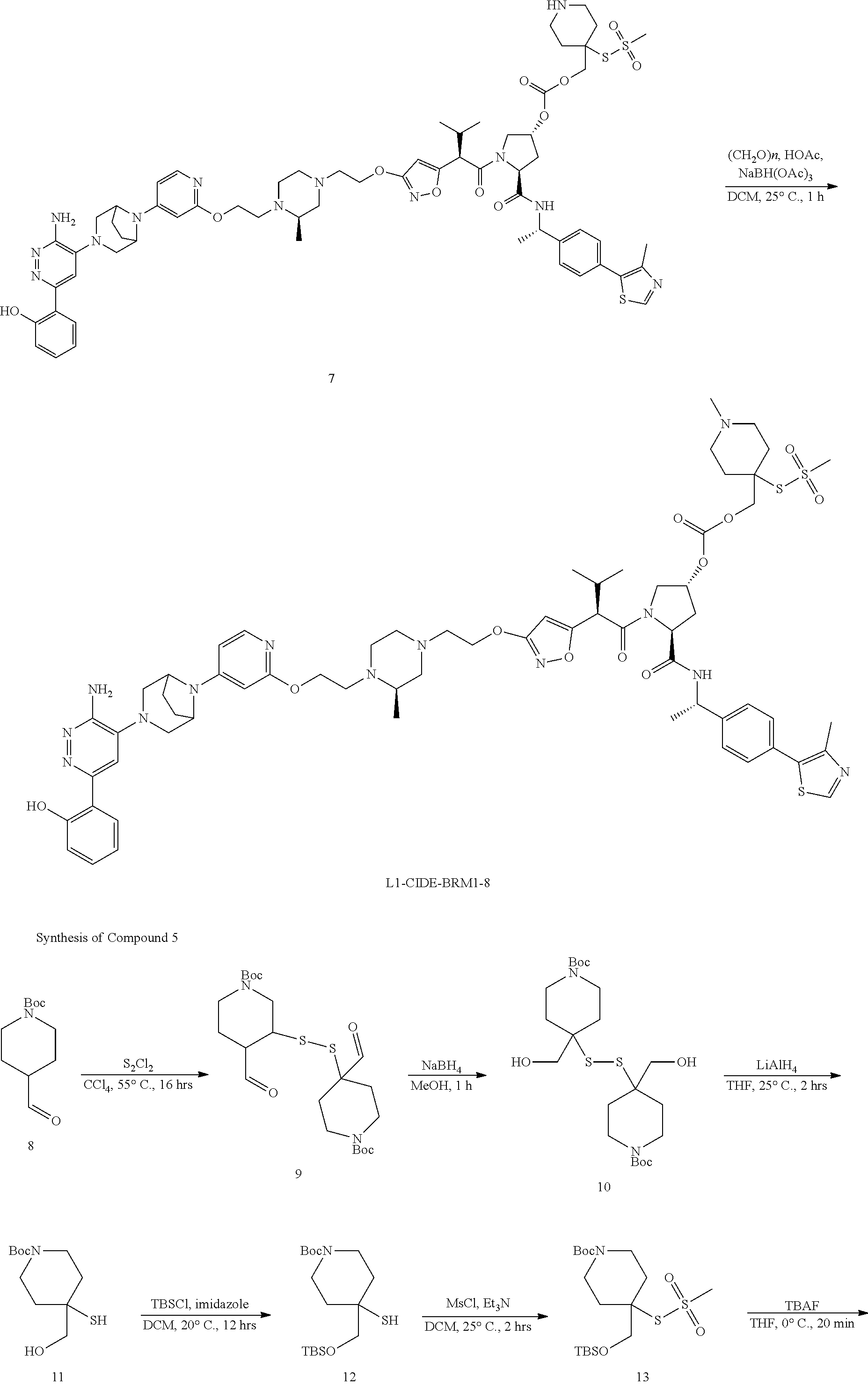

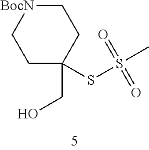 [SMILES: CC(C)(C)OC(=O)N1CCC(CO)(SS(C)(=O)=O)CC1]

### Experimentals

### General procedure for preparation of Compound 2

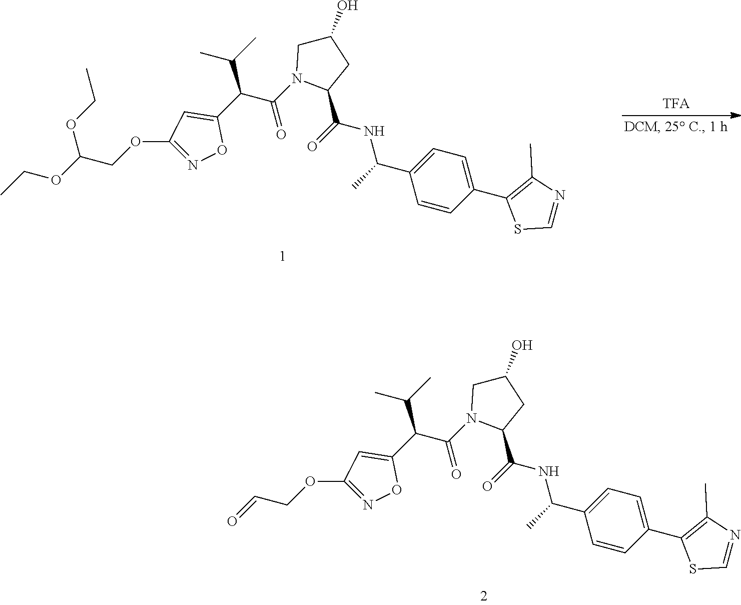 [SMILES: CCOC(COc1cc([C@H](C(=O)N2C[C@H](O)C[C@H]2C(=O)N[C@@H](C)c2ccc(-c3scnc3C)cc2)C(C)C)on1)OCC ; Cc1ncsc1-c1ccc([C@H](C)NC(=O)[C@@H]2C[C@@H](O)CN2C(=O)[C@@H](c2cc(OCC=O)no2)C(C)C)cc1]

A solution of compound 1 (120.00 mg, 0.20 mmol) in Dichloromethane (2.00 mL) and trifluoroacetic acid (0.40 mL) was stirred at 25° C. for 1 h. TLC (10% MeOH in DCM, Rf=0.6) showed most starting material was consumed and a new spot was formed. Then to the mixture was added water (3.00 mL) and adjusted pH=9 with sat.NaHCO₃ (8.00 mL). Then the mixture was extracted with Dichloromethane (15 mL×3). The organic layers were concentrated to give the crude compound 2 (90.00 mg, 85.3%) as a white solid. LCMS (5-95, AB, 1.5 min): R_{T}=0.789 min, m/z=541.3 [M+H]⁺.

### General Procedure for Preparation of Compound 4

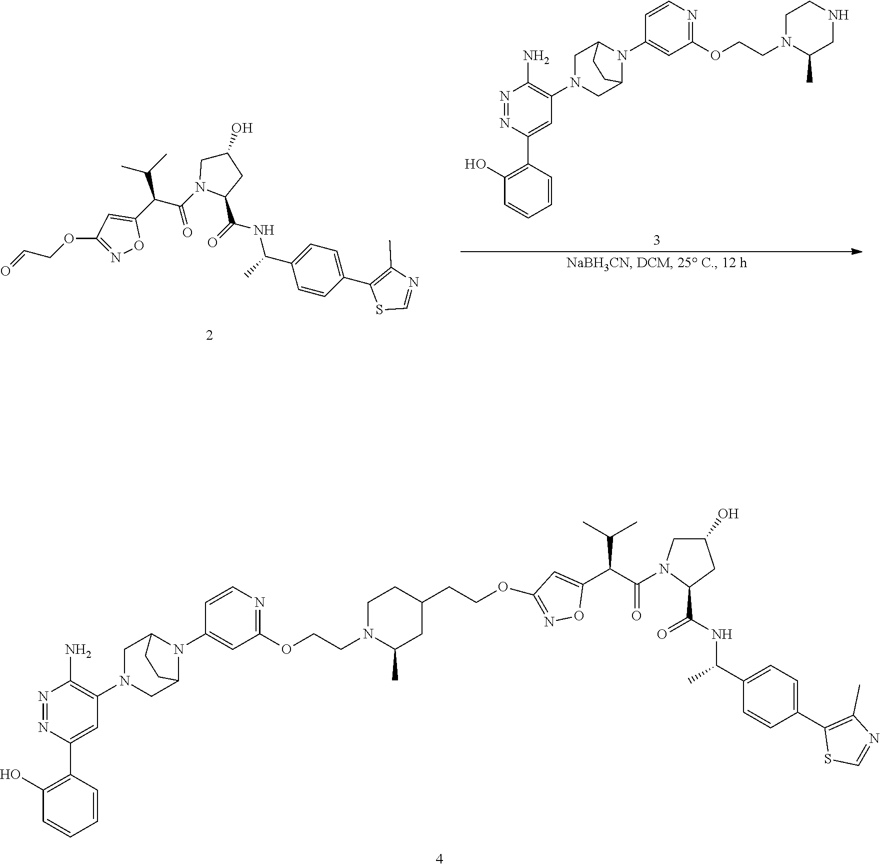 [SMILES: Cc1ncsc1-c1ccc([C@H](C)NC(=O)[C@@H]2C[C@@H](O)CN2C(=O)[C@@H](c2cc(OCC=O)no2)C(C)C)cc1 ; C[C@@H]1CNCCN1CCOc1cc(N2C3CCC2CN(c2cc(-c4ccccc4O)nnc2N)C3)ccn1 ; Cc1ncsc1-c1ccc([C@H](C)NC(=O)[C@@H]2C[C@@H](O)CN2C(=O)[C@@H](c2cc(OCCC3CCN(CCOc4cc(N5C6CCC5CN(c5cc(-c7ccccc7O)nnc5N)C6)ccn4)[C@H](C)C3)no2)C(C)C)cc1]

To a solution of compound 3 (75.50 mg, 0.14 mmol) and compound 2 (90.00 mg, 0.14 mmol) in Methyl alcohol (5.00 mL) and Dichloromethane (5.00 mL) was added sodiumcyanoborohydride (12.9 mg, 0.20 mmol) and sodium acetate (16.80 mg, 0.20 mmol). The mixture was stirred at 25° C. for 12 h. TLC (12% MeOH in DCM, Rf=0.6) showed most starting material was consumed and a new spot was formed. The mixture was filtered and the filtrate was concentrated and purified by Pre-TLC (12% MeOH in DCM) to afford compound 4 (40.00 mg, 28.1%) as a white solid. LCMS (5-95, AB, 1.5 min): R_{T}=0.755 min, m/z=1041.2 [M+H]⁺.

### General Procedure for Preparation of Compound 6

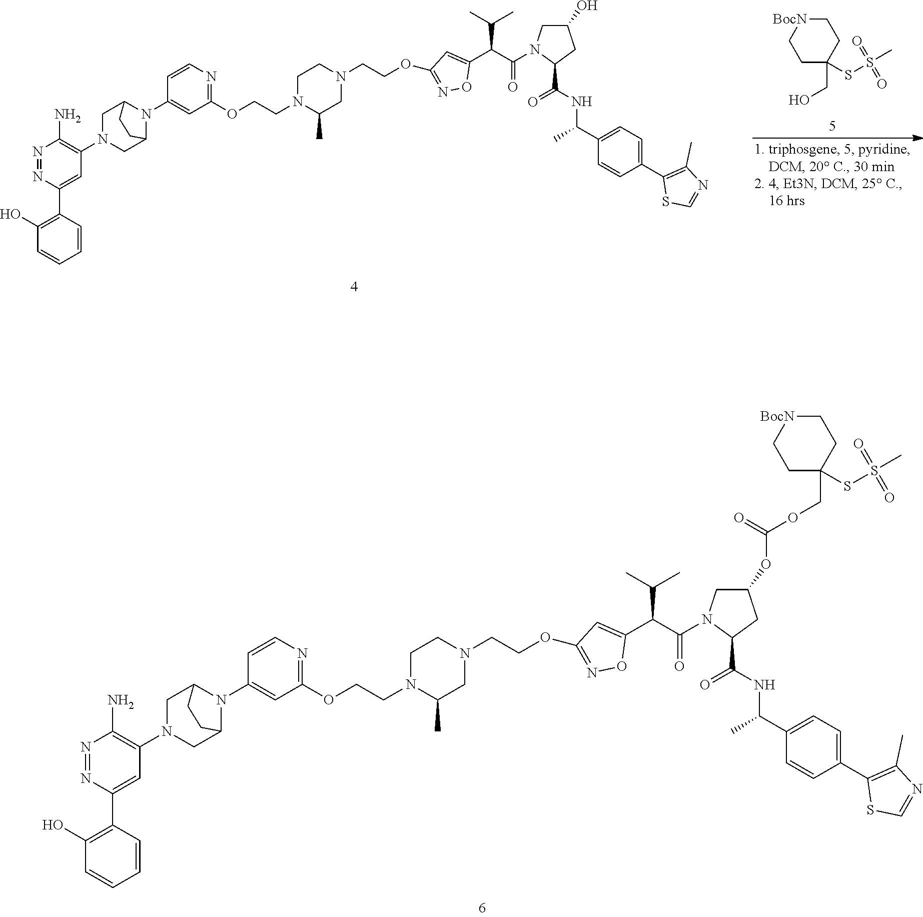 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1 ; CC(C)(C)OC(=O)N1CCC(CO)(SS(C)(=O)=O)CC1 ; Cc1ncsc1-c1ccc([C@H](C)NC(=O)[C@@H]2C[C@@H](OC(=O)OCC3(SS(C)(=O)=O)CCN(C(=O)OC(C)(C)C)CC3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a mixture of triphosgene (18.3 mg, 0.062 mmol) and 4A molecular sieves in Dichloromethane (2.0 mL) was added a solution of tert-butyl 4-(hydroxymethyl)-4-methylsulfonylsulfanyl-piperidine-1-carboxylate (20.0 mg, 0.062 mmol) and pyridine (0.02 mL, 0.184 mmol) in Dichloromethane (2.0 mL). The reaction mixture was stirred at 20° C. for 30 min. The reaction mixture was concentrated to give the crude product which was used directly in next step.

To above crude product and 4 Å MS (100 mg) in anhydrous Dichloromethane (5.00 mL) was added a solution of N,N-Diisopropylethylamine (0.02 mL, 0.09 mmol) and compound 4 (30.00 mg, 0.03 mmol) in anhydrous N,N-Dimethylformamide (2.00 mL). The mixture was stirred at 25° C. for 16 hrs. TLC (11% MeOH in DCM, Rf=0.6) showed most starting material was consumed and a new spot was formed. The mixture was filtered and concentrated to give the crude product, which was purified by Pre-TLC (11% MeOH in DCM) to afford compound 6 (25.00 mg, 62.3%) as a pale yellow solid. LCMS (10-80, AB, 7.0 min): R_{T}=3.096 min, m/z=697.1 [M/2+H]⁺

### General Procedure for Preparation of Compound 7

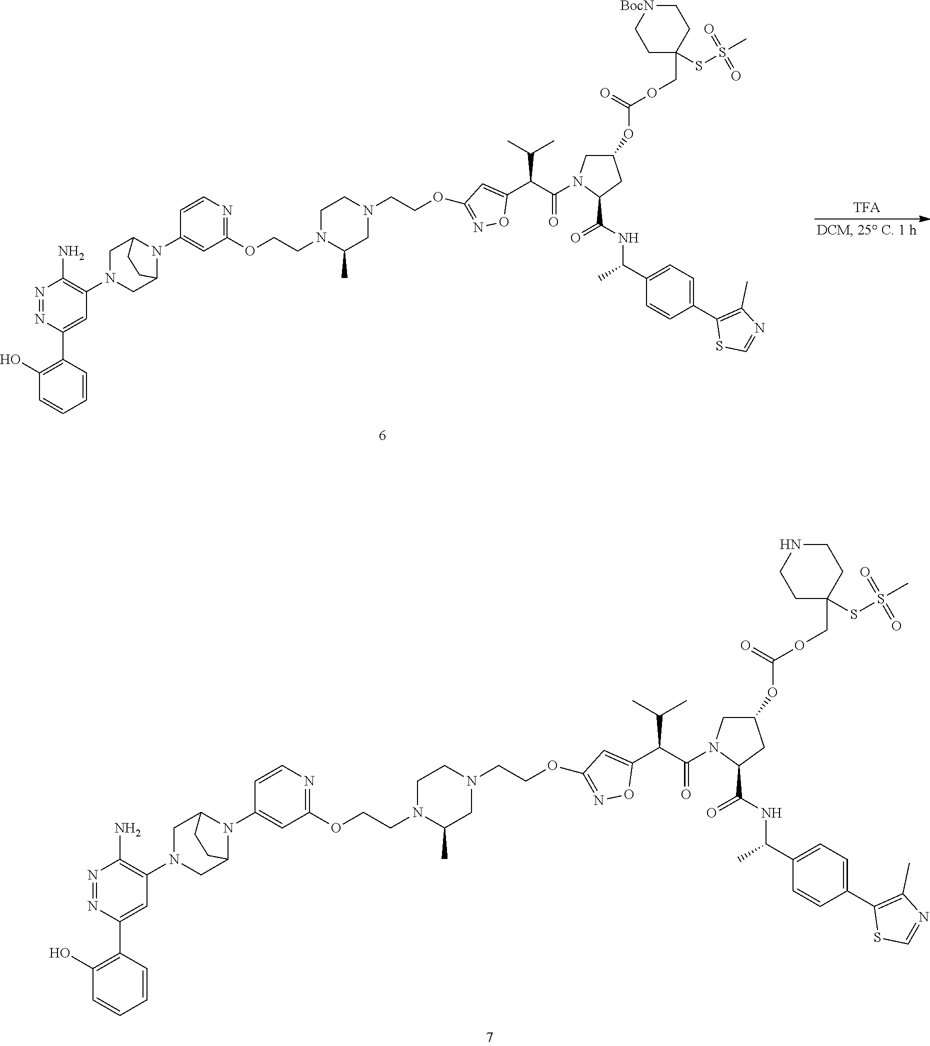 [SMILES: Cc1ncsc1-c1ccc([C@H](C)NC(=O)[C@@H]2C[C@@H](OC(=O)OCC3(SS(C)(=O)=O)CCN(C(=O)OC(C)(C)C)CC3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1 ; Cc1ncsc1-c1ccc([C@H](C)NC(=O)[C@@H]2C[C@@H](OC(=O)OCC3(SS(C)(=O)=O)CCNCC3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a solution of compound 6 (20.00 mg, 0.01 mmol) in Dichloromethane (1.00 mL) was added trifluoroacetic acid (0.80 mL, 10.38 mmol). The mixture was stirred at 25° C. for 1 h. The mixture was concentrated to give the crude product 7 as TFA salt which was used for next step directly.

### General Procedure for Preparation of L1-CIBE-BRM1-8

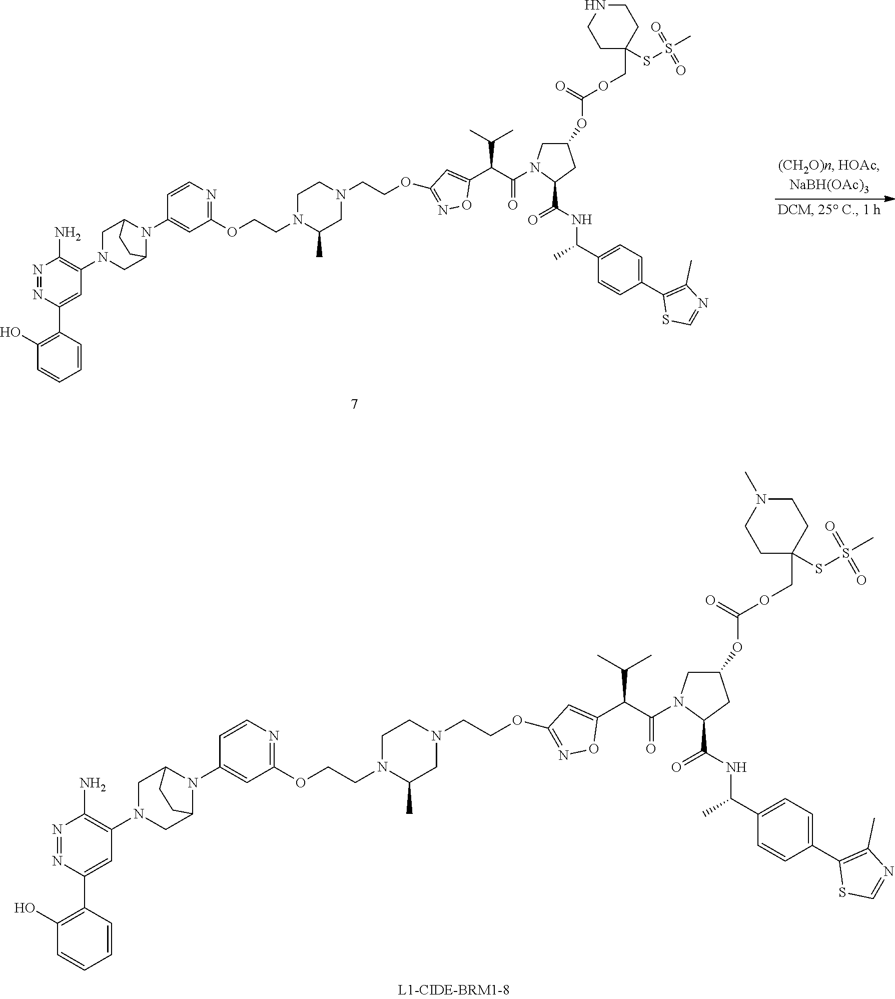 [SMILES: Cc1ncsc1-c1ccc([C@H](C)NC(=O)[C@@H]2C[C@@H](OC(=O)OCC3(SS(C)(=O)=O)CCNCC3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1 ; Cc1ncsc1-c1ccc([C@H](C)NC(=O)[C@@H]2C[C@@H](OC(=O)OCC3(SS(C)(=O)=O)CCN(C)CC3)CN2C(=O)[C@@H](c2cc(OCCN3CCN(CCOc4cc(N5C6CCC5CN(c5cc(-c7ccccc7O)nnc5N)C6)ccn4)[C@H](C)C3)no2)C(C)C)cc1]

To a solution of formaldehyde (4.3 mg, 0.14 mmol) and compound 7 (20.20 mg, 0.01 mmol) in Dichloromethane (2 mL) and Methyl alcohol (1 mL) was added acetic acid (1.00 mg). The mixture was added at 25° C. for 30 min. Then sodiumtriacetoxyborohydride (9.2 mg, 0.04 mmol) was added. The mixture was stirred at 25° C. for 1 h. Then the mixture was diluted with DCM (15 mL) and washed with sat. NaHCO₃ (5 mL) and the organic layer was concentrated and the residue was purified by reverse phase chromatography (acetonitrile 14-44/0.225% FA in water) to afford L1-CIDE-BRM1-8 (3.60 mg, 18.2%) as a white solid.

¹H NMR (400 MHz, DMSO-d6): δ=8.99 (s, 1H), 8.49 (d, J=8.0 Hz, 1H), 8.15 (s, 1H), 7.91 (d, J=8.0 Hz, 1H), 7.78 (d, J=6.0 Hz, 1H), 7.49-7.36 (m, 6H), 7.22-7.20 (m, 1H), 6.88-6.85 (m, 2H), 6.54-6.52 (m, 1H), 6.13 (d, J=8.0 Hz, 2H), 5.97-5.94 (m, 2H), 5.25-5.21 (m, 1H), 4.93-4.89 (m, 1H), 4.52-4.43 (m, 6H), 4.26-4.21 (m, 6H), 3.87 (br s, 1H), 3.72 (d, J=9.2 Hz, 1H), 3.52 (s, 3H), 3.02-2.96 (m, 4H), 2.61 (br s, 4H), 2.46-2.33 (m, 10H), 2.20-2.14 (m, 6H), 1.96-1.89 (m, 10H), 1.38 (d, J=7.2 Hz, 3H), 0.99-0.95 (m, 6H), 0.81 (d, J=6.4 Hz, 3H). LCMS (5-95, AB, 1.5 min): R_{T}=0.781 min, m/z=1306.5 [M+H]⁺.

### General Procedure for Preparation of Compound 9

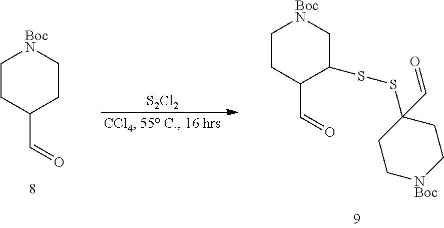 [SMILES: CC(C)(C)OC(=O)N1CCC(C=O)CC1 ; "S2Cl2" ; CC(C)(C)OC(=O)N1CCC(C=O)(SSC2CN(C(=O)OC(C)(C)C)CCC2C=O)CC1]

To a mixture of tert-butyl 4-formyl-1-piperidinecarboxylate (9870.7 mg, 46.28 mmol) in Carbon tetrachloride (150 mL) was added disulfurdichloride (1.48 mL, 18.51 mmol) at 55° C., the mixture was stirred at 55° C. for 16 hrs, TLC (10% Methyl alcohol in Dichloromethane, Rf=0.5) indicated the reaction was completed. The mixture was filtrated and the organic layer was concentrated in vacuum. The residue was added water (30 mL) and extracted with Dichloromethane (3×50 mL), the organic layers were combined and dried over Na₂SO4, filtered and concentrated to give the crude which was purified by Pre-TLC (10% Methyl alcohol in Dichloromethane, Rf=0.5) to afford tert-butyl 4-[(1-tert-butoxycarbonyl-4-formyl-4-piperidyl)disulfanyl]-4-formyl-piperidine-1-carboxylate (5.5 g, 60.8%) as a white solid. ¹H NMR (400 MHz, chloroform-d₆): δ=9.06 (s, 2H), 3.73 (br s, 4H), 3.18-3.11 (m, 4H), 2.07-2.02 (m, 4H), 1.74-1.69 (m, 4H), 1.46 (s, 18H).

### General Procedure for Preparation of Compound 10

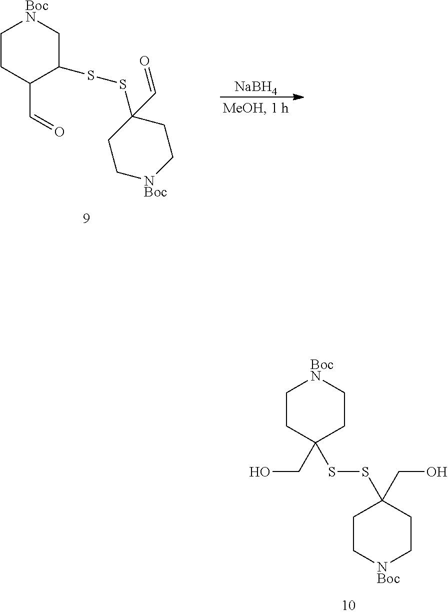 [SMILES: CC(C)(C)OC(=O)N1CCC(C=O)(SSC2CN(C(=O)OC(C)(C)C)CCC2C=O)CC1 ; "NaBH4" ; CC(C)(C)OC(=O)N1CCC(CO)(SSC2(CO)CCN(C(=O)OC(C)(C)C)CC2)CC1]

To a mixture of tert-butyl 4-[(1-tert-butoxycarbonyl-4-formyl-4-piperidyl)disulfanyl]-4-formyl-piperidine-1-carboxylate (3000.0 mg, 6.14 mmol) in Methyl alcohol (40 mL) was added sodium borohydride (696.7 mg, 18.42 mmol), the mixture was stirred at 25° C. for 1 h, TLC (10% Methyl alcohol in Dichloromethane, Rf=0.5) showed a new spot, the reaction was quenched by addition of water (30 mL) and the resulted mixture was extracted with Dichloromethane (3×30 mL), the organic layers were combined and dried with Na₂SO4, filtered and concentrated to give the crude product which was purified by chromatography on silica eluting with 0-3% Methyl alcohol in Dichloromethane to afford tert-butyl 4-[[1-tert-butoxycarbonyl-4-(hydroxymethyl)-4-piperidyl]disulfanyl]-4-(hydroxymethyl)piperidine-1-carboxylate (3000 mg, 99%) as a white solid. ¹H NMR (400 MHz, chloroform-d₆): δ=3.75-3.72 (m, 4H), 3.59 (s, 4H), 3.32-3.26 (m, 4H), 1.75-1.67 (m, 8H), 1.46 (s, 18H).

### General Procedure for Preparation of Compound 11

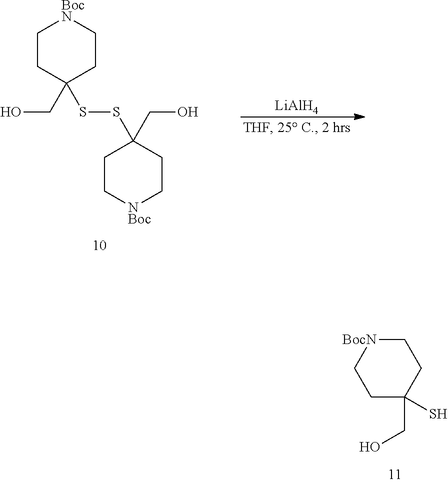 [SMILES: CC(C)(C)OC(=O)N1CCC(CO)(SSC2(CO)CCN(C(=O)OC(C)(C)C)CC2)CC1 ; "LiAlH4" ; CC(C)(C)OC(=O)N1CCC(S)(CO)CC1]

To a suspension of Lithium aluminum hydride (1232.4 mg, 32.47 mmol) in Tetrahydrofuran (40 mL) was added a solution of tert-butyl 4-[[1-tert-butoxycarbonyl-4-(hydroxymethyl)-4-piperidyl]disulfanyl]-4-(hydroxymethyl)piperidine-1-carboxylate (3200.0 mg, 6.49 mmol) in Tetrahydrofuran (40 mL) dropwisely. The formed mixture was stirred for 2 hours at 25° C. under nitrogen. The reaction was quenched with aqueous NH₄Cl (10 mL), and extracted with Ethyl acetate (30 mL×3). The organic layer was dried over anhydrous sodium sulfate, concentrated under vacuum to afford the crude product tert-butyl 4-(hydroxymethyl)-4-sulfanyl-piperidine-1-carboxylate (2700 mg, 100%), which was used to next step directly. ¹H NMR (400 MHz, chloroform-d₆): δ=3.96-3.92 (m, 2H), 3.52 (s, 2H) 3.27-3.21 (m, 2H), 1.64-1.61 (m, 4H), 1.47 (s, 9H).

### General Procedure for Preparation of Compound 12

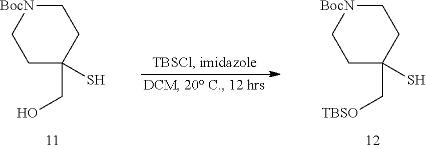 [SMILES: CC(C)(C)OC(=O)N1CCC(S)(CO)CC1 ; CC(C)(C)OC(=O)N1CCC(S)(CO[Si](C)(C)C(C)(C)C)CC1]

To a solution of imidazole (1783.5 mg, 26.2 mmol) and tert-butyl 4-(hydroxymethyl)-4-sulfanyl-piperidine-1-carboxylate (2700.0 mg, 10.92 mmol) in Dichloromethane (40 mL) was added tert-butyldimethylchlorosilane (2467.8 mg, 16.37 mmol) in Dichloromethane (40 mL). The mixture was stirred continuously at 20° C. for 12 hours. The TLC (20% ethyl acetate in petroleum ether, Rf=0.58) indicated the reaction was completed. Then the mixture was washed with water (20 mL), the organic layer was dried over anhydrous sodium sulfate and concentrated under vacuo, the crude product was purified by chromatography on silica (10% ethyl acetate in petroleum ether, Rf=0.58) to afford tert-butyl 4-[[tert-butyl(dimethyl)silyl]oxymethyl]-4-sulfanyl-piperidine-1-carboxylate (3800 mg, 96.3%). ¹H NMR (400 MHz, chloroform-d₆): δ=3.96-3.92 (m, 2H), 3.52 (s, 2H), 3.20-3.13 (m, 2H), 1.72-1.65 (m, 2H), 1.52-1.49 (m, 2H), 1.45 (s, 9H), 0.90 (s, 9H), 0.06 (s, 6H).

### General Procedure for Preparation of Compound 13

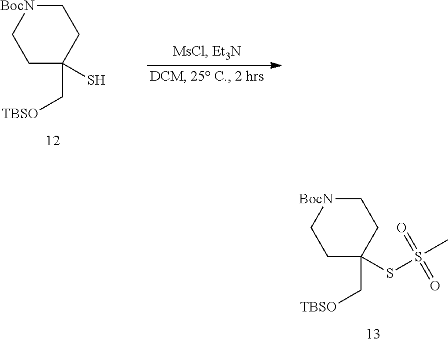 [SMILES: CC(C)(C)OC(=O)N1CCC(S)(CO[Si](C)(C)C(C)(C)C)CC1 ; CC(C)(C)OC(=O)N1CCC(CO[Si](C)(C)C(C)(C)C)(SS(C)(=O)=O)CC1]

To a solution of methanesulfonyl chloride (2.51 g, 21.91 mmol) in Dichloromethane (20 mL) under N2 protection was added a solution of tert-butyl 4-[[tert-butyl(dimethyl)silyl]oxymethyl]-4-sulfanyl-piperidine-1-carboxylate (3.8 g, 10.51 mmol) and triethylamine (5.45 mL, 42.03 mmol) in Dichloromethane (20 mL) dropwisely. The mixture was stirred at 25° C. for 2 hours. TLC (20% ethyl acetate in Petroleum ether, Rf=0.3) showed a new spot. The reaction was quenched with water (30 mL) and extracted with Dichloromethane (30 mL×3). The organic layer was concentrated and purified by column on silica eluting with 0-10% ethyl acetate in Petroleum ether o afford tert-butyl 4-[[tert-butyl(dimethyl)silyl]oxymethyl]-4-methylsulfonylsulfanyl-piperidine-1-carboxylate (1670 mg, 36.1%) as a white solid. ¹H NMR (400 MHz, chloroform-d₆): δ=3.94-3.88 (m, 4H), 3.39 (s, 3H), 3.26-3.20 (m, 1H), 1.99-1.95 (m, 2H), 1.86-1.80 (m, 2H), 1.46 (s, 9H), 0.91 (s, 9H), 0.10 (s, 6H). LCMS (5-95, AB, 1.5 min): R_{T}=1.126 min, m/z=340.1[M−100+H]⁺.

### General Procedure for Preparation of Compound 5

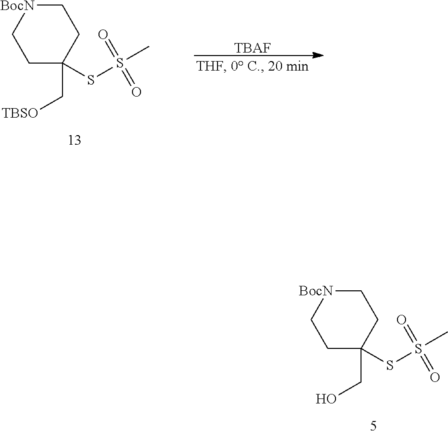 [SMILES: CC(C)(C)OC(=O)N1CCC(CO[Si](C)(C)C(C)(C)C)(SS(C)(=O)=O)CC1 ; "TBAF" ; CC(C)(C)OC(=O)N1CCC(CO)(SS(C)(=O)=O)CC1]

To a solution of tert-butyl 4-[[tert-butyl(dimethyl)silyl]oxymethyl]-4-methylsulfonylsulfanyl-piperidine-1-carboxylate (1500.00 mg, 3.41 mmol) in Tetrahydrofuran (10.0 mL) was added t etrabutylammoniumfluoride (5.12 mL, 5.12 mmol, 1 mol/L in THF). The mixture was stirred at 0° C. for 20 min. TLC (60% EtOAc in petroleum ether, Rf=0.4) showed most starting material was consumed. To the mixture was added EtOAc (70 mL), the organic layer was washed with water (25 mL×2), Brine (25 mL) and the organic layer was concentrated to give the crud e product, which was purified by flash column on silica eluting 0-60% EtOAc in petroleum ether to afford tert-butyl 4-(hydroxymethyl)-4-methylsulfonylsulfanyl-piperidine-1-carboxylat e (670.00 mg, 60.4%) as a colorless oil. ¹H NMR (400 MHz, chloroform-d₆): δ=3.96 (s, 2H), 3.89-3.85 (m, 2H), 3.43 (s, 3H), 3.31-3.28 (m, 2H), 2.12-2.08 (m, 2H), 1.82-1.76 (m, 2H), 1.47 (s, 9H).

### Synthesis Example 9

### Synthesis of L1-CIDE-BRM1-9

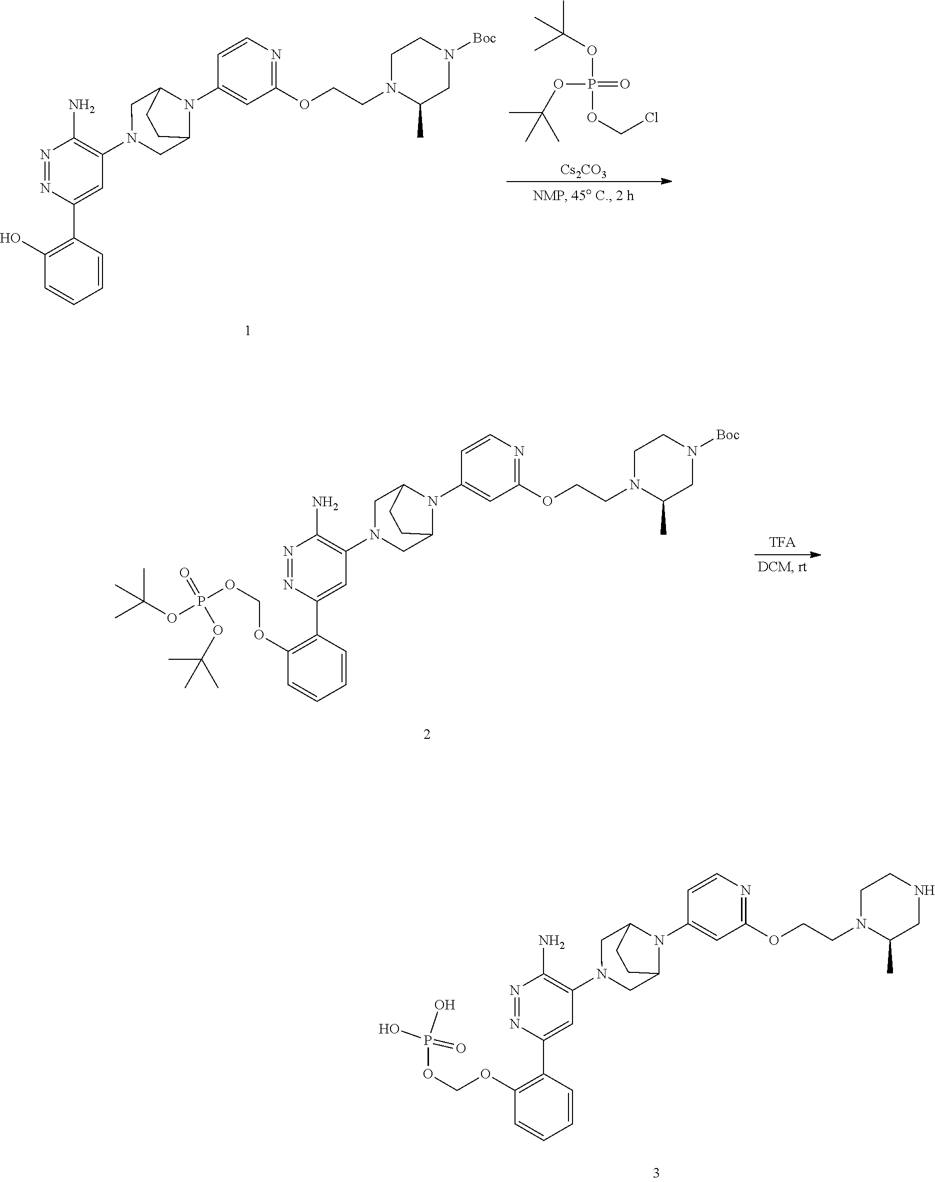

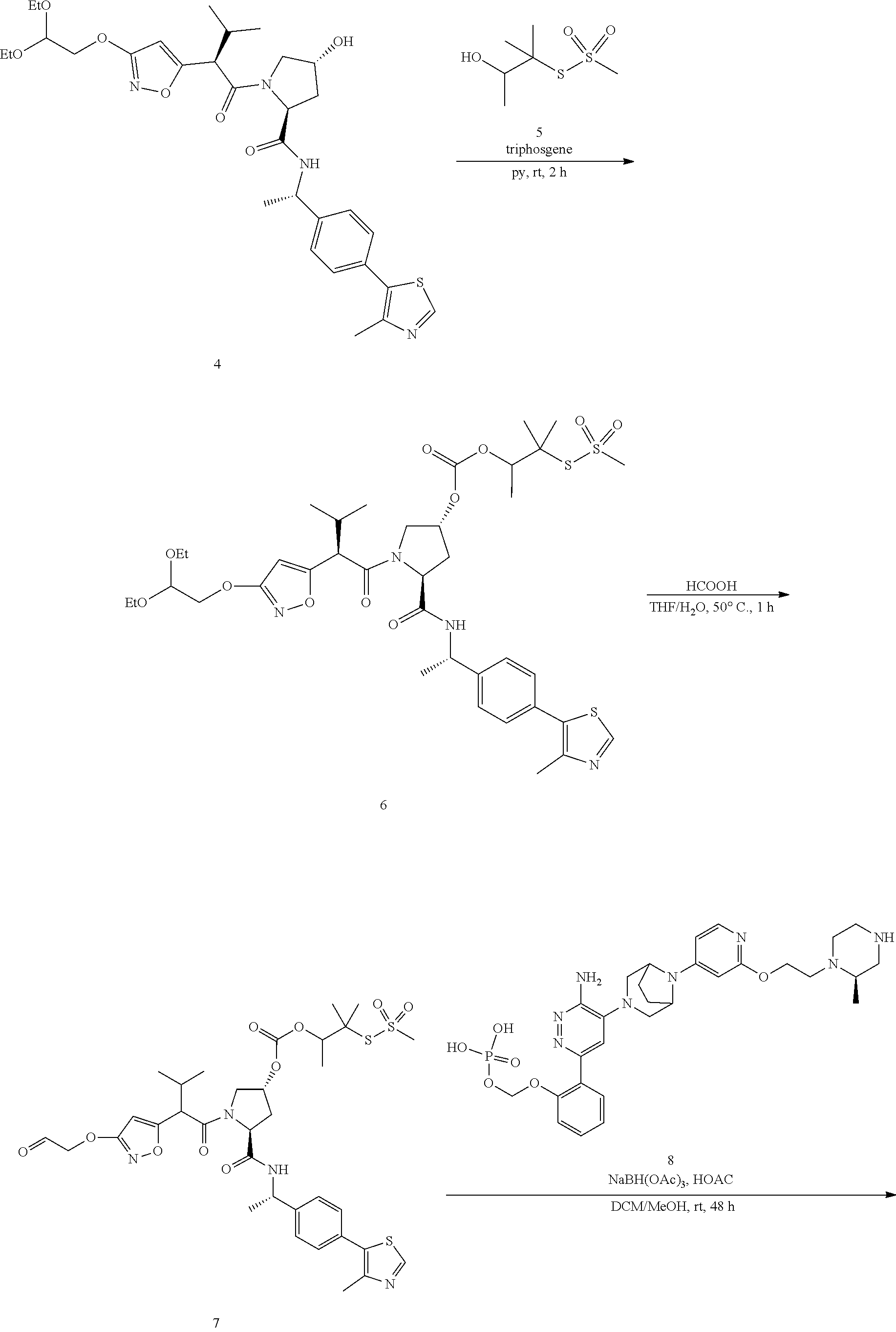 [SMILES: CCOC(COc1cc([C@H](C(=O)N2C[C@H](O)C[C@H]2C(=O)N[C@@H](C)c2ccc(-c3scnc3C)cc2)C(C)C)on1)OCC ; CC(O)C(C)(C)SS(C)(=O)=O ; CCOC(COc1cc([C@H](C(=O)N2C[C@H](OC(=O)OC(C)C(C)(C)SS(C)(=O)=O)C[C@H]2C(=O)N[C@@H](C)c2ccc(-c3scnc3C)cc2)C(C)C)on1)OCC ; "HCOOH" ; Cc1ncsc1-c1ccc([C@H](C)NC(=O)[C@@H]2C[C@@H](OC(=O)OC(C)C(C)(C)SS(C)(=O)=O)CN2C(=O)C(c2cc(OCC=O)no2)C(C)C)cc1 ; C[C@@H]1CNCCN1CCOc1cc(N2C3CCC2CN(c2cc(-c4ccccc4OCOP(=O)(O)O)nnc2N)C3)ccn1]

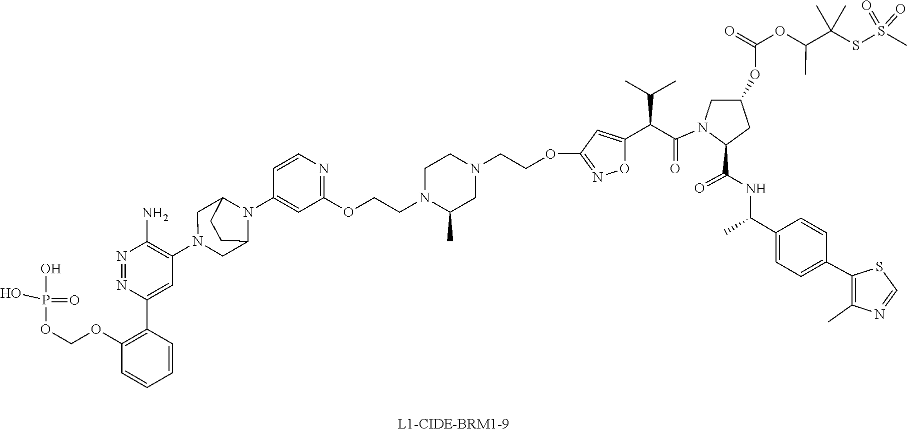 [SMILES: Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1]

### Step 1: (3R)-tert-butyl 4-(2-((4-(3-(3-amino-6-(2-(((di-tert-butoxyphosphoryl)oxy)methoxy)phenyl)pyridazin-4-yl)-3,8-diazabicyclo[3.2.1]octan-8-yl)pyridin-2-yl)oxy)ethyl)-3-methylpiperazine-1-carboxylate

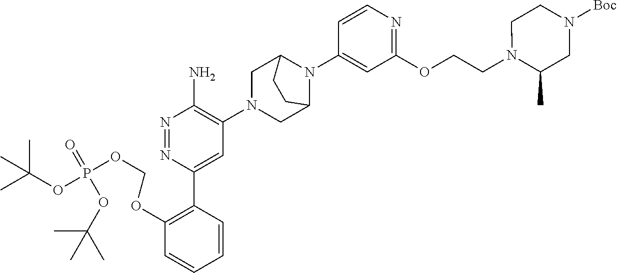 [SMILES: C[C@@H]1CN(C(=O)OC(C)(C)C)CCN1CCOc1cc(N2C3CCC2CN(c2cc(-c4ccccc4OCOP(=O)(OC(C)(C)C)OC(C)(C)C)nnc2N)C3)ccn1]

A solution of (3R)-tert-butyl 4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (300 mg, 0.49 mmol) in 1-methylpyrrolidin-2-one (8.0 mL) was added cesium carbonate (0.32 g, 0.97 mmol) and di-tert-butyl (chloromethyl) phosphate (0.19 g, 0.73 mmol). The reaction mixture was stirred at 45° C. for 12 h. The reaction mixture was quenched by the water (20 mL) and extracted with ethyl acetate (20 mL×3). The combined organic layers were washed with brine (10 mL×2), dried over anhydrous sodium sulfate, filtered and concentrated under reduced pressure to give a residue. The residue was purified by silica gel chromatography (silica gel, 100-200 mesh, 0-2% methanol in dichloromethane) to give the title compound (200 mg, 49% yield) as a yellow oil.

LCMS (ESI) m/z: 839.3 [M+H]⁺.

### Step 2: (2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl dihydrogen phosphate

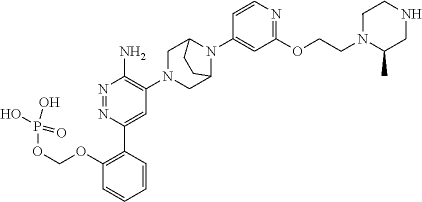 [SMILES: C[C@@H]1CNCCN1CCOc1cc(N2C3CCC2CN(c2cc(-c4ccccc4OCOP(=O)(O)O)nnc2N)C3)ccn1]

To a solution of (3R)-tert-butyl 4-(2-((4-(3-(3-amino-6-(2-(((di-tert-butoxyphosphoryl)oxy)methoxy)phenyl)pyridazin-4-yl)-3,8-diazabicyclo[3.2.1]octan-8-yl)pyridin-2-yl)oxy)ethyl)-3-methylpiperazine-1-carboxylate (200 mg, 0.24 mmol) was added 5% trifluoroacetic acid in hexafluoroisopropanol (5.0 mL). The reaction mixture was stirred at 20° C. for 3 h. The reaction was concentrated purified by Column Boston Green ODS 150*30 mm*5 um condition water (0.225% formic acid)—acetonitrile (5%-35%) to afford the title compound (100 mg, 56.6% yield) as yellow solid.

LCMS (ESI) m/z: 627.3 [M+H]⁺.

### Step 3: S-(3-(((((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)-2-methylbutan-2-yl) methanesulfonothioate

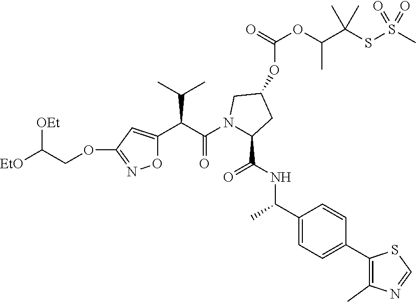 [SMILES: CCOC(COc1cc([C@H](C(=O)N2C[C@H](OC(=O)OC(C)C(C)(C)SS(C)(=O)=O)C[C@H]2C(=O)N[C@@H](C)c2ccc(-c3scnc3C)cc2)C(C)C)on1)OCC]

To a mixture of (2S, 4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (380 mg, 0.62 mmol) and triethylamine (250 mg, 2.47 mmol) and 4 Å MS (50 mg) in anhydrous dichloromethane (5.0 mL) was added S-(3-((chlorocarbonyl)oxy)-2-methylbutan-2-yl) methanesulfonothioate (322 mg, 1.24 mmol) slowly at 20° C. for 16 h. The mixture was purified by pre-TLC (7% methanol in dichloromethane) to give the title compound (150 mg, 28.9%) as a white solid.

LCMS (ESI) m/z: 839.7 [M+H]⁺.

### Step 4: S-(2-methyl-3-(((((3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate

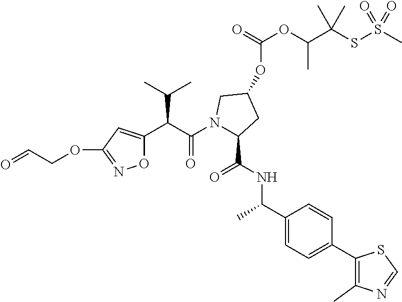 [SMILES: Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCC=O)no2)C(C)C)cc1]

A solution of S-(3-(((((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl)oxy)carbonyl)oxy)-2-methylbutan-2-yl) methanesulfonothioate (150 mg, 0.18 mmol) in water (3.0 mL) and formic acid (8.0 mL) was stirred at 50° C. for 2 h. The reaction mixture was concentrated to dryness to afford the title compound (135 mg, 98.7% yield) as a white solid.

LCMS (ESI) m/z: 765.5 [M+H]⁺.

### Step 5: S-(3-(((((3R,5S)-1-((2R)-2-(3-(2-((3R)-4-(2-((4-(3-(3-amino-6-(2-((phosphonooxy) methoxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methyl thiazol-5-yl)phenyl)ethyl)carbamoyl)pyrrolidin-3-yl)oxy)carbonyl)oxy)-2-methylbutan-2-yl) methanesulfonothioate

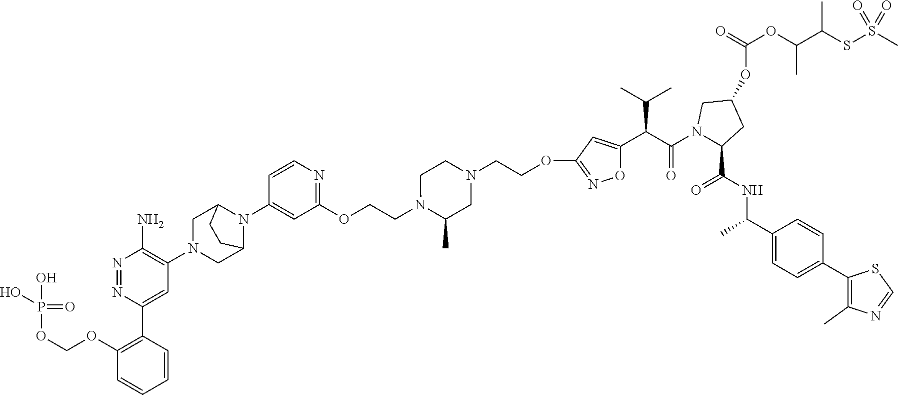 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1]

To solution of (2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl dihydrogen phosphate (138 mg, 0.19 mmol) and S-(2-methyl-3-(((((3R, 5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (130 mg, 0.17 mmol) in dichloromethane (5.0 mL) and methanol (5.0 mL) was added sodium triacetoxyborohydride (720 mg, 3.40 mmol). The reaction mixture was stirred at 40° C. for 48 h. The reaction mixture was purified by Column Welch Xtimate C18 150*25 mm*5 um Condition water (0.225% formic acid)-acetonitrile 20-50%) to give the title compound (54.2 mg, 21.3%) as a white solid.

LCMS (ESI) m/z: 1375.5 [M+H]⁺.

¹H NMR (400 MHz, DMSO-d₆) δ (ppm) 8.98 (s, 1H), 8.52 (d, J=8.0 Hz, 1H), 8.14 (s, 2H), 7.74 (d, J=6.0 Hz, 1H), 7.61 (d, J=8.0 Hz, 1H), 7.47-7.30 (m, 5H), 7.29-7.24 (m, 1H), 7.03 (t, J=7.2 Hz, 1H), 6.50 (d, J=6.0 Hz, 1H), 6.26 (s, 1H), 6.12 (d, J=2.4 Hz, 1H), 5.79-5.75 (m, 1H), 5.51-5.46 (m, 2H), 5.23-5.18 (m, 1H), 5.06-4.85 (m, 3H), 4.48-4.35 (m, 5H), 4.26-4.23 (m, 2H), 3.89-3.83 (m, 2H), 3.75-3.71 (m, 2H), 3.09-2.99 (m, 5H), 2.97-2.87 (m, 4H), 2.84-2.77 (m, 3H), 2.73-2.71 (m, 2H), 2.46-2.44 (m, 3H), 2.30-2.11 (m, 5H), 2.05-1.94 (m, 3H), 1.57-1.35 (m, 9H), 1.33-1.22 (m, 6H), 1.11-1.04 (m, 4H), 0.98-0.91 (m, 3H), 0.86-0.77 (m, 3H).

### Synthesis Example 10

### Synthesis of L1-CIDE-BRM1-10

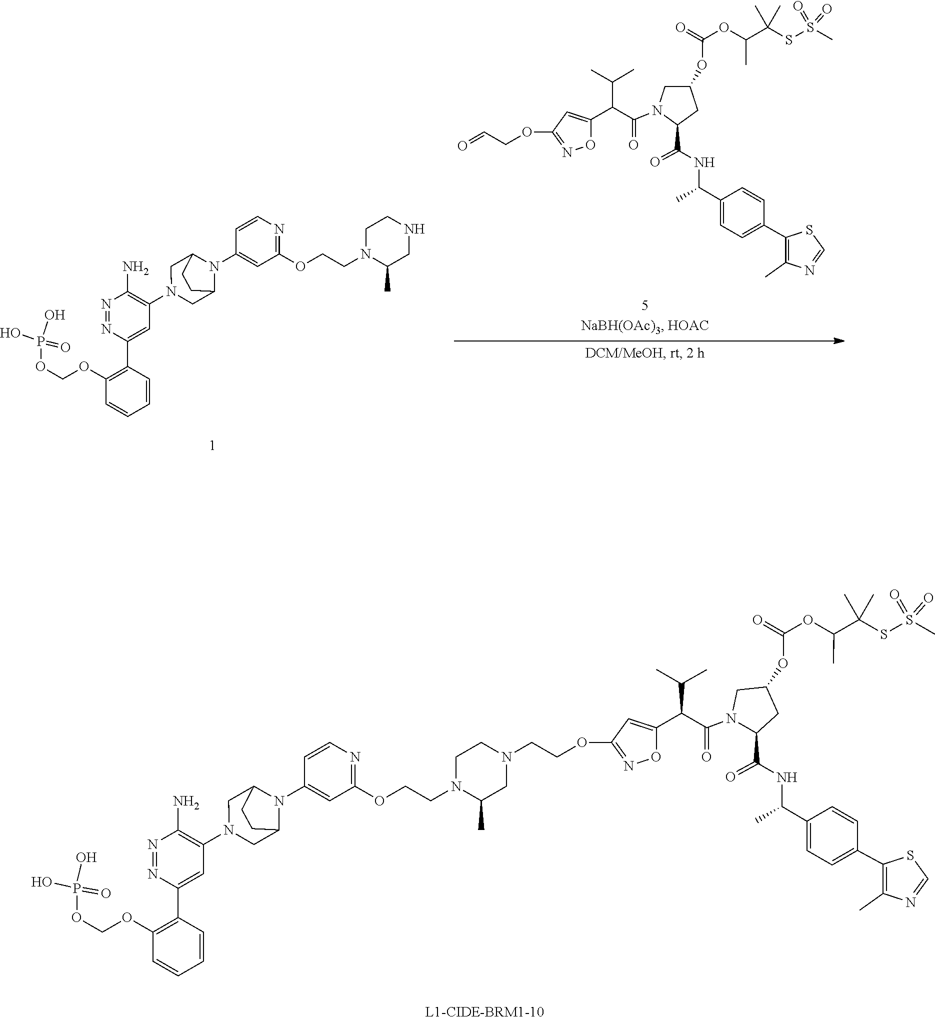 [SMILES: C[C@@H]1CNCCN1CCOc1cc(N2C3CCC2CN(c2cc(-c4ccccc4OCOP(=O)(O)O)nnc2N)C3)ccn1 ; Cc1ncsc1-c1ccc([C@H](C)NC(=O)[C@@H]2C[C@@H](OC(=O)OC(C)C(C)(C)SS(C)(=O)=O)CN2C(=O)C(c2cc(OCC=O)no2)C(C)C)cc1 ; Cc1ncsc1-c1ccc([C@H](C)NC(=O)[C@@H]2C[C@@H](OC(=O)OC(C)C(C)(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1]

### Step 1: S-(3-(((((3R,5S)-1-((2R)-2-(3-(2-((3R)-4-(2-((4-(3-(3-amino-6-(2-((phosphonooxy) methoxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methyl thiazol-5-yl)phenyl)ethyl)carbamoyl)pyrrolidin-3-yl)oxy)carbonyl)oxy)butan-2-yl) methanesulfonothioate

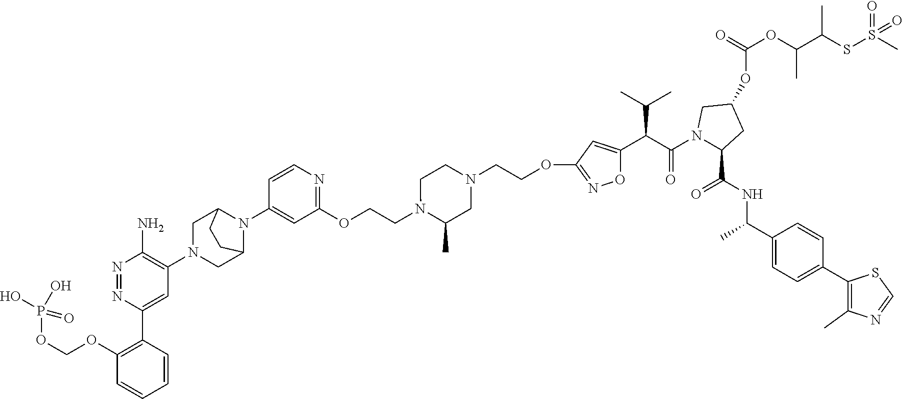 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCCN3CCN(CCOc4cc(N5C6CCC5CN(c5cc(-c7ccccc7OCOP(=O)(O)O)nnc5N)C6)ccn4)[C@H](C)C3)no2)C(C)C)cc1]

To a solution of S-(3-(((((3R, 5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl)oxy)carbonyl)oxy)butan-2-yl) methanesulfonothioate (100 mg, 0.13 mmol) and (2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl dihydrogen phosphate (99.2 mg, 0.16 mmol) in dichloromethane (1.00 mL) and methanol (1.00 mL) was added sodium triacetoxyborohydride (615 mg, 2.9 mmol). The reaction mixture was stirred at 20° C. for 3 h. The mixture was purified by Column Phenomenex Gemini-NX C18 75*30 mm*3 um Condition water (0.225% formic acid)—acetonitrile 10%-40%) to give the title compound (110 mg, 51.4%) as a white solid.

LCMS (ESI) m/z: 1375.5 [M+H]⁺.

¹H NMR (400 MHz, DMSO-d₆) δ (ppm) 8.99 (s, 1H), 8.51 (d, J=6.4 Hz, 1H), 8.15 (s, 2H), 7.82-7.72 (m, 1H), 7.64 (d, J=7.6 Hz, 1H), 7.52-7.32 (m, 6H), 7.30-7.24 (m, 1H), 7.08-7.03 (m, 1H), 6.56-6.46 (m, 1H), 6.33-6.30 (m, 1H), 6.14 (s, 1H), 5.91-5.86 (m, 1H), 5.55-5.50 (m, 2H), 5.21-5.18 (m, 1H), 4.98-4.87 (m, 2H), 4.51-4.31 (m, 5H), 4.30-4.22 (m, 2H), 3.91-3.84 (m, 2H), 3.77-3.73 (m, 2H), 3.58-3.51 (m, 2H), 3.18-3.11 (m, 4H), 3.07-2.97 (m, 4H), 2.95-2.81 (m, 5H), 2.79-2.72 (m, 2H), 2.47-2.45 (m, 3H), 2.37-2.14 (m, 5H), 2.10-1.88 (m, 3H), 1.48-1.23 (m, 9H), 1.20-1.05 (m, 3H), 0.98-0.93 (m, 3H), 0.88-0.76 (m, 3H).

### Synthesis Example 11

### Synthesis of L1-CIDE-BRM1-11

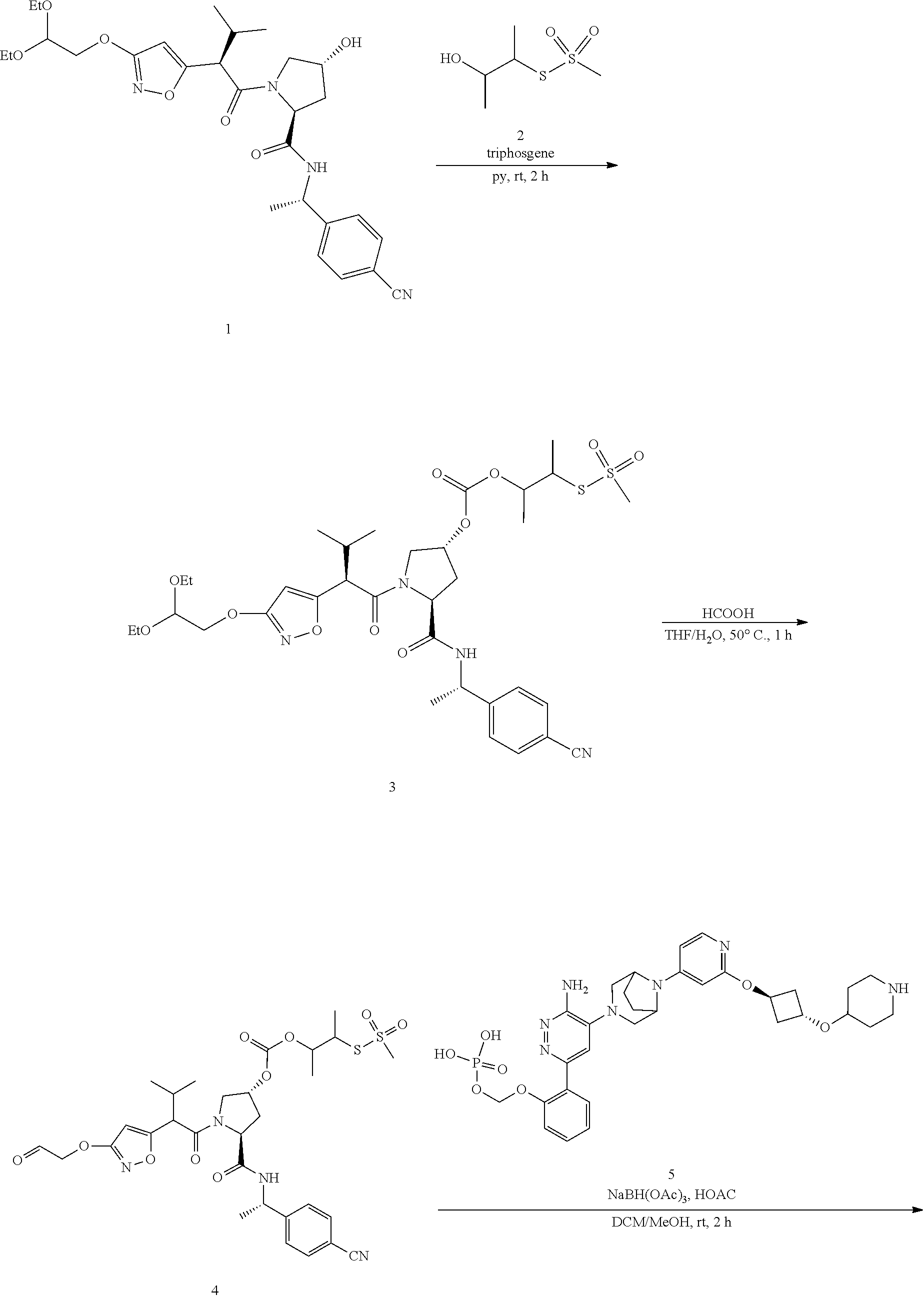

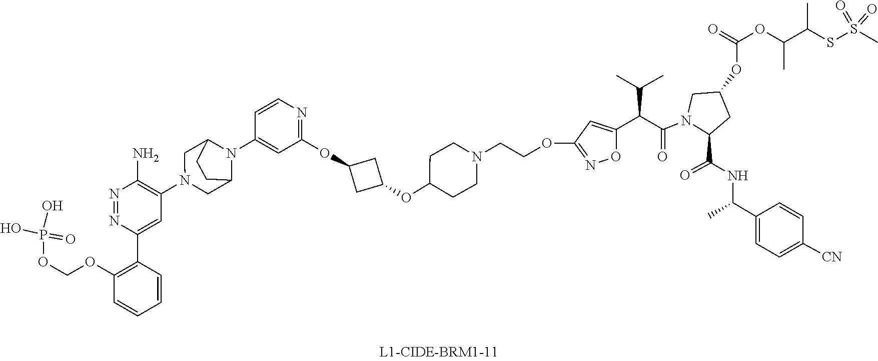 [SMILES: CC(OC(=O)O[C@@H]1C[C@@H](C(=O)N[C@@H](C)c2ccc(C#N)cc2)N(C(=O)[C@@H](c2cc(OCCN3CCC(O[C@H]4C[C@H](Oc5cc(N6C7CCC6CN(c6cc(-c8ccccc8OCOP(=O)(O)O)nnc6N)C7)ccn5)C4)CC3)no2)C(C)C)C1)C(C)SS(C)(=O)=O]

### Step 1: S-(3-((chlorocarbonyl)oxy)butan-2-yl) methanesulfonothioate

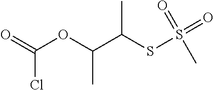

To a solution of S-(3-hydroxybutan-2-yl) methanesulfonothioate (200 mg, 1.09 mmol) and pyridine (343 mg, 4.34 mmol) in dichloromethane (4.0 mL) was added triphosgene (129 mg, 0.43 mmol) in dichloromethane (2.0 mL). The reaction mixture was stirred at 25° C. for 30 min. The reaction mixture was concentrated to dryness to give the title compound (250 mg, 93.4% yield) as a yellow oil which was used directly in next step.

### Step 2: S-(3-(((((3R, 5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl)oxy)carbonyl)oxy)butan-2-yl) methanesulfonothioate

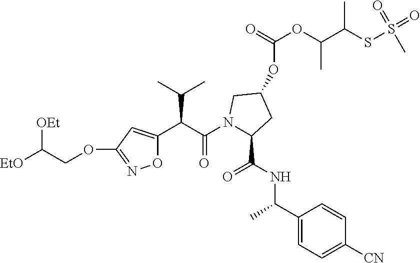 [SMILES: CCOC(COc1cc([C@H](C(=O)N2C[C@H](OC(=O)OC(C)C(C)SS(C)(=O)=O)C[C@H]2C(=O)N[C@@H](C)c2ccc(C#N)cc2)C(C)C)on1)OCC]

To a mixture of (2S, 4R)—N—((S)-1-(4-cyanophenyl)ethyl)-1-((R)-2-(3-(2,2-diethoxyethoxy)isoxazol-5-yl)-3-methylbutanoyl)-4-hydroxypyrrolidine-2-carboxamide (260 mg, 0.48 mmol), triethylamine (194 mg, 1.92 mmol) and 4 Å MS (80 mg) in anhydrous DCM (4.0 mL) was added S-(3-((chlorocarbonyl)oxy)butan-2-yl) methanesulfonothioate (250 mg, 1.01 mmol) in anhydrous dichloromethane (2.0 mL) slowly at 20° C. for 16 h. The reaction mixture was filtered and the filtrate was purified by flash chromatography (silica gel, 100-200 mesh, 0-70% ethyl acetate in petroleum ether) to afford the title compound (150 mg, 41.6%) as yellow oil.

LCMS (ESI) m/z: 752.9 [M+H]⁺.

### Step 3: S-(3-(((((3R, 5S)-5-(((S)-1-(4-cyanophenyl)ethyl)carbamoyl)-1-((R)-3-methyl-2-(3-(2-oxoethoxy)isoxazol-5-yl)butanoyl)pyrrolidin-3-yl)oxy)carbonyl)oxy)butan-2-yl) methanesulfonothioate

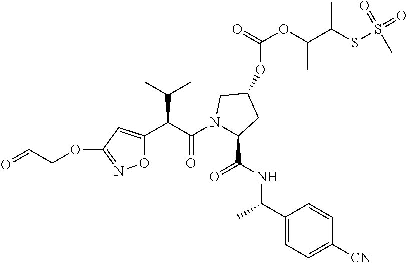 [SMILES: CC(OC(=O)O[C@@H]1C[C@@H](C(=O)N[C@@H](C)c2ccc(C#N)cc2)N(C(=O)[C@@H](c2cc(OCC=O)no2)C(C)C)C1)C(C)SS(C)(=O)=O]

A solution of S-(3-(((((3R, 5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl)oxy)carbonyl)oxy)butan-2-yl) methanesulfonothioate (150 mg, 0.20 mmol) in formic acid (5.0 mL) and water (1.0 mL) was stirred at 50° C. for 16 h. The reaction mixture was concentrated to dryness to afford the title compound (100 mg, 73.9% yield) as a yellow oil.

LCMS (ESI) m/z: 679.5 [M+H]⁺.

### Step 4: S-(3-(((((3R,5S)-1-((2R)-2-(3-(2-(4-((1r,3r)-3-((4-(3-(3-amino-6-(2-((phosphonooxy)methoxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl)oxy)carbonyl)oxy)butan-2-yl) methanesulfonothioate

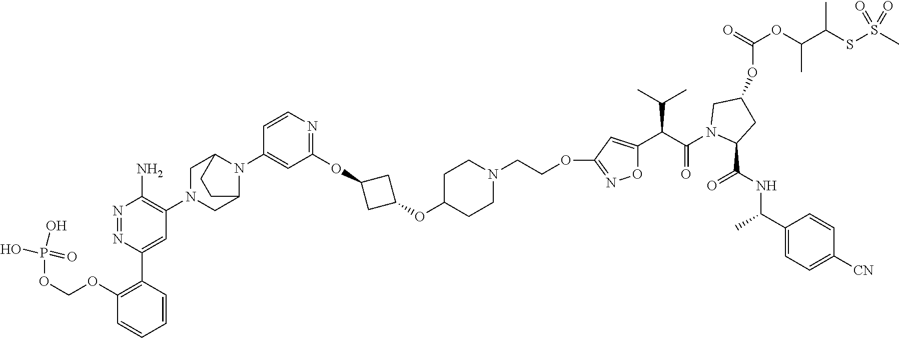 [SMILES: CC(OC(=O)O[C@@H]1C[C@@H](C(=O)N[C@@H](C)c2ccc(C#N)cc2)N(C(=O)[C@@H](c2cc(OCCN3CCC(O[C@H]4C[C@H](Oc5cc(N6C7CCC6CN(c6cc(-c8ccccc8OCOP(=O)(O)O)nnc6N)C7)ccn5)C4)CC3)no2)C(C)C)C1)C(C)SS(C)(=O)=O]

A solution of (2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl dihydrogen phosphate (169 mg, 0.22 mmol) and S-(3-(((((3R,5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl)oxy)carbonyl)oxy)butan-2-yl) methane sulfonothioate (150 mg, 0.22 mmol) in dichloromethane (5.0 mL) and methanol (5.0 mL) was added sodium triacetoxyborohydride (1.40 g, 6.63 mmol). The reaction mixture was stirred at 20° C. for 48 h. The reaction mixture was purified by Column Phenomenex Gemini-NX C18 75*30 mm*3 um Condition water (0.225% formic acid)—acetonitrile 10-40%) to give the title compound (25.6 mg, 8.8% yield) as a white solid.

LCMS (ESI) m/z: 659.1 [M/2+H]⁺.

¹H NMR (400 MHz, DMSO-d₆) δ (ppm) 8.58 (d, J=8.0 Hz, 1H), 8.14 (s, 1H), 7.82-7.74 (m, 3H), 7.55 (d, J=8.0 Hz, 1H), 7.50-7.43 (m, 3H), 7.39-7.34 (m, 1H), 7.22 (s, 1H), 7.15-7.09 (m, 1H), 6.54-6.51 (m, 1H), 6.37-6.21 (m, 2H), 6.16-6.10 (m, 2H), 5.57-5.52 (m, 2H), 5.18-5.12 (m, 2H), 4.99-4.87 (m, 2H), 4.52-4.44 (m, 2H), 4.40-4.22 (m, 4H), 3.87-3.82 (m, 2H), 3.74-3.70 (m, 1H), 3.60-3.50 (m, 2H), 3.15-3.00 (m, 2H), 2.96-2.79 (m, 4H), 2.69-2.64 (m, 1H), 2.35-2.18 (m, 9H), 2.16-2.09 (m, 2H), 2.02-1.74 (m, 6H), 1.53-1.23 (m, 12H), 0.95-0.91 (m, 3H), 0.85-0.74 (m, 3H).

### Synthesis Example 12

### Synthesis of L1-CIDE-BRM1-12

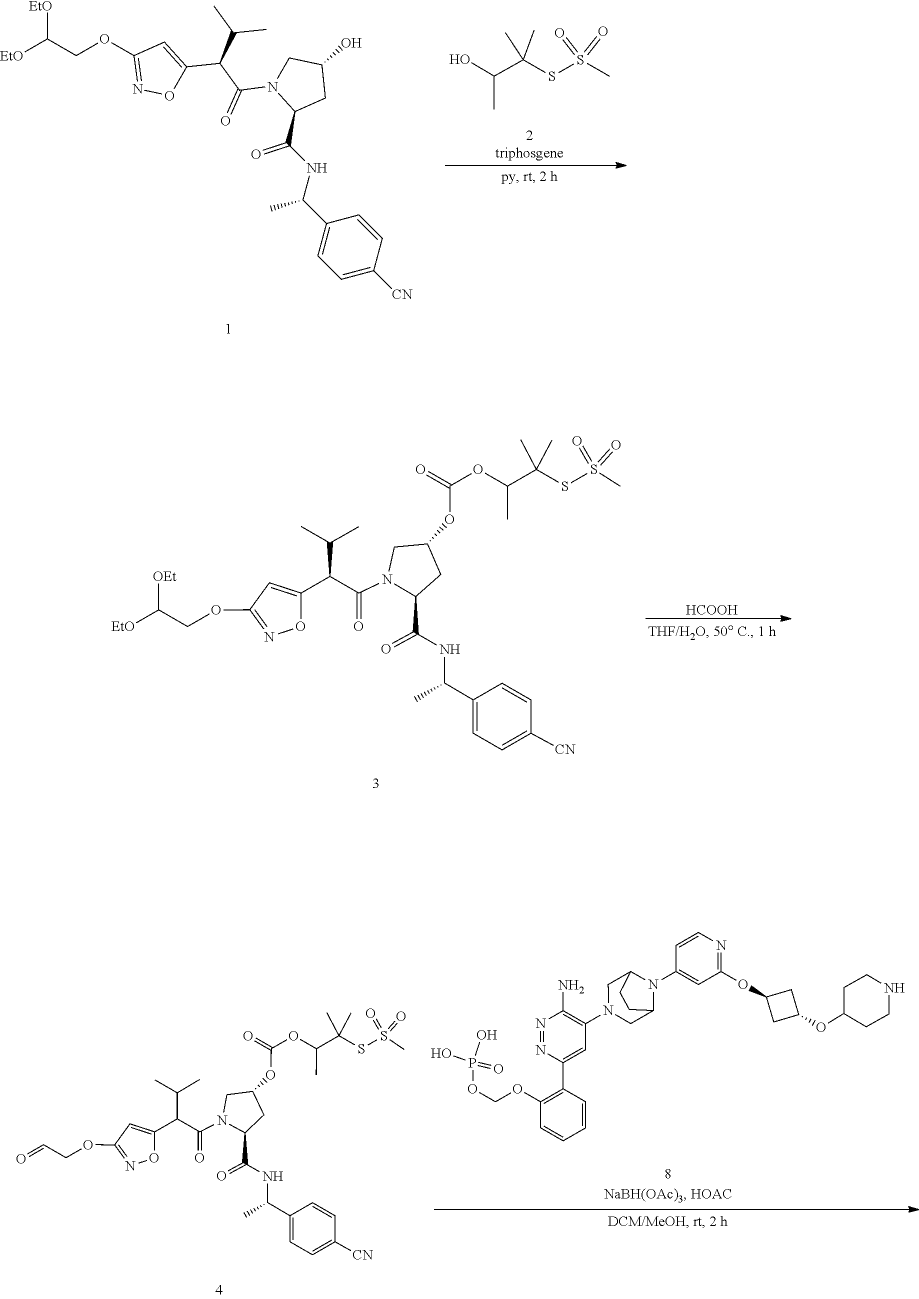 [SMILES: CCOC(COc1cc([C@H](C(=O)N2C[C@H](O)C[C@H]2C(=O)N[C@@H](C)c2ccc(C#N)cc2)C(C)C)on1)OCC ; CC(O)C(C)(C)SS(C)(=O)=O ; CCOC(COc1cc([C@H](C(=O)N2C[C@H](OC(=O)OC(C)C(C)(C)SS(C)(=O)=O)C[C@H]2C(=O)N[C@@H](C)c2ccc(C#N)cc2)C(C)C)on1)OCC ; "HCOOH" ; CC(C)C(C(=O)N1C[C@H](OC(=O)OC(C)C(C)(C)SS(C)(=O)=O)C[C@H]1C(=O)N[C@@H](C)c1ccc(C#N)cc1)c1cc(OCC=O)no1 ; Nc1nnc(-c2ccccc2OCOP(=O)(O)O)cc1N1CC2CCC(C1)N2c1ccnc(O[C@H]2C[C@H](OC3CCNCC3)C2)c1]

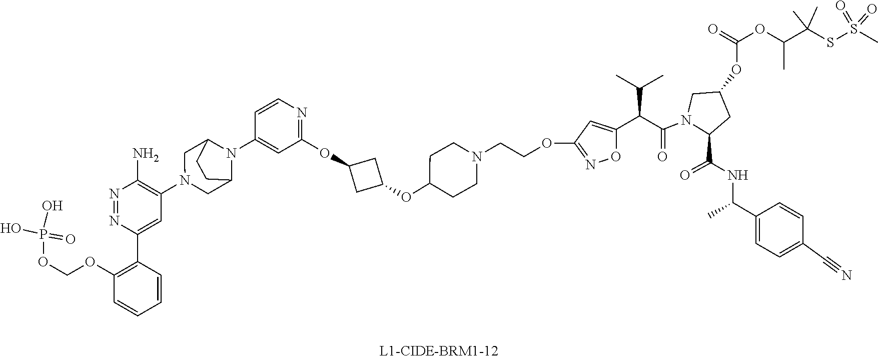 [SMILES: CC(C)[C@@H](C(=O)N1C[C@H](OC(=O)OC(C)C(C)(C)SS(C)(=O)=O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)O)nnc5N)C6)ccn4)C3)CC2)no1]

### Step 1: S-(3-(((((3R,5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl)oxy)carbonyl)oxy)-2-methylbutan-2-yl) methanesulfonothioate

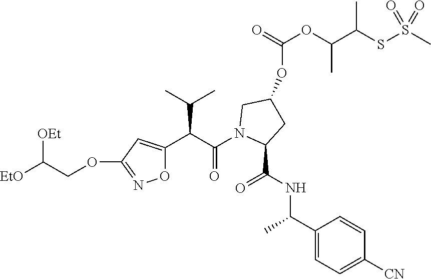 [SMILES: CCOC(COc1cc([C@H](C(=O)N2C[C@H](OC(=O)OC(C)C(C)SS(C)(=O)=O)C[C@H]2C(=O)N[C@@H](C)c2ccc(C#N)cc2)C(C)C)on1)OCC]

To a mixture of (2S, 4R)—N—((S)-1-(4-cyanophenyl)ethyl)-1-((R)-2-(3-(2,2-diethoxyethoxy)isoxazol-5-yl)-3-methylbutanoyl)-4-hydroxypyrrolidine-2-carboxamide (300 mg, 0.55 mmol), pyridine (175 mg, 2.21 mmol) and 4 Å MS (100 mg) in anhydrous dichloromethane (5.0 mL) was added S-(3-((chlorocarbonyl)oxy)-2-methylbutan-2-yl) methane sulfonothioate (259 mg, 1.00 mmol) in anhydrous dichloromethane (2 mL) slowly at 20° C. for 16 h. The reaction mixture was purified by pre-TLC (7% methanol in dichloromethane) to give the title compound (60.0 mg, 14.2%) as a white solid.

LCMS (ESI) m/z: 722.1 [M+H]⁺.

### Step 3: S-(3-(((((3R,5S)-5-(((S)-1-(4-cyanophenyl)ethyl)carbamoyl)-1-((R)-3-methyl-2-(3-(2-oxoethoxy)isoxazol-5-yl)butanoyl)pyrrolidin-3-yl)oxy)carbonyl)oxy)-2-methylbutan-2-yl) methanesulfonothioate

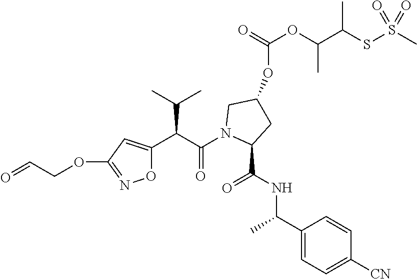 [SMILES: CC(OC(=O)O[C@@H]1C[C@@H](C(=O)N[C@@H](C)c2ccc(C#N)cc2)N(C(=O)[C@@H](c2cc(OCC=O)no2)C(C)C)C1)C(C)SS(C)(=O)=O]

A solution of S-(3-(((((3R, 5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl)oxy)carbonyl)oxy)-2-methylbutan-2-yl) methanesulfonothioate (60.0 mg, 0.08 mmol) in water (1.00 mL) and formic acid (5.00 mL). The reaction mixture was stirred at 50° C. for 2 h. The resulting residue was concentrated to afford the title compound (50 mg, 92.2%) as white solid. LCMS (ESI) m/z: 693.2 [M+H]⁺.

### Step 4: S-(3-(((((3R,5S)-1-((2R)-2-(3-(2-(4-((1r,3r)-3-((4-(3-(3-amino-6-(2-((phosphonooxy)methoxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl)oxy)carbonyl)oxy)-2-methylbutan-2-yl) methanesulfonothioate

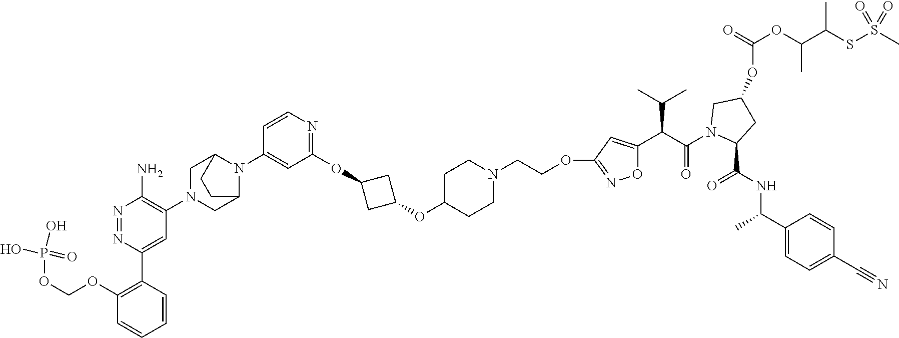 [SMILES: CC(OC(=O)O[C@@H]1C[C@@H](C(=O)N[C@@H](C)c2ccc(C#N)cc2)N(C(=O)[C@@H](c2cc(OCCN3CCC(O[C@H]4C[C@H](Oc5cc(N6C7CCC6CN(c6cc(-c8ccccc8OCOP(=O)(O)O)nnc6N)C7)ccn5)C4)CC3)no2)C(C)C)C1)C(C)SS(C)(=O)=O]

A solution of (2-(6-amino-5-(8-(2-((1r, 3r)-3-(piperidin-4-yloxy)cyclobutoxy)pyridin-4-yl)-3,8-diazabicyclo[3.2.1]octan-3-yl)pyridazin-3-yl)phenoxy)methyl dihydrogen phosphate (138.51 mg, 0.18 mmol) and S-(3-(((((3R, 5S)-5-(((S)-1-(4-cyanophenyl)ethyl)carbamoyl)-1-((R)-3-methyl-2-(3-(2-oxoethoxy)isoxazol-5-yl)butanoyl)pyrrolidin-3-yl)oxy)carbonyl)oxy)-2-methylbutan-2-yl) methanesulfonothioate (125 mg, 0.18 mmol) in dichloromethane (5.00 mL) and methanol (5.00 mL) was added sodium triacetoxyborohydride (38.0 mg, 0.18 mmol). The reaction mixture was stirred at 20° C. for 36 h. The reaction mixture was purified by Column Welch Xtimate C18 150*25 mm*5 um condition water (0.225% formic acid)—acetonitrile 17-47%) to give the title compound (15.9 mg, 50.4% yield) as a white solid.

LCMS (ESI) m/z: 666.1 [M/2+H]⁺.

¹H NMR (400 MHz, DMSO-d₆) δ (ppm) 8.61-8.56 (m, 1H), 7.81-7.73 (m, 3H), 7.57-7.42 (m, 4H), 7.38-7.32 (m, 1H), 7.22 (s, 1H), 7.15-7.08 (m, 1H), 6.54-6.50 (m, 1H), 6.29-6.25 (m, 2H), 6.15-6.10 (m, 2H), 5.58-5.52 (m, 2H), 5.22-5.11 (m, 2H), 5.04-5.01 (m, 1H), 4.94-4.91 (m, 1H), 4.49-4.45 (m, 2H), 4.41-4.36 (m, 1H), 4.28-4.25 (m, 3H), 3.90-3.82 (m, 2H), 3.75-3.71 (m, 2H), 3.56-3.51 (m, 1H), 3.10-2.82 (m, 5H), 2.31-2.18 (m, 7H), 2.16-2.09 (m, 2H), 2.02-1.77 (m, 5H), 1.56-1.40 (m, 11H), 1.39-1.20 (m, 8H), 1.16-1.12 (m, 1H), 0.95-0.91 (m, 3H), 0.85-0.76 (m, 4H).

### Synthesis Example 13

### Synthesis of L1-CIDE-BRM1-13

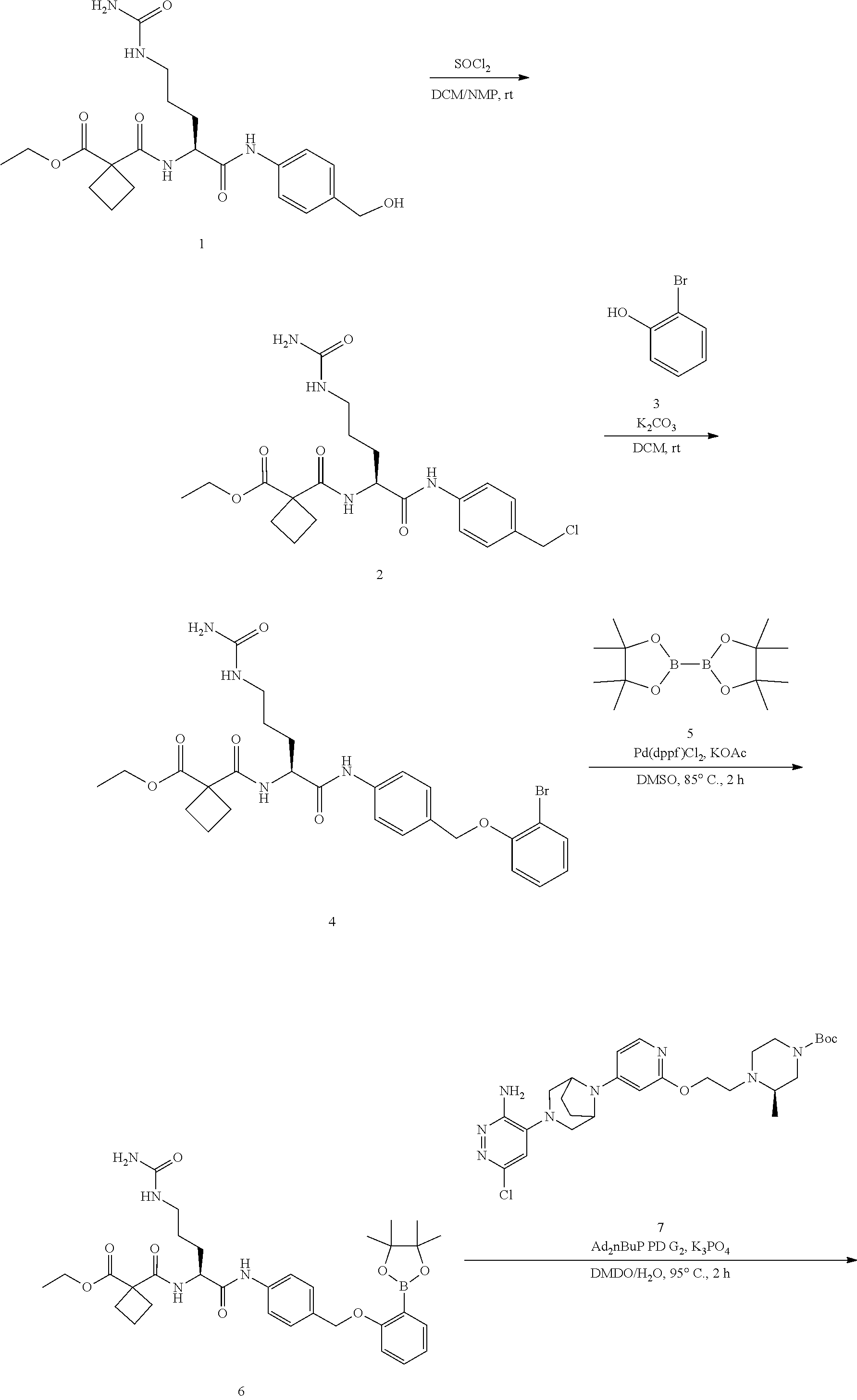

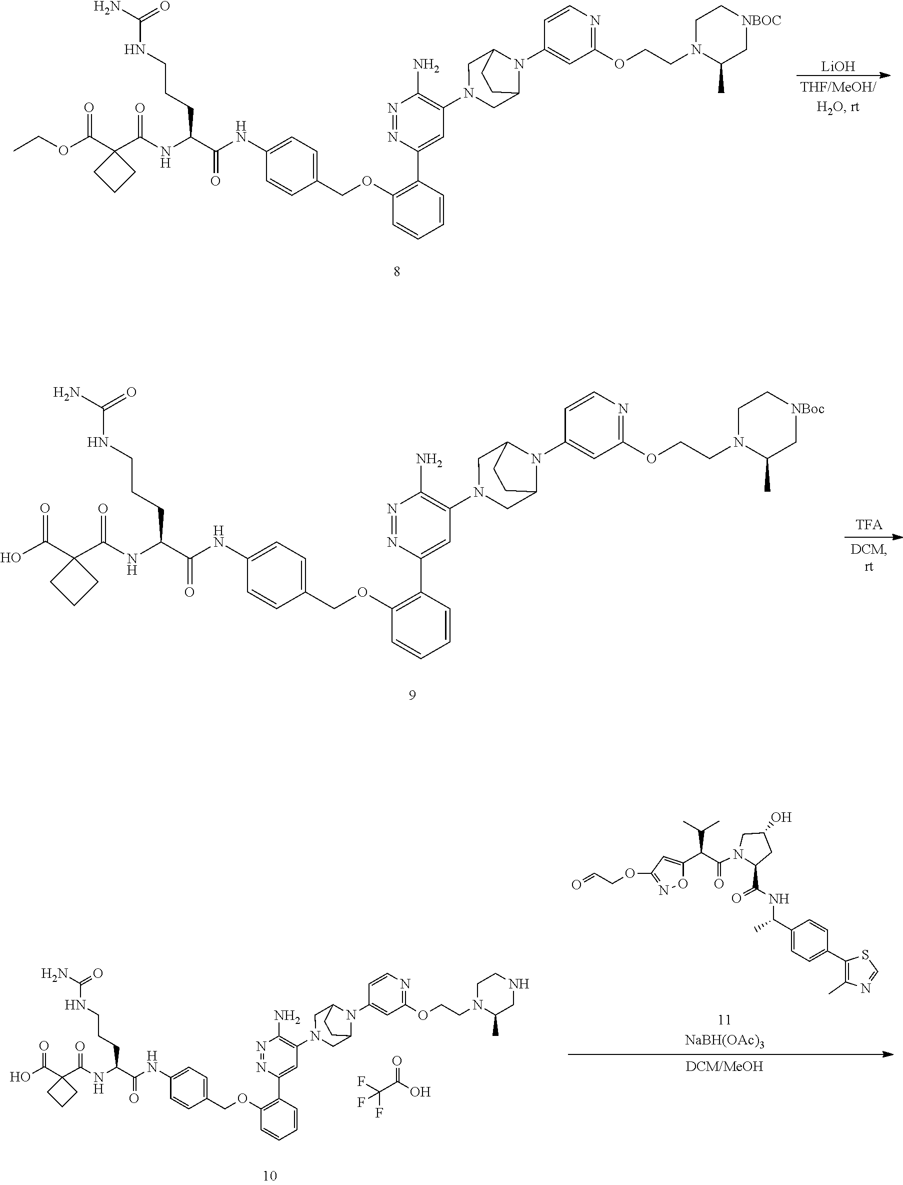

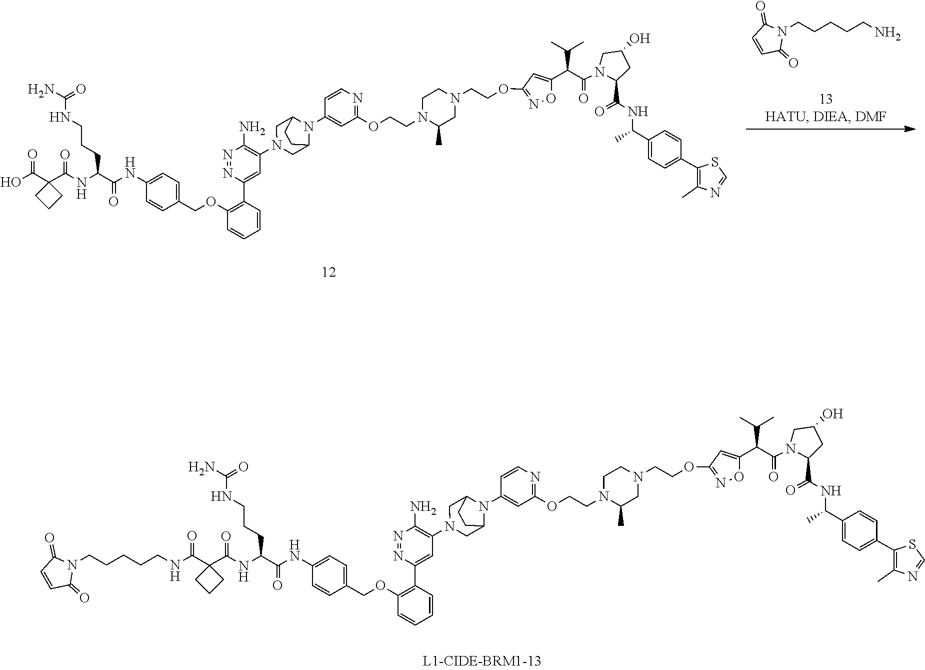 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)O)CCC8)cc7)nnc5N)C6)ccn4)[C@H](C)C3)no2)C(C)C)cc1 ; NCCCCCN1C(=O)C=CC1=O ; Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1]

### Step 1: (S)-ethyl 1-((1-((4-(chloromethyl)phenyl)amino)-1-oxo-5-ureidopentan-2-yl)carbamoyl)cyclobutanecarboxylate

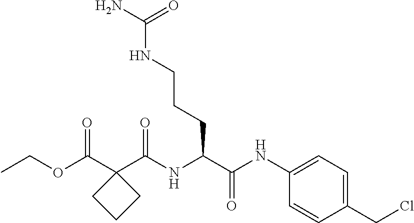 [SMILES: CCOC(=O)C1(C(=O)N[C@@H](CCCNC(N)=O)C(=O)Nc2ccc(CCl)cc2)CCC1]

To a solution of (S)-ethyl 1-((1-((4-(hydroxymethyl)phenyl)amino)-1-oxo-5-ureidopentan-2-yl)carbamoyl)cyclobutanecarboxylate (1.4 g, 3.22 mmol) in dichloromethane (50.0 mL) and N MP (1.0 mL) was added thionyl chloride (0.70 mL, 9.67 mmol) at 25° C. The reaction mixture was stirred at 25° C. for 3 h. The reaction mixture was purified by flash chromatography (silica gel, 100-200 mesh, 0-5% methanol in dichloromethane) to afford the title compound (1.40 g, 96% yield) as a yellow oil.

### Step 2: (S)-ethyl 1-((1-((4-((2-bromophenoxy)methyl)phenyl)amino)-1-oxo-5-ureidopentan-2-yl)carbamoyl)cyclobutanecarboxylate

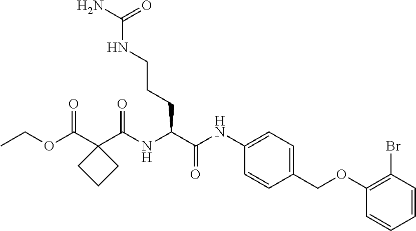 [SMILES: CCOC(=O)C1(C(=O)N[C@@H](CCCNC(N)=O)C(=O)Nc2ccc(COc3ccccc3Br)cc2)CCC1]

To a solution of (S)-ethyl 1-((1-((4-(chloromethyl)phenyl)amino)-1-oxo-5-ureidopentan-2-yl)carbamoyl)cyclobutanecarboxylate (1.40 g, 3.09 mmol) and potassium carbonate₃ (1.07 g, 7.73 mmol) in N,N-dimethylformamide (60 mL) was added 2-bromophenol (0.54 mL, 4.64 mmol) at 25° C. The reaction was stirred at 25° C. for 3 h. The reaction was diluted with water (30.0 mL) and extracted with dichloromethane (50.0 mL×3). The combined organic layers were washed with brine (20.0 mL×2), dried over sodium sulfate, filtered and concentrated to dryness. The residue was purified by flash chromatography (silica gel, 100-200 mesh, 0-5% methanol in dichloromethane) to afford the title compound (1.1 g, 60% yield) as a white solid.

LCMS (ESI) m/z: 590.7 [M+H]⁺.

### Step 3: (S)-ethyl 1-((1-oxo-1-((4-((2-(4,4,5,5-tetramethyl-1,3,2-dioxaborolan-2-yl)phenoxy)methyl)phenyl)amino)-5-ureidopentan-2-yl)carbamoyl)cyclobutanecarboxylate

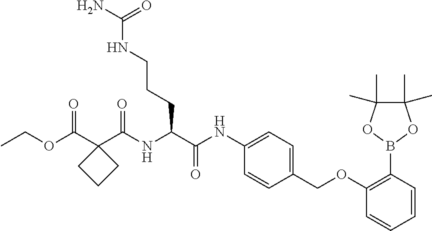 [SMILES: CCOC(=O)C1(C(=O)N[C@@H](CCCNC(N)=O)C(=O)Nc2ccc(COc3ccccc3B3OC(C)(C)C(C)(C)O3)cc2)CCC1]

To a solution of (S)-ethyl 1-((1-((4-((2-bromophenoxy)methyl)phenyl)amino)-1-oxo-5-ureido pentan-2-yl)carbamoyl)cyclobutanecarboxylate (1.10 g, 1.87 mmol) and bis(pinacolato)diboron (711 mg, 2.80 mmol) in dimethyl sulfoxide (20.0 mL) was added Pd(dppf)Cl₂ (137 mg, 0.19 mmol) and potassium acetate (549 mg, 5.60 mmol). The mixture was stirred at 100° C. un der N₂ for 3 h. The mixture crude was used directly in next step.

LCMS (ESI) m/z: 637.1 [M+H]⁺.

### Step 4: (3R)-tert-butyl 4-(2-((4-(3-(3-amino-6-(2-((4-((S)-2-(1-(ethoxycarbonyl)cyclobutanecarboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate

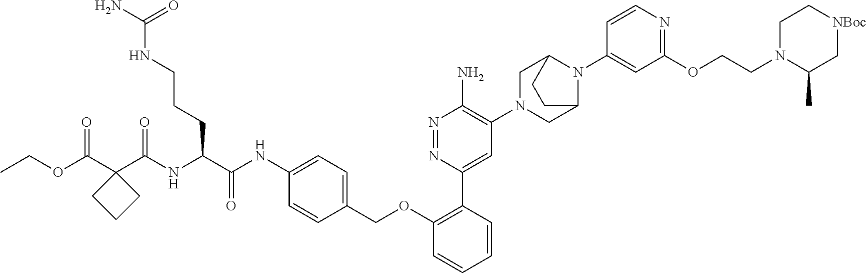 [SMILES: CCOC(=O)C1(C(=O)N[C@@H](CCCNC(N)=O)C(=O)Nc2ccc(COc3ccccc3-c3cc(N4CC5CCC(C4)N5c4ccnc(OCCN5CCN(C(=O)OC(C)(C)C)C[C@H]5C)c4)c(N)nn3)cc2)CCC1]

To a solution of (3R)-tert-butyl 4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (750 mg, 1.28 mmol) and tripotassium orthophosphate (814 mg, 3.84 mmol) in dimethyl sulfoxide (15.0 mL) and H₂O (1.00 mL) was added chloro[(di(1-adamantyl)-n-butylphosphine)-2-(2-aminobiphenyl)]palladium(II) (171 mg, 0.26 mmol) and (S)-ethyl 1-((1-oxo-1-((4-((2-(4,4,5,5-tetramethyl-1,3,2-dioxaborolan-2-yl)phenoxy)methyl)phenyl)amino)-5-ureidopentan-2-yl)carbamoyl)cyclobutanecarboxylate (832 mg, 1.31 mmol). The mixture was stirred at 100° C. under N₂ for 3 h. The reaction was purified by silica gel column (silica gel, 100-200 mesh, 0-15% methanol in dichloromethane) to afford the title compound (400 mg, 28% yield) as a dark oil.

LCMS (ESI) m/z: 1251.0 [M+H]⁺.

### Step 5: 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid

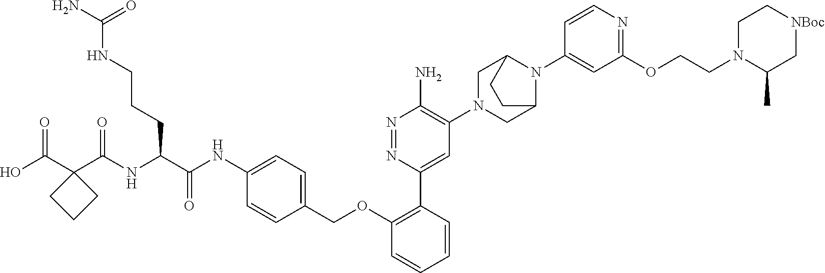 [SMILES: C[C@@H]1CN(C(=O)OC(C)(C)C)CCN1CCOc1cc(N2C3CCC2CN(c2cc(-c4ccccc4OCc4ccc(NC(=O)[C@H](CCCNC(N)=O)NC(=O)C5(C(=O)O)CCC5)cc4)nnc2N)C3)ccn1]

To a solution of (3R)-tert-butyl 4-(2-((4-(3-(3-amino-6-(2-((4-((S)-2-(1-(ethoxycarbonyl)cyclobutanecarboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate (400 mg, 0.39 mmol) in methanol (5.0 mL) and water (2.0 mL) was added lithium hydroxide monohydrate (92.7 mg, 3.87 mmol). The mixture was stirred at 25° C. under N₂ for 3 h. The reaction mixture was concentrated to dryness to afford the title compound (300 mg, 77.1% yield) as a dark solid.

LCMS (ESI) m/z: 1005.6 [M+H]⁺.

### Step 6: 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid 2,2,2-trifluoroacetate

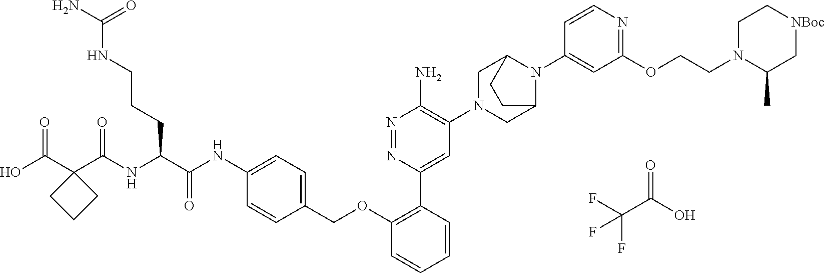 [SMILES: C[C@@H]1CN(C(=O)OC(C)(C)C)CCN1CCOc1cc(N2C3CCC2CN(c2cc(-c4ccccc4OCc4ccc(NC(=O)[C@H](CCCNC(N)=O)NC(=O)C5(C(=O)O)CCC5)cc4)nnc2N)C3)ccn1 ; O=C(O)C(F)(F)F]

To a solution of 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid (300 mg, 0.3 mmol) in dichloromethane (20.0 mL) was added trifluoroacetic acid (0.20 mL, 3.00 mmol). The solution was stirred at 20° C. for 5 h and concentrated to dryness. The crude product was purified by Prep-HPLC with the following conditions (column: Welch Xtimate C18 100*40 mm*3 um; mobile phase: 12-42% water (0.075% trifluoroacetic acid)—acetonitrile) to afford the title compound (89 mg, 32.9%) as a white solid.

LCMS (ESI) m/z: 905.5 [M+H]⁺.

### Step 7: 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid

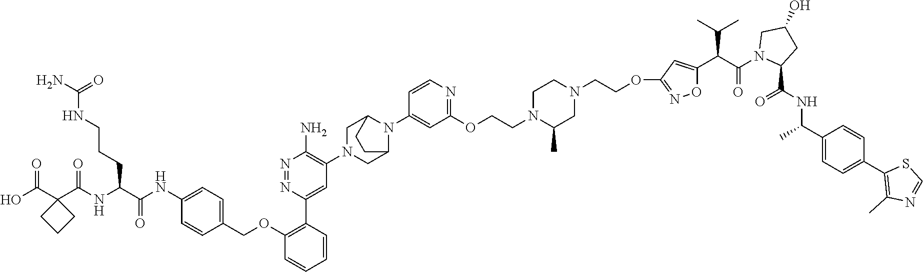 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)O)CCC8)cc7)nnc5N)C6)ccn4)[C@H](C)C3)no2)C(C)C)cc1]

To a solution of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (86.02 mg, 0.16 mmol) and 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid 2,2,2-trifluoroacetate (96.0 mg, 0.11 mmol) in dichloromethane (1.50 mL) and methanol (1.50 mL) was added sodium cyanoborohydride (13.6 mg, 0.21 mmol). The reaction mixture was stirred at 20° C. for 3 h. The resulting solution was purified by Phenomenex Gemini-NX 80*40 mm*3 um (acetonitrile 17-47%/0.05% NH₃H₂O in water) to afford the title compound (89.0 mg, 58.7% yield) as a white solid. LCMS (ESI) m/z: 1429.9 [M+H]⁺.

### Step 8: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)-N-(5-(2,5-dioxo-2,5-dihydro-1H-pyrrol-1-yl)pentyl)cyclobutane-1,1-dicarboxamide

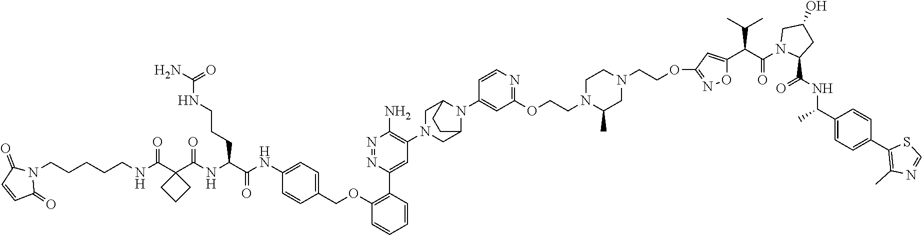 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1]

To a mixture of 1-(((2S)-1-((4-((2-(6-amino-5-(8-(2-(2-((R)-4-(2-((5-((R)-1-((2S, 4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid (50.0 mg, 0.03 mmol) and 1-(5-aminopentyl)-1H-pyrrole-2,5-dione 2,2,2-trifluoroacetic acid (12.4 mg, 0.04 mmol) in N,N-dimethylformamide (4.0 mL) was added N,N-diisopropylethylamine (0.02 mL, 0.10 mmol) and 2-(7-azabenzotriazol-1-yl)-N,N,N,N-tetramethyluronium hexafluorophosphate (16.0 mg, 0.04 mmol). The mixture was stirred at 25° C. for 3 h. The reaction mixture was concentrated to dryness by oil pump. The residue was purified by prep-HPLC (Boston Green ODS 150*30 mm*5 um, water (0.075% trifluoroacetic acid)—acotonitrile, 20-50%) to afford the title compound (33.6 mg, 59.7% yield) as a white solid. ¹H NMR (400 MHz, DMSO-d₆): δ (ppm) 10.19 (s, 1H), 9.02-8.86 (m, 1H), 8.39 (d, J=8.0 Hz, 1H), 7.94 (d, J=6.8 Hz, 1H), 7.87-7.78 (m, 2H), 7.65 (d J=8.0 Hz, 2H), 7.51-7.41 (m, 5H), 7.36 (d, J=6.8 Hz, 5H), 7.29-7.09 (m, 1H), 6.97 (s, 2H), 6.76 (s, 1H), 6.42 (s, 1H), 6.15-5.96 (m, 2H), 5.07 (s, 2H), 4.93-4.86 (m, 1H), 4.71-4.60 (m, 2H), 4.52-4.24 (m, 9H), 3.66 (s, 4H), 3.33 (d, J=7.2 Hz, 5H), 3.09-2.96 (m, 8H), 2.45 (s, 3H), 2.42-2.34 (m, 4H), 2.29-2.14 (m, 2H), 2.04 (d, J=11.2 Hz, 3H), 1.91 (s, 2H), 1 0.83-1.55 (m, 5H), 1.51-1.30 (m, 9H), 1.19 (d, J=5.8 Hz, 5H), 0.96 (d, J=6.4 Hz, 3H), 0.8 6-0.75 (m, 3H).

LCMS (ESI) m/z: 797.5 [M/2+H]⁺.

### Synthesis Example 14

### Synthesis of L1-CIDE-BRM1-14

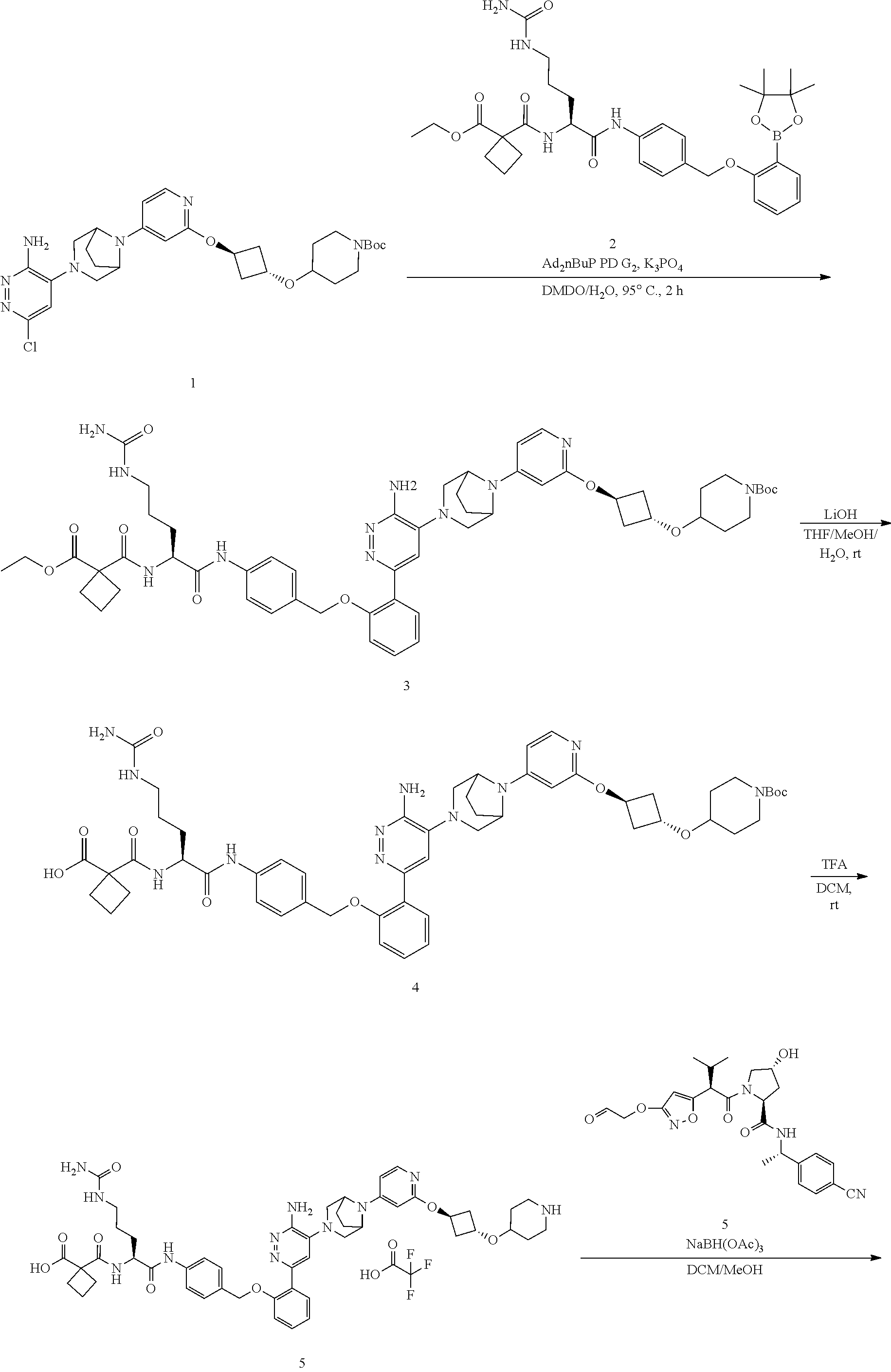

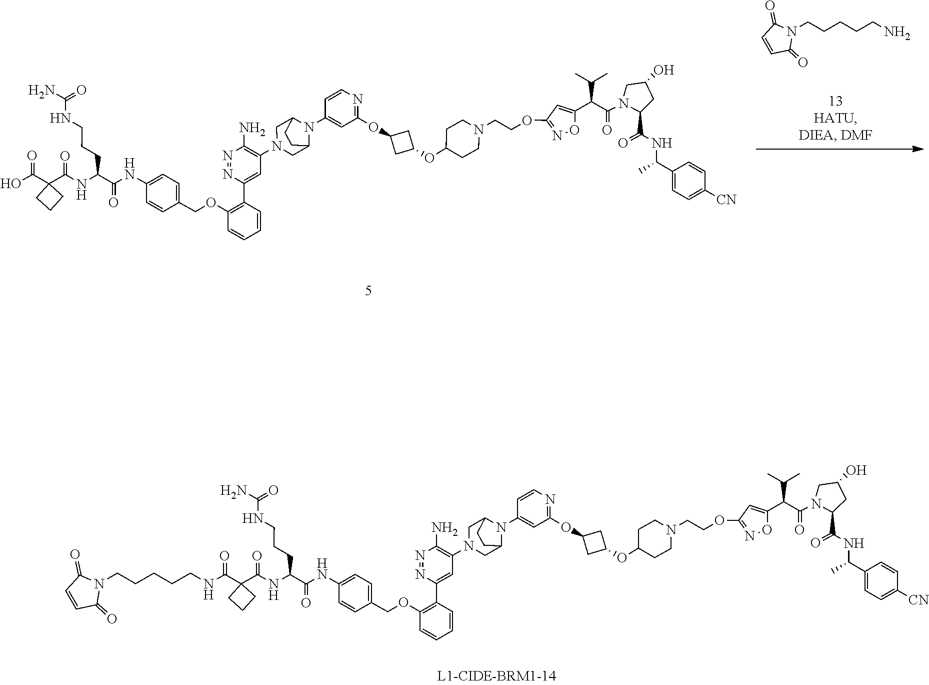 [SMILES: NCCCCCN1C(=O)C=CC1=O ; CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCNC(N)=O)NC(=O)C8(C(=O)O)CCC8)cc7)nnc5N)C6)ccn4)C3)CC2)no1 ; CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)C3)CC2)no1]

### Step 1: tert-butyl 4-((1r,3r)-3-((4-(3-(3-amino-6-(2-((4-((S)-2-(1-(ethoxycarbonyl)cyclobutanecarboxamido)-5-ureidopentanamido)benzyl)oxy)phenyl)pyridazin-4-yl)-3,8-diazabicyclo[3.2.1]octan-8-yl)pyridin-2-yl)oxy)cyclobutoxy)piperidine-1-carboxylate

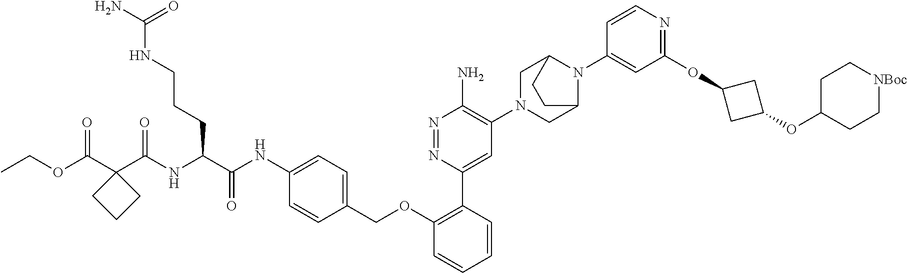 [SMILES: CCOC(=O)C1(C(=O)N[C@@H](CCCNC(N)=O)C(=O)Nc2ccc(COc3ccccc3-c3cc(N4CC5CCC(C4)N5c4ccnc(O[C@H]5C[C@H](OC6CCN(C(=O)OC(C)(C)C)CC6)C5)c4)c(N)nn3)cc2)CCC1]

To a solution of tert-butyl 4-((1r, 3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate (450 mg, 0.77 m mol) and tripotassium orthophosphate (0.19 mL, 2.3 mmol) in dimethyl sulfoxide (20 mL) was added chloro[(di(1-adamantyl)-n-butylphosphine)-2-(2-aminobiphenyl)]palladium(II) (103 mg, 0.15 mmol) and (S)-ethyl 1-((1-oxo-1-((4-((2-(4,4,5,5-tetramethyl-1,3,2-dioxaborolan-2-yl)phenoxy)methyl)phenyl)amino)-5-ureidopentan-2-yl)carbamoyl)cyclobutanecarboxylate (977 mg, 1.54 mmol). The mixture was stirred at 100° C. under N₂ for 3 h. The reaction mixture was diluted with water (10 mL) and extracted with dichloromethane (20 mL×3). The organics were washed with brine (30 mL×2), dried over sodium sulfate, filtered and concentrate d to afford the title compound (400 mg, 49.1%) as a dark solid.

LCMS (ESI) m/z: 1060.7 [M+H]⁺.

### Step 2: 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid

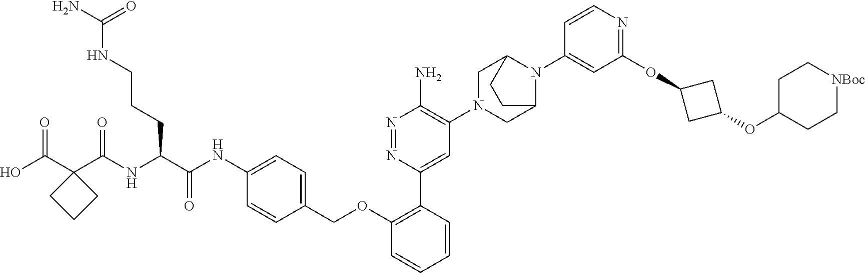 [SMILES: CC(C)(C)OC(=O)N1CCC(O[C@H]2C[C@H](Oc3cc(N4C5CCC4CN(c4cc(-c6ccccc6OCc6ccc(NC(=O)[C@H](CCCNC(N)=O)NC(=O)C7(C(=O)O)CCC7)cc6)nnc4N)C5)ccn3)C2)CC1]

To a solution of tert-butyl 4-((1r, 3r)-3-((4-(3-(3-amino-6-(2-((4-((S)-2-(1-(ethoxycarbonyl)cyclobutanecarboxamido)-5-ureidopentanamido)benzyl)oxy)phenyl)pyridazin-4-yl)-3,8-diaza bicyclo[3.2.1]octan-8-yl)pyridin-2-yl)oxy)cyclobutoxy)piperidine-1-carboxylate (450 mg, 0.4 2 mmol) in methanol (5.0 mL) and water (2.0 mL) was added lithium hydroxide monohydrate (102 mg, 4.24 mmol). The mixture was stirred at 25° C. for 3 h. The reaction mixture was concentrated to afford the title compound (438 mg, 97.5% yield) as a dark solid. LCMS (ESI) m/z: 1032.6 [M+H]⁺.

### Step 3: 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid 2,2,2-trifluoroacetate

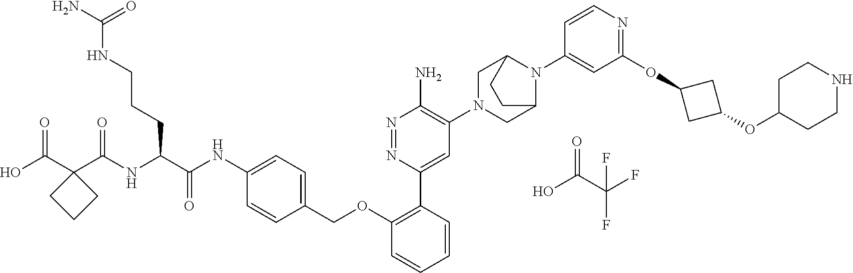 [SMILES: NC(=O)NCCC[C@H](NC(=O)C1(C(=O)O)CCC1)C(=O)Nc1ccc(COc2ccccc2-c2cc(N3CC4CCC(C3)N4c3ccnc(O[C@H]4C[C@H](OC5CCNCC5)C4)c3)c(N)nn2)cc1 ; O=C(O)C(F)(F)F]

To a mixture of 1-(((2S)-1-((4-((2-(6-amino-5-(8-(2-((1r, 3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid (400 mg, 0.39 mmol) in dichloromethane (4.0 mL) was added trifluoroacetic acid (0.30 m L, 3.90 mmol). The mixture was stirred at 20° C. for 5 h. The reaction was concentrated to dryness and the residue was purified by prep-HPLC (Boston Green ODS 150*30 mm*5 um, water (0.075% trifluoroacetic acid)—acetonitrile 12%-42%) to afford the title compound (360 m g, 88.9% yield).

LCMS (ESI) m/z: 932.6 [M+H]⁺.

### Step 4: 1-(((2S)-1-((4-((2-(6-amino-5-(8-(2-((1r,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid

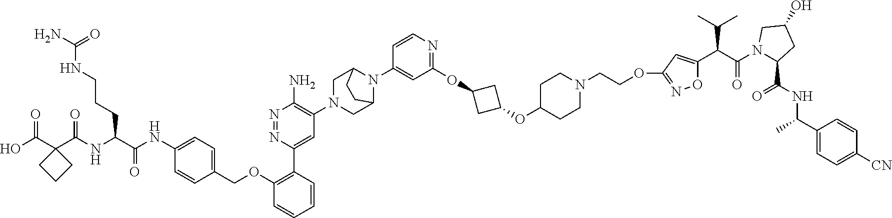 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCNC(N)=O)NC(=O)C8(C(=O)O)CCC8)cc7)nnc5N)C6)ccn4)C3)CC2)no1]

To a solution of (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (313.21 mg, 0.5800 mmol) and 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid 2,2,2-trifluoroacetate (360 mg, 0.39 mmol) in dicloromethane (0.50 mL) and methanol (0.50 mL) was added sodium cyanoborohydride (49.3 mg, 0.77 mmol) and sodium acetate (6.00 mg, 0.07 mmol), one drop acetic acid. The reaction mixture was stirred at 20° C. for 3 h. The resulting residue was purified by Phenomenex Gemini-NX 80*40 mm*3 um (acetonitrile 17-47/0.05% NH₃H₂O in water, 20%-50%) to afford the title compound (250 mg, 46.7%) as a white solid.

LCMS (ESI) m/z: 1384.8 [M+H]⁺.

### Step 5: N-((2S)-1-((4-((2-(6-amino-5-(8-(2-((1r,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)-N-(5-(2,5-dioxo-2,5-dihydro-1H-pyrrol-1-yl)pentyl)cyclobutane-1,1-dicarboxamide

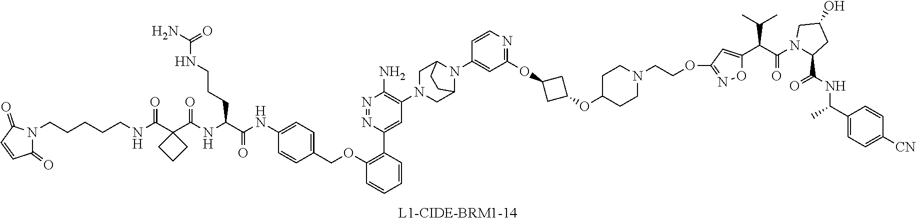 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)C3)CC2)no1]

To a mixture of 1-(((2S)-1-((4-((2-(6-amino-5-(8-(2-((1r, 3r)-3-((1-(2-((5-((R)-1-((2S, 4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid (50.0 mg, 0.04 mmol) and 1-(5-aminopentyl)-1H-pyrrole-2,5-dione 2,2,2-trifluoroacetate (12.8 mg, 0.04 mmol) in N,N-dimethylformamide (4.00 mL) was added N,N-diisopropylethylamine (0.02 mL, 0.1100 mmol), 2-(7-azabenzotriazol-1-yl)-N,N,N,N-tetramethyluronium hexafluorophosphate (16.5 mg, 0.04 mmol). The mixture was stirred at 25° C. for 3 h. The mixture was concentrated by oil pump. The residue was purified by prep-HPLC (Boston Green ODS 150*30 mm*5 um, water (0.075% trifluoroacetic acid)—acetonitrile 20%-50%) to afford the title compound (38.5 mg, 65.4%) as a white solid.

LCMS (ESI) m/z: 775.3 [M/2+H]⁺.

¹H NMR (400 MHz, DMSO-d₆) δ (ppm) 10.19 (s, 1H), 9.02-8.86 (m, 1H), 8.39 (d, J=8.0 Hz, 1H), 7.94 (d, J=6.8 Hz, 1H), 7.87-7.78 (m, 2H), 7.65 (d, J=8.0 Hz, 2H), 7.51-7.41 (m, 5H), 7.36 (d, J=6.8 Hz, 5H), 7.29-7.09 (m, 1H), 6.97 (s, 2H), 6.76 (s, 1H), 6.42 (s, 1H), 6.15-5.96 (m, 2H), 5.07 (s, 2H), 4.93-4.86 (m, 1H), 4.71-4.60 (m, 2H), 4.52-4.24 (m, 9H), 3.66 (s, 4H), 3.33 (d, J=7.2 Hz, 5H), 3.09-2.96 (m, 8H), 2.45 (s, 3H), 2.42-2.34 (m, 4H), 2.29-2.14 (m, 2H), 2.04 (d, J=11.2 Hz, 3H), 1.91 (s, 2H), 1.83-1.55 (m, 5H), 1.51-1.30 (m, 9H), 1.19 (d, J=5.8 Hz, 5H), 0.96 (d, J=6.4 Hz, 3H), 0.86-0.75 (m, 3H).

### Synthesis Example 15

### Synthesis of L1-CIDE-BRM1-15

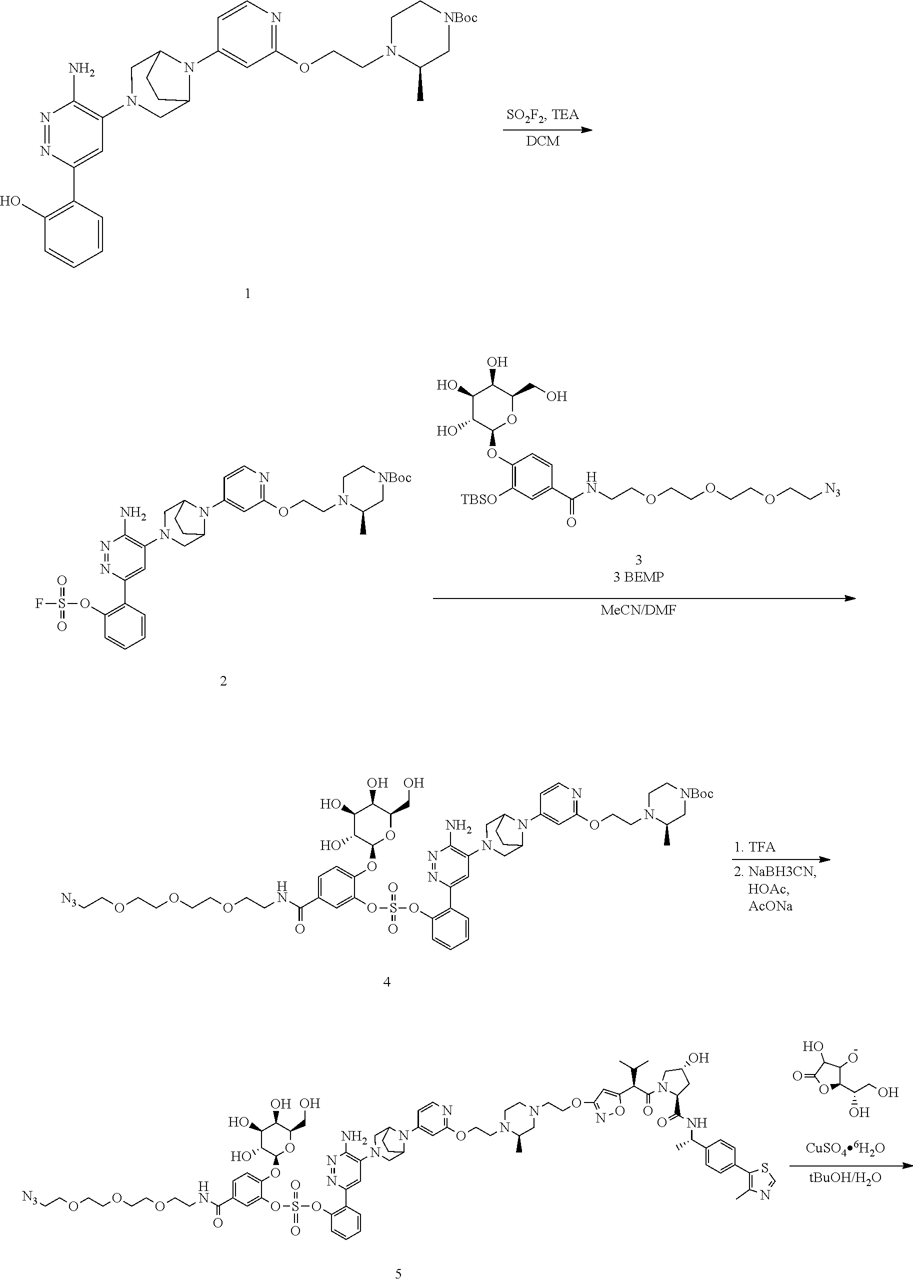

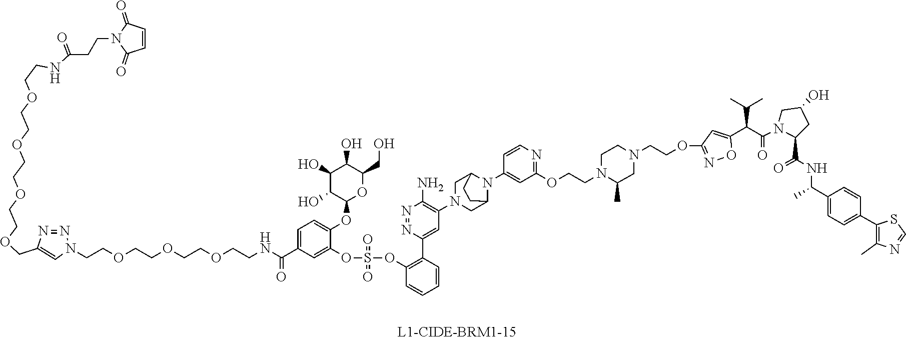 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OS(=O)(=O)Oc7cc(C(=O)NCCOCCOCCOCCn8cc(COCCOCCOCCOCCNC(=O)CCN9C(=O)C=CC9=O)nn8)ccc7O[C@@H]7O[C@H](CO)[C@H](O)[C@H](O)[C@H]7O)nnc5N)C6)ccn4)[C@H](C)C3)no2)C(C)C)cc1]

### Step 1: (3R)-tert-butyl 4-(2-((4-(3-(3-amino-6-(2-((fluorosulfonyl)oxy)phenyl)pyridazin-4-yl)-3,8-diazabicyclo[3.2.1]octan-8-yl)pyridin-2-yl)oxy)ethyl)-3-methylpiperazine-1-carboxylate

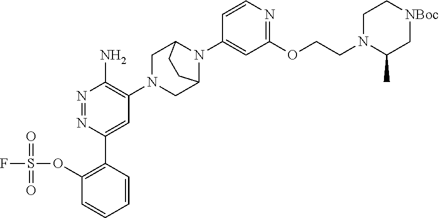 [SMILES: C[C@@H]1CN(C(=O)OC(C)(C)C)CCN1CCOc1cc(N2C3CCC2CN(c2cc(-c4ccccc4OS(=O)(=O)F)nnc2N)C3)ccn1]

A solution of (3R)-tert-butyl 4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (500 mg, 0.81 mmol) in dichloromethane (3.0 mL) was stirred at 0° C. for 16 h under SO₂F₂ balloon. The mixture was concentrated to afford the title compound (566 mg, 99.8%) as a yellow oil. The crude was used to the next step directly. LCMS (ESI) m/z: 713.5 [M+Na]⁺.

### Step 2: N-(2-(2-(2-(2-azidoethoxy)ethoxy)ethoxy)ethyl)-3-((tert-butyldimethylsilyl)oxy)-4-(((2S,3R,4S,5R,6R)-3,4,5-trihydroxy-6-(hydroxymethyl)tetrahydro-2H-pyran-2-yl)oxy)benzamide

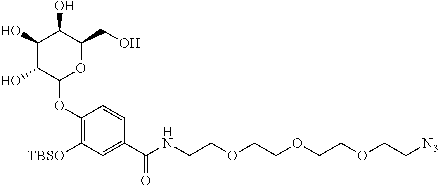 [SMILES: CC(C)(C)[Si](C)(C)Oc1cc(C(=O)NCCOCCOCCOCCN=[N+]=[N-])ccc1OC1O[C@H](CO)[C@H](O)[C@H](O)[C@H]1O]

A solution of N-(2-(2-(2-(2-azidoethoxy)ethoxy)ethoxy)ethyl)-3-hydroxy-4-(((2S, 3R, 4S, 5R, 6R)-3,4,5-trihydroxy-6-(hydroxymethyl)tetrahydro-2H-pyran-2-yl)oxy)benzamide (320 mg, 0 0.70 mmol) in dichloromethane (10.0 mL) was added 2,6-lutidine (0.24 mL, 2.09 mmol) and tert-butyldimethylsilyl trifluoromethanesulfonate (0.32 mL, 1.40 mmol) and stirred at 0° C. for 2 h. The reaction was concentrated and the residue was purified by Column Phenomenex Ge mini-NX C18 75*30 mm*3 um Condition water (0.05% NH₃H₂O+10 mM NH₄HCO₃)—Acetonitrile (35%-65%) to give the title compound (140 mg, 35%) as a white solid.

LCMS (ESI) m/z: 573.4 [M+H]⁺.

### Step 3: (3R)-tert-butyl 4-(2-((4-(3-(3-amino-6-(2-(((5-((2-(2-(2-(2-azidoethoxy)ethoxy)eth oxy)ethyl)carbamoyl)-2-(((2S,3R,4S,5R,6R)-3,4,5-trihydroxy-6-(hydroxymethyl)tetrahydro-2H-pyran-2-yl)oxy)phenoxy)sulfonyl)oxy)phenyl)pyridazin-4-yl)-3,8-diazabicyclo[3.2 0.1]octan-8-yl)pyridin-2-yl)oxy)ethyl)-3-methylpiperazine-1-carboxylate

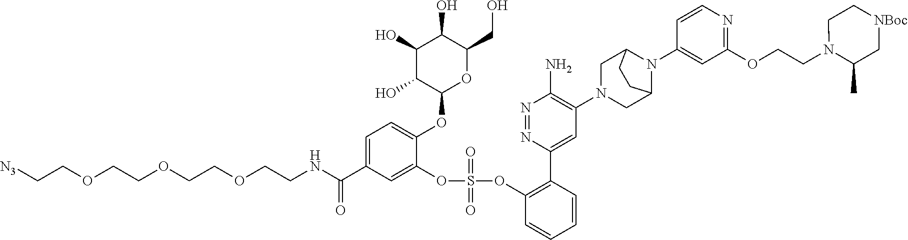 [SMILES: C[C@@H]1CN(C(=O)OC(C)(C)C)CCN1CCOc1cc(N2C3CCC2CN(c2cc(-c4ccccc4OS(=O)(=O)Oc4cc(C(=O)NCCOCCOCCOCCN=[N+]=[N-])ccc4O[C@@H]4O[C@H](CO)[C@H](O)[C@H](O)[C@H]4O)nnc2N)C3)ccn1]

A solution of 1M 2-tert-butylimino-2-diethylamino-1,3-dimethyl-perhydro-1,3,2-diazaphosphorine in acetonitrle (0.86 mL, 0.86 mmol), (3R)-tert-butyl 4-(2-((4-(3-(3-amino-6-(2-((fluorosulfonyl)oxy)phenyl)pyridazin-4-yl)-3,8-diazabicyclo[3.2.1]octan-8-yl)pyridin-2-yl)oxy)ethyl)-3-methylpiperazine-1-carboxylate (300 mg, 0.43 mmol) and N-(2-(2-(2-(2-azidoethoxy)ethoxy)ethoxy)ethyl)-3-((tert-butyldimethylsilyl)oxy)-4-(((2S,3R,4S,5R,6R)-3,4,5-trihydroxy-6-(hydroxymethyl)tetrahydro-2H-pyran-2-yl)oxy)benzamide (274 mg, 0.43 mmol) in acetonitrile (5.0 mL) and N,N-dimethylformamide (1.00 mL) was stirred at 24° C. for 16 h. The crude mixture was concentrated to dryness and the reside was purified by prep-HPLC (Welch Xtimate C18 150*25 mm*5 um/water (10 mM NH₄HCO₃)— acetonitrile/40-70%) to afford the title corn pound (200 mg, 39% yield) as a white solid.

LCMS (ESI) m/z: 1195.6 [M+H]⁺.

### Step 4: 2-(6-amino-5-(8-(2-(2-((R)-2-methylpiperazin-1-yl)ethoxy)pyridin-4-yl)-3,8-diaza bicyclo[3.2.1]octan-3-yl)pyridazin-3-yl)phenyl (5-((2-(2-(2-(2-azidoethoxy)ethoxy)ethoxy)ethyl)carbamoyl)-2-(((2S,3R,4S,5R,6R)-3,4,5-trihydroxy-6-(hydroxymethyl)tetrahydro-2H-pyran-2-yl)oxy)phenyl) sulfate 2,2,2-trifluoroacetate

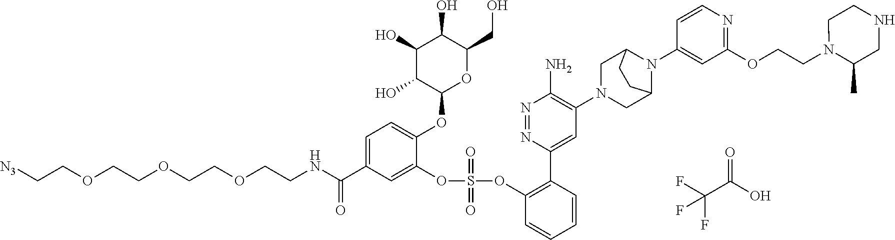 [SMILES: C[C@@H]1CNCCN1CCOc1cc(N2C3CCC2CN(c2cc(-c4ccccc4OS(=O)(=O)Oc4cc(C(=O)NCCOCCOCCOCCN=[N+]=[N-])ccc4O[C@@H]4O[C@H](CO)[C@H](O)[C@H](O)[C@H]4O)nnc2N)C3)ccn1 ; O=C(O)C(F)(F)F]

A solution of (3R)-tert-butyl 4-(2-((4-(3-(3-amino-6-(2-(((5-((2-(2-(2-(2-azidoethoxy)ethoxy) ethoxy)ethyl)carbamoyl)-2-(((2S, 3R, 4S, 5R, 6R)-3,4,5-trihydroxy-6-(hydroxymethyl)tetrahydro-2H-pyran-2-yl)oxy)phenoxy)sulfonyl)oxy)phenyl)pyridazin-4-yl)-3,8-diazabicyclo[3.2.1]octan-8-yl)pyridin-2-yl)oxy)ethyl)-3-methylpiperazine-1-carboxylatee (205 mg, 0.17 mmol) i n 5% trifluoroacetic acid in hexafluoroisopropanol (6.00 mL) was stirred at 15° C. for 2 h. The mixture was concentrated to afford the title compound (207 mg, 99.8%) as a white solid. The crude was used to the next step directly. LCMS (ESI) m/z: 1095.4 [M+H-TFA]⁺.

### Step 4: 2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenyl (5-((2-(2-(2-(2-azidoethoxy)ethoxy)ethoxy)ethyl)carbamoyl)-2-(((2S,3R,4S,5R,6R)-3,4,5-trihydroxy-6-(hydroxymethyl)tetrahydro-2H-pyran-2-yl)oxy)phenyl) sulfate

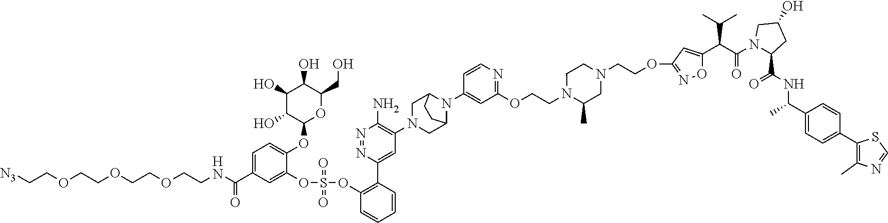 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OS(=O)(=O)Oc7cc(C(=O)NCCOCCOCCOCCN=[N+]=[N-])ccc7O[C@@H]7O[C@H](CO)[C@H](O)[C@H](O)[C@H]7O)nnc5N)C6)ccn4)[C@H](C)C3)no2)C(C)C)cc1]

To a solution of 2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenyl (5-((2-(2-(2-(2-azidoethoxy)ethoxy)ethoxy)ethyl)carbamoyl)-2-(((2S,3R,4S,5R,6R)-3,4,5-trihydroxy-6-(hydroxymethyl)tetrahydro-2H-pyran-2-yl)oxy)phenyl) sulfate 2,2,2-trifluoroacetate (207 mg, 0.17 mmol) and (2S, 4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (184 mg, 0.34 mmol) in methanol (1.00 mL) and dichloromethane (1.00 mL) was added sodiumcyanoborohydride (11.8 mg, 0.19 mmol) and acetoxysodium (42.1 mg, 0.51 mmol). The reaction was stirred at 20° C. for 3 h. The crud e was purified by prep-HPLC (Welch Xtimate C18 150*25 mm*5 um/water (10 mM NH₄HCO 3)—acetonitrile/30-60%) to afford the title compound (145 mg, 52.3%) as a white solid. LC MS (ESI) m/z: 810.9 [1/2M+H]⁺.

### Step 5: 2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenyl (5-((2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)ethoxy)ethoxy)ethoxy)ethyl)carbamoyl)-2-(((2S,3R,4S,5R,6R)-3,4,5-trihydroxy-6-(hydroxymethyl)tetrahydro-2H-pyran-2-yl)oxy)phenyl) sulfate

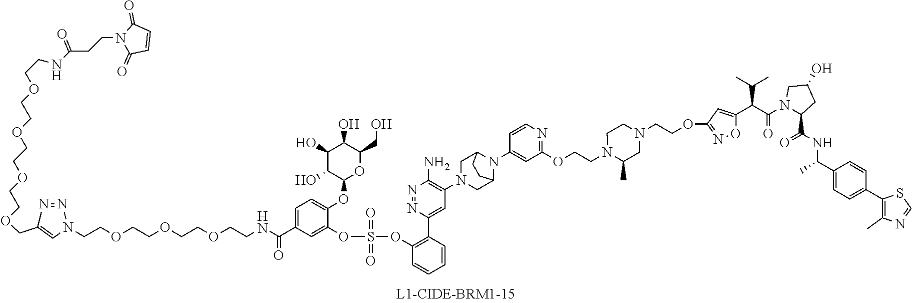 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OS(=O)(=O)Oc7cc(C(=O)NCCOCCOCCOCCn8cc(COCCOCCOCCOCCNC(=O)CCN9C(=O)C=CC9=O)nn8)ccc7O[C@@H]7O[C@H](CO)[C@H](O)[C@H](O)[C@H]7O)nnc5N)C6)ccn4)[C@H](C)C3)no2)C(C)C)cc1]

To a solution of 2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenyl (5-((2-(2-(2-(2-azidoethoxy)ethoxy)ethoxy)ethyl)carbamoyl)-2-(((2S,3R,4S,5R,6R)-3,4,5-trihydroxy-6-(hydroxymethyl)tetrahydro-2H-pyran-2-yl)oxy)phenyl) sulfate (45.0 mg, 0.03 mmol) and 3-(2,5-dioxopyrrol-1-yl)-N-[2-[2-[2-(2-prop-2-ynoxyethoxy)ethoxy]ethoxy]ethyl]propanamide (20.0 mg, 0.05 mmol) in Dimethyl sulfoxide (1.00 mL) and water (1.00 mL) was added a solution of copper sulfate (12.4 mg, 0.06 mmol) in water (0 0.2 mL) and a solution of sodiumascorbate (11.01 mg, 0.06 mmol) in water (0.2 mL) at 0° C. The reaction was stirred at 26° C. for 1 h under N₂ atmosphere. The crude was purified by pre p-HPLC (Welch Xtimate C18 100*40 mm*3 um/water (0.075% trifluoroacetic acid)-acetonitrile/15-45%) to get the title compound (22.6 mg, 40.6%) as a white solid.

LCMS (ESI) m/z: 1001.9 [1/2M+H]⁺.

### Synthesis Example 16

### Synthesis of L1-CIDE-BRM1-16

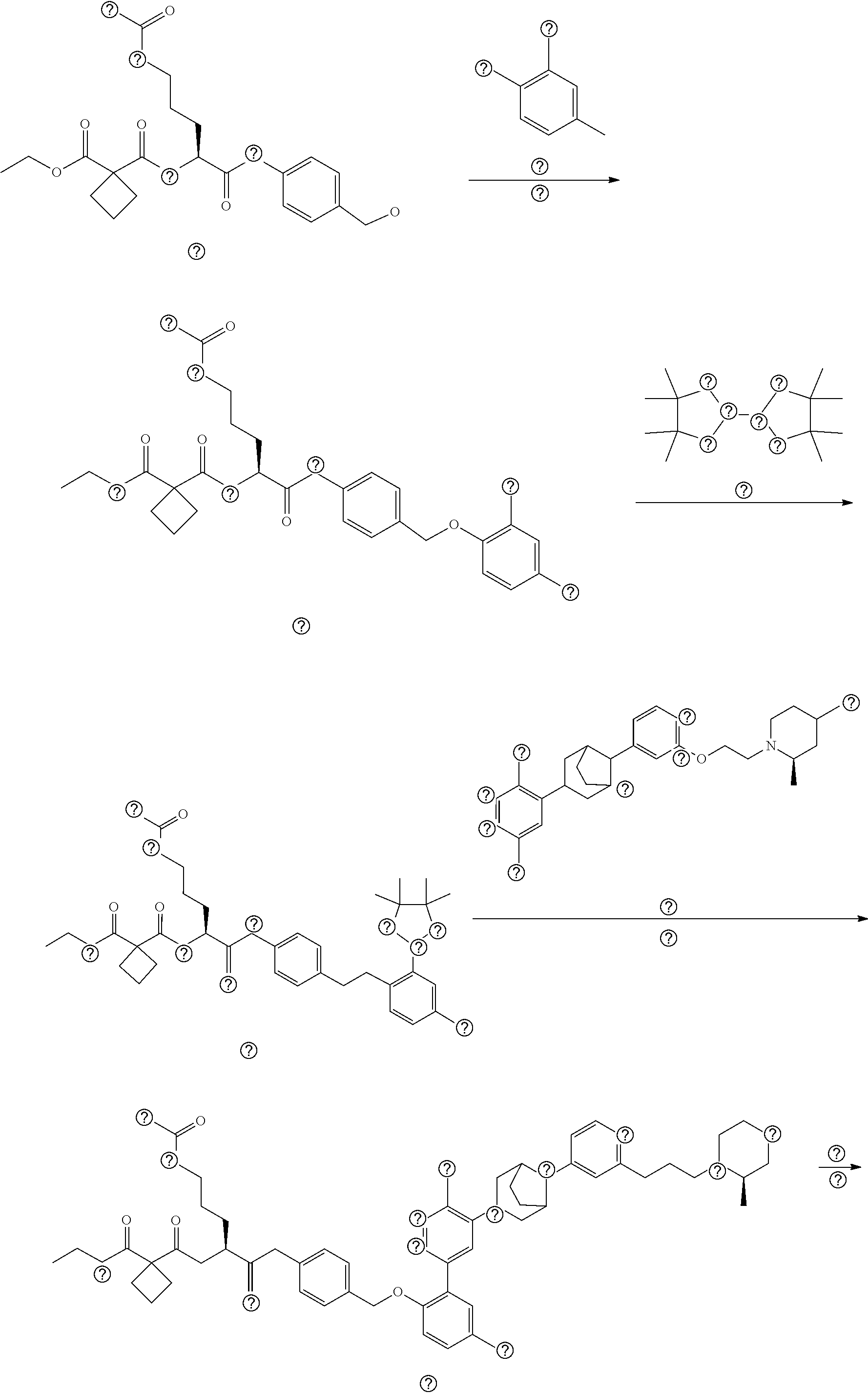

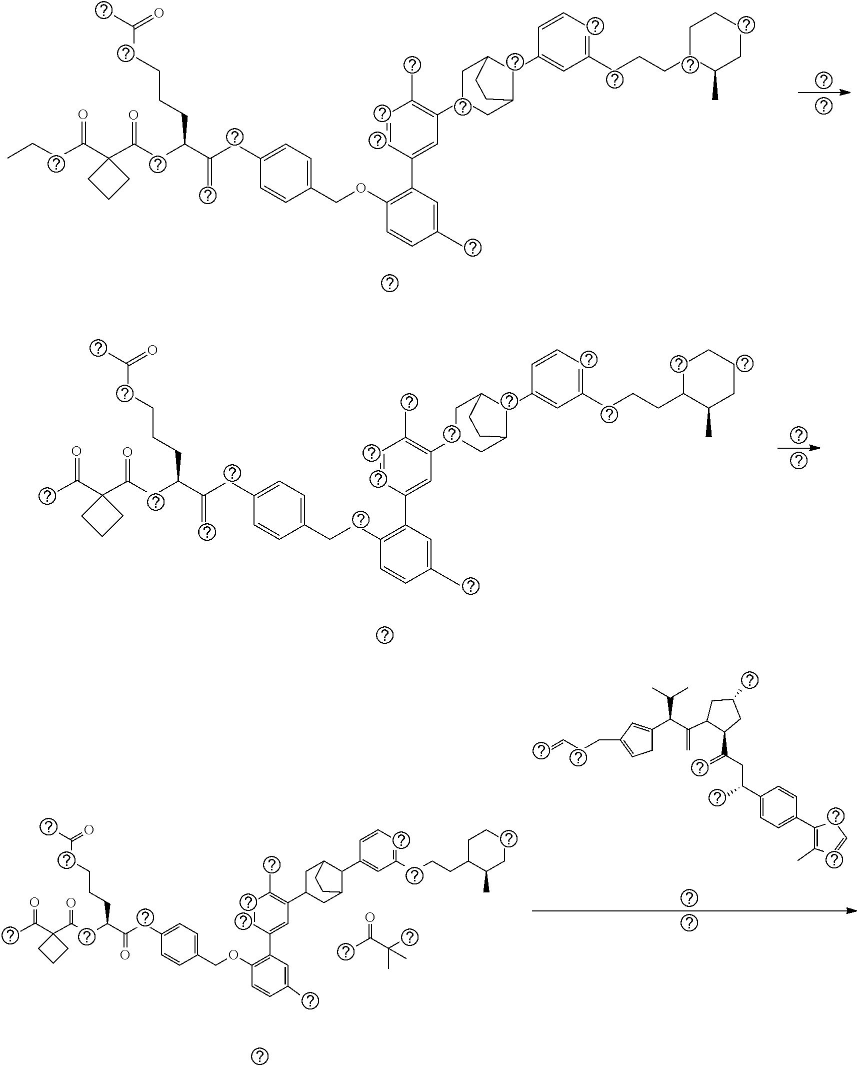

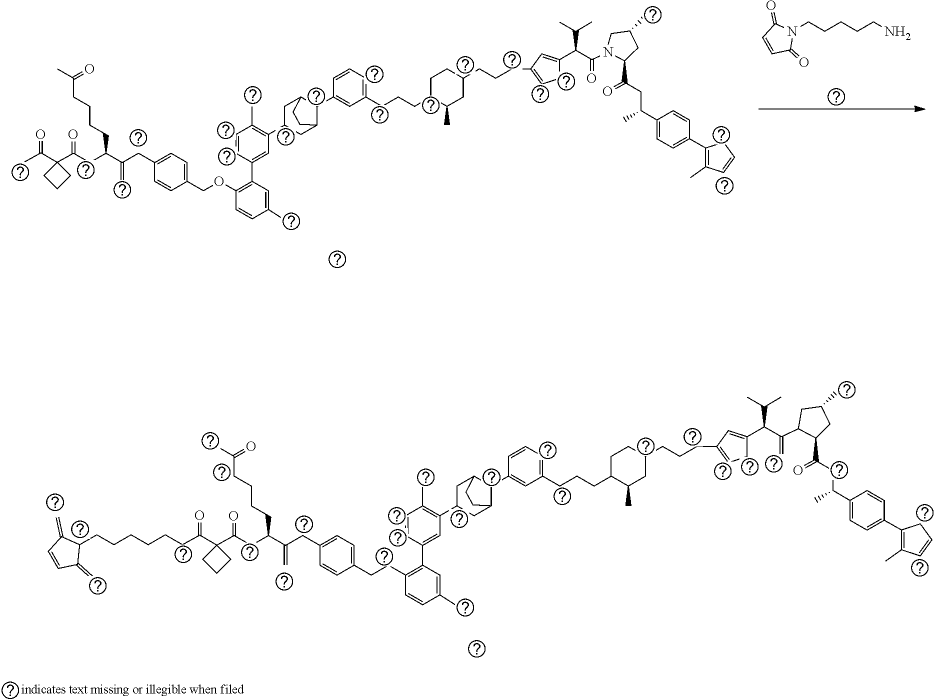 [SMILES: C=C(Cc1ccc(COc2ccc(C)cc2-c2ccc(C)c(C3CC4CCC(C3)C4c3cccc(CCCC4CCC(CCCC5=CCC([C@H](C(=O)N6C[C@H](C)C[C@H]6C(=O)C[C@@H](C)c6ccc(C7=C(C)C=CC7)cc6)C(C)C)=C5)C[C@H]4C)c3)c2)cc1)[C@H](CCCCC(C)=O)CC(=O)C1(C(C)=O)CCC1 ; NCCCCCN1C(=O)C=CC1=O ; C=C1C=CC(=C)C1CCCCCCC(=O)C1(C(=O)C[C@@H](CCCCC(C)=O)C(=C)Cc2ccc(CCc3ccc(C)cc3-c3ccc(C)c(C4CC5CCC(C4)C5c4cccc(CCCC5CCC(CCCC6=CCC([C@H](C(=C)C7C[C@H](C)C[C@H]7C(=O)C[C@@H](C)c7ccc(C8=C(C)C=CC8)cc7)C(C)C)=C6)C[C@H]5C)c4)c3)cc2)CCC1]

### Step 1: (S)-ethyl 1-((1-((4-((2-bromo-4-fluorophenoxy)methyl)phenyl)amino)-1-oxo-5-ureidopentan-2-yl)carbamoyl)cyclobutanecarboxylate

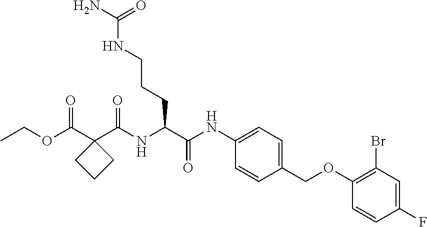 [SMILES: CCOC(=O)C1(C(=O)N[C@@H](CCCNC(N)=O)C(=O)Nc2ccc(COc3ccc(F)cc3Br)cc2)CCC1]

To a solution of (S)-ethyl 1-((1-((4-(chloromethyl)phenyl)amino)-1-oxo-5-ureidopentan-2-yl)carbamoyl)cyclobutanecarboxylate (1.00 g, 2.21 mmol) and potassium carbonate (0.76 g, 5.52 mmol) in N,N-dimethylformamide (60.0 mL) was added 2-bromo-4-fluoro-phenol (0.63 g, 3.31 mmol) at 25° C. The reaction was stirred at 25° C. for 3 h. The reaction mixture was diluted with water (30.0 mL) and extracted with dichloromethane (50 mL×3). The combined organics were washed with brine (20 mL×2), dried over sodium sulfate, filtered and concentrated to dryness. The residue was purified by flash chromatography (silica gel, 100-200 mesh, 0-5% methanol in dichloromethane) to afford the title compound (1.2 g, 89.5%) as a white solid.

### Step 2: (S)-ethyl 1-((1-((4-((4-fluoro-2-(4,4,5,5-tetramethyl-1,3,2-dioxaborolan-2-yl)phenoxy)methyl)phenyl)amino)-1-oxo-5-ureidopentan-2-yl)carbamoyl)cyclobutanecarboxylate

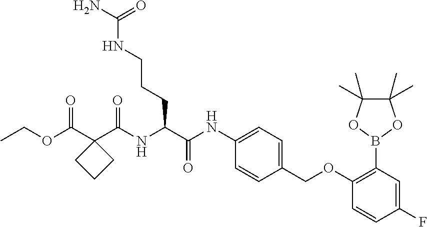 [SMILES: CCOC(=O)C1(C(=O)N[C@@H](CCCNC(N)=O)C(=O)Nc2ccc(COc3ccc(F)cc3B3OC(C)(C)C(C)(C)O3)cc2)CCC1]

To a solution of (S)-ethyl 1-((1-((4-((2-bromo-4-fluorophenoxy)methyl)phenyl)amino)-1-oxo-5-ureidopentan-2-yl)carbamoyl)cyclobutanecarboxylate (1.00 g, 1.65 mmol) and 4,4,4′,4′,5,5,5′,5′-octamethyl-2,2′-bi(1,3,2-dioxaborolane) (627 mg, 2.47 mmol) in 1,4-dioxane (5.0 mL) was added 1,1′-bis(diphenylphosphino)ferrocene palladium dichloride (120 mg, 0.16 mmol) and sodium acetate (485 mg, 4.94 mmol). The mixture was stirred at 100° C. under N₂ for 3 h. The mixture crude was used directly in next step.

LCMS (ESI) m/z: 655.4 [M+H]⁺.

### Step 3: (3R)-tert-butyl 4-(2-((4-(3-(3-amino-6-(2-((4-((S)-2-(1-(ethoxycarbonyl)cyclobutanecarboxamido)-5-ureidopentanamido)benzyl)oxy)-5-fluorophenyl)pyridazin-4-yl)-3,8-diazabicyclo[3.2.1]octan-8-yl)pyridin-2-yl)oxy)ethyl)-3-methylpiperazine-1-carboxylate

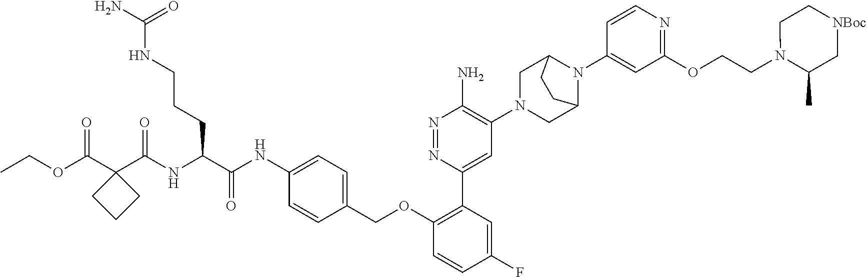 [SMILES: CCOC(=O)C1(C(=O)N[C@@H](CCCNC(N)=O)C(=O)Nc2ccc(COc3ccc(F)cc3-c3cc(N4CC5CCC(C4)N5c4ccnc(OCCN5CCN(C(=O)OC(C)(C)C)C[C@H]5C)c4)c(N)nn3)cc2)CCC1]

To a solution of (3R)-tert-butyl 4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (1.11 g, 1.99 mmol) and potassium carbonate (0.38 mL, 4.58 mmol) in dimethyl sulfoxide (5 mL) was added [2-(2-aminophenyl)phenyl]-chloro-palladium; bis(1-adamantyl)-butyl-phosphane (102.15 mg, 0.15 mmol) and (S)-ethyl 1-((1-((4-((4-fluoro-2-(4,4,5,5-tetramethyl-1,3,2-dioxaborolan-2-yl)phenoxy)methyl)phenyl)amino)-1-oxo-5-ureidopentan-2-yl)carbamoyl)cyclobutanecarboxylate (1.00 g, 1.53 mmol). The reaction mixture was stirred at 100° C. under N₂ for 3 h. The reaction was purified by a flash chromatography (silica gel, 100-200 mesh, 0-10% methanol in dichloromethane) to afford the title compound (360 mg, 22.4%) as a dark solid.

LCMS (ESI) m/z: 1095 [M+H]⁺.

### Step 4:1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid

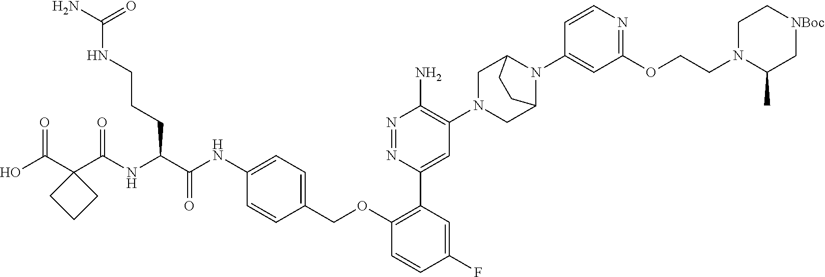 [SMILES: C[C@@H]1CN(C(=O)OC(C)(C)C)CCN1CCOc1cc(N2C3CCC2CN(c2cc(-c4cc(F)ccc4OCc4ccc(NC(=O)[C@H](CCCNC(N)=O)NC(=O)C5(C(=O)O)CCC5)cc4)nnc2N)C3)ccn1]

To a solution of (3R)-tert-butyl 4-(2-((4-(3-(3-amino-6-(2-((4-((S)-2-(1-(ethoxycarbonyl)cyclobutanecarboxamido)-5-ureidopentanamido)benzyl)oxy)-5-fluorophenyl)pyridazin-4-yl)-3,8-diazabicyclo[3.2.1]octan-8-yl)pyridin-2-yl)oxy)ethyl)-3-methylpiperazine-1-carboxylate (360 mg, 0.34 mmol) in tetrahydrofuran (2.00 mL), methanol (5.0 mL) and water (2.00 mL) was added lithium hydroxide monohydrate (41.0 mg, 1.71 mmol). The reaction mixture was stirred at 100° C. under N₂ atmosphere for 3 h. The reaction mixture was concentrated to dryness to give the title compound (350 mg, 99.9%) as a white solid.

### Step 5: 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid 2,2,2-trifluoroacetate

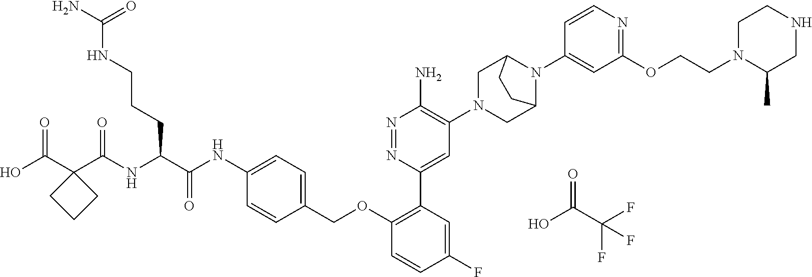 [SMILES: C[C@@H]1CNCCN1CCOc1cc(N2C3CCC2CN(c2cc(-c4cc(F)ccc4OCc4ccc(NC(=O)[C@H](CCCNC(N)=O)NC(=O)C5(C(=O)O)CCC5)cc4)nnc2N)C3)ccn1 ; O=C(O)C(F)(F)F]

To a solution of 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid (340 mg, 0.33 mmol) in dichloromethane (5.0 mL) was added trifluoroacetic acid (0.05 mL, 0.66 mmol). The reaction mixture was stirred at 20° C. for 3 h. The reaction mixture was concentrated to dryness and the residue was purified by prep-HPLC (Boston Green ODS 150*30 mm*5 um, water (0.075% trifluoroacetic acid)-acetonitrile 12%-42%) to afford the title compound (80 mg, 26.1%) as a white solid.

### Step 6: 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid

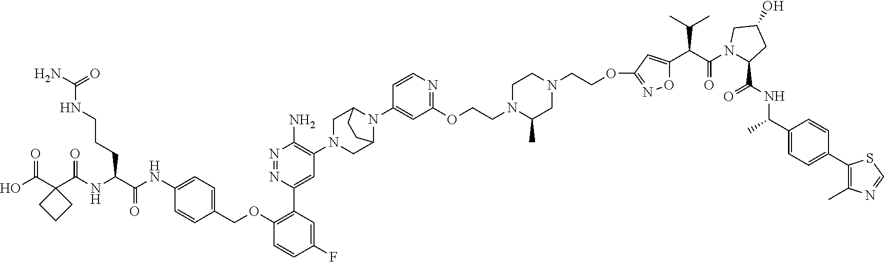 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7cc(F)ccc7OCc7ccc(NC(=O)[C@H](CCCNC(N)=O)NC(=O)C8(C(=O)O)CCC8)cc7)nnc5N)C6)ccn4)[C@H](C)C3)no2)C(C)C)cc1]

To a solution of (2S, 4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (70.3 mg, 0.13 mmol) and 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid 2,2,2-trifluoroacetate (80.0 mg, 0.09 mmol) in dichloromethane (0.6 mL) and methanol (0.6 mL) was added sodium cyanoborohydride (11.1 mg, 0.17 mmol) and sodium acetate (6.0 mg, 0.07 mmol) and one drop acetic acid. The reaction mixture was stirred at 20° C. for 3 h. The resulting residue was purified by Phenomenex Gemini-NX 80*40 mm*3 um (acetonitrile 17-47/0.05% NH₃H₂O in water) to afford the title compound (80 mg, 63.8% yield) as a white solid.

LCMS (ESI) m/z: 1448.8 [M+H]⁺.

### Step 7: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)-N-(5-(2,5-dioxo-2,5-dihydro-1H-pyrrol-1-yl)pentyl)cyclobutane-1,1-dicarboxamide

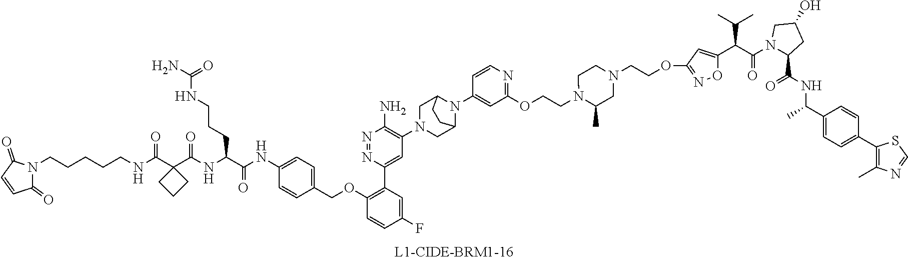 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7cc(F)ccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1]

To a mixture of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)-4-fluorophenoxy)methyl)phenyl)amino)-1-oxo-5-ureidopentan-2-yl)carbamoyl)cyclobutanecarboxylic acid (80.0 mg, 0.06 mmol) and 1-(5-aminopentyl)pyrrole-2,5-dione; 2,2,2-trifluoroacetic acid (19.6 mg, 0.07 mmol) in N,N-dimethylformamide (4.00 mL) was added 2-(7-Azabenzotriazol-1-yl)-N,N,N′,N′-tetramethyluronium hexafluorophosphate (25.2 mg, 0.07 mmol), N,N-diisopropylethylamine (0.03 mL, 0.1700 mmol). The reaction mixture was stirred at 25° C. for 3 h. The reaction mixture was concentrated to dryness by oil pump. The residue was purified by prep-HPLC (Boston Green ODS 150*30 mm*5 um, water (0.075% trifluoroacetic acid)-acetonitrile 20%-50%) to afford the title compound (68.2 mg, 75% yield) as a white solid.

LCMS (ESI) m/z: 806.7 [M/2+H]⁺.

¹H NMR (400 MHz, DMSO-d₆) δ (ppm) 10.19 (s, 1H), 9.02-8.86 (m, 1H), 8.39 (d, J=8.0 Hz, 1H), 7.94 (d, J=6.8 Hz, 1H), 7.87-7.78 (m, 2H), 7.65 (d, J=8.0 Hz, 2H), 7.51-7.41 (m, 5H), 7.36 (d, J=6.8 Hz, 5H), 7.29-7.09 (m, 1H), 6.97 (s, 2H), 6.76 (s, 1H), 6.42 (s, 1H), 6.15-5.96 (m, 2H), 5.07 (s, 2H), 4.93-4.86 (m, 1H), 4.71-4.60 (m, 2H), 4.52-4.24 (m, 9H), 3.66 (s, 4H), 3.33 (d, J=7.2 Hz, 5H), 3.09-2.96 (m, 8H), 2.45 (s, 3H), 2.42-2.34 (m, 4H), 2.29-2.14 (m, 2H), 2.04 (d, J=11.2 Hz, 3H), 1.91 (s, 2H), 1.83-1.55 (m, 5H), 1.51-1.30 (m, 9H), 1.19 (d, J=5.8 Hz, 5H), 0.96 (d, J=6.4 Hz, 3H), 0.86-0.75 (m, 3H).

### Synthesis Example 17

### Synthesis of L1-CIDE-BRM1-17

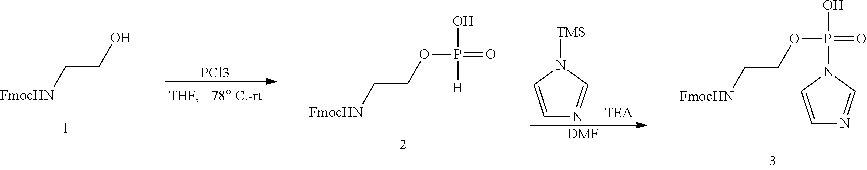

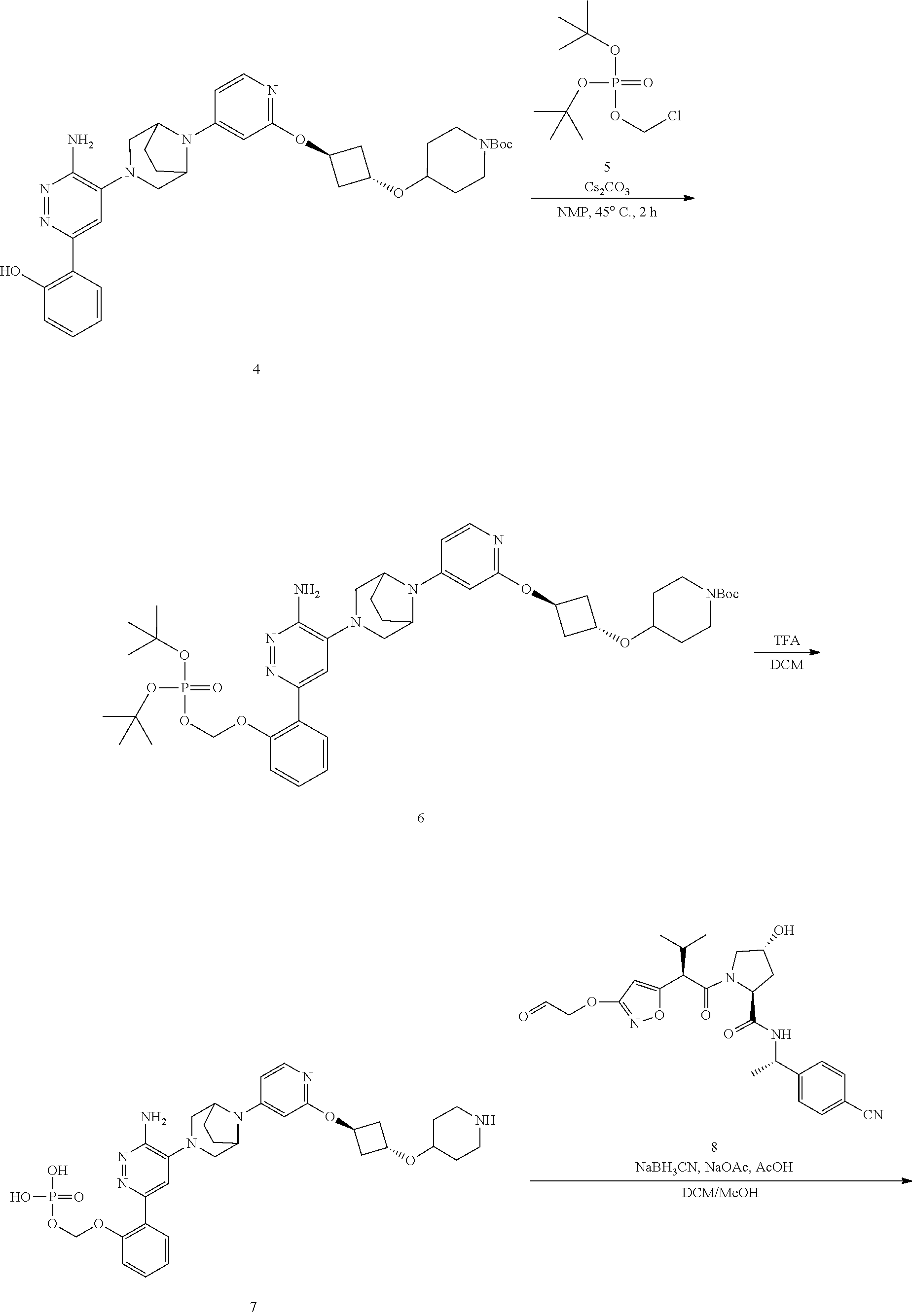 [SMILES: CC(C)(C)OC(=O)N1CCC(O[C@H]2C[C@H](Oc3cc(N4C5CCC4CN(c4cc(-c6ccccc6O)nnc4N)C5)ccn3)C2)CC1 ; CC(C)(C)OP(=O)(OCCl)OC(C)(C)C ; "5Cs2CO3" ; CC(C)(C)OC(=O)N1CCC(O[C@H]2C[C@H](Oc3cc(N4C5CCC4CN(c4cc(-c6ccccc6OCOP(=O)(OC(C)(C)C)OC(C)(C)C)nnc4N)C5)ccn3)C2)CC1 ; Nc1nnc(-c2ccccc2OCOP(=O)(O)O)cc1N1CC2CCC(C1)N2c1ccnc(O[C@H]2C[C@H](OC3CCNCC3)C2)c1 ; CC(C)[C@@H](C(=O)N1C[C@H](O)C[C@H]1C(=O)N[C@@H](C)c1ccc(C#N)cc1)c1cc(OCC=O)no1]

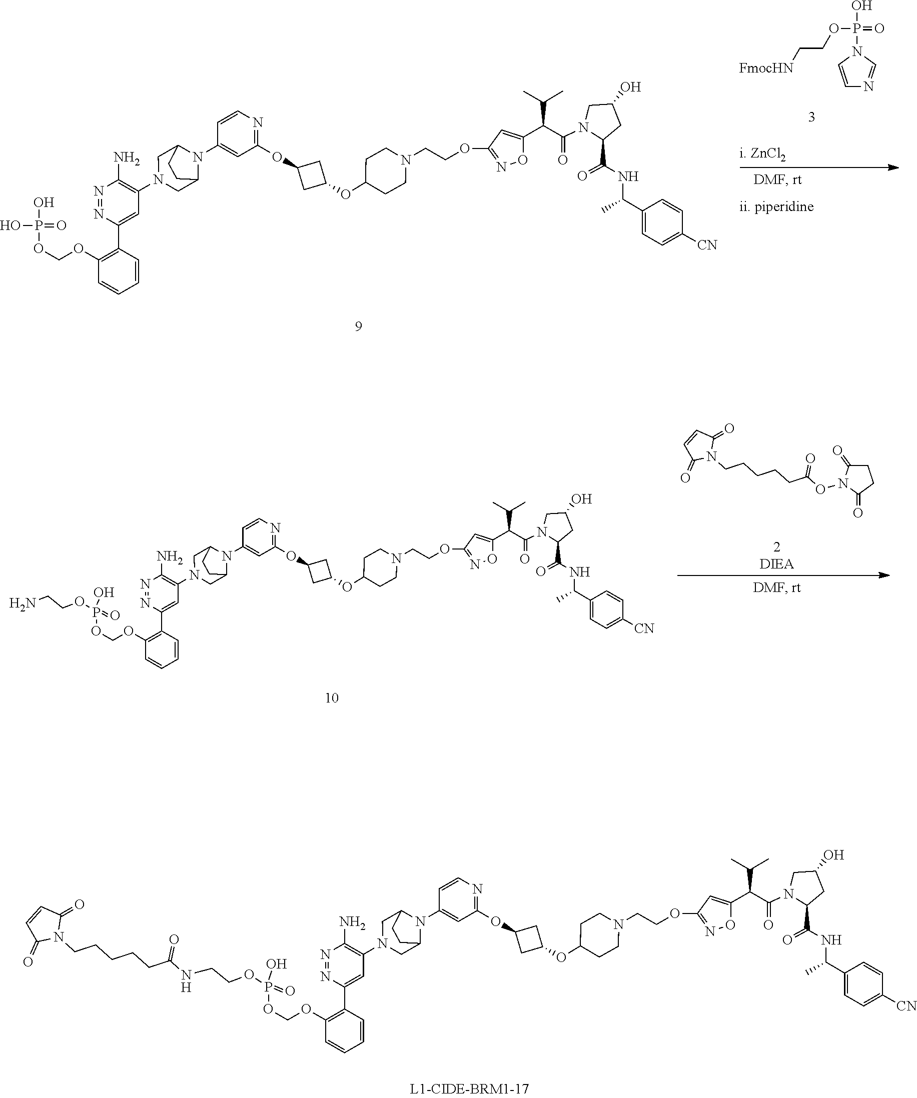

### Step 1: (9H-fluoren-9-yl)methyl (2-((hydroxyhydrophosphoryl)oxy)ethyl)carbamate

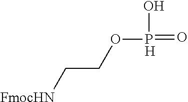

To a solution of (9H-fluoren-9-yl)methyl (2-hydroxyethyl)carbamate (1.0 g, 3.53 mmol) in tetrahydrofuran (3.00 mL) was added phosphorus trichloride (0.73 mL, 8.44 mmol) in tetrahydrofuran (5.0 mL) and triethylamine (1.1 mL, 7.89 mmol) in tetrahydrofuran (3.0 mL) at −78° C. The reaction mixture was stirred at −78° C. for 20 min then allowed warm to 25° C. The resulted mixture was stirred at 25° C. for 12 h. The reaction was diluted with water (20 mL) and extracted with ethyl acetate (10 mL×3). The organics were washed with brine (20 mL×2), dried over sodium sulfate, filtered and concentrated to afford the title compound (1.20 g, 97.9%) as a white solid.

LCMS (ESI) m/z: 695.3 [2M+H]⁺.

### Step 2: (9H-fluoren-9-yl)methyl (2-((hydroxy(1H-imidazol-1-yl)phosphoryl)oxy)ethyl) carbamate

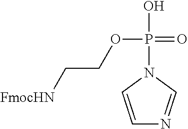

To a solution of (9H-fluoren-9-yl)methyl (2-((hydroxyhydrophosphoryl)oxy)ethyl)carbamate (0.50 g, 1.44 mmol) and triethylamine (0.6 mL, 4.32 mmol) in carbon tetrachloride (5.0 mL) and acetonitrile (5.0 mL) was added 1-(trimethylsilyl)-1H-imidazole (0.61 g, 4.32 mmol) at 25° C. The reaction mixture was stirred at 25° C. for 40 min. The mixture was treated with m ethanol (0.1 mL) and stirred at 25° C. for 10 min. The solvent was removed and the residue w as washed with methyl tert-butyl ether/ethyl acetate=5/1 (3.0 mL), the precipitate was filtered and washed with tert-butyl ether (3.00 mL), obtained the title compound (590 mg, 99.1% yield) as a yellow oil.

LCMS (ESI) m/z: 414.3 [M+H]⁺.

### Step 3: tert-butyl 4-((1r,3r)-3-((4-(3-(3-amino-6-(2-(((di-tert-butoxyphosphoryl)oxy)methoxy)phenyl)pyridazin-4-yl)-3,8-diazabicyclo[3.2.1]octan-8-yl)pyridin-2-yl)oxy)cyclobutoxy)piperidine-1-carboxylate

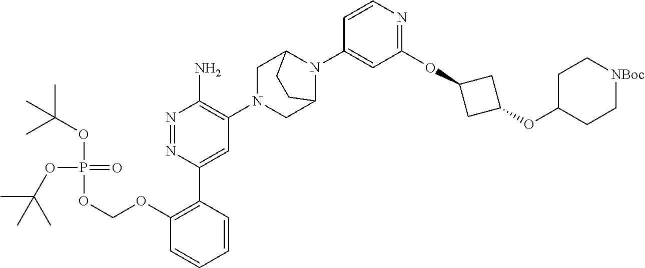 [SMILES: CC(C)(C)OC(=O)N1CCC(O[C@H]2C[C@H](Oc3cc(N4C5CCC4CN(c4cc(-c6ccccc6OCOP(=O)(OC(C)(C)C)OC(C)(C)C)nnc4N)C5)ccn3)C2)CC1]

To a solution of tert-butyl 4-((1r, 3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (1.70 g, 2.64 mmol) in N,N-dimethylformamide (36.0 mL) was added cesium carbonate (1.72 g, 5.28 mmol) and di-tert-butyl (chloromethyl) phosphate (1.02 g, 3.96 mmol). The reaction mixture was stirred at 70° C. for 12 h. The reaction mixture was quenched by water (150 mL) an d extracted with ethyl acetate (80 mL×3). The combined organic layers were washed with brine (100 mL×2), dried over anhydrous sodium sulfate, filtered and concentrated to dryness under reduced pressure. The residue was purified by silica gel chromatography (petroleum ether:ethyl acetate=100:1 to 50:1) to afford the title compound (1.05 g, 45.9% yield) as a colorless oil.

LCMS (ESI) m/z: 866.4 [M+H]⁺.

### Step 4: (2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl dihydrogen phosphate 2,2,2-trifluoroacetic acid

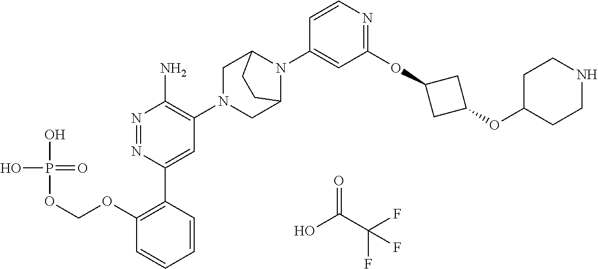 [SMILES: Nc1nnc(-c2ccccc2OCOP(=O)(O)O)cc1N1CC2CCC(C1)N2c1ccnc(O[C@H]2C[C@H](OC3CCNCC3)C2)c1 ; O=C(O)C(F)(F)F]

To a solution of tert-butyl 4-((1r, 3r)-3-((4-(3-(3-amino-6-(2-(((di-tert-butoxyphosphoryl)oxy) methoxy)phenyl)pyridazin-4-yl)-3,8-diazabicyclo[3.2.1]octan-8-yl)pyridin-2-yl)oxy)cyclobut oxy)piperidine-1-carboxylate (1.05 g, 1.21 mmol) in dichloremethane (36.0 mL) was added trifluoroacetic acid (0.09 mL, 1.21 mmol). The reaction mixture was stirred at 20° C. for 12 h. The reaction was concentrated to afford the title compound (930 mg, 99%) as yellow oil.

LCMS (ESI) m/z: 654.4 [M+H]⁺.

### Step 5: (2-(6-amino-5-(8-(2-((1r,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl dihydrogen phosphate

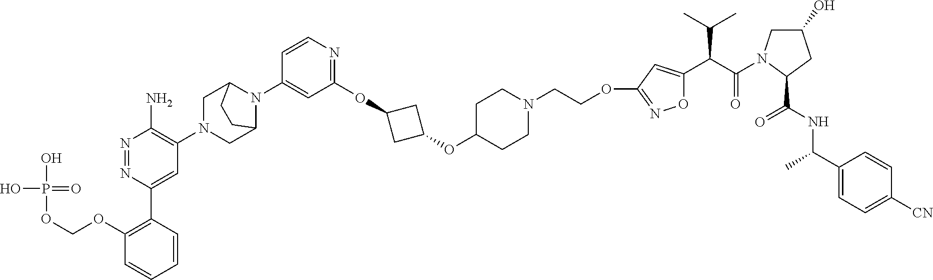 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)O)nnc5N)C6)ccn4)C3)CC2)no1]

To a solution of (2S, 4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (568 mg, 1.21 mmol) in dichloromethane (0.6 mL) and methanol (0.6 mL) was added (2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl dihydrogen phosphate 2,2,2-trifluoroacetic acid (930 mg, 1.21 mmol), sodium cyanoborohydride (155 mg, 2.42 mmol) and sodium acetate (596 mg, 7.26 mmol) and one drop acetic acid. The reaction mixture was stirred at 20° C. for 3 h. The reaction was purified by Prep-HPLC with the following conditions: Column, Phenomenex Gemini-NX 80*40 mm*3 um; mobile phase: 11-41% (water (0.05% NH₃H₂O)—acetonitrile); Detector, UV 254 nm to afford the title compound (700 mg, 52.2% yield) as a white solid. LCMS (ESI) m/z: 1106.5 [M+H]⁺.

### Step 6: (9H-fluoren-9-yl)methyl (2-((((((2-(6-amino-5-(8-(2-((1r,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methoxy)(hydroxy)phosphoryl)oxy)(hydroxy)phosphoryl)oxy)ethyl)carbamate

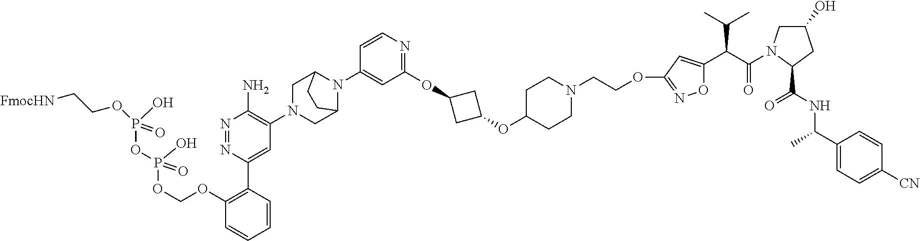

To a solution of (2-(6-amino-5-(8-(2-((1r, 3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl dihydrogen phosphate (200 mg, 0.18 mmol) in N,N-dimethylformamide (36.0 mL) was added 1M zinc dichloride in tetrahydrofuran (1.45 mL, 1.45 mmol) and (9H-fluoren-9-yl)methyl (2-((hydroxy(1H-imidazol-1-yl)phosphoryl)oxy)ethyl)carbamate (149 mg, 0.36 mmol). The reaction mixture was stirred at 20° C. for 12 h. The crude product was purified by Prep-HPLC with the following conditions: Column, Phenomenex Gemini-NX 80*40 mm*3 um; mobile phase: 9-39% water (0.05% NH₃H₂O)—acetonitrile); Detector, UV 254 nm to afford the title compound (200 mg, 76.2% yield) as a white solid.

LCMS (ESI) m/z: 726.7 [M/2+H]⁺.

### Step 7: 2-aminoethoxy(hydroxy)phosphoryl] [2-[6-amino-5-[8-[2-[3-[[1-[2-[5-[rac-(1R)-2-methyl-1-[rac-(2S,4R)-4-hydroxy-2-[[rac-(1S)-1-(4-cyanophenyl)ethyl]carbamoyl]pyrrolidine-1-carbonyl]propyl]isoxazol-3-yl]oxyethyl]-4-piperidyl]oxy]cyclobutoxy]-4-pyridyl]-3,8-diazabicyclo[3.2.1]octan-3-yl]pyridazin-3-yl]phenoxy]methyl hydrogen phosphate

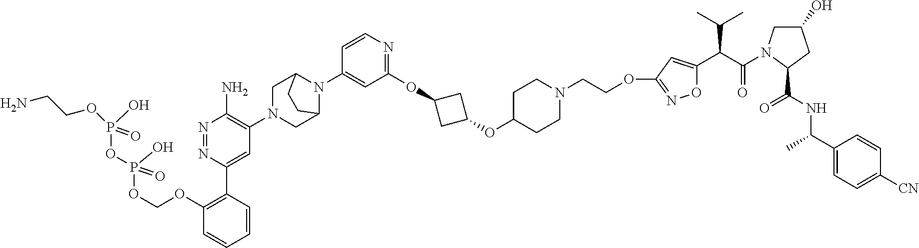 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)OP(=O)(O)OCCN)nnc5N)C6)ccn4)C3)CC2)no1]

To a solution of (9H-fluoren-9-yl)methyl (2-((((((2-(6-amino-5-(8-(2-((1r, 3r)-3-((1-(2-((5-((R)-1-((2S, 4R)-2-(((S)-1-(4-cyanophenyl)ethyl)carbamoyl)-4-hydroxypyrrolidin-1-yl)-3-meth yl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methoxy)(hydroxy)phosphoryl)oxy)(hydroxy)phosphoryl)oxy)ethyl)carbamate (200 mg, 0.14 mmol) in N,N-dimethylformamide (10.0 mL) was added piperidine (0.10 mL, 1.40 mmol). The reaction mixture was stirred at 20° C. for 12 h. It was quenched with 1N HCl (1.00 ml) and the resulting residue was purified by Phenomenex Gemini-NX 80*40 mm*3 um (acetonitrile 19-49/water (0.05% NH₃H₂O)—actonitrlie, 20-50%) to afford the title compound (40.0 mg, 22.4% yield) as a white solid.

LCMS (ESI) m/z: 1229.7 [M+H]⁺.

### Step 8: (2S,4R)-tert-Butyl 2-(((S)-1-(4-cyanophenyl)ethyl)carbamoyl)-4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)pyrrolidine-1-carboxylate

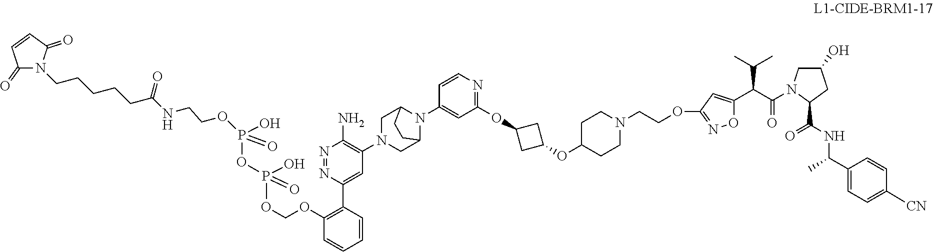 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCOP(=O)(O)OP(=O)(O)OCCNC(=O)CCCCCN7C(=O)C=CC7=O)nnc5N)C6)ccn4)C3)CC2)no1]

To a mixture of 2-aminoethoxy(hydroxy)phosphoryl] [2-[6-amino-5-[8-[2-[3-[[1-[2-[5-[rac-(1R)-2-methyl-1-[rac-(2S,4R)-4-hydroxy-2-[[rac-(1S)-1-(4-cyanophenyl)ethyl]carbamoyl]pyrrolidine-1-carbonyl]propyl]isoxazol-3-yl]oxyethyl]-4-piperidyl]oxy]cyclobutoxy]-4-pyridyl]-3,8-diazabicyclo[3.2.1]octan-3-yl]pyridazin-3-yl]phenoxy]methyl hydrogen phosphate (120 mg, 0.10 mmol) in anhydrous tetrahydrofuran (12.0 mL) was added N,N-diisopropylethylamine (18.7 uL, 0.11 mmol), followed by 2,5-dioxopyrrolidin-1-yl 6-(2,5-dioxo-2,5-dihydro-1H-pyrrol-1-yl)hexanoate (33.1 mg, 0.11 mmol). The reaction solution was stirred at 25° C. for 16 h. The solution was filtered, and concentrated to dryness. The residue was purified by prep-HPLC (Boston Green ODS 150*30 mm*5 um, water (0.075% trifluoroacetic acid)—acetonitrile 20%-50%) to afford the title compound (60.8 mg, 36.8%) as a white solid.

LCMS (ESI) m/z: 1423.0 [M+H]⁺.

### Synthesis Example 18

### Synthesis of L1-CIDE-BRM1-18

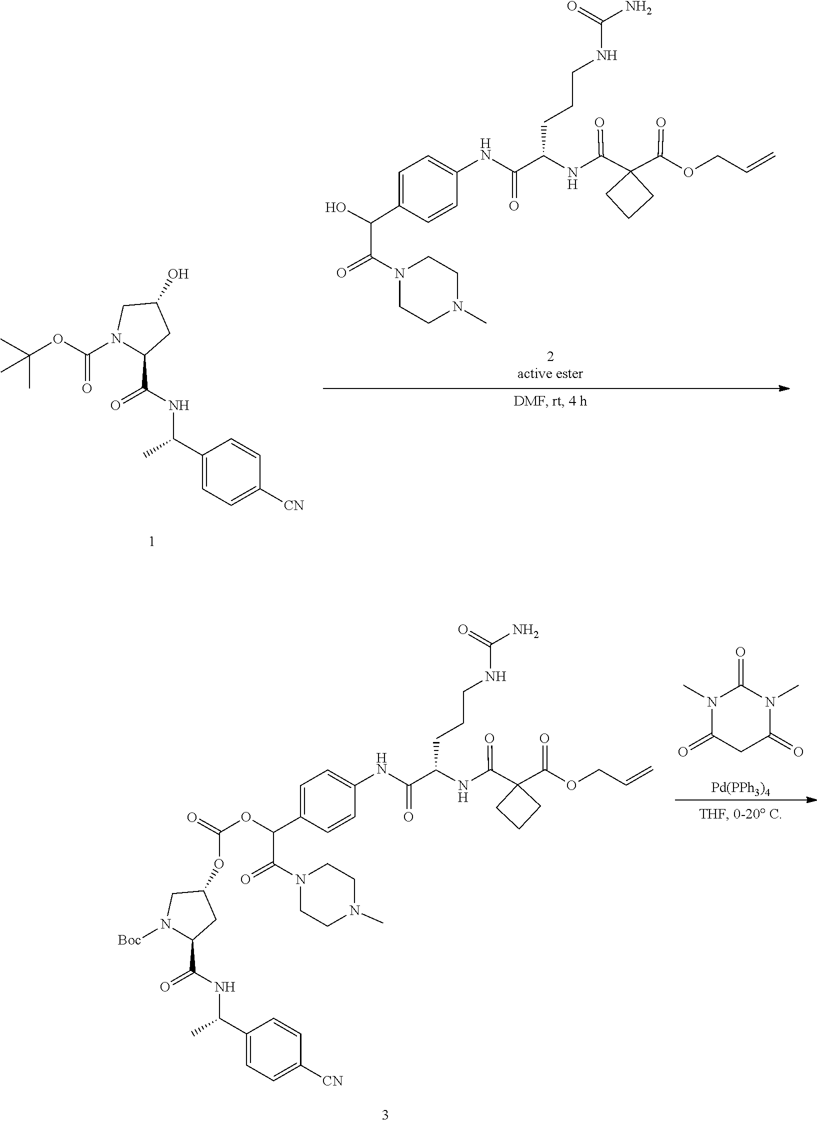

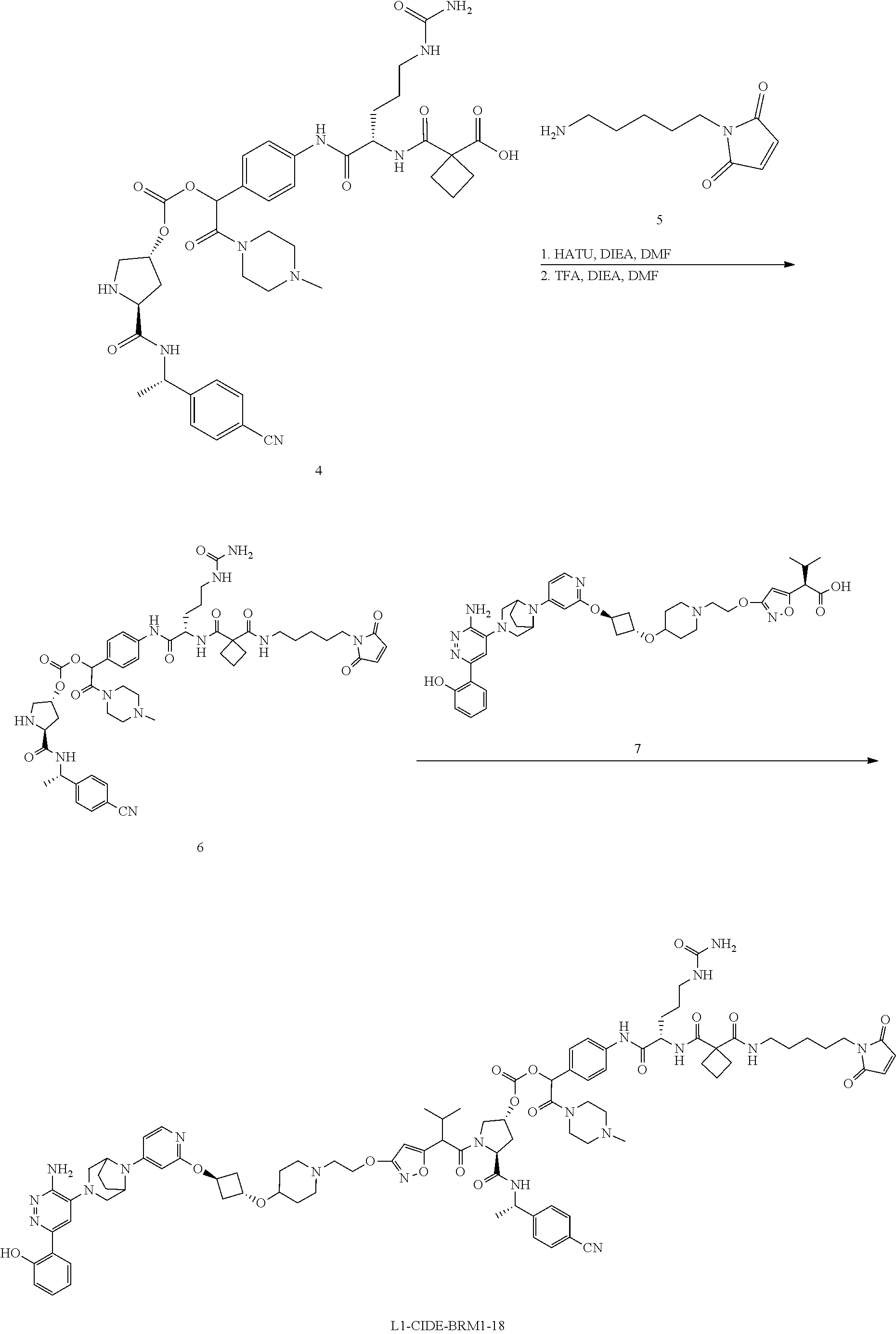

### Step 1: (2S, 4R)-tert-butyl 2-(((S)-1-(4-cyanophenyl)ethyl)carbamoyl)-4-(((4-nitrophenoxy)carbonyl)oxy)pyrrolidine-1-carboxylate

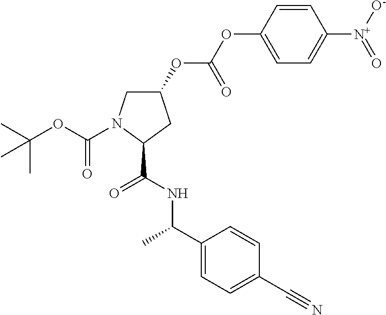 [SMILES: C[C@H](NC(=O)[C@@H]1C[C@@H](OC(=O)Oc2ccc([N+](=O)[O-])cc2)CN1C(=O)OC(C)(C)C)c1ccc(C#N)cc1]

To a mixture of (2S, 4R)-tert-butyl 2-(((S)-1-(4-cyanophenyl)ethyl)carbamoyl)-4-hydroxypyrrolidine-1-carboxylate (1.00 g, 2.78 mmol) in dichloromethane (10.0 mL) was added 2,6-lutidine (0.49 mL, 4.17 mmol) and 4-nitrophenylchloroformate (673 mg, 3.34 mmol). The reaction mixture was stirred at 25° C. for 18 h. The crude mixture was concentrated to get the title compound (1.46 g, 36.8%) as a yellow solid. The crude was used to the next step immediately LCMS (ESI) m/z: 425.1 [M-Boc+H]⁺.

### Step 2: (2S,4R)-tert-butyl 4-(((1-(4-((S)-2-(1-((allyloxy)carbonyl)cyclobutanecarboxamido)-5-ureidopentanamido)phenyl)-2-(4-methylpiperazin-1-yl)-2-oxoethoxy)carbonyl)oxy)-2-(((S)-1-(4-cyanophenyl)ethyl)carbamoyl)pyrrolidine-1-carboxylate

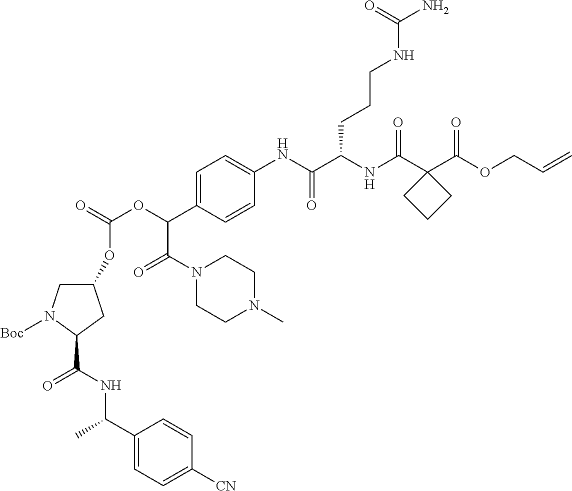 [SMILES: C=CCOC(=O)C1(C(=O)N[C@@H](CCCNC(N)=O)C(=O)Nc2ccc(C(OC(=O)O[C@@H]3C[C@@H](C(=O)N[C@@H](C)c4ccc(C#N)cc4)N(C(=O)OC(C)(C)C)C3)C(=O)N3CCN(C)CC3)cc2)CCC1]

To a mixture of (2S, 4R)-tert-butyl 2-(((S)-1-(4-cyanophenyl)ethyl)carbamoyl)-4-(((4-nitrophenoxy)carbonyl)oxy)pyrrolidine-1-carboxylate (1.46 g, 2.78 mmol) and allyl 1-(((2S)-1-((4-(1-hydroxy-2-(4-methylpiperazin-1-yl)-2-oxoethyl)phenyl)amino)-1-oxo-5-ureidopentan-2-yl)carbamoyl)cyclobutanecarboxylate (1.59 g, 2.78 mmol) in N,N-dimethylformamide (15.0 mL) was added 4-dimethylaminopyridine (680 mg, 5.56 mmol). The reaction mixture was stirred at 25° C. for 18 h. The crude was filtrated and purified by prep-HPLC (Phenomenex Gemini-NX 80*30 mm*3 um/water (10 mM NH₄HCO₃)—acetonitrile/10%-80%) to get the title compound (400 mg, 15%) a yellow solid. LCMS (ESI) m/z: 958.5 [M+H]⁺.

### Step 3: 1-(((2S)-1-((4-(1-(((((3R,5S)-1-(tert-butoxycarbonyl)-5-(((S)-1-(4-cyanophenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid

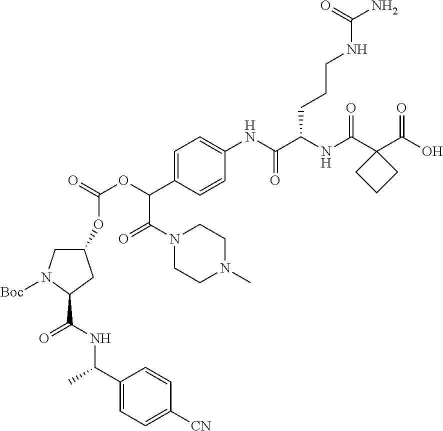 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)O)CCC3)cc2)CN1C(=O)OC(C)(C)C)c1ccc(C#N)cc1]

To a solution of (2S, 4R)-tert-butyl 4-(((1-(4-((S)-2-(1-((allyloxy)carbonyl)cyclobutanecarboxamido)-5-ureidopentanamido)phenyl)-2-(4-methylpiperazin-1-yl)-2-oxoethoxy)carbonyl)oxy)-2-(((S)-1-(4-cyanophenyl)ethyl)carbamoyl)pyrrolidine-1-carboxylate (260 mg, 0.27 mmol) and 1,3-dimethylpyrimidine-2,4,6(1H, 3H, 5H)-trione (212 mg, 1.36 mmol) in dichloromethane (2.00 mL) and methanol (2.00 mL) was added tetrakis(triphenylphosphine)palladium (62.7 mg, 0.05 mmol) at 25° C. The reaction mixture was stirred under nitrogen atmosphere at 25° C. for 16 h. The crude was concentrated and purified by Prep-HPLC with the following conditions: Column: Phenomenex Gemini-NX 80*30 mm*3 um, mobile phase: water (10 mM NH₄ HCO₃)—acetonitrile 10%-80% to afford the title compound (110 mg, 44.2% yield) as a yell ow solid.

LCMS (ESI) m/z: 918.6 [M+H]⁺.

### Step 4: (2S,4R)-tert-butyl 2-(((S)-1-(4-cyanophenyl)ethyl)carbamoyl)-4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)pyrrolidine-1-carboxylate

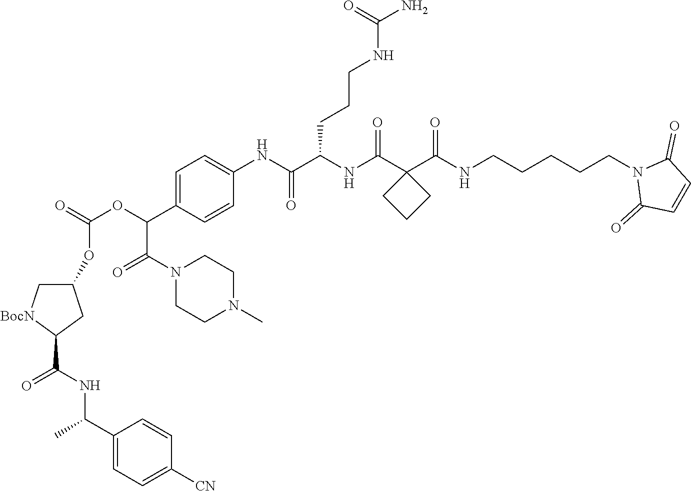 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)CN1C(=O)OC(C)(C)C)c1ccc(C#N)cc1]

To a mixture of 1-(((2S)-1-((4-(1-(((((3R,5S)-1-(tert-butoxycarbonyl)-5-(((S)-1-(4-cyanophenyl)ethyl)carbamoyl)pyrrolidin-3-yl)oxy)carbonyl)oxy)-2-(4-methylpiperazin-1-yl)-2-oxoethyl)phenyl)amino)-1-oxo-5-ureidopentan-2-yl)carbamoyl)cyclobutanecarboxylic acid (110 mg, 0.12 mmol) and 1-(5-aminopentyl)-1H-pyrrole-2,5-dione 2,2,2-trifluoroacetic acid (43.0 mg, 0.15 mmol) in N,N-dimethylformamide (3 mL) was added N,N-diisopropylethylamine (0.06 mL, 0.36 mmol), 2-(7-azabenzotriazol-1-yl)-N,N,N,N-tetramethyluronium hexafluorophosphate (54.7 mg, 0.14 mmol). The reaction mixture was stirred at 25° C. for 3 h. The mixture was concentrated by oil pump. The residue was purified by prep-HPLC (Boston Green ODS 15 0*30 mm*5 um, water (0.075% TFA)—acetonitrile 28%-58%) to afford the title compound (9 0 mg, 69.4%) as a white solid. LCMS (ESI) m/z: 1082.6 [M+H]⁺.

### Step 5: (3R,5S)-5-(((S)-1-(4-cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate 2,2,2-trifluoroacetate

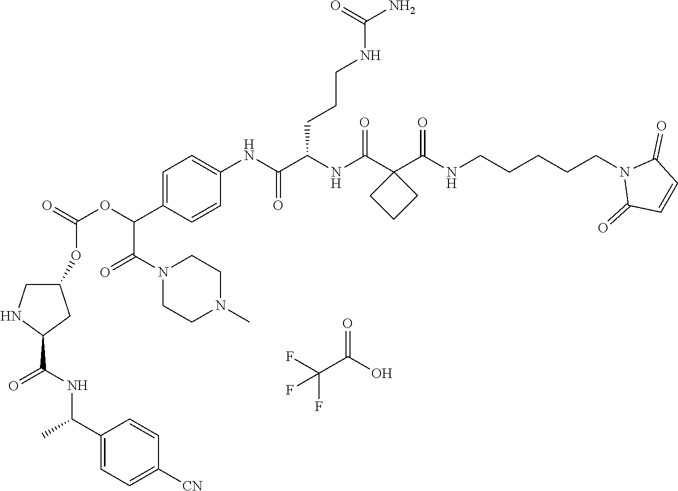 [SMILES: C[C@H](NC(=O)[C@@H]1C[C@@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)CN1)c1ccc(C#N)cc1 ; O=C(O)C(F)(F)F]

A solution of (2S, 4R)-tert-butyl 2-(((S)-1-(4-cyanophenyl)ethyl)carbamoyl)-4-(((1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethoxy)carbonyl)oxy)pyrrolidine-1-carboxylate (90 mg, 0.08 mmol) in 5% trifluoroacetic acid in hexafluoroisopropanol (5 mL 10) was stirred at 25° C. for 2 h. The mixture was concentrated to afford the title compound (91.0 mg, 99.8% yield) as a yellow oil. LCMS (ESI) m/z: 982.4 [M-TFA+H]⁺.

### Step 6: (3R,5S)-1-(2-(3-(2-(4-((1r,3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate

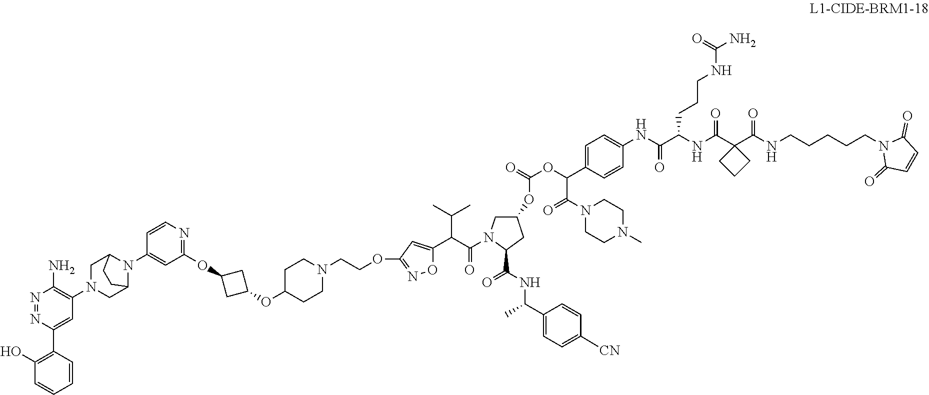 [SMILES: CC(C)C(C(=O)N1C[C@H](OC(=O)OC(C(=O)N2CCN(C)CC2)c2ccc(NC(=O)[C@H](CCCNC(N)=O)NC(=O)C3(C(=O)NCCCCCN4C(=O)C=CC4=O)CCC3)cc2)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7O)nnc5N)C6)ccn4)C3)CC2)no1]

To a mixture of (3R,5S)-5-(((S)-1-(4-cyanophenyl)ethyl)carbamoyl)pyrrolidin-3-yl (1-(4-((S)-2-(1-((5-(2,5-dioxo-2,5-dihydro-1H-pyrrol-1-yl)pentyl)carbamoyl)cyclobutanecarboxamido)-5-ureidopentanamido)phenyl)-2-(4-methylpiperazin-1-yl)-2-oxoethyl) carbonate 2,2,2-trifluoroacetate (91.0 mg, 0.08 mmol) and 2-(3-(2-(4-((1r,3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoic acid (81.5 mg, 0.11 mmol) in N,N-dimethylformamide (2.50 mL) was added N,N-diisopropylethylamine (0.08 mL, 0.50 mmol) and 2-(7-azabenzotriazol-1-yl)-N,N,N′,N′-tetramethyluronium hexafluorophosphate (41.0 mg, 0.11 mmol). The mixture was stirred at 25° C. for 16 h. After concentration, the crude was purified by Pre p-HPLC with the following conditions: Column: Welch Xtimate C18 100*40 mm*3 um Condition water (0.075% trifluoroacetic acid)—acetonitrile 15-45%) to afford the title compound (80.6 mg, 52% yield) as a white solid. LCMS (ESI) m/z: 860.4 [1/M+H]+

### Intermediate 1: Ethyl (S)-1-((1-((4-((2-bromophenoxy)methyl)phenyl)amino)-6-(dimethylamino)-1-oxohexan-2-yl)carbamoyl)cyclobutane-1-carboxylate

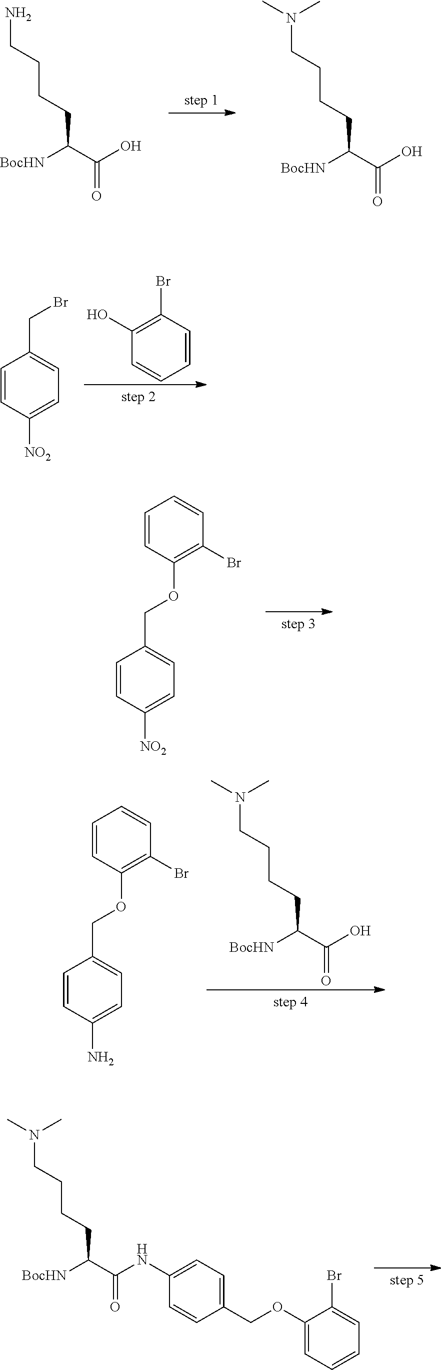

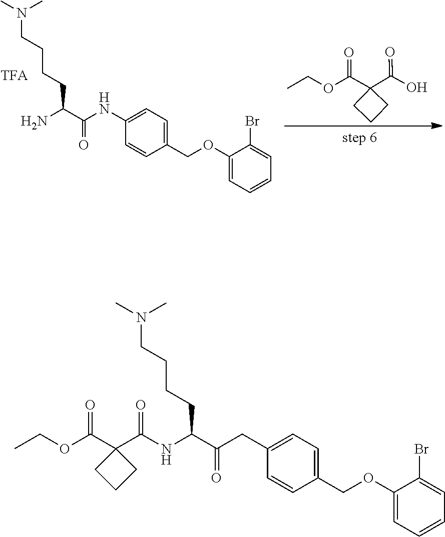 [SMILES: CN(C)CCCC[C@H](N)C(=O)Nc1ccc(COc2ccccc2Br)cc1 ; CCOC(=O)C1(C(=O)O)CCC1 ; CCOC(=O)C1(C(=O)N[C@@H](CCCCN(C)C)C(=O)Cc2ccc(COc3ccccc3Br)cc2)CCC1]

### Step 1: N²-(tert-butoxycarbonyl)-N⁶,N⁶-dimethyl-L-lysine

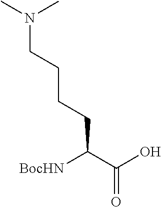 [SMILES: CN(C)CCCC[C@H](NC(=O)OC(C)(C)C)C(=O)O]

Under hydrogen (3 atm), a solution of (tert-butoxycarbonyl)-L-lysine (20.0 g, 81.2 mmol), CH₂O (12.2 g, 162 mmol) and Pd/C (2.00 g) in methyl alcohol (100 mL) was stirred at room temperature for 4 hours. After filtration, the filtrate was concentrated under reduced pressure. The residue was washed with Et₂O. The solids were collected by filtration to afford 20.6 g (92% yield) of the title compound as a white solid. LCMS (ESI) [M+H]+=275.

### Step 2: 1-Bromo-2-((4-nitrobenzyl)oxy)benzene

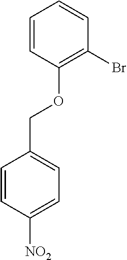 [SMILES: O=[N+]([O-])c1ccc(COc2ccccc2Br)cc1]

A solution of 2-bromophenol (52.6 g, 304 mmol), 1-(bromomethyl)-4-nitrobenzene (65.7 g, 304 mmol) and K₂CO₃ (83.9 g, 608 mmol) in DMF (700 mL) was stirred at room temperature for 1 hour. EtOAc was added and water was used to wash for three times. The organic layer was dried over anhydrous Na₂SO₄ and concentrated under vacuum to afford 73.8 g (78% yield) of the title compound as a yellow solid. ¹H NMR (300 MHz, DMSO-d6) δ 8.34-8.24 (m, 2H), 7.81-7.70 (m, 2H), 7.62 (dd, J=7.9, 1.6 Hz, 1H), 7.36 (ddd, J=8.3, 7.3, 1.6 Hz, 1H), 7.20 (dd, J=8.3, 1.5 Hz, 1H), 6.94 (td, J=7.6, 1.4 Hz, 1H), 5.39 (s, 2H).

### Step 3: 4-((2-bromophenoxy)methyl)aniline

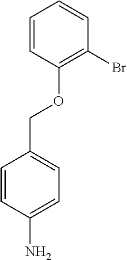 [SMILES: Nc1ccc(COc2ccccc2Br)cc1]

Under nitrogen, to a solution of 1-bromo-2-((4-nitrobenzyl)oxy)benzene (43.0 g, 139.5 mmol) and K₂CO₃ (115 g, 837 mmol) in acetonitrile (800 mL) and water (400 mL) was added Na₂S₂O₄ (242 g, 1395 mmol) in portions at 0° C. The mixture was stirred at room temperature for 6 hours. EtOAc was used to extract the product once. The organic layer was dried over anhydrous Na₂SO₄ and concentrated under vacuum to afford 35 g (crude) of the title compound as a yellow solid. LCMS (ESI) [M+H]⁺=278.

### Step 4: tert-Butyl (S)-(1-((4-((2-bromophenoxy)methyl)phenyl)amino)-6-(dimethylamino)-1-oxohexan-2-yl)carbamate

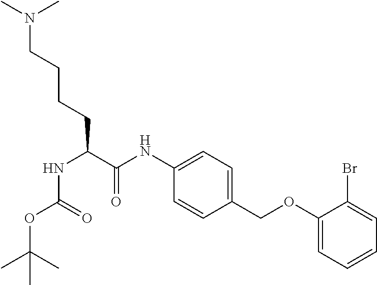 [SMILES: CN(C)CCCC[C@H](NC(=O)OC(C)(C)C)C(=O)Nc1ccc(COc2ccccc2Br)cc1]

Under nitrogen, to a solution of N²-(tert-butoxycarbonyl)-N⁶,N⁶-dimethyl-L-lysine (13.3 g, 48.4 mmol) and NMM (10.3 g, 96.9 mmol) in tetrahydrofuran (200 mL) was added iso-butyl chloroformate (7.91 g, 58.1 mmol) dropwise at −25° C. The reaction was stirred at −25° C. for 0.5 hours. Then a solution of 4-((2-bromophenoxy)methyl)aniline (16.1 g, crude) in tetrahydrofuran (120 mL) was added at −25° C. The reaction was stirred at room temperature for 4 hours. The solvent was concentrated under vacuum. DCM was added and washed with water. The organic layer was dried over anhydrous Na₂SO₄ and concentrated under vacuum. The residue was purified by flash chromatography on silica gel (gradient: 0-9% MeOH/DCM) to afford 6.70 g (25% yield) of the title compound as a white solid. LCMS (ESI) [M+H]⁺=534.

### Step 5: (S)-2-amino-N-(4-((2-bromophenoxy)methyl)phenyl)-6-(dimethylamino)hexanamide (2,2,2-trifluoroacetic acid salt)

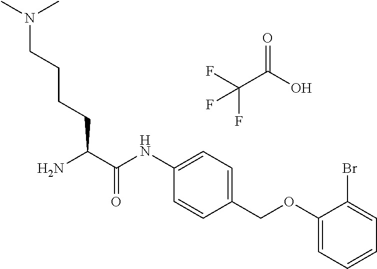 [SMILES: CN(C)CCCC[C@H](N)C(=O)Nc1ccc(COc2ccccc2Br)cc1 ; O=C(O)C(F)(F)F]

A solution of tert-butyl (S)-(1-((4-((2-bromophenoxy)methyl)phenyl)amino)-6-(dimethylamino)-1-oxohexan-2-yl)carbamate (4.00 g, 7.48 mmol) in 5% TFA/HFIP (50 mL) was stirred at room temperature for 3 hours. The solvent was concentrated under vacuum and used in next step directly. LCMS (ESI) [M+H]⁺=434.

### Step 6: Ethyl (S)-1-((1-((4-((2-bromophenoxy)methyl)phenyl)amino)-6-(dimethylamino)-1-oxohexan-2-yl)carbamoyl)cyclobutane-1-carboxylate

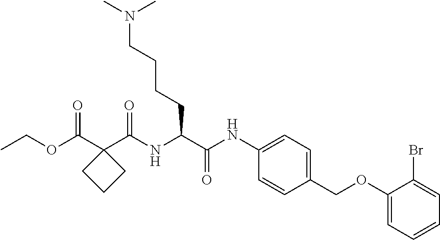 [SMILES: CCOC(=O)C1(C(=O)N[C@@H](CCCCN(C)C)C(=O)Nc2ccc(COc3ccccc3Br)cc2)CCC1]

To a solution of (S)-2-amino-N-(4-((2-bromophenoxy)methyl)phenyl)-6-(dimethylamino)hexanamide (2,2,2-trifluoroacetic acid salt) (crude from step 5), 1-(ethoxycarbonyl)cyclobutane-1-carboxylic acid (1.55 g, 8.98 mmol) and DIPEA (9.65 g, 74.8 mmol) in DMF (20 mL) was added HATU (3.41 g, 8.98 mmol) at 0° C. The mixture was stirred at room temperature for 0.5 hour. The crude was purified by pre-packed C18 column (gradient: 0-100% MeOH in water (0.05% NH₄HCO₃) to afford 2.70 g (61% yield) of the title compound as a red solid. LCMS (ESI) [M+H]⁺=588. ¹H NMR (300 MHz, DMSO-d6) δ 9.87 (s, 1H), 7.72 (d, J=7.9 Hz, 1H), 7.54-7.42 (m, 3H), 7.30 (d, J=8.6 Hz, 2H), 7.21 (ddd, J=8.8, 7.3, 1.6 Hz, 1H), 7.07 (dd, J=8.4, 1.5 Hz, 1H), 6.77 (td, J=7.6, 1.4 Hz, 1H), 5.02 (s, 2H), 4.30 (q, J=8.0 Hz, 1H), 4.00 (q, J=7.1 Hz, 2H), 2.35-2.21 (m, 2H), 2.03 (t, J=6.8 Hz, 2H), 1.96 (s, 6H), 1.80-1.41 (m, 4H), 1.30-1.14 (m, 6H), 1.06 (t, J=7.1 Hz, 3H).

### Intermediate 5: tert-butyl (3R)-4-(2-((4-(3-(3-amino-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate

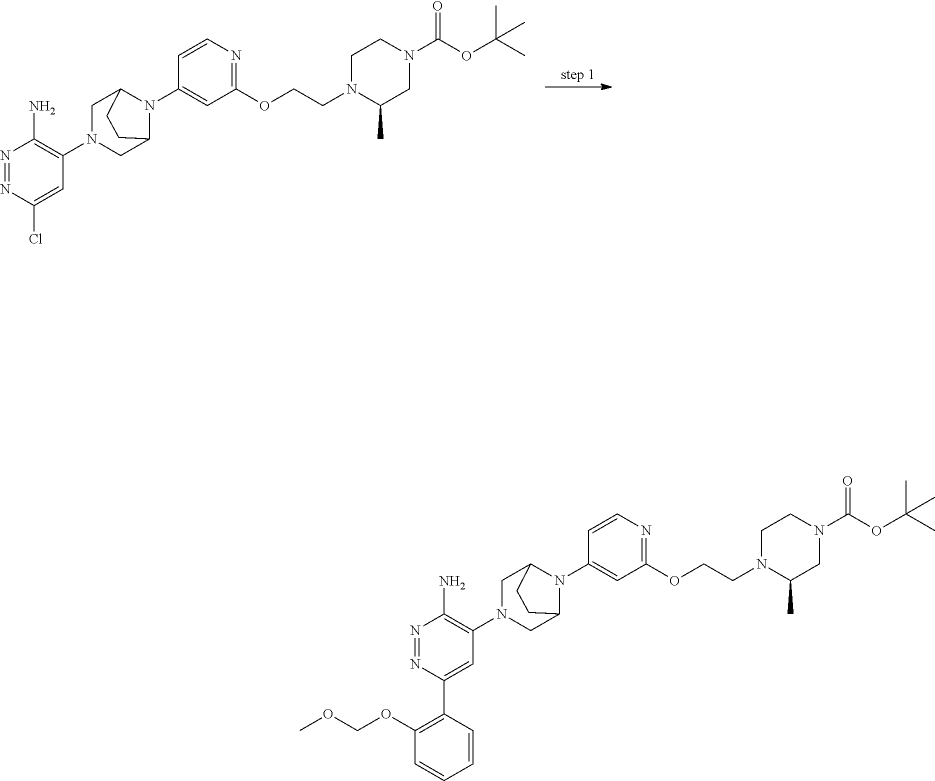 [SMILES: C[C@@H]1CN(C(=O)OC(C)(C)C)CCN1CCOc1cc(N2C3CCC2CN(c2cc(Cl)nnc2N)C3)ccn1 ; COCOc1ccccc1-c1cc(N2CC3CCC(C2)N3c2ccnc(OCCN3CCN(C(=O)OC(C)(C)C)C[C@H]3C)c2)c(N)nn1]

### Step 1: tert-butyl (3R)-4-(2-((4-(3-(3-amino-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate

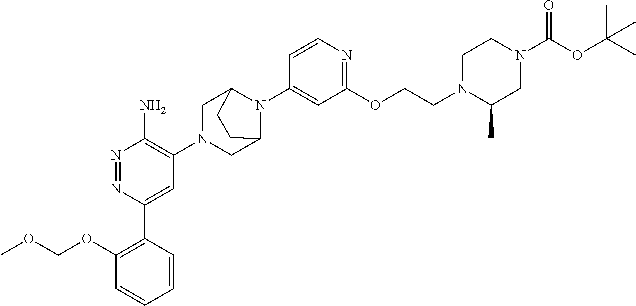 [SMILES: COCOc1ccccc1-c1cc(N2CC3CCC(C2)N3c2ccnc(OCCN3CCN(C(=O)OC(C)(C)C)C[C@H]3C)c2)c(N)nn1]

Under nitrogen, a solution of tert-butyl (3R)-4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (1.00 g, 1.79 mmol), (2-(methoxymethoxy)phenyl)boronic acid (391 mg, 2.15 mmol), Pd(PPh₃)₄ (413 mg, 0.358 mmol) and K₂CO₃ (741 mg, 5.37 mmol) in dioxane (10 mL) and water (2 mL) was stirred at 100° C. for 1 hour. The reaction was diluted with water and extracted with dichloromethane. The organic layers were combined, dried over anhydrous sodium sulfate and concentrated under vacuum. The residue was purified by flash chromatography on silica gel (gradient: 0%-10% methanol/dichloromethane) to yield 670 mg (57% yield) of the title compound as a yellow solid. LC-MS: (ESI, m/z): [M+H]⁺=661. ¹H NMR (300 MHz, DMSO-d₆) δ 7.76 (d, J=5.9 Hz, 1H), 7.58 (dd, J=7.6, 1.8 Hz, 1H), 7.34 (ddd, J=9.0, 7.3, 1.8 Hz, 1H), 7.18-7.01 (m, 3H), 6.51 (dd, J=6.1, 2.0 Hz, 1H), 6.12 (d, J=2.0 Hz, 1H), 5.72 (s, 2H), 5.14 (s, 2H), 4.47 (s, 2H), 4.25 (t, J=6.1 Hz, 2H), 3.53 (d, J=12.8 Hz, 2H), 3.22 (s, 3H), 3.14-2.67 (m, 8H), 2.64-2.56 (m, 1H), 2.46-2.36 (m, 1H), 2.32-2.22 (m, 1H), 2.22-2.13 (m, 2H), 2.00-1.90 (m, 2H), 1.38 (s, 9H), 0.96 (d, J=6.2 Hz, 3H).

### Synthesis Example 19

### Synthesis of L1-CIDE-BRM1-19

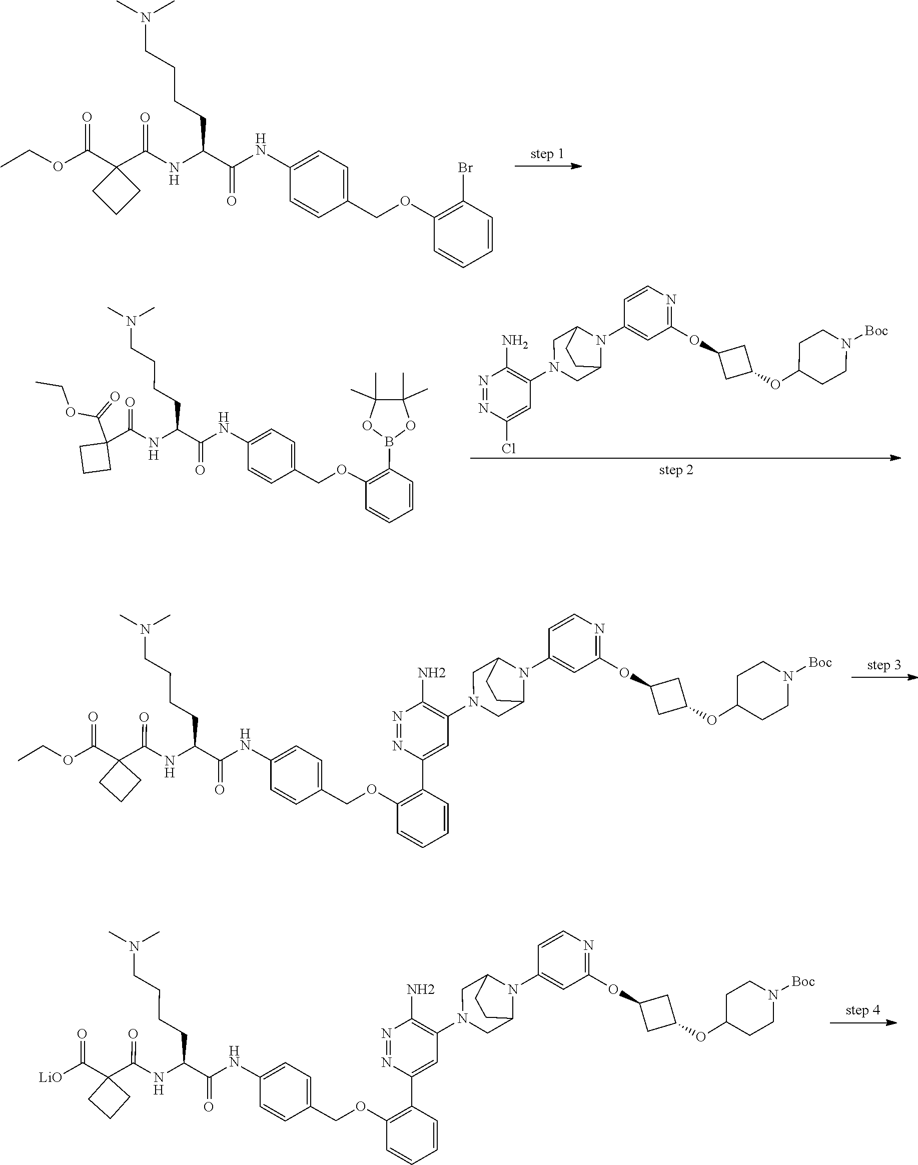

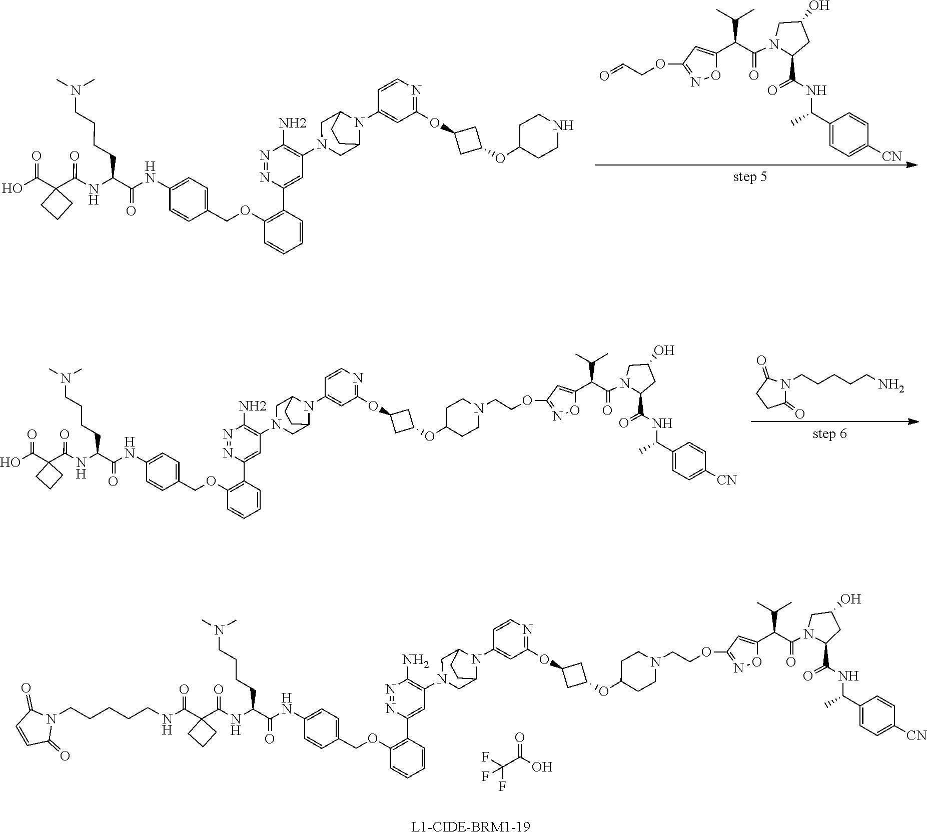

### Step 1: Ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate

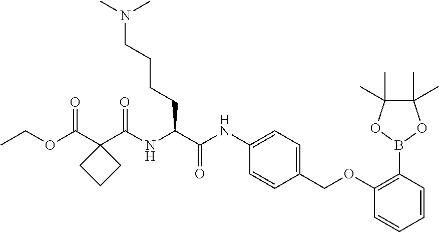 [SMILES: CCOC(=O)C1(C(=O)N[C@@H](CCCCN(C)C)C(=O)Nc2ccc(COc3ccccc3B3OC(C)(C)C(C)(C)O3)cc2)CCC1]

Under nitrogen, a solution of ethyl (S)-1-((1-((4-((2-bromophenoxy)methyl)phenyl)amino)-6-(dimethylamino)-1-oxohexan-2-yl)carbamoyl)cyclobutane-1-carboxylate (500 mg, 0.852 mmol), B₂Pin₂ (649 mg, 2.55 mmol), Pd(dppf)Cl₂ (124 mg, 0.170 mmol) and KOAc (250 mg, 2.55 mmol) in 1,4-dioxane (5 mL) was stirred at 80° C. for 2 hours. The reaction was diluted with DCM and washed with water. The organic layer was dried over anhydrous sodium sulfate and concentrated under vacuum. The residue was purified by flash chromatography on silica gel (gradient: 0%-20% MeOH/DCM (contain 0.3% 7 M NH₃/MeOH)) to yield 390 mg (72% yield) of the title compound as a yellow solid. LC-MS: (ESI, m/z): [M+H]⁺=636.

### Step 2: tert-Butyl 4-((1r,3r)-3-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidine-1-carboxylate

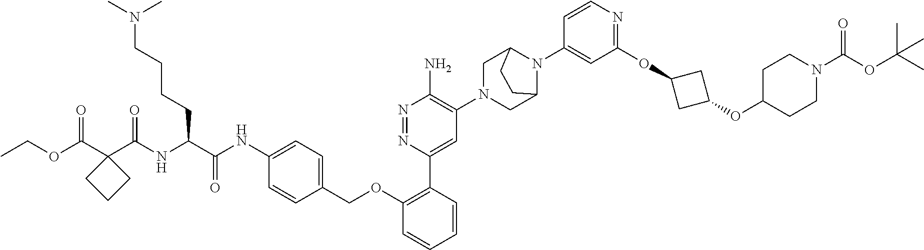 [SMILES: CCOC(=O)C1(C(=O)N[C@@H](CCCCN(C)C)C(=O)Nc2ccc(COc3ccccc3-c3cc(N4CC5CCC(C4)N5c4ccnc(O[C@H]5C[C@H](OC6CCN(C(=O)OC(C)(C)C)CC6)C5)c4)c(N)nn3)cc2)CCC1]

Under nitrogen, a solution of ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate (390 mg, 0.614 mmol), tert-butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate (395 mg, 0.676 mmol), Ad₂nBuPPdG2 (41.0 mg, 0.061 mmol) and K₂CO₃ (260 mg, 1.22 mmol) in dioxane (5.0 mL) and H₂O (1.2 mL) was stirred at 95° C. for 2 hours. The resulting solution diluted with water and extracted with EtOAc. Organic layer was concentrated under vacuum. The residue was purified by pre-packed C18 column (solvent gradient: 0-100% MeOH in water (0.1% NH₄HCO₃)) to yield 310 mg (47% yield) of the title compound as a white solid. LC-MS: (ESI, m/z): [M+H]⁺=1060.

### Step 3: lithium 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate

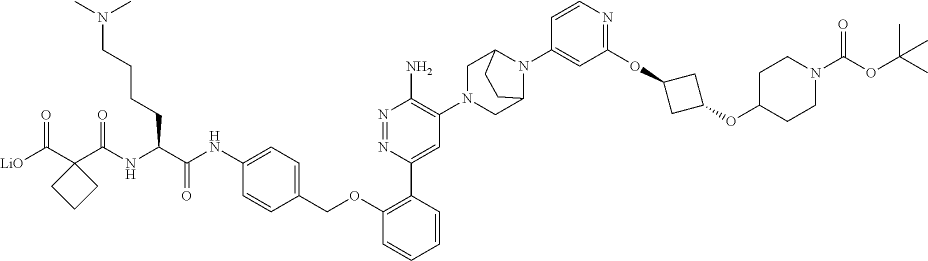

A solution of tert-butyl 4-((1r,3r)-3-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidine-1-carboxylate (270 mg, 0.255 mmol) and LiOH·H₂O (32.1 mg, 0.765 mmol) in THF (2 mL) and H₂O (2 mL) was stirred at room temperature for 1 h. The resulting mixture was concentrated under vacuum. The crude was used in next step directly. LC-MS: (ESI, m/z): [M+H]⁺=1032.

### Step 4: 1-(((2S)-1-((4-((2-(6-Amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

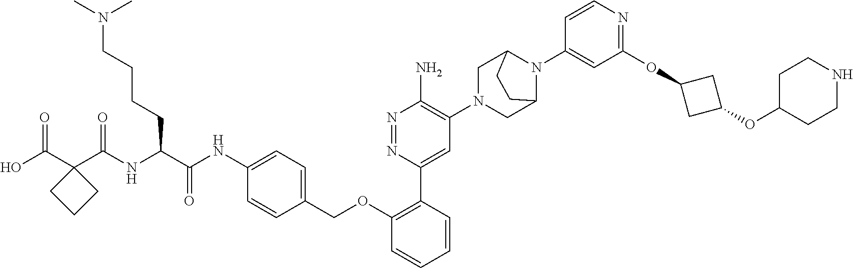 [SMILES: CN(C)CCCC[C@H](NC(=O)C1(C(=O)O)CCC1)C(=O)Nc1ccc(COc2ccccc2-c2cc(N3CC4CCC(C3)N4c3ccnc(O[C@H]4C[C@H](OC5CCNCC5)C4)c3)c(N)nn2)cc1]

A solution of lithium 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate (crude from last step) in TFA (0.75 mL) and HFIP (14 mL) was stirred at room temperature for 30 min. The resulting mixture was concentrated under vacuum. The obtained crude product was purified by pre-packed C18 column (solvent gradient: 0-100% MeOH in water (0.1% NH₄HCO₃)) to yield 170 mg (71% yield) of the title compound as a yellow solid. LC-MS: (ESI, m/z): [M+H]⁺=932.

### Step 5: 1-(((2S)-1-((4-((2-(6-Amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

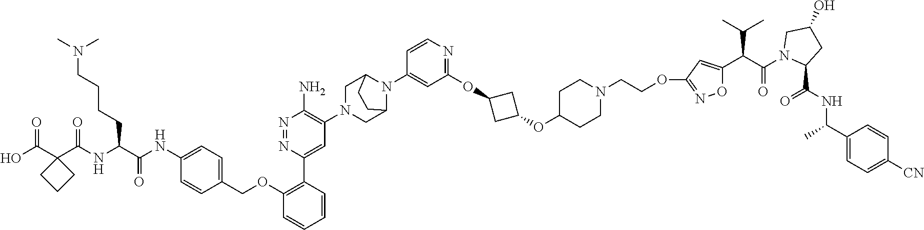 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)C3)CC2)no1]

Under nitrogen, a solution of 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (170.0 mg, 0.183 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (111 mg, 0.237 mmol), HOAc (21.9 mg, 0.365 mmol) in DCM (1.5 mL) and MeOH (0.5 mL) was stirred at room temperature for 1 hour. Then NaBH₃CN (17.3 mg, 0.274 mmol) was added at 0° C. and stirred at room temperature temperature for 30 min. The reaction was quenched with water. The resulting solution was concentrated under vacuum. The obtained crude product was purified by pre-packed C18 column (gradient: 0-100% MeOH in water (0.05% NH₄HCO₃)) to yield 180 mg (71% yield) of the title compound as a white solid. LC-MS: (ESI, m/z): [M+H]⁺=1384.

### Step 5: N-((2S)-1-((4-((2-(6-Amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-(2,5-dioxo-2,5-dihydro-1H-pyrrol-1-yl)pentyl)cyclobutane-1,1-dicarboxamide (formic acid salt)

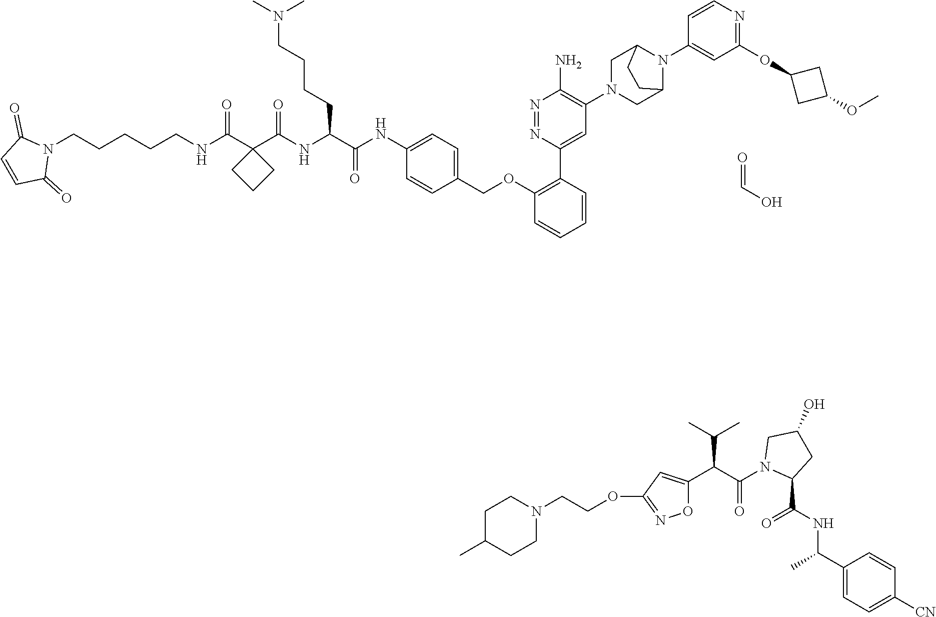

To a solution of 1-(((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (65.0 mg, 0.047 mmol), 1-(5-aminopentyl)-1H-pyrrole-2,5-dione (2,2,2-trifluoroacetic acid salt) (25.6 mg, crude), DIPEA (90.9 mg, 0.705 mmol) in DMF (1.5 mL) was added HATU (21.4 mg, 0.056 mmol) at room temperature. The resulting solution was stirred at room temperature for 30 min. The resulting solution was purified by Prep-HPLC (Xselect CSH F-Phenyl OBD column, 19×250 mm 5 μm; Mobile Phase A: Water (0.05% FA), Mobile Phase B: ACN; Flow rate: 60 mL/min; Gradient: 2% B to 29% B in 7 min; 254 nm; R_{TI}: 6.5 min) to yield 5.9 mg (8% yield) of L1-CIDE-BRM1-19 as a white solid. LC-MS: (ESI, m/z): [M+H]⁺=1548. ¹H NMR (300 MHz, DMSO-d₆) δ 10.24 (s, 1H), δ 8.47 (d, J=7.4 Hz, 1H), 8.17 (s, 1H), 7.93-7.54 (m, 8H), 7.55-7.30 (m, 5H), 7.26-7.09 (m, 2H), 7.08-6.87 (m, 3H), 6.43-5.88 (m, 3H), 5.59 (s, 2H), 5.22-4.86 (m, 5H), 4.50-4.12 (m, 8H), 3.72-3.54 (m, 3H), 3.13-2.97 (m, 4H), 2.63 (s, 6H), 2.45-2.35 (m, 5H), 2.25-2.02 (m, 16H), 2.02-1.56 (m, 11H), 1.57-1.25 (m, 13H), 1.29-1.12 (m, 4H), 0.95 (d, J=6.8 Hz, 3H), 0.79 (d, J=6.8 Hz, 3H).

### Synthesis Example 20

### Synthesis of L1-CIDE-BRM1-20

### 4-((S)-2-(1-((5-(2,5-Dioxo-2,5-dihydro-1H-pyrrol-1-yl)pentyl)carbamoyl)cyclobutane-1-carboxamido)-5-ureidopentanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (formic acid salt)

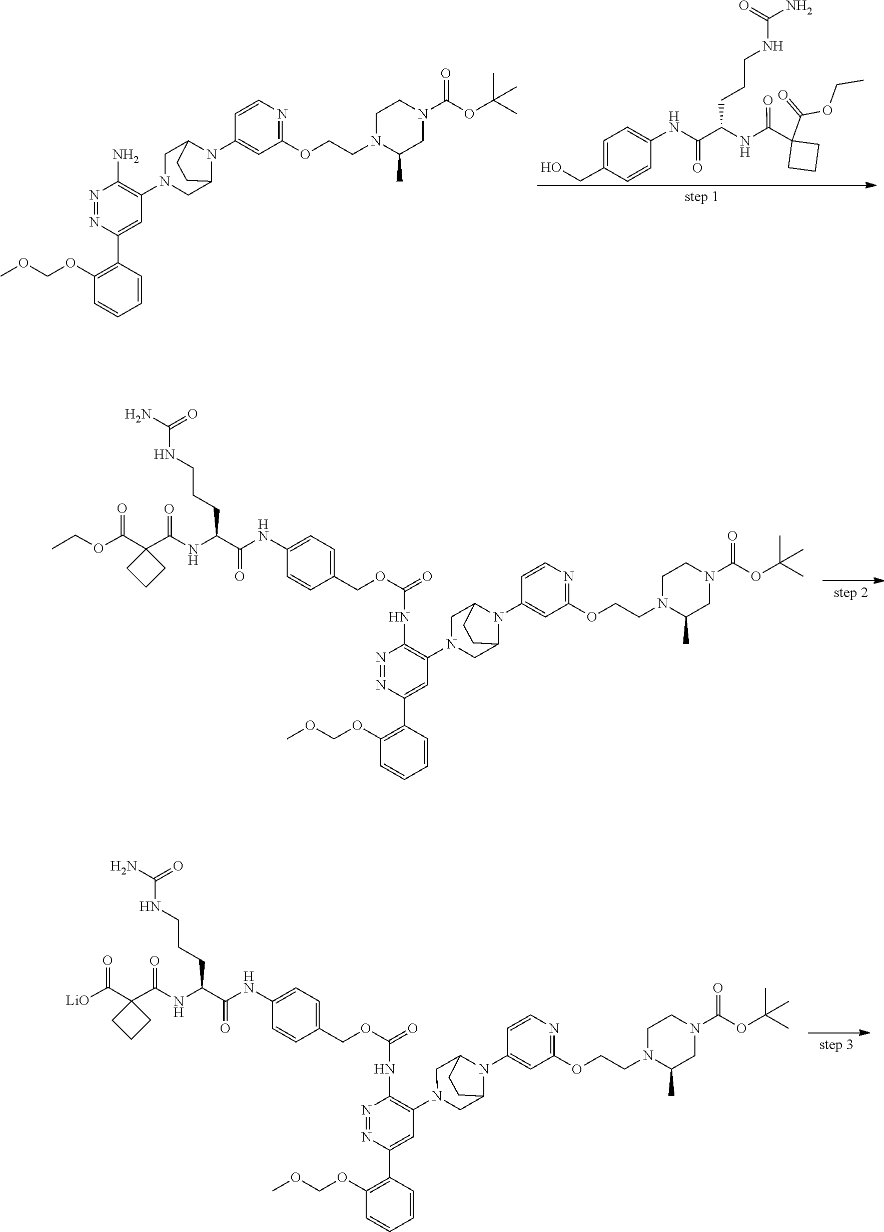

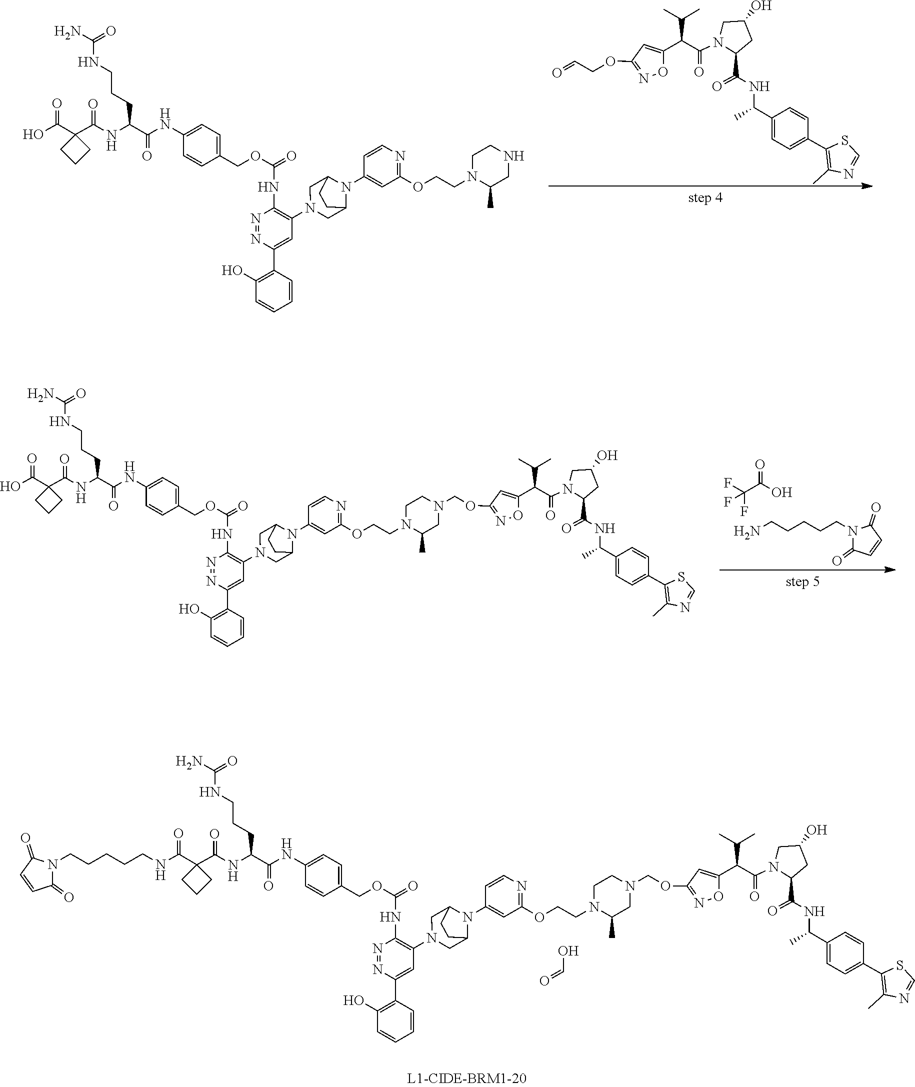

### Step 1: tert-Butyl (3R)-4-(2-((4-(3-(3-((((4-((S)-2-(1-(ethoxycarbonyl)cyclobutane-1-carboxamido)-5-ureidopentanamido)benzyl)oxy)carbonyl)amino)-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate

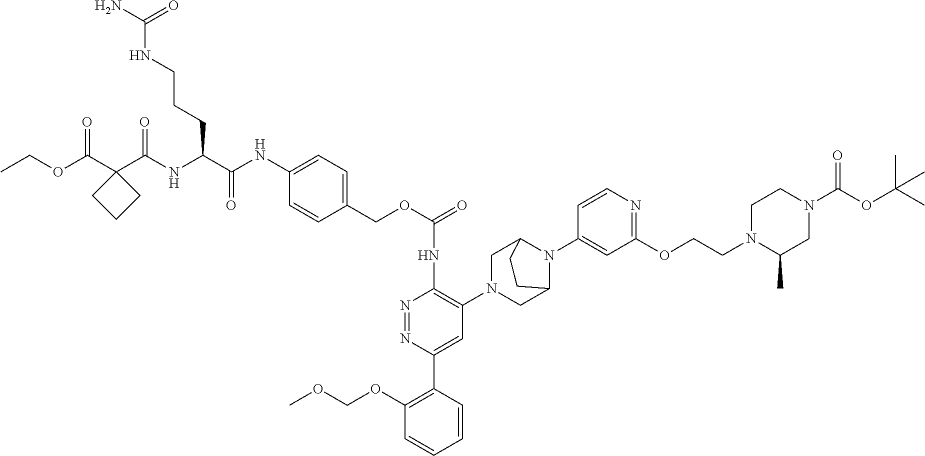 [SMILES: CCOC(=O)C1(C(=O)N[C@@H](CCCNC(N)=O)C(=O)Nc2ccc(COC(=O)Nc3nnc(-c4ccccc4OCOC)cc3N3CC4CCC(C3)N4c3ccnc(OCCN4CCN(C(=O)OC(C)(C)C)C[C@H]4C)c3)cc2)CCC1]

Under nitrogen, to a solution of tert-butyl (3R)-4-(2-((4-(3-(3-amino-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate (500 mg, 0.756 mmol, provided by Genetech), ethyl (S)-1-((1-((4-(hydroxymethyl)phenyl)amino)-1-oxo-5-ureidopentan-2-yl)carbamoyl)cyclobutane-1-carboxylate (986 mg, 2.26 mmol, provided by Genetech) and DIPEA (488 mg, 3.78 mmol) in THF (25 mL) was added triphosgene (85.5 mg, 0.288 mmol) at 0° C. The reaction was stirred at room temperature for 0.5 h. Solvent was evaporated and residue was purified by flash chromatography on silica gel (gradient: 0%-13% methanol/dichloromethane) then purified by pre-packed C18 column (solvent gradient: 0-100% ACN in water (0.05% NH₄HCO₃)) to yield 191 mg (22%) of the title compound as a while solid. LC-MS: (ESI, m/z): [M+H]⁺=1122.

### Step 2: lithium 1-(((2S)-1-((4-((((4-(8-(2-(2-((R)-4-(tert-butoxycarbonyl)-2-methylpiperazin-1-yl)ethoxy)pyridin-4-yl)-3,8-diazabicyclo[3.2.1]octan-3-yl)-6-(2-(methoxymethoxy)phenyl)pyridazin-3-yl)carbamoyl)oxy)methyl)phenyl)amino)-1-oxo-5-ureidopentan-2-yl)carbamoyl)cyclobutane-1-carboxylate

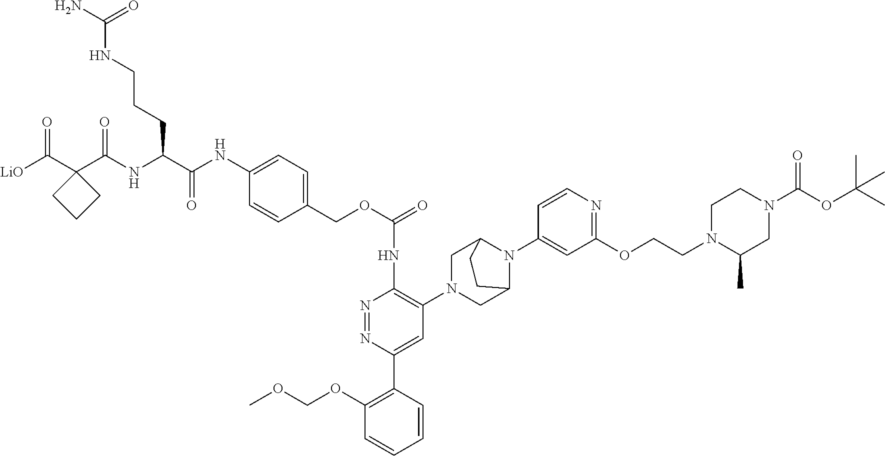

A solution of tert-butyl (3R)-4-(2-((4-(3-(3-((((4-((S)-2-(1-(ethoxycarbonyl)cyclobutane-1-carboxamido)-5-ureidopentanamido)benzyl)oxy)carbonyl)amino)-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate (130 mg, 0.115 mmol) and LiOH (14.0 mg, 0.350 mmol) in THF (3 mL) and water (3 mL) was stirred at room temperature for 1 h. THF was removed under vacuum and then freeze dried to get 140 mg (crude) of the title compound as a white solid. LC-MS: (ESI, m/z): [M+H]⁺=1094.

### Step 3: 1-(((2S)-1-((4-((((6-(2-Hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxo-5-ureidopentan-2-yl)carbamoyl)cyclobutane-1-carboxylic acid

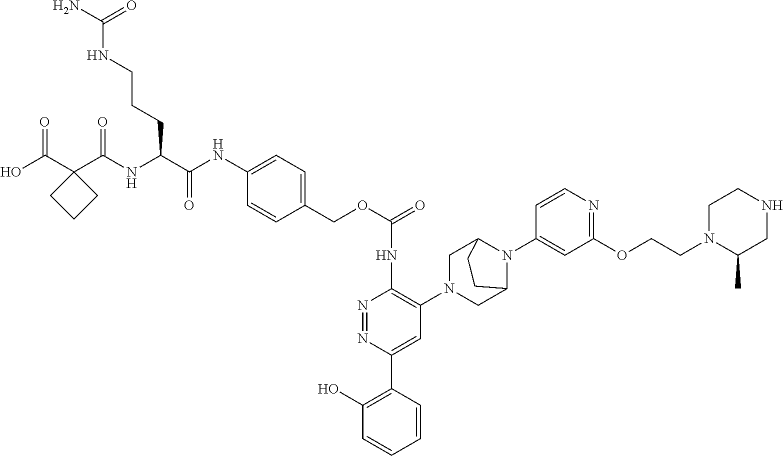 [SMILES: C[C@@H]1CNCCN1CCOc1cc(N2C3CCC2CN(c2cc(-c4ccccc4O)nnc2NC(=O)OCc2ccc(NC(=O)[C@H](CCCNC(N)=O)NC(=O)C4(C(=O)O)CCC4)cc2)C3)ccn1]

Under nitrogen, a solution of lithium 1-(((2S)-1-((4-((((4-(8-(2-(2-((R)-4-(tert-butoxycarbonyl)-2-methylpiperazin-1-yl)ethoxy)pyridin-4-yl)-3,8-diazabicyclo[3.2.1]octan-3-yl)-6-(2-(methoxymethoxy)phenyl)pyridazin-3-yl)carbamoyl)oxy)methyl)phenyl)amino)-1-oxo-5-ureidopentan-2-yl)carbamoyl)cyclobutane-1-carboxylate (240 mg, 0.219 mmol) in concentrated HCl (2 ml), THF (2 ml) and i-propanol (2 mL) was stirred at room temperature for 0.5 h. The reaction solution was concentrated under vacuum and the remaining aqueous solution was purified by pre-packed C18 column (solvent gradient: 0-100% ACN in water (0.05% NH₄HCO₃)) to yield 150 mg of the title compound as a yellow solid. LC-MS: (ESI, m/z): [M+H]⁺=949.

### Step 4: 1-(((2S)-1-((4-((((4-(8-(2-(2-((R)-4-(2-((5-((R)-1-((2S,4R)-4-Hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxo-5-ureidopentan-2-yl)carbamoyl)cyclobutane-1-carboxylic acid

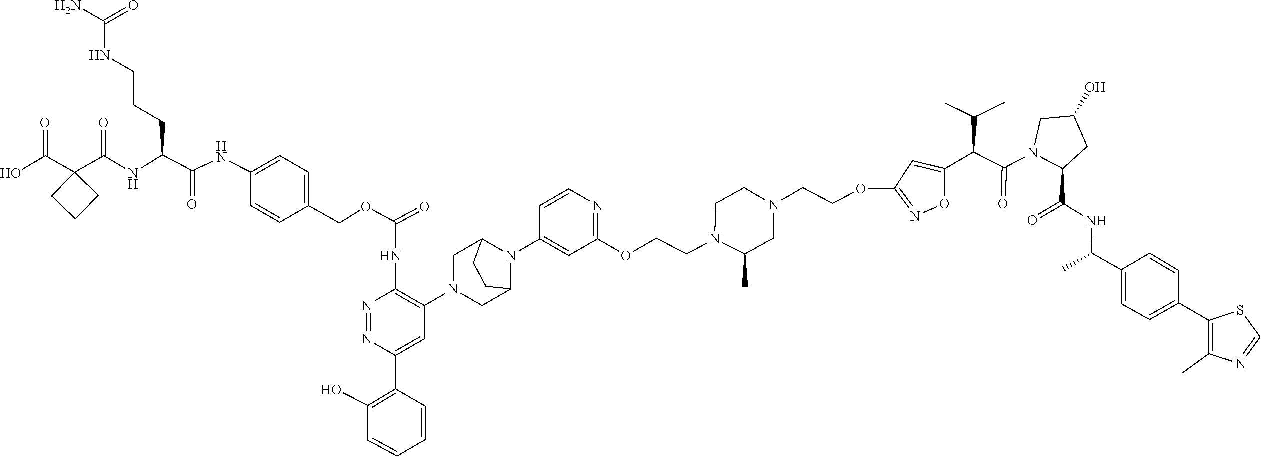 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCNC(N)=O)NC(=O)C7(C(=O)O)CCC7)cc5)C6)ccn4)[C@H](C)C3)no2)C(C)C)cc1]

Under nitrogen, a solution of 1-(((2S)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxo-5-ureidopentan-2-yl)carbamoyl)cyclobutane-1-carboxylic acid (150 mg, 0.158 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (112 mg, 0.207 mmol), CH₃COOH (19.8 mg, 0.329 mmol) in methanol (3 mL) and DCM (1 ml) was stirred at 30° C. for 1 hour. Then NaBH₃CN (19.5 mg, 0.513 mmol) was added and stirred at 30° C. for 0.5 hours. The reaction solution was concentrated under vacuum. The residue was purified by pre-packed C18 column (solvent gradient: 0-100% methanol in water (0.05% NH₄HCO₃)) to yield 180 mg (77%) of the title compound as a yellow solid. LC-MS: (ESI, m/z): [M+H]⁺=1474.

### Step 5: 4-((S)-2-(1-((5-(2,5-Dioxo-2,5-dihydro-1H-pyrrol-1-yl)pentyl)carbamoyl)cyclobutane-1-carboxamido)-5-ureidopentanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (formic acid salt)

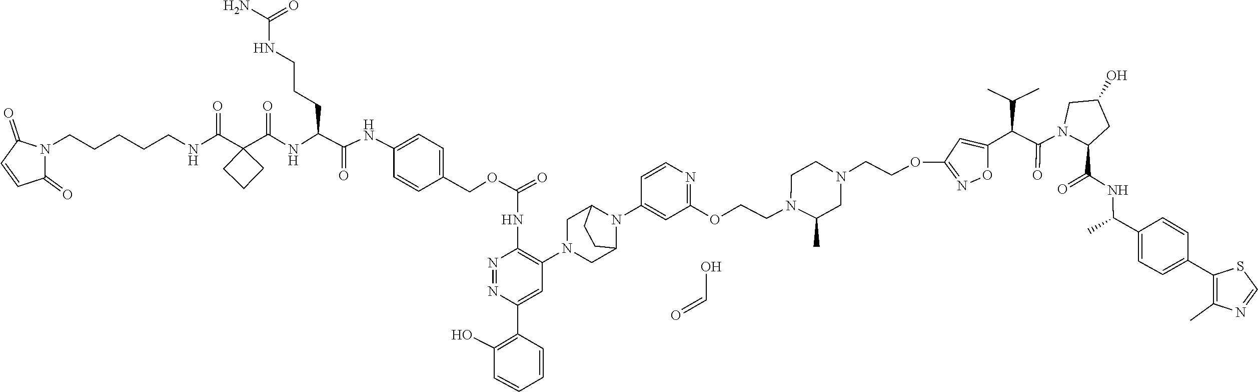 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCNC(N)=O)NC(=O)C7(C(=O)NCCCCCN8C(=O)C=CC8=O)CCC7)cc5)C6)ccn4)[C@H](C)C3)no2)C(C)C)cc1 ; O=CO]

To a solution of 1-(((2S)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxo-5-ureidopentan-2-yl)carbamoyl)cyclobutane-1-carboxylic acid (180 mg, 0.122 mmol), 1-(5-aminopentyl)-1H-pyrrole-2,5-dione (2,2,2-trifluoroacetic acid salt) (67 mg, crude) and DIPEA (158 mg, 1.22 mmol) in DMF (3 mL) was added HATU (67.0 mg, 0.176 mmol) at room temperature. The reaction was stirred at room temperature for 1 h. The resulting solution was purified by Prep-HPLC (Column: XBridge Prep OBD C18 Column, 30*150 mm, 5 μm; Mobile Phase A: Water (0.1% FA), Mobile Phase B: ACN; Flow rate: 60 mL/min; Gradient: 8% B to 38% B in 7 min; Wavelength: 254 nm; R_{T1} (min): 6.5 min) to yield 49.7 mg (24.0% yield) of L1-CIDE-BRM1-20 as a white solid.

LC-MS: (ESI, m/z): [M+H]⁺=1638. ¹H NMR (300 MHz, DMSO-d₆) δ 13.39 (s, 1H), 10.13 (s, 1H), 9.94 (s, 1H), 8.99 (s, 1H), 8.40 (d, J=7.7 Hz, 1H), 8.14 (s, 1H), 8.03 (d, J=7.9 Hz, 1H), 7.82 (dd, J=8.0, 5.8 Hz, 3H), 7.70-7.61 (m, 3H), 7.51-7.41 (m, 2H), 7.41-7.28 (m, 5H), 6.96 (d, J=16.1 Hz, 4H), 6.54 (d, J=6.1 Hz, 1H), 6.17 (s, 1H), 6.10 (s, 1H), 5.96 (dd, J=10.3, 4.5 Hz, 1H), 5.41 (s, 2H), 5.09 (d, J=5.3 Hz, 3H), 4.91 (t, J=7.1 Hz, 1H), 4.50-4.34 (m, 4H), 4.32-4.28 (m, 5H), 3.74-3.61 (m, 2H), 3.55-3.40 (m, 4H), 3.39-3.35 (m, 3H), 3.19-2.89 (m, 9H), 2.70-2.60 (m, 2H), 2.48-2.38 (m, 9H), 2.23-2.12 (m, 1H), 2.10-1.95 (m, 2H), 1.88 (s, 4H), 1.80-1.70 (m, 4H), 1.68-1.57 (m, 1H), 1.52-1.30 (m, 10H), 1.28-1.16 (m, 2H), 1.10-0.90 (m, 6H), 0.82 (d, J=6.6 Hz, 3H).

### Synthesis Example 21

### Synthesis of L1-CIDE-BRM1-21

### N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-(2,5-dioxo-2,5-dihydro-1H-pyrrol-1-yl)pentyl)cyclobutane-1,1-dicarboxamide; 2,2,2-trifluoroacetic acid

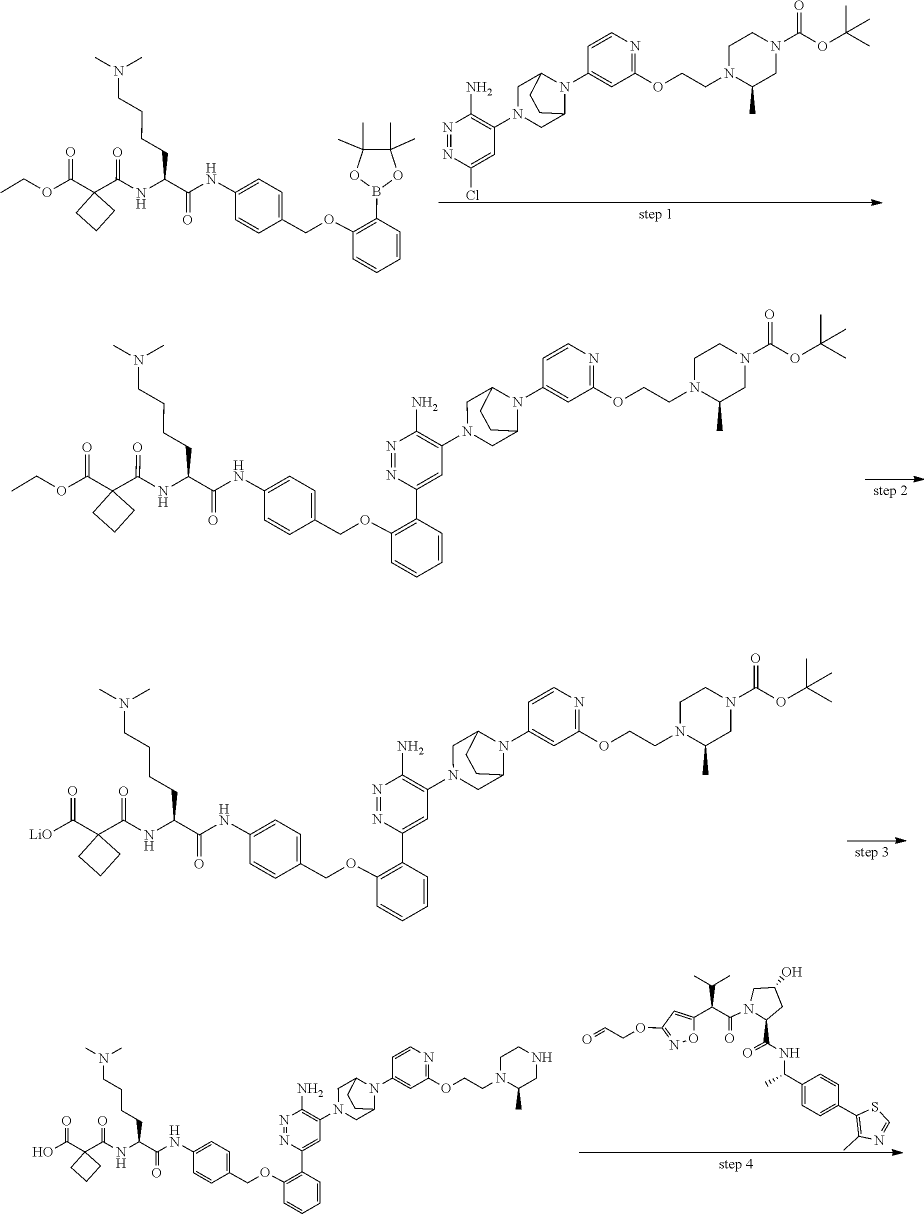

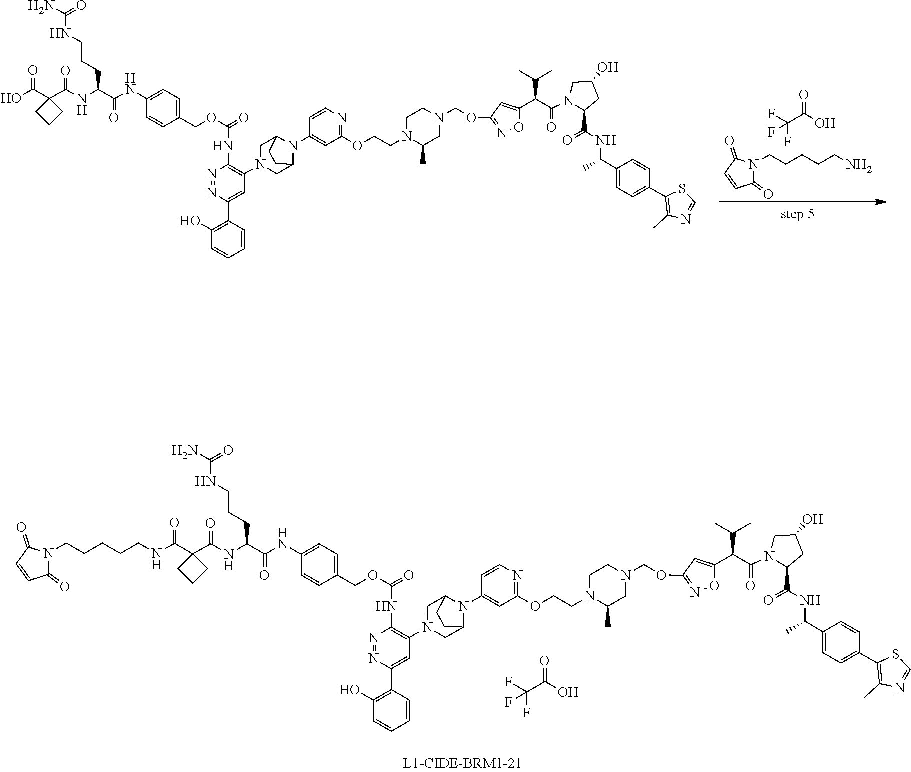

### Step 1: tert-Butyl (3R)-4-(2-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate

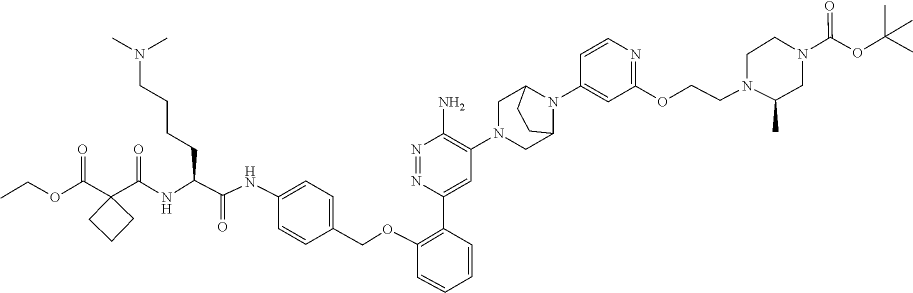 [SMILES: CCOC(=O)C1(C(=O)N[C@@H](CCCCN(C)C)C(=O)Nc2ccc(COc3ccccc3-c3cc(N4CC5CCC(C4)N5c4ccnc(OCCN5CCN(C(=O)OC(C)(C)C)C[C@H]5C)c4)c(N)nn3)cc2)CCC1]

Under nitrogen, a solution of ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate (316 mg, 0.570 mmol), tert-butyl (3R)-4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (538.8 mg, 0.850 mmol), K₃PO₄ (240 mg, 1.13 mmol) and Ad₂nBuPPdG2 (37.8 mg, 0.0600 mmol) in 1,4-dioxane (4 mL) and water (1 mL) was stirred at 95° C. for 3 hours. Water was added and EtOAc was used to extract for three times. The organic solvent was combined and concentrated under vacuum. The residue was purified by pre-packed C18 column (solvent gradient: 0-100% ACN in water (0.05% NH₄HCO₃)) to afford 230 mg (39% yield) of the title compound as a red solid. LCMS (ESI) [M+H]⁺=1032

### Step 2: lithium 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate

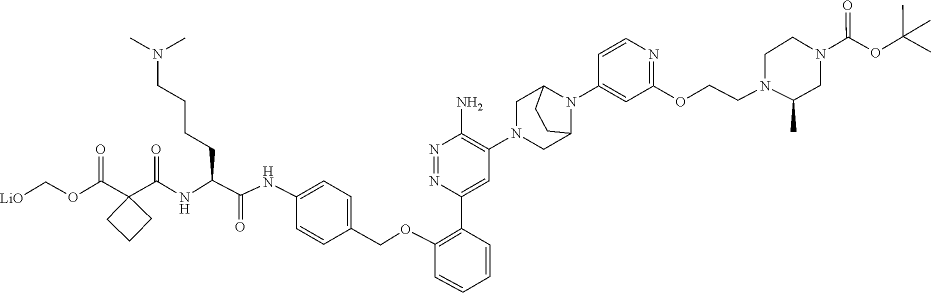

A solution of tert-butyl (3R)-4-(2-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate (210 mg, 0.200 mmol) and LiOH·H₂O (25.6 mg, 0.610 mmol) in tetrahydrofuran (3 mL) and water (1 mL) was stirred at room temperature for 1 hour. The solvent was concentrated under vacuum to afford 254 mg (crude) of the title compound as a yellow solid.

### Step 3: 1-(((2S)-1-((4-((2-(6-Amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

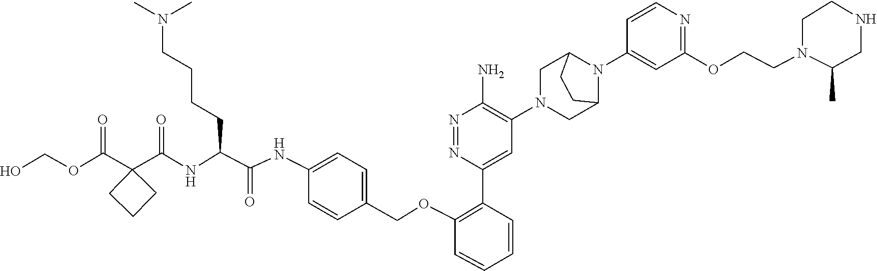 [SMILES: C[C@@H]1CNCCN1CCOc1cc(N2C3CCC2CN(c2cc(-c4ccccc4OCc4ccc(NC(=O)[C@H](CCCCN(C)C)NC(=O)C5(C(=O)OCO)CCC5)cc4)nnc2N)C3)ccn1]

A solution of lithium 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate (254 mg, 0.250 mmol) in 5% TFA/HFIP (20 mL) was stirred at room temperature for 3 hours. The solvent was concentrated under vacuum. The residue was purified by pre-packed C18 column (solvent gradient: 0-100% MeOH in water (0.05% NH₄HCO₃)) to yield 102 mg (44% yield) of the title compound as a red solid.

LCMS (ESI) [M+H]⁺=905.

### Step 4: 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

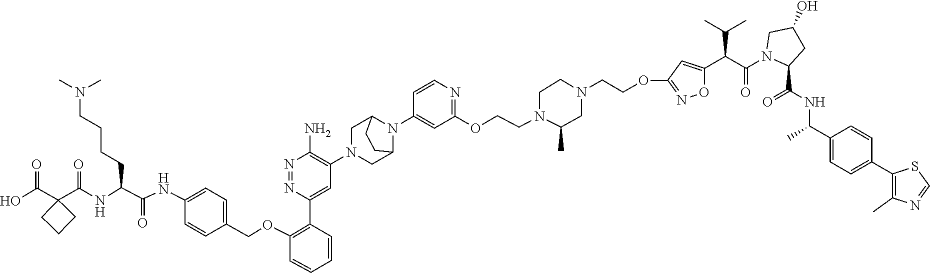 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (102 mg, 0.110 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (61.0 mg, 0.110 mmol) and CH3COOH (13.6 mg, 0.230 mmol) in methyl alcohol (1.2 mL) and dichloromethane (0.4 mL) was stirred at room temperature for 1 hour. Then NaBH₃CN (21.3 mg, 0.340 mmol) was added and stirred at room temperature for 0.5 hours. Water was added to quench the reaction. The solvent was concentrated under vacuum. The residue was purified by pre-packed C18 column (solvent gradient: 0-100% MeOH in water (0.05% NH₄HCO₃)) to afford 80.0 mg (49% yield) of the title compound as a yellow solid. LCMS (ESI) [M+H]⁺=1429.

### Step 4: N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-(2,5-dioxo-2,5-dihydro-1H-pyrrol-1-yl)pentyl)cyclobutane-1,1-dicarboxamid (2,2,2-trifluoroacetic acid salt)

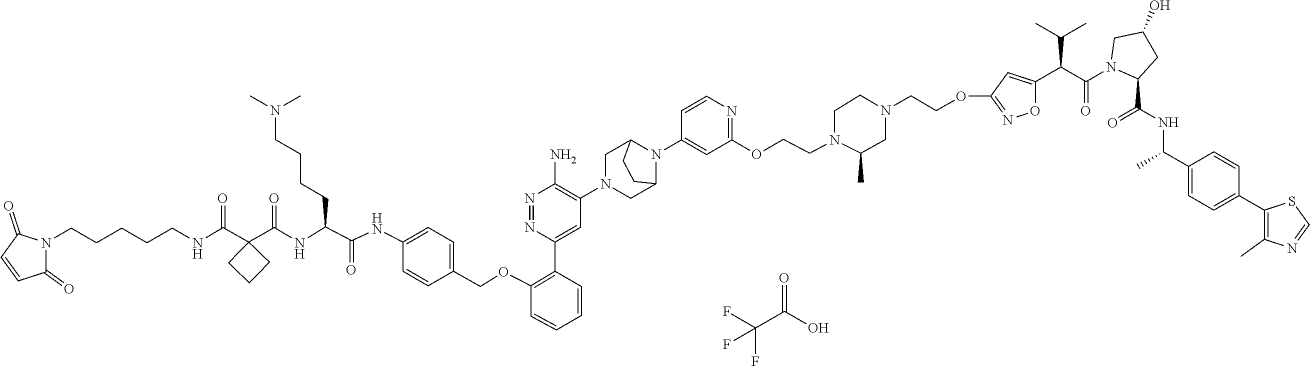 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)[C@H](C)C3)no2)C(C)C)cc1 ; O=C(O)C(F)(F)F]

To a solution of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.i]octan-3-yl)pyridazin-3-yl)phenoxy)methyl)phenyl)amino)-6-(dimethylamino)-1-oxohexan-2-yl)carbamoyl)cyclobutane-1-carboxylic acid (60.0 mg, 0.0400 mmol), 1-(5-aminopentyl)-1H-pyrrole-2,5-dione (2,2,2-trifluoroacetic acid) (23.0 mg, crude) and DIPEA (163 mg, 1.26 mmol) in DMF (2 mL) was added HATU (24.0 mg, 0.0600 mmol) at room temperature. The reaction was stirred at room temperature for 0.5 hour. The product was purified by Prep-HPLC (Column: XBridge Prep Phenyl OBD Column, 19*250 mm, 5 μm; Mobile Phase A: Water (0.05% FA), Mobile Phase B: ACN; Flow rate: 25 mL/min; Gradient: 17% B to 25% B in 10 min, 25% B; Wavelength: 254 nm; R_{T}I (min): 8.77). Then it was purified again by Prep-HPLC(Column: Xselect CSH C18 OBD Column 30*150 mm 5 μm; Mobile Phase A: Water (0.05% TFA), Mobile Phase B: ACN; Flow rate: 60 mL/min; Gradient: 13% B to 38% B in 8 min, 38% B; Wavelength: 254/220 nm; RT₁ (min): 8) to afford 5.0 mg (7% yield) of L1-CIDE-BRM1-21 as a yellow solid. LCMS (ESI) [M+H]⁺=1593. ¹H NMR (300 MHz, DMSO-d6) δ 10.21 (s, 1H), 9.53 (s, 1H), 8.99 (s, 1H), 8.39 (d, J=7.8 Hz, 1H), 7.95-7.80 (m, 3H), 7.67 (d, J=8.1 Hz, 2H), 7.63-7.50 (m, 2H), 7.50-7.43 (m, 8H), 7.19-7.08 (m, 1H), 7.10-6.90 (m, 3H), 6.64 (s, 1H), 6.25 (s, 1H), 6.11 (s, 1H), 5.09 (s, 2H), 4.91 (t, J=7.1 Hz, 1H), 4.48 (br, 4H), 4.41-4.30 (m, 4H), 3.73-3.65 (m, 6H), 3.47-3.31 (m, 7H), 3.13-2.88 (m, 12H), 2.75 (d, J=4.4 Hz, 7H), 2.48-2.38 (m, 8H), 2.25-2.15 (m, 1H), 2.05-1.59 (m, 11H), 1.50-1.29 (m, 8H), 1.31-1.11 (m, 6H), 0.96 (d, J=6.4 Hz, 3H), 0.87-0.76 (m, 3H).

### Synthesis Example 22

### Conjugation of L1-CIDE to an Antibody

The cysteine-engineered antibody (THIOMAB™), in 10 mM succinate, pH 5, 150 mM NaCl, 2 mM EDTA, is pH-adjusted to pH 7.5-8.5 with 1M Tris. 3-16 equivalents of a L1-CIDE (containing a thiol-reactive maleimide group) is dissolved in DMF or DMA (concentration=10 mM) and is added to a reduced, reoxidized, and pH-adjusted antibody. The reaction is incubated at room temperature or 37° C. and are monitored until completion (1 to about 24 hours) as determined by LC-MS analysis of the reaction mixtures. When the reactions are complete, the Ab-CIDEs are purified by one or any combination of several methods, the goal being to remove remaining unreacted linker-drug intermediates and aggregated proteins (if present at significant levels). In one example, the Ab-CIDEs are diluted with 10 mM histidine-acetate, pH 5.5 until the final pH is approximately 5.5 and are purified by S cation exchange chromatography using either HiTrap S columns connected to an Akta purification system (GE Healthcare) or S maxi spin columns (Pierce). Alternatively, the Ab-CIDEs are purified by gel filtration chromatography using an S200 column connected to an Akta purification system or Zeba spin columns. Dialysis is used to purify the conjugates.

The THIOMAB™ Ab-CIDEs are formulated into 20 mM His/acetate, pH 5, with 240 mM sucrose using either gel filtration or dialysis. The purified Ab-CIDEs are concentrated by centrifugal ultrafiltration and filtered through a 0.2-μm filter under sterile conditions and are frozen at −20° C. for storage.

### Biological Example 1

### Cell-based Assays

### Immunofluorescence Detection of BRM

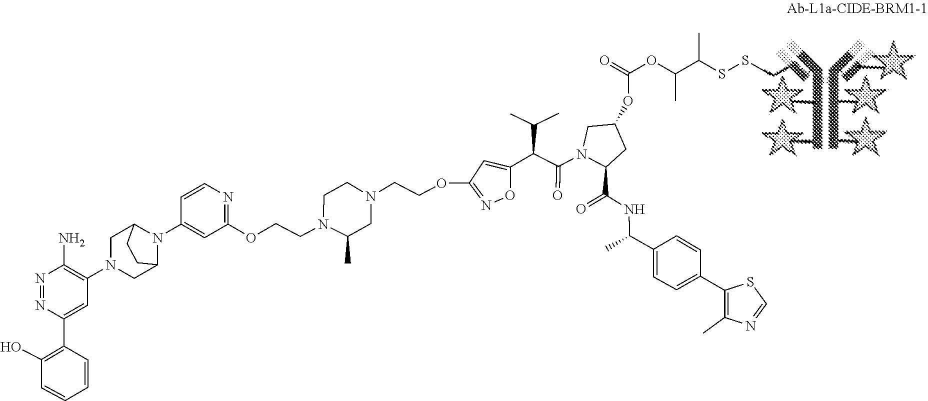

Conjugation to CD22 had a DAR of 5.8. Conjugation to EpCAM had a DAR of 5.9.

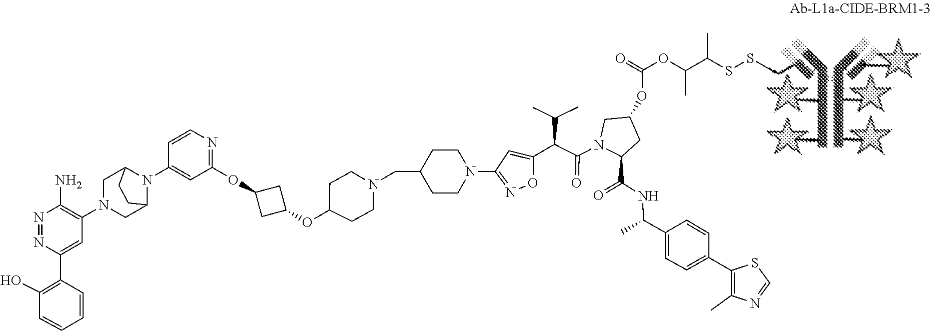 [SMILES: CSSC(C)C(C)OC(=O)O[C@@H]1C[C@@H](C(=O)N[C@@H](C)c2ccc(-c3scnc3C)cc2)N(C(=O)[C@@H](c2cc(N3CCC(CN4CCC(O[C@H]5C[C@H](Oc6cc(N7C8CCC7CN(c7cc(-c9ccccc9O)nnc7N)C8)ccn6)C5)CC4)CC3)no2)C(C)C)C1]

Conjugation to CD22 had a DAR of 5.8. Conjugation to EpCAM had a DAR of 5.9. CD-22: Thio Hu Anti-CD22 10F4v3 high DAR [LC:K149C HC: Y373C HC:L174C] MeMe disulfide BRM CIDE; EpCAM: Thio Hu Anti-Her2 7C2 high DAR [LC:K149C HC:L174C HC:Y373C] MeMe disulfide BRM CIDE

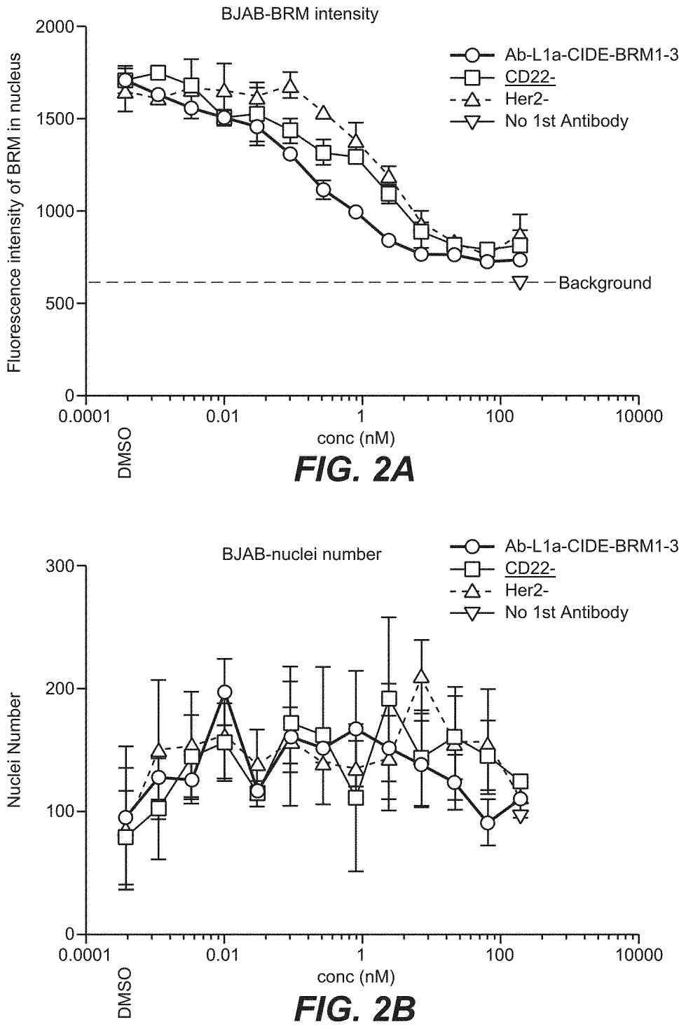

FIGS. 1a and 1b show the activity of Ab-L1a-CIDE-BRM1-1. FIGS. 2a and 2b show the activity of Ab-L1a-CIDE-BRM1-3.

### Biological Example 2

### PK/PD BJAB Tumor Assays

The PK/PD effects of anti-CD22-BRM Ab-CIDEs were evaluated in a mouse xenograft model of BJAB-luc human non-Hodgkin's lymphoma. The BJAB-luc was obtained from Genentech cell line repository. This cell line was authenticated by short tandem repeat (STR) profiling using the Promega PowerPlex 16 System and compared with external STR profiles of cell lines to confirm cell line ancestry.

To establish the model, tumor cells (20 million in 0.2 mL of Hank's Balanced Salt Solution) were inoculated subcutaneously to the flank of female C.B-17 SCID mice (Charles River Laboratories). When tumors reached the desired volume (300-400 mm3), mice were randomized into groups of n=5 each with similar distribution of tumor sizes, and received a single intravenous injection of vehicle (histidine buffer) or the test article through the tail vein. All anti-CD22-BRM Ab-CIDEs and the unconjugated antibody were formulated in the histidine buffer (20 mM histidine acetate pH 5.5, 240 mM sucrose, 0.02% Tween 20). The unconjugated BRM CIDE was formulated in 10% hydroxypropyl-beta-cyclodextrin, 50 mM sodium acetate, pH4.

At four days post-dose, mice were euthanized and tumors and whole blood were collected. Tumors were excised and split into two aliquots prior to being flash frozen in liquid nitrogen. One aliquot was used to measure level of released BRM CIDE and the other aliquot was used to evaluate the modulation of downstream PD markers. Whole blood was collected by terminal cardiac puncture under a surgical plane of anesthesia, and into tubes containing lithium heparin. Blood was allowed to sit on wet ice until centrifugation (within 15 min of collection). Samples were centrifuged at 10,000 rpm for 5 min at 4° C., and plasma was collected, placed on dry ice, and stored at −70° C. until analysis for linker stability and total antibody pharmacokinetics.

Western Blotting of Xenograft Tissue

On dry ice frozen tissue was cut into 15-30 mg pieces and then transferred to a 1.5 mL Eppendorf Safe-Lock tube with one 3.2 mm (NextAdvance (3.2 mm, SSB32)) stainless steel bead. RIPA buffer (350 uL) supplemented with 0.5 M NaCl and freshly added 1×Halt protease and phosphatase inhibitor was added and the tube was place into the Bullet Blender tissue homogenizer. Samples were homogenized at highest speed for 3 minutes. Tubes were spun at 4° C. at top speed for 5 minutes in bench top centrifuge and lysate was transferred to fresh tube. Protein concentration was determined using Pierce BCA protein assay. Protein lysate were prepared with sample buffer and reducing reagent, and incubated for 3 minutes at 95° C. Protein (12 ug) was separated on a 3-8% Tris acetate gel with tris-acetate running buffer followed by transfer to a nitrocellulose membrane using an iBlot transfer device (25V, 10 minutes). Following blocking membrane with 5% Milk in TBS-T for 30 minutes, primary antibodies were added at 1/1000. Membranes were blotted for SMARCA2 (BRM) (rabbit, Cell signaling technologies Cat #11966) and HDAC1 (mouse, Cell signaling technologies Cat #5356) and incubated over night at 4° C. on rocker. The next day membranes were washed on rocker for 30 minutes with TBS-T at room temperature, changing the wash buffer at least 3 times. Membrane were then incubated with Licor secondary at 1/5000 in TBS-T for 1 hour at room temperature on rocker. Blots were washed with TBS-T for 1 hour, changing the wash buffer at least 6 times. Images were capture on Licor imaging system.

Murine tumor assays were performed. Table 1 shows the study arms and parameters.

TABLE 1
BJAB tumor (CB17-SCID mice), PK/PD study

| | | | | | PD Time | |
|---|---|---|---|---|---|---|
| Arm | Compound | mg/kg | Freq. | Route | Point | #Mice |
| 1 | Vehicle control | NA | once | IV | 96 h | 5 |
| 2 | Unconjugated mAb control | 10 | once | IV | 96 h | 5 |
| 3 | CD22-L1a-CIDE-BRM1-1 DAR6 | 1 | once | IV | 96 h | 5 |
| 4 | EpCAM-L1a-CIDE-BRM1-1 DAR6 | 1 | once | IV | 96 h | 5 |
| 5 | CD22-L1a-CIDE-BRM1-1 DAR6 | 10 | once | IV | 96 h | 5 |
| 6 | EpCAM-L1a-CIDE-BRM1-1 DAR6 | 10 | once | IV | 96 h | 5 |
| 7 | CD22-L1a-CIDE-BRM1-3 DAR6 | 1 | once | IV | 96 h | 5 |
| 8 | HER2-L1a-CIDE-BRM1-3 DAR6 | 1 | once | IV | 96 h | 5 |
| 9 | CD22-L1a-CIDE-BRM1-3 DAR6 | 10 | once | IV | 96 h | 5 |
| 10 | HER2-L1a-CIDE-BRM1-3 DAR6 | 10 | once | IV | 96 h | 5 |
| 11 | CIDE-BRM1-3 unconjuated control | 20 | once | IV | 96 h | 5 |

Split tumor tissue 2-ways for (1) BRM, BRG, PBRM1 PD, and (2) tumor PK
Plasma PK timepoints same as those for PD listed above
Antibody Conjugates, IV Formulation: Histidine buffer, dose volume=5 mL/kg
CIDE-BRM1-3, IV Formulation: 10% HP-b-CD and 50 mM Sodium Acetate in Water pH 4.0 dose vol=5 mL/kg

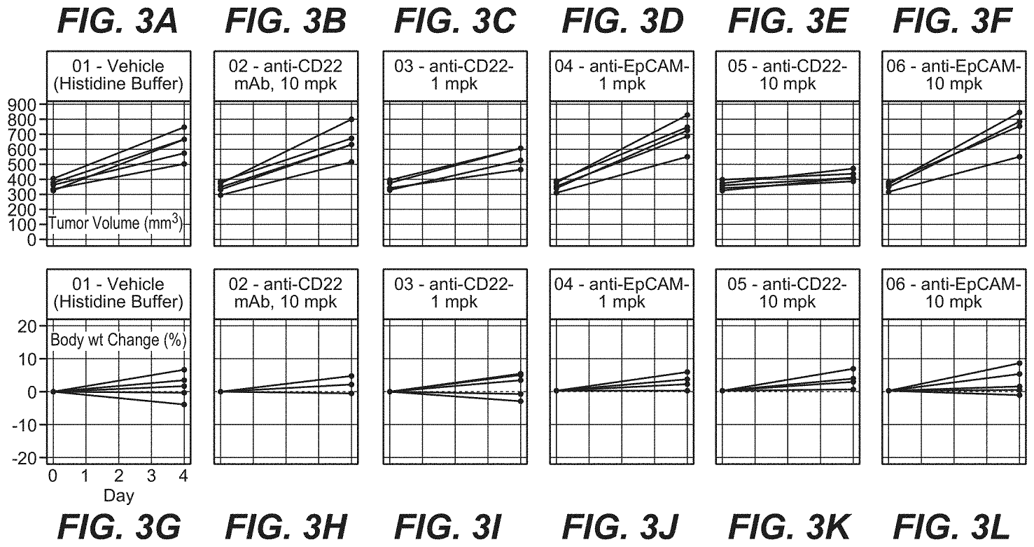

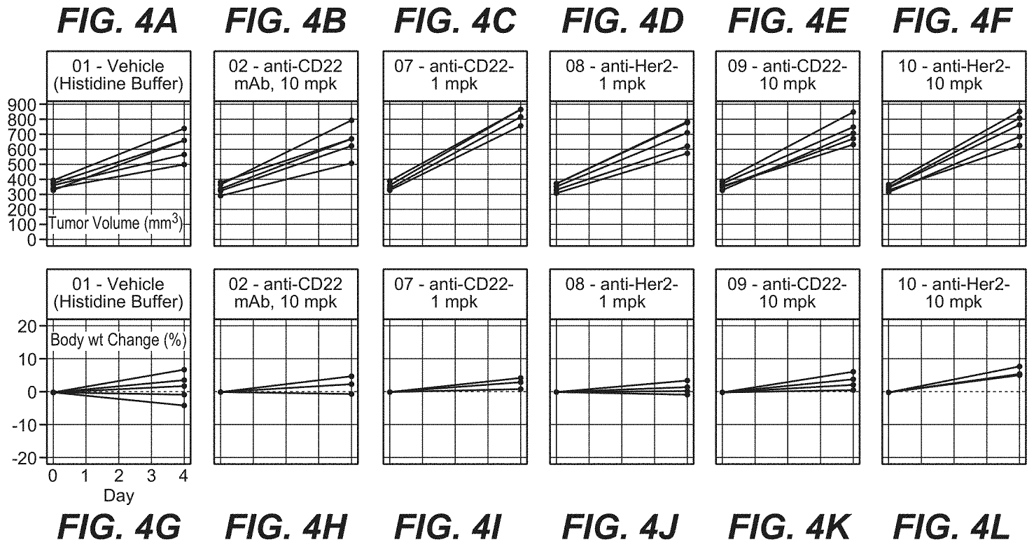

Dose and antigen-dependent anti-tumor activity for Ab-L1a-CIDE-BRM1-1 are shown in FIGS. 3A-3L, and for Ab-L1a-CIDE-BRM1-3 in FIGS. 4A-4L.

### Biological Example 3

### Target Protein Degradation Assays

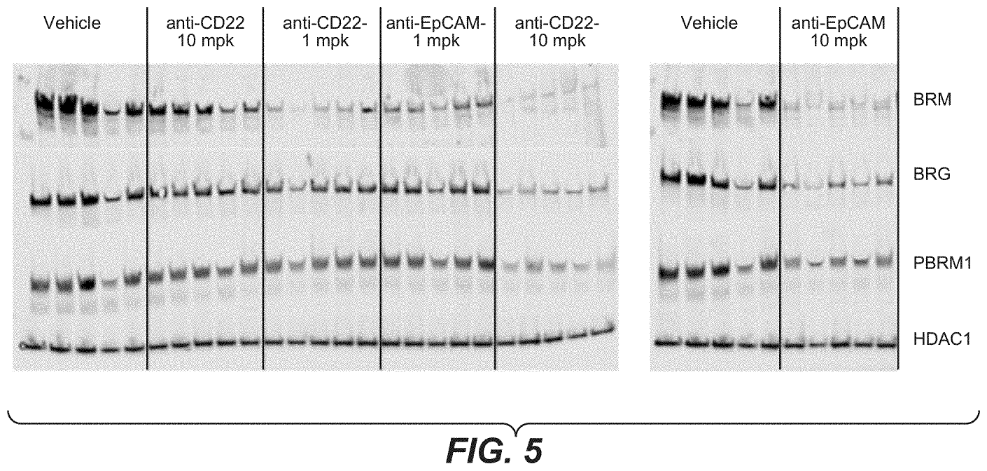

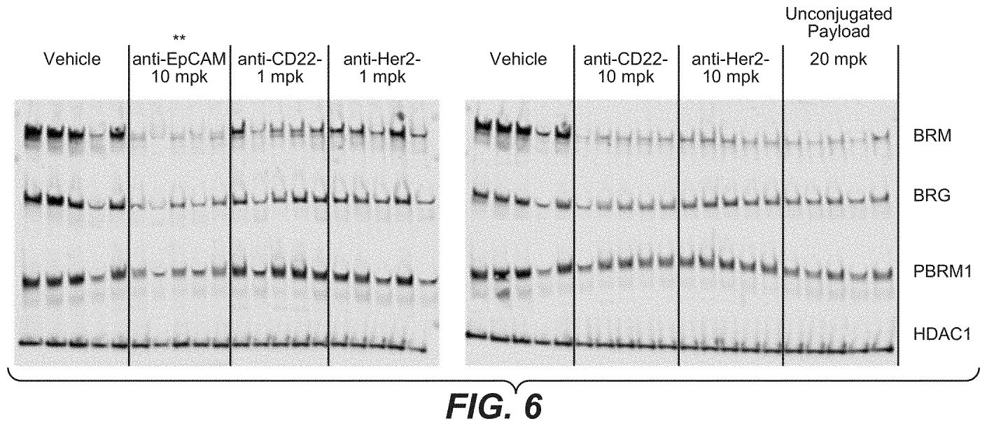

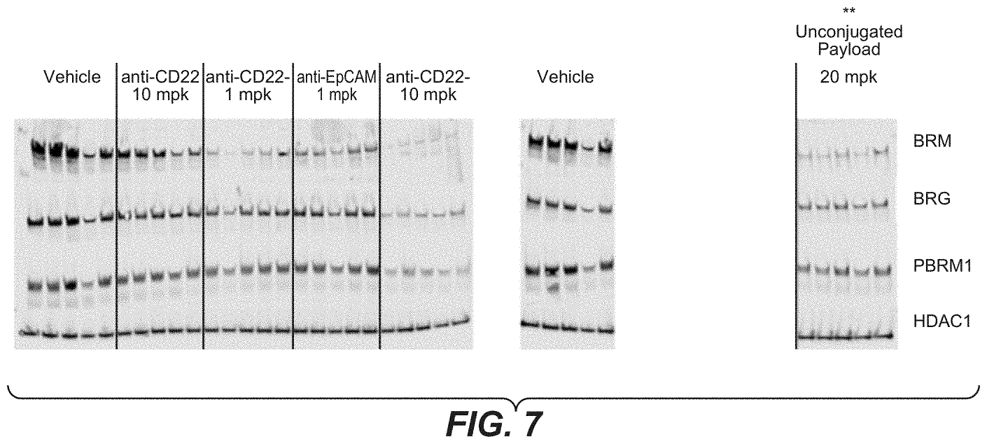

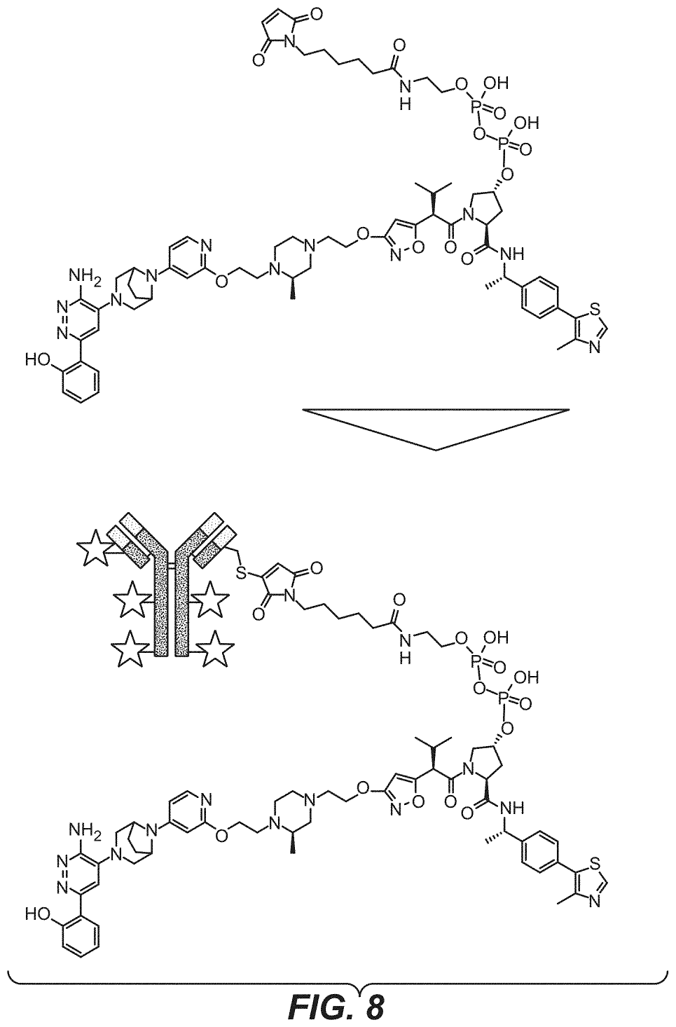

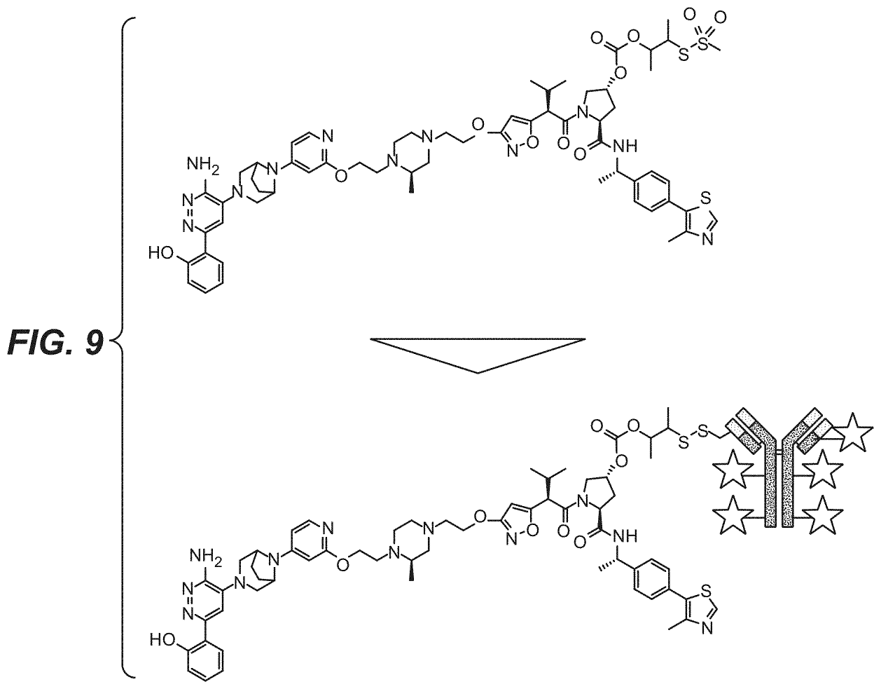

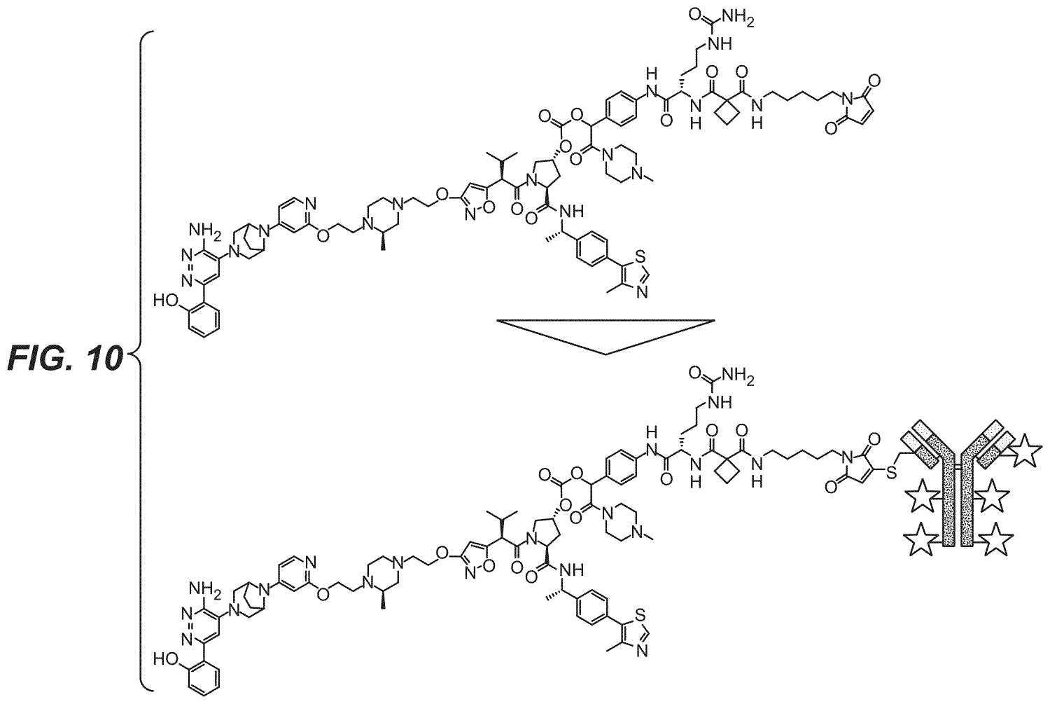

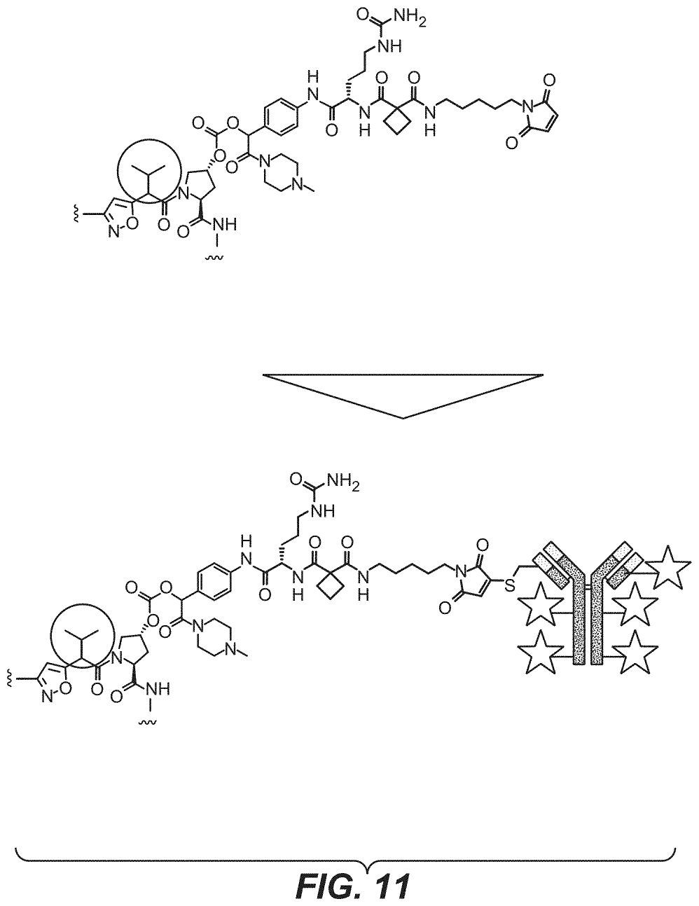

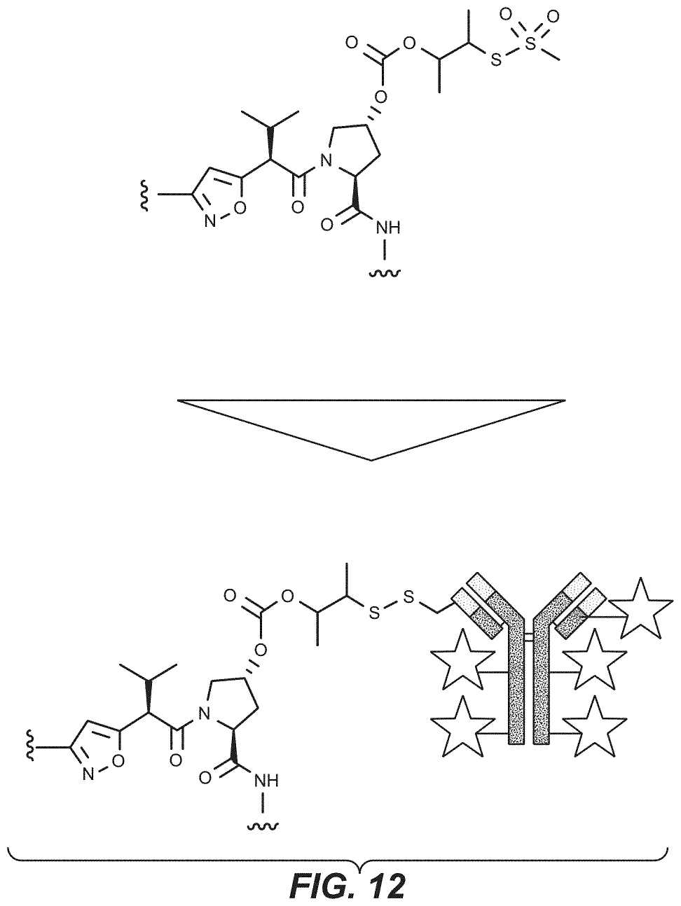

The data report on an improved PD response. FIG. 5 depicts data showing that for Ab-L1a-CIDE-BRM1-1, BRM and BRG1 degradation correlates with anti-tumor activity. FIG. 6 depicts data showing that for Ab-L1a-CIDE-BRM1-3, BRM and BRG1 degradation is less correlated with anti-tumor activity. FIG. 7 depicts data showing antibody conjugation strategy increases degradation activity. The time point for all these data is 96 hours. The Ab-L1a-CIDE-BRM1-1 degrades better than unconjugated CIDE-BRM1-3, while both compounds have similar BRM degradation properties in cell assays in unconjugated form (CIDE-BRM1-3 vs. CIDE-BRM1-1 assays described in WO2019195201). This effect demonstrates that the linking strategies described herein can modulate the degradation properties.

### Biological Example 4

### Cell Assays to Determine DC50 and Dmax

Cell based assays were run in two cell lines to determine the DC50 and Dmax of Ab-L1-CIDE. BJAB, HCC515 and H1944 cells were plated in 384 well plates at 5000, 4000 and 2500 cells/well, respectively. The next day Ab-CIDEs were added. Following 24 h of drug treatment the cells were fixed with 4% formaldehyde for 15 minutes. The plates were washed three time with PBS. The cells were incubated with IF blocking solution (10% FCS, 1% BSA, 0.1% Triton, 0.01% Azide, X-100 in PBS). After 1.5 h a 2× solution of primary antibody diluted in IF blocking buffer: BRM (Cell signaling Cat #11966, 1:2000) was added. The plates were incubated over night at 4° C. The following morning cells were washed three time with PBS. Cells were then incubated with secondary antibodies (rabbit-Alexa 488 A21206 (1:2000)) for 1 h at room temperature in the dark. Hoechst H3570 at 1:5000 was added to the wells and the plates were incubated for an additional 30 minutes. Plates were wash 3×PBS and image on Opera Phenix™ High Content Screening System. Using nuclear staining as a mask, nuclear BRM mean signal intensity was quantified.

The data are shown in Table 2 below. The data evidence the successful antibody targeting strategies disclosed herein. Negative controls: Anti-gD and anti-TROP2 do not interact with NCI-H1944 cells, whereas anti-TfR2 does. Anti-gD further does not interact with HCC515 cells, whereas anti-TfR2 and anti-TROP2 do. The data show that several Ab-L1-CIDEs have both desirably low DC50 and desirably high Dmax values.

TABLE 2

| | | | NCI-HI944 | | HCC515 | |
|---|---|---|---|---|---|---|
| | | | DC50 | | DC50 | |
| Ab-L1-CIDE-BRM | Target | DAR | (μg/mL) | Dmax | (μg/mL) | Dmax |
| Thio Hu Anti-gD 5B6 high DAR | gD | 5.66 | 1.56 | 95 | 0.59 | 93 |
| [HC:L174C HC:Y373C LC:K149C] diMe Me | | | | | | |
| disulfide BRM degrader phos Ab-L1-CIDE-BRM1-9 | | | | | | |
| Thio Hu Anti-TfR2.NG2LH high DAR | TfR2 | 5.87 | 0.74 | 94 | 0.12 | 96 |
| [HC:L174C HC:Y373C LC:K149C] diMe Me | | | | | | |
| disulfide BRM degrader phos Ab-L1-CIDE-BRM1-9 | | | | | | |
| Thio Hu Anti-gD 5B6 LALA.PG high DAR | gD | 5.90 | 9.37 | 93 | 2.65 | 94 |
| [LC:K149C HC:Y373C:HC:L174C] MC-sqCit-BRM | | | | | | |
| degrader Ab-L1-CIDE-BRM1-20 | | | | | | |
| Thio Hu Anti-TfR2.LALA PG high DAR | TfR2 | 6.14 | 0.10 | 95 | 0.02 | 95 |
| [LC:K149C:HC:Y373C:HC:A140C] MC-sqCit- | | | | | | |
| BRM degrader Ab-L1-CIDE-BRM1-20 | | | | | | |
| Thio Hu Anti-TROP2 LALA.PG high DAR | TROP2 | 6.00 | 6.45 | 93 | <0.01 | 95 |
| [LC:K149C:HC:Y373C:HC:A140C] MC-sqCit- | | | | | | |
| BRM degrader Ab-L1-CIDE-BRM1-20 | | | | | | |
| Thio Hu Anti-gD 5B6 LALA.PG high DAR | gD | 5.89 | NA | 32 | 7.60 | 86 |
| [LC:K149C:HC:Y373C:HC:A140C] MC-sqDML- | | | | | | |
| BRM degrader Ab-L1-CIDE-BRM1-19 | | | | | | |
| Thio Hu Anti-TfR2 LALA.PG high DAR | TfR2 | 5.79 | 0.36 | 80 | 0.33 | 84 |
| [LC:K149C:HC:Y373C:HC:A140C] MC-sqDML- | | | | | | |
| BRM degrader Ab-L1-CIDE-BRM1-19 | | | | | | |
| Thio Hu Anti-TROP2 LALA.PG high DAR | TROP2 | 5.73 | NA | 23 | >50 | 82 |
| [LC:K149C:HC:Y373C:HC:A140C] MC-sqDML- | | | | | | |
| BRM degrader Ab-L1-CIDE-BRM1-19 | | | | | | |
| Thio Hu Anti-gD 5B6 high DAR | gD | 5.96 | >10 | NA | >10 | NA |
| [HC:L174C HC:Y373C LC:K149C] MC-gluc BRM | | | | | | |
| degrader Ab-L1-CIDE-BRM1-15 | | | | | | |
| Thio Hu Anti-TfR2.NG2LH high DAR | TfR2 | 5.97 | >10 | NA | >10 | NA |
| [HC:L174C HC:Y373C LC:K149C] MC-gluc BRM | | | | | | |
| degrader Ab-L1-CIDE-BRM1-15 | | | | | | |
| Thio Hu Anti-TROP2 high DAR | TROP2 | 5.93 | >10 | NA | >10 | NA |
| [HC:L174C HC:Y373C LC:K149C] MC-gluc BRM | | | | | | |
| degrader Ab-L1-CIDE-BRM1-15 | | | | | | |
| Thio Hu Anti-TfR1.NG2LH.FcRn null high DAR | TfR1 | 5.96 | 0.07 | 77 | not measured | |
| [HC:L174C HC:Y373C LC:K149C] MC-sqCit BRM | | | | | | |
| CIDE Ab-L1-CIDE-BRM1-13 | | | | | | |

### Biological Example 5

### Lysosomal Release Assays

Lysosomal release assays were run to measure the release of the degrader from the L1 moeity in an environment that mimics intracellular milieu. For the degrader to be active at binding BRM and ferrying it to the ubiquitin ligase, the L1 must first be released.

The assay was run using an L1 bound to the BRM-binding compound, named “L1-BRM1-#” that corresponds to the respective CIDE. The test determine whether cleavage of the covalent attachment of the L1 to the BRM portion occurred. Linker-drugs (10 ptM) were incubated with human liver lysosomes (0.17 mg/mL) and cysteine (5 mM) in 100 mM citric acid buffer pH 5.5 for 24 h. The samples were analyzed by Q Exactive Orbitrap mass specrometers using LC mobile phase containing (A) 0.1% formic acid in water and (B) 0.1% formic acid in acetonitrile in a gradient.

The results of the assay are shown in Table 3 below. The results show that a direct linking strategy of L1 to the BRM portion releases in a cellular environment. One conjugate, L1-CIDE-BRM1-15 does not contain an antibody linker of the linker-1 type described herein. Of the DACs tested, L1-CIDE-BRM1-15 did not release the degrader in lysosomal extracts. This finding supports the requirement of selective linking strategies for the degrader to the Ab, such as the L1 linkers of the linker-1 types described herein.

TABLE 3

| | Release in |
|---|---|
| L1-BRM | lysosomal extract |
| L1-BRM1-15 (BRM from CIDE-BRM1-15) | No |
| L1-BRM1-9 (BRM from CIDE-BRM1-9) | Yes |
| L1-BRM1-13 (BRM from CIDE-BRM1-13 | Yes |
| L1-BRM1-20 (BRM from CIDE-BRM1-20 | Yes |

*“No” indicates <15% free drug observed after 24 h incubation at 37° C. in lysosomal extract. “Yes” indicates >50% free drug observed after 24 h incubation at 37° C. in lysosomal extract.

Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise.

It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A conjugate having the structure:
Ab-(L1-D)ₚ,
wherein,
Ab is an antibody;
D is a CIDE, or prodrug thereof, having the structure: 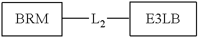
wherein,
BRM is a residue of a BRM-binding compound,
E3LB is a residue of an E3 ligase-binding compound, and
L2 is a moiety covalently linking BRM with E3LB;
L1 is a linker-1 covalently linking Ab to one of BRM, E3LB or L2; and
p is 1 to 16.

2-7. (canceled)

8. The conjugate of claim 1, wherein L1 is selected from the group consisting of 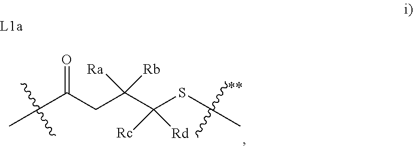
wherein
wherein R^{a}, R^{b}, R^{c}, and R^{d} are independently selected from the group consisting of H, optionally substituted branched or linear C₁-C₅ alkyl, and optionally substituted C₃-C₆ cycloalkyl, or R^{a} and R^{b} taken together or R^{c} and R^{d} taken together with the carbon atom to which they are bound form an optionally substituted C₃-C₆ cycloalkyl ring or a 3 to 6-membered heterocycloalkyl ring; 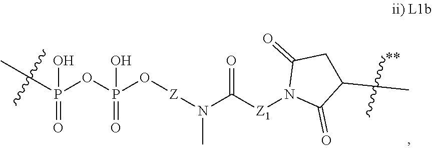
wherein,
Z and Z₁ are each independently a C₁₋₁₂ alkylene or —[CH₂]_{g}—[—O—CH₂]ₕ—, wherein g is 0, 1 or 2, and h is 1-5;
R_{z} is H or C₁₋₃alkyl; and, 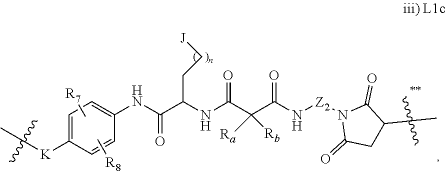
wherein
Z₂ is a C₁₋₁₂ alkylene or —[CH₂]_{g}—[—O—CH₂]ₕ—, wherein g is 0, 1 or 2, and h is 1-5;
w is 1, 2, 3, 4 or 5;
J is —N(Rₓ)(R_{y}), —C(O)NH₂, —NH—C(O)—NH₂, —NH—NH—NH₂, wherein, Rₓ and R_{y} are each independently selected from hydrogen and C₁-3 alkyl;
K is selected from —CH₂—, —CH(R)—, —CH(R)—O-\hat{ }, —C(O)—, \hat{ }—C(O)—O—CH(R)—, —CH₂—O—C(O)-\hat{ }, —CH₂—O—C(O)—NH-\hat{ }, \hat{ }—O—C(L1c)-C(O)—NRₓR_{y}—, \hat{ }-C(L1c)-C(O)—NRₓR_{y}—, —CH₂—O—C(O)—NH—CH₂—, —CH₂—O—C(O)—R—[CH₂]q-O-\hat{ }, —CH₂—O—C(O)—R—[CH₂]q-\hat{ }, wherein \hat{ } indicates the attachment to CIDE, wherein R is hydrogen, C₁₋₃alkyl, N(Rₓ)(R_{y}), —O—N(Rₓ)(R_{y}) or C(O)—N(Rₓ)(R_{y}), wherein q is 0, 1, 2, or 3, and Rₓ and R_{y} are each independently selected from hydrogen and C₁₋₃alkyl, or Rₓ and R_{y} together with the nitrogen to which each is attached form an optionally substituted 5- to 7-member heterocyclyl;
Ra and Rb are each independently selected from hydrogen and C₁₋₃alkyl or Ra and Rb together with the nitrogen to which each is attached form an optionally substituted C₃₋₆cycloalkyl; and
R₇ and R₈ are each independently hydrogen, halo, C₁₋₅ alkyl, C₁₋₅ alkoxy or hydroxyl.

9-16. (canceled)

17. The conjugate of claim 1, wherein D has the structure 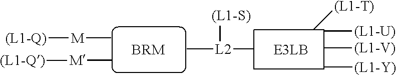
wherein L1 is attached at one attachment point selected from L1-Q, L1-Q′, L1-S, L1-T, and optionally L1-U, L1-V and L1-Y, if present, wherein
L1Q is at 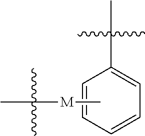
on BRM, wherein M is O;
L1-Q′ is at 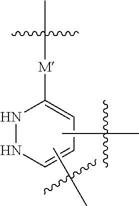
on BRM, wherein M′ is —NH;
L1-S is at 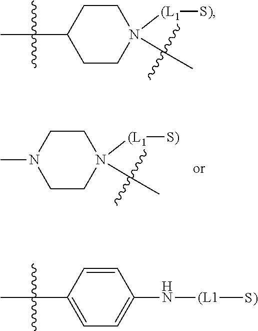
on L2;
L1-T is at 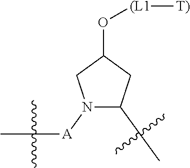
on E3LB, wherein, A is a group covalently bound to L2;
L1-U and L1-V are at 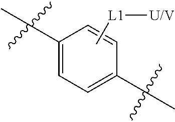
on E3LB; and
L1-Y is at 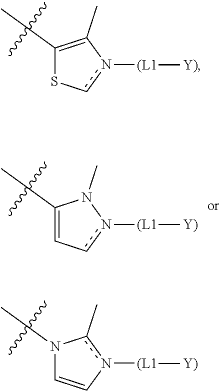
on E3LB, wherein, [symbol] is a single or double bond.

18. The conjugate of claim 8, wherein K of L1c is selected from the group consisting of:

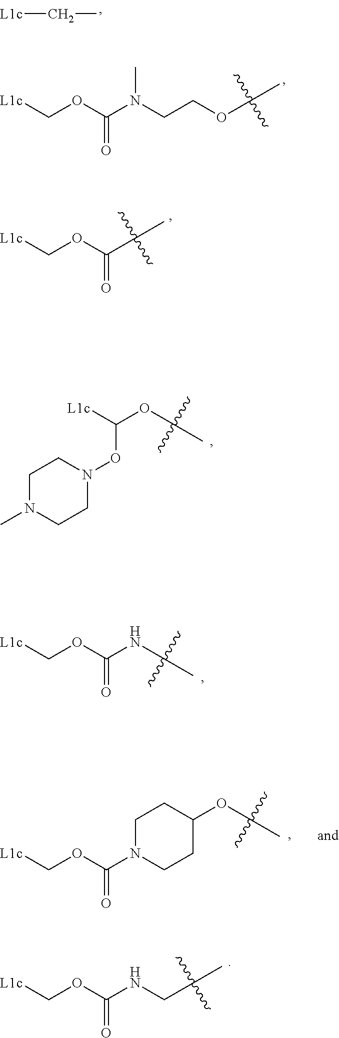

19. The conjugate of claim 17, wherein D has the structure: 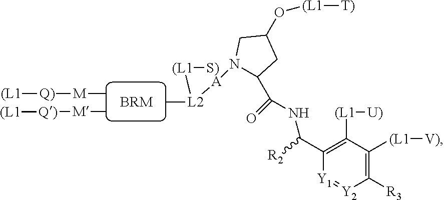
wherein:
R₃ is cyano, 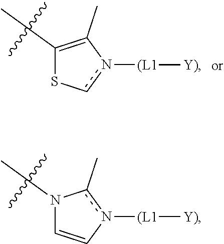
wherein, [symbol] is a single or double bond.

[symbol]

20. The conjugate of claim 19, wherein D has the structure: 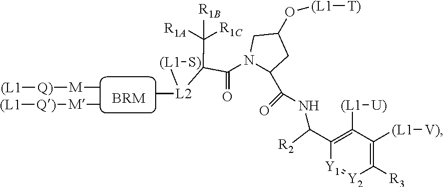
wherein, R_{1A}, R_{1B} and R_{1C} are each independently hydrogen, or C₁₋₅ alkyl; or two of R_{1A}, R_{1B} and R_{1C} together with the carbon to which each is attached form a C₁₋₅ cycloalkyl.

21-22. (canceled)

23. The conjugate of claim 17, wherein D has the structure:

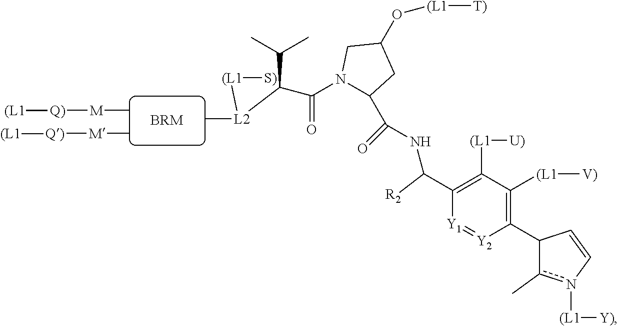

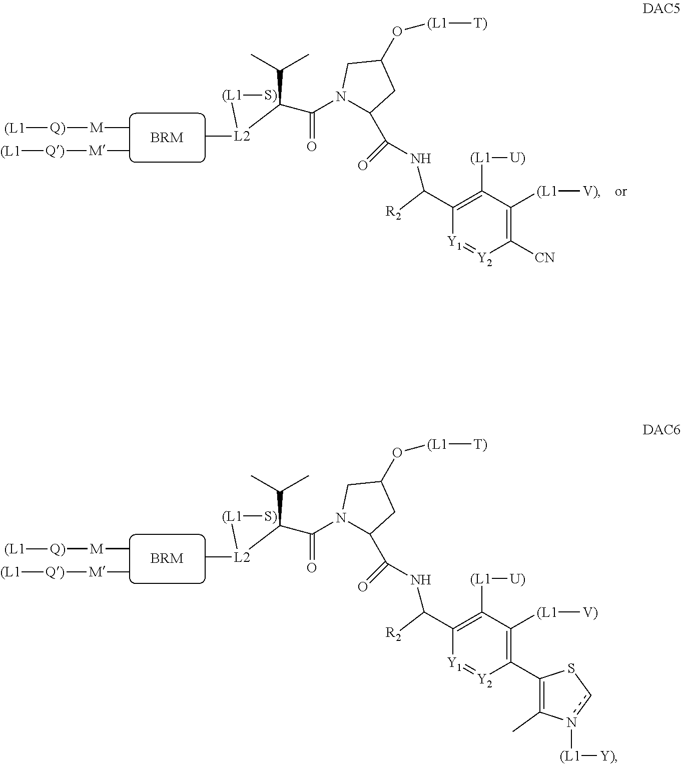

24-30. (canceled)

31. The conjugate of claim 17, wherein D has the structure:

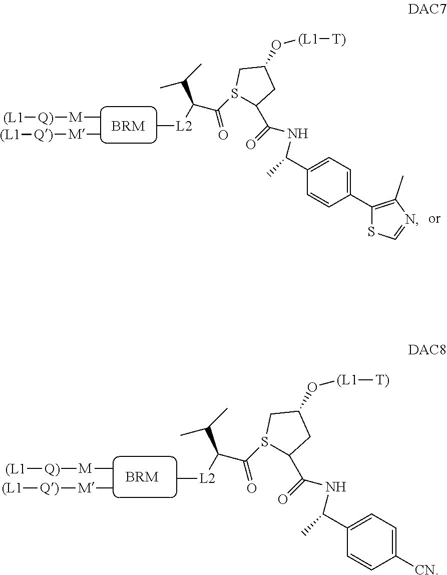

32-41. (canceled)

42. The conjugate of claim 8, wherein L1 is selected from the group consisting of: 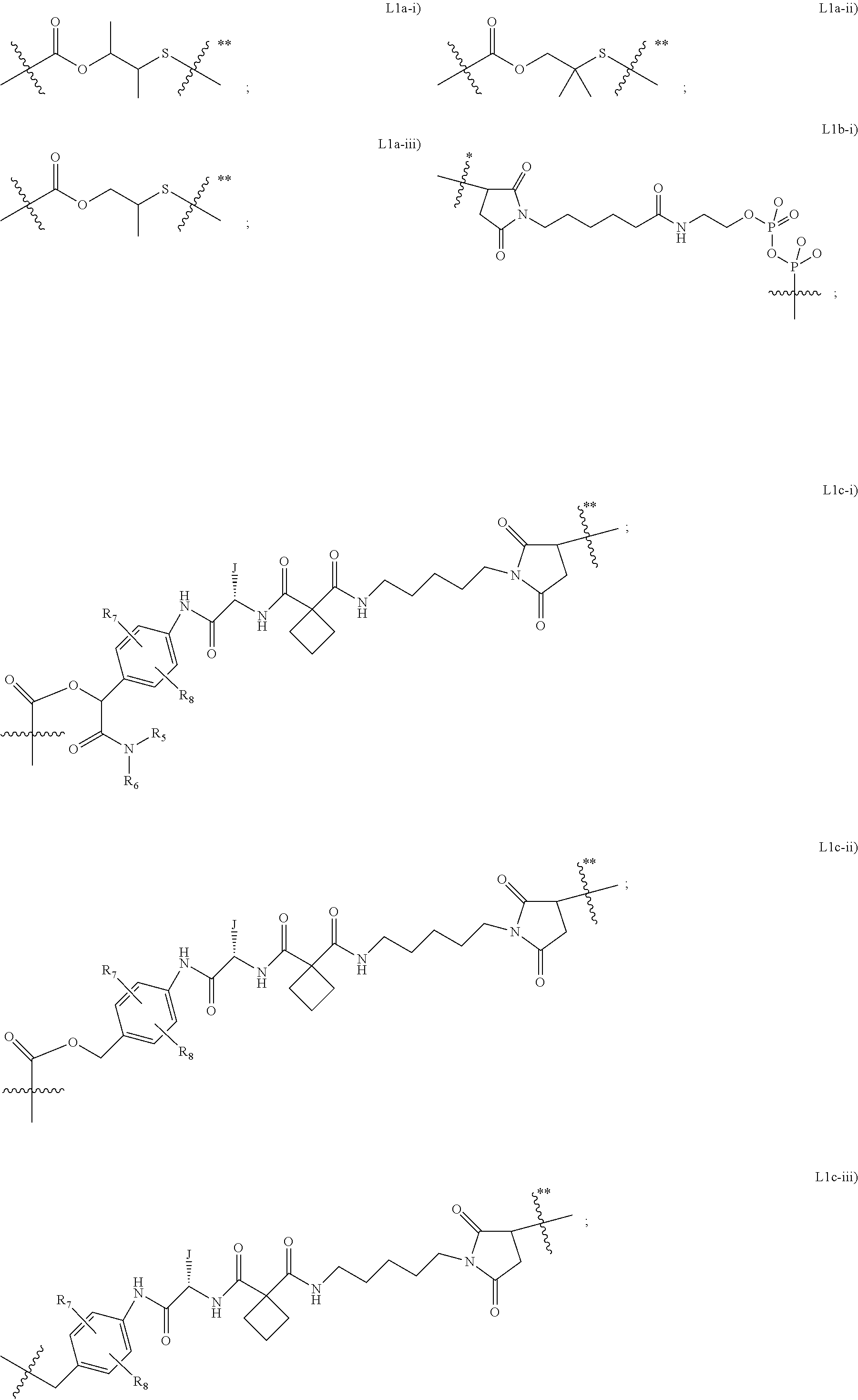
wherein,
J is —CH₂—CH₂—CH₂—NH—C(O)—NH₂; CH₂—CH₂—CH₂—CH₂—NH₂; —CH₂—CH₂—CH₂—CH₂—NH—CH₃; or CH₂—CH₂—CH₂—CH₂—N(CH₃)₂;
R₅ and R₆ are independently hydrogen or C₁₋₅ alkyl; or R₅ and R₆ together with the nitrogen to which each is attached form an optionally substituted 5- to 7-member heterocyclyl; and
R₇ and R₈ are each independently hydrogen, halo, C₁₋₅ alkyl, C₁₋₅ alkoxy or hydroxy.

43-48. (canceled)

49. The conjugate of claim 1, wherein: 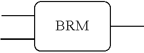
is a residue of a BRM-binding compound having a structure of Formula I: 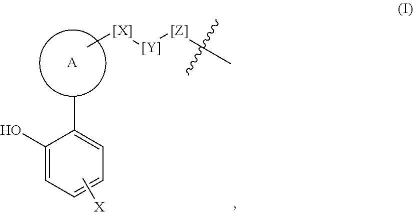
or a stereoisomer or tautomer thereof, or a pharmaceutically acceptable salt of any of the foregoing, wherein:
wherein X is hydrogen or halogen; 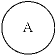
is selected from the group consisting of: 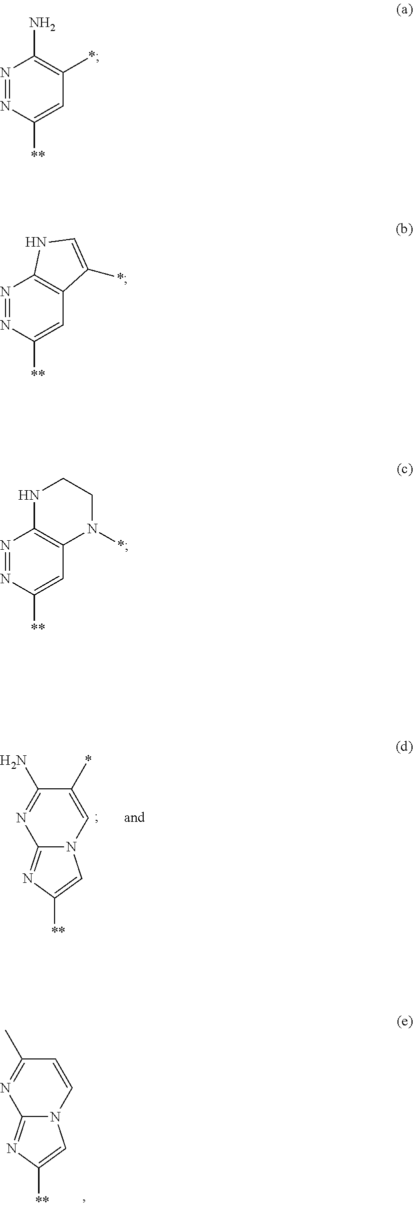
wherein, for (a)-(e), * denotes the point of attachment to [X], or, if [X] is absent, * denotes the point of attachment to [Y], and ** denotes the point of attachment to the phenyl ring; and wherein:
(i) [X] is 3-15 membered heterocyclyl or 5-20 membered heteroaryl,
provided that, when 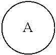
is (a), then [X] is not 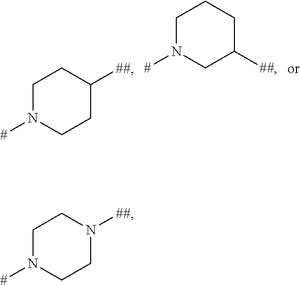
wherein #denotes the point of attachment to 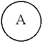
and ##denotes the point of attachment to L2,
[Y] is absent, and
[Z] is absent; or
(ii) [X] is 3-15 membered heterocyclyl or 5-20 membered heteroaryl, wherein the 3-15 membered heterocyclyl of [X] is optionally substituted with one or more —OH or C₁₋₆alkyl,
[Y] is absent, and
[Z] is 3-15 membered heterocyclyl or 5-20 membered heteroaryl,
provided that, when 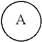
is (a) and [X] is 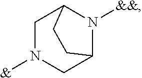
wherein & denotes the point of attachment to 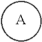
and && denotes the point of attachment to [Z], then [Z] is not 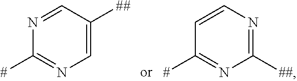
wherein #denotes the point of attachment to [X] and ##denotes the point of attachment to L2; or
(iii) [X] is 3-15 membered heterocyclyl or 5-20 membered heteroaryl,
[Y] is methylene, wherein the methylene of [Y] is optionally substituted with one or more methyl group, and
[Z] is 3-15 membered heterocyclyl; or
(iv) [X] is absent,
[Y] is ethenylene, wherein the ethenylene of [Y] is optionally substituted with one or more halo, and
[Z] is 5-20 membered heteroaryl,
provided that 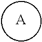
is (a), (b), (d), or (e); or
(v) [X] is absent,
[Y] is ethynylene, and
[Z] is 5-20 membered heteroaryl,
provided that 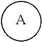
is (a), (b), (d), or (e); or
(vi) [X] is absent,
[Y] is cyclopropyl or cyclobutyl, and
[Z] is 5-20 membered heteroaryl,
provided that 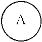
is (a), (b), (d), or (e).

50. The conjugate of claim 49, wherein the residue of the BRM-binding compound has the structure of formula (I-A), formula (I-B), formula (I-C), formula (I-D), or formula (I-E): 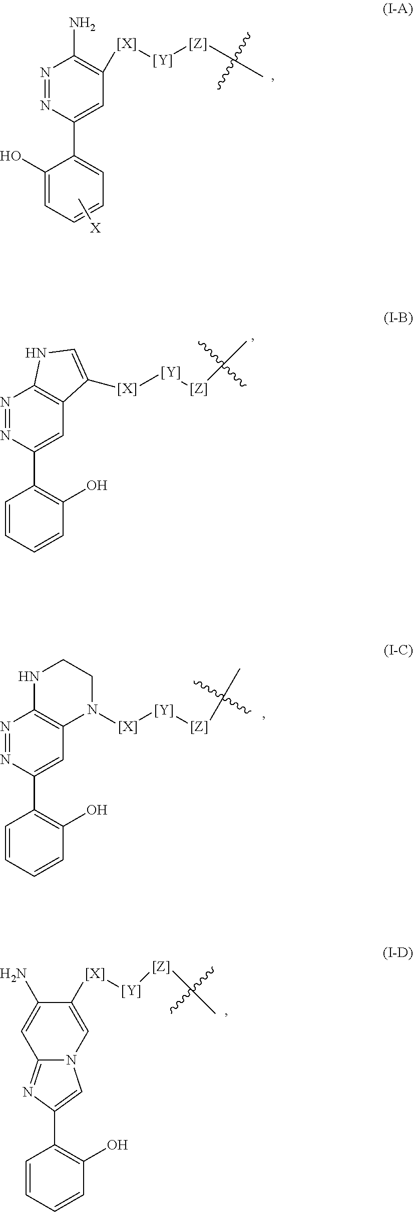
wherein X is hydrogen or halogen,
or

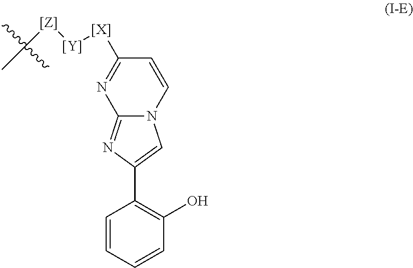

51-56. (canceled)

57. The conjugate of claim 1, wherein BRM is a residue of: 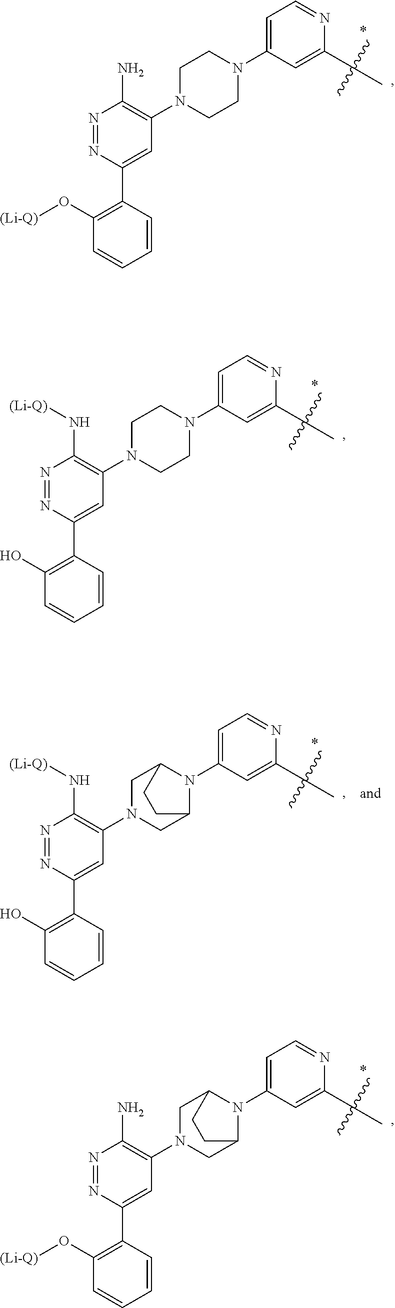
wherein, 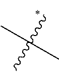
is the point of attachment to L2.

58. The conjugate of claim 1, wherein: L2 is a linker-2 covalently bound to E3LB and BRM, said L2 having the formula: 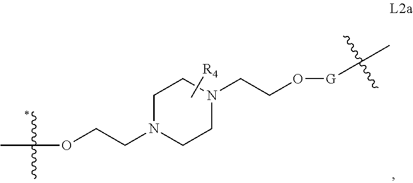
wherein, R₄ is hydrogen or methyl, 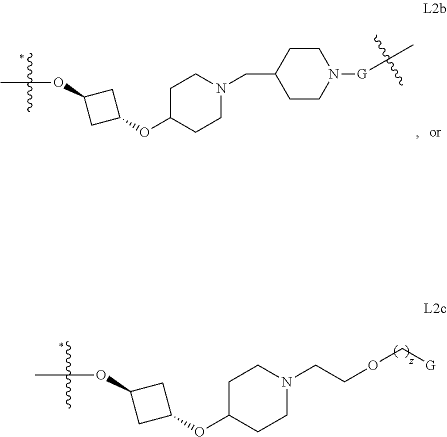
wherein,
z is one or zero, 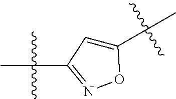 [SMILES: *c1cc(*)on1]
G is N or C(O)NH; and, 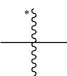
is the point of attachment to BRM.

59-61. (canceled)

62. The conjugate of claim 1, wherein the conjugate is the product of the conjugation reaction of the Ab with a compound selected from the group consisting of:

| | |
|---|---|
| 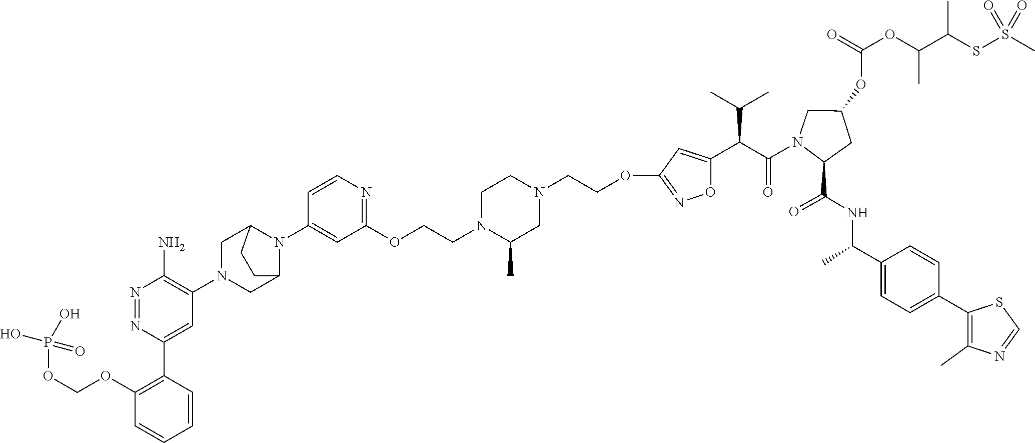 | L1- CIDE- BRM1- 10 |
| 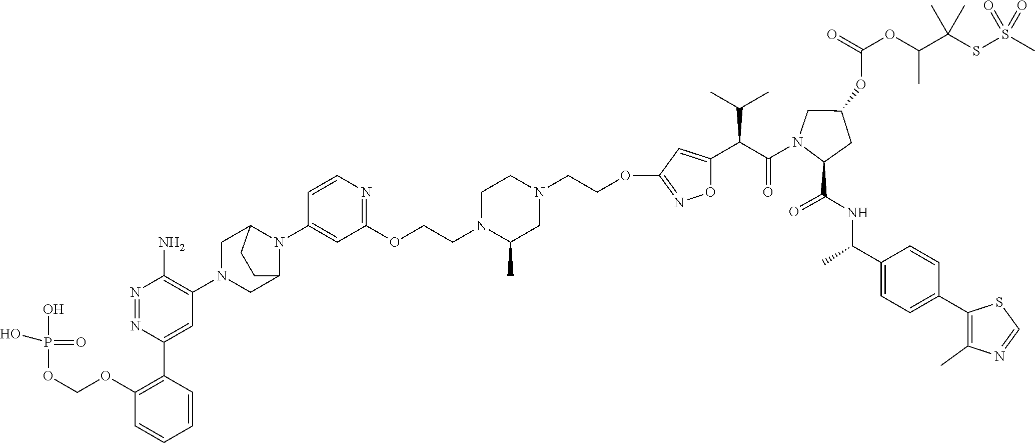 | L1- CIDE- BRM1- 9 |
| 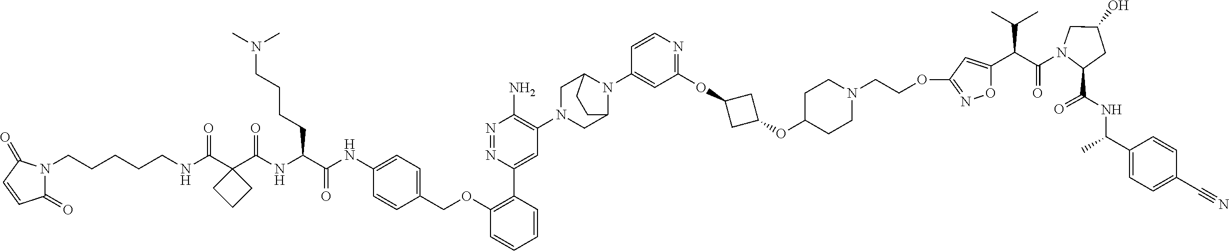 | L1- CIDE- BRM1- 19 |
| 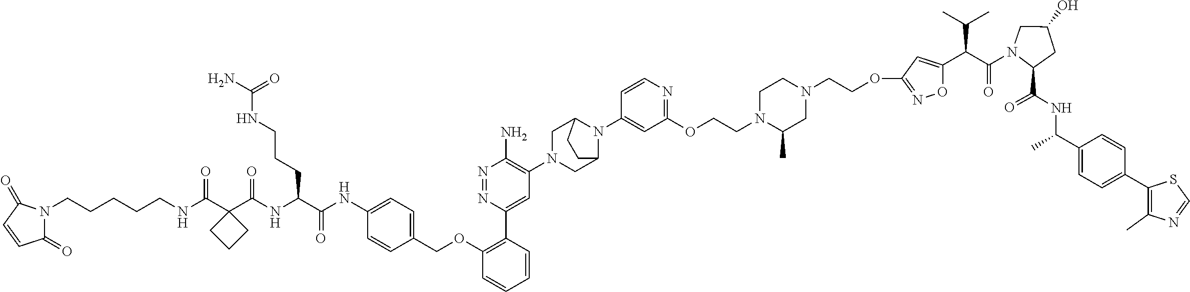 | L1- CIDE- BRM1- 13 |
| 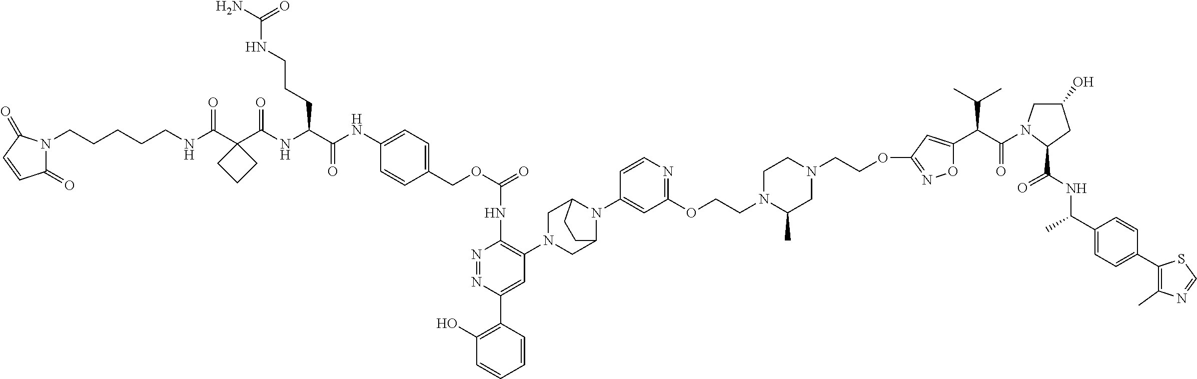 | L1- CIDE- BRM1- 20 |
| 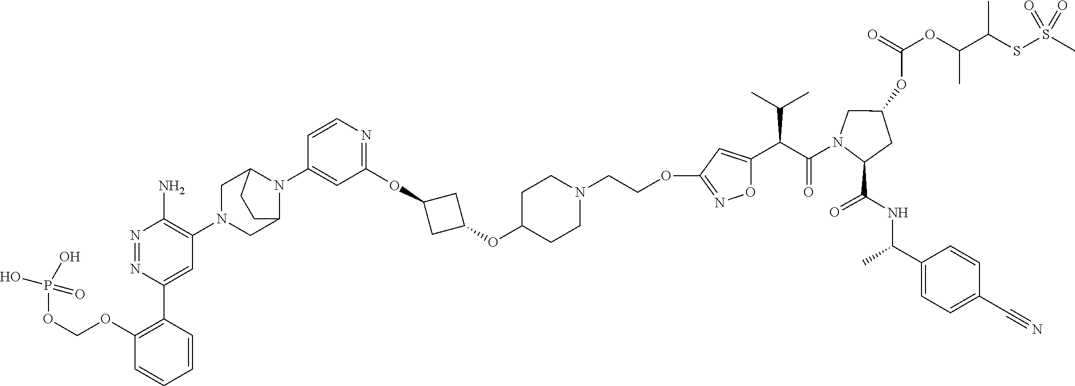 | L1- CIDE- BRM1- 11 |
| 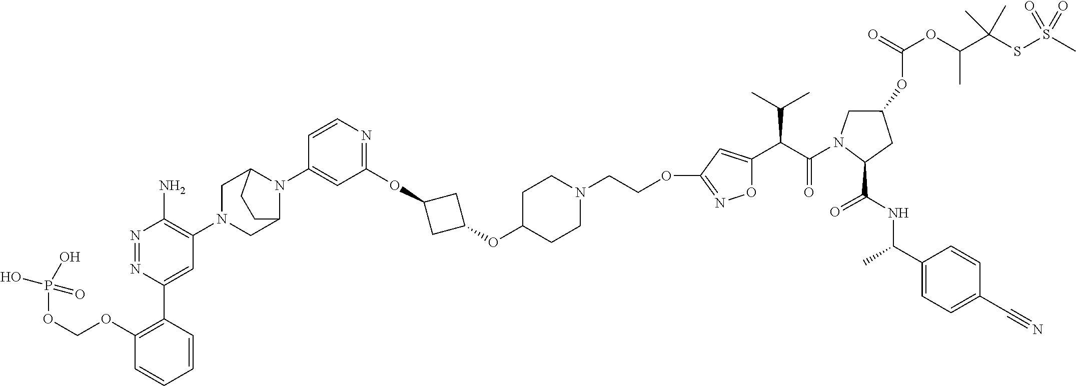 | L1- CIDE- BRM1- 12 |
| 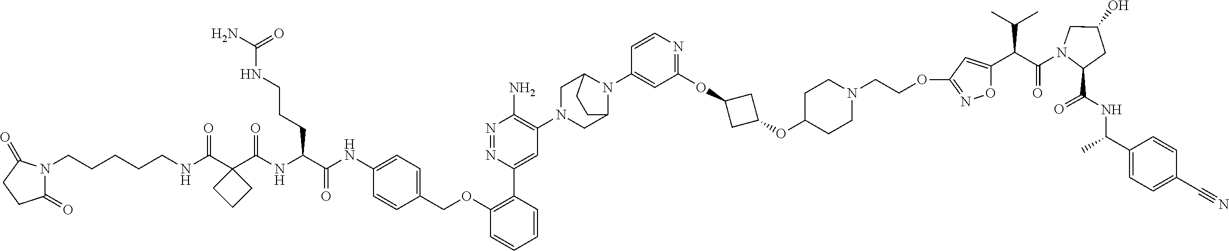 | L1- CIDE- BRM1- 14 |
| 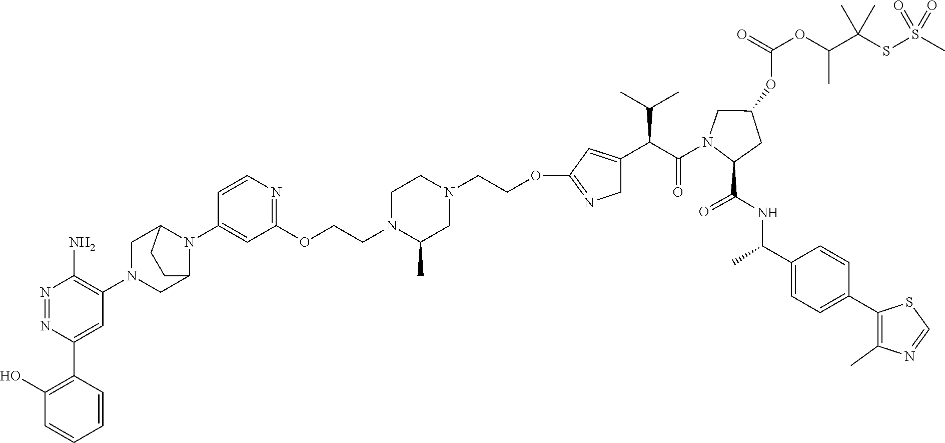 | L1- CIDE- BRM1- 7 |
| 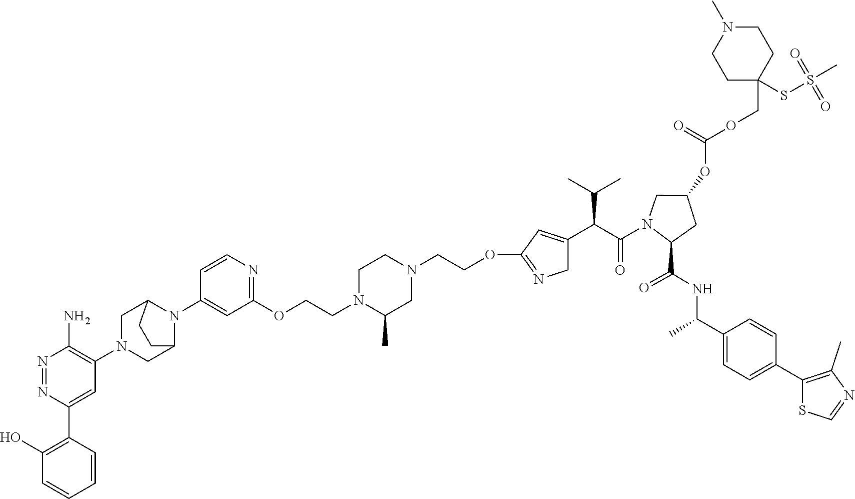 | L1- CIDE- BRM1- 8 |
| 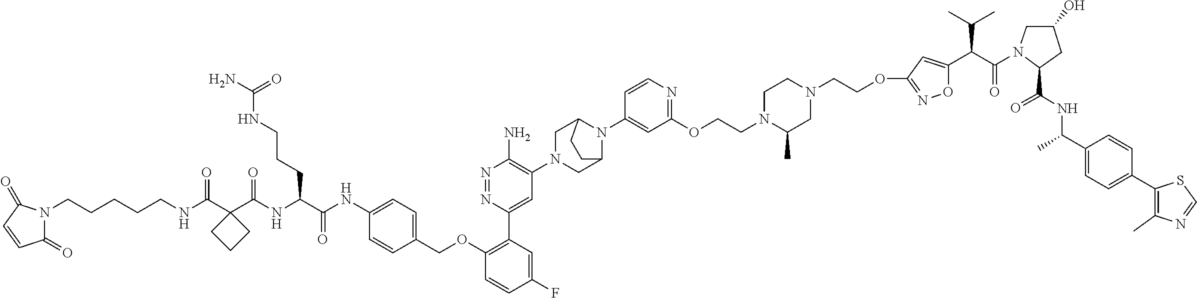 | L1- CIDE- BRM1- 16 |
| 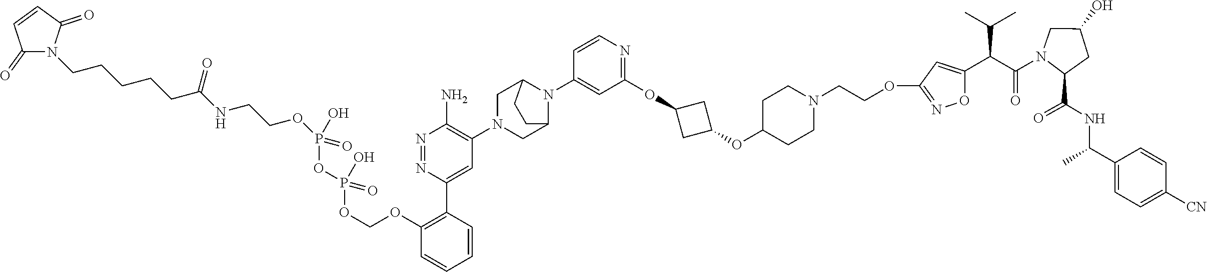 | L1- CIDE- BRM1- 17 |
| 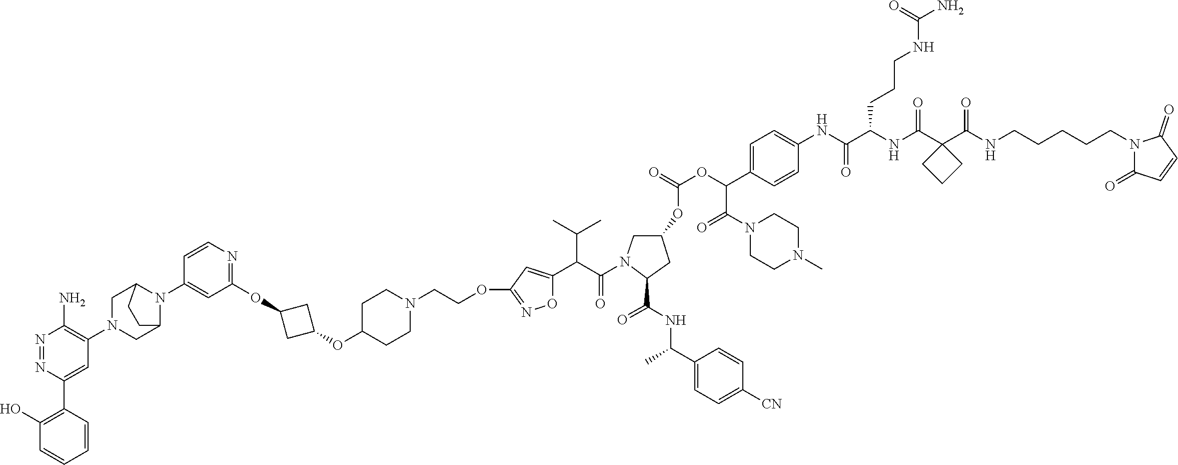 | L1- CIDE- BRM1- 18 |
| 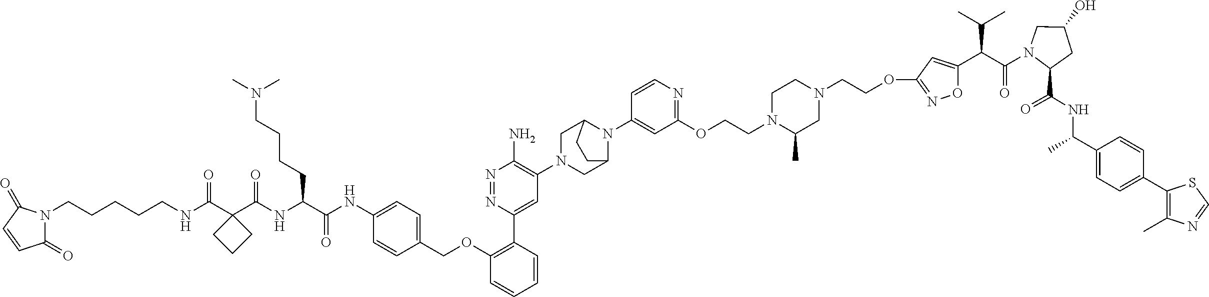 | L1- CIDE- BRM1- 21 |
| 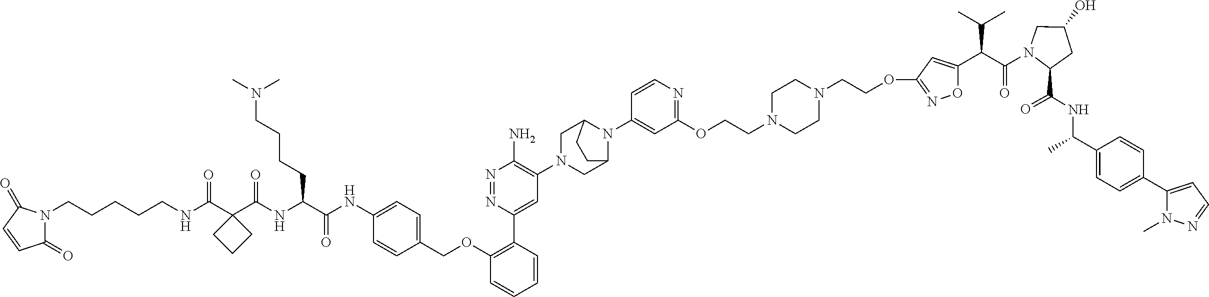 | L1- CIDE- BRM1- 22 |

63. The conjugate of claim 1, selected from the group consisting of:

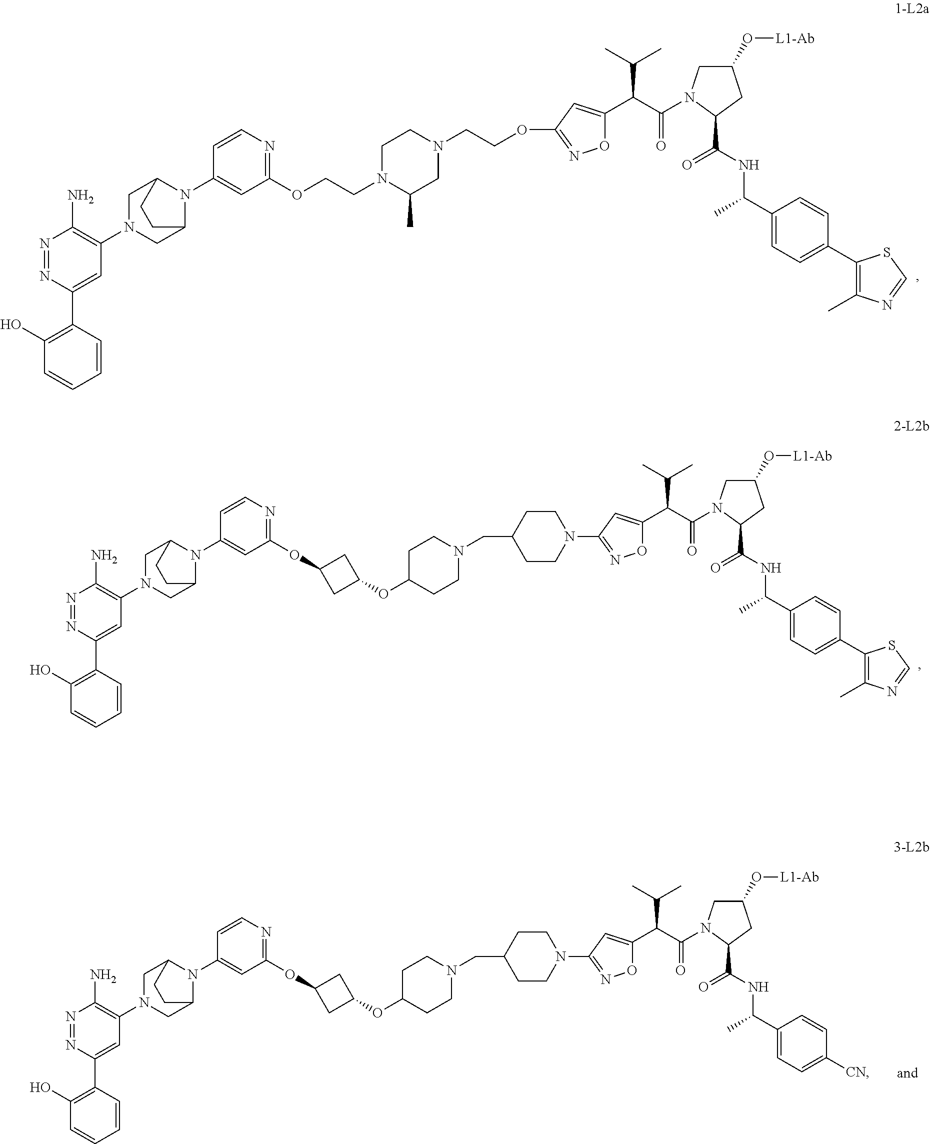

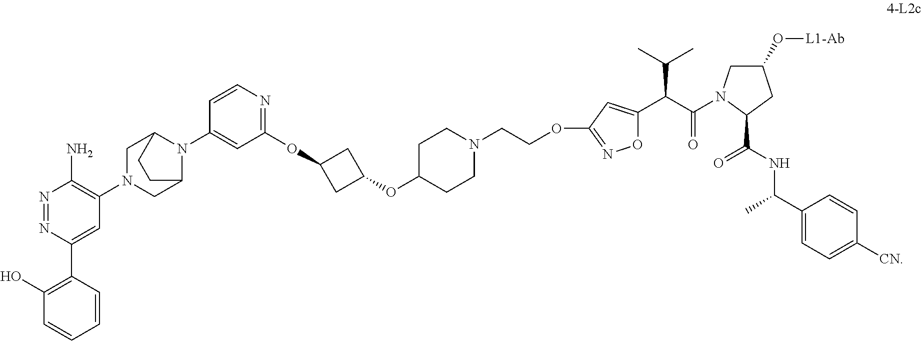

64. The conjugate of claim 1, selected from the group consisting of:

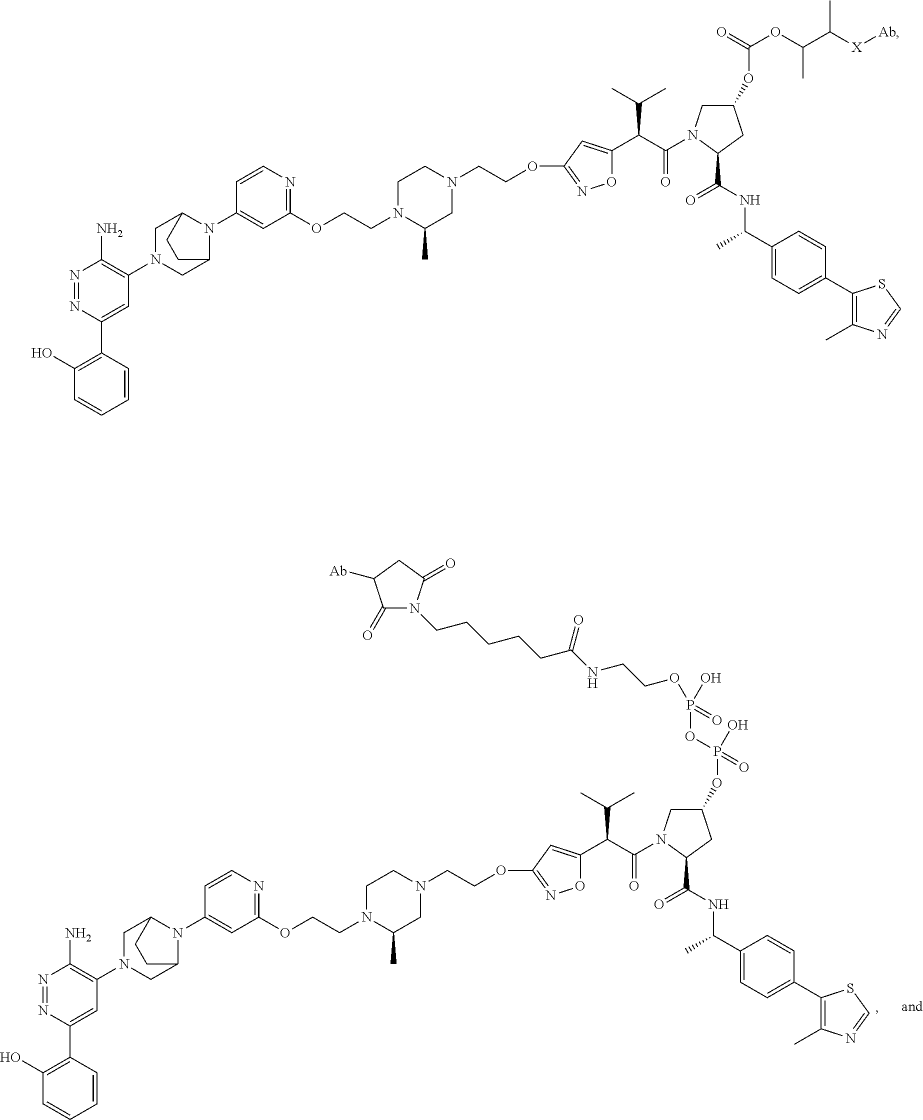

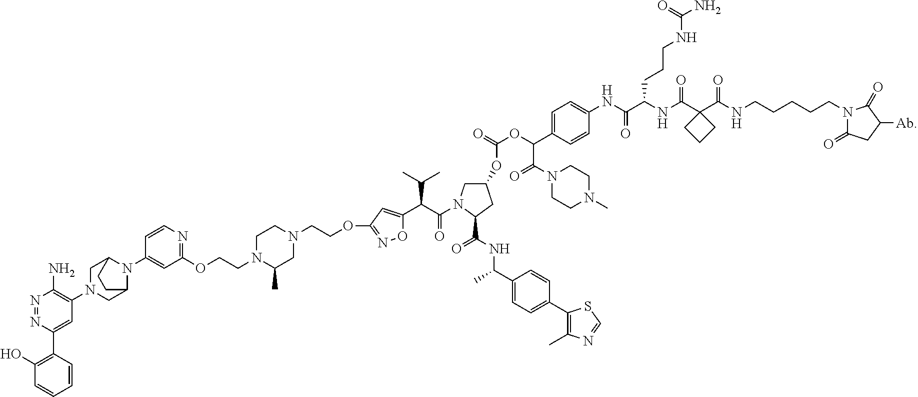

65. (canceled)

66. The conjugate of claim 1, wherein p has a value from about 5 to about 10.

67. A pharmaceutical composition comprising the conjugate of claim 1 and one or more pharmaceutically acceptable excipients.

68. A method of treating a disease in a human in need thereof, comprising administering to said human a therapeutically effective amount of the conjugate of claim 1.

69-71. (canceled)

72. A method of reducing the level of a target BRM protein in a subject comprising, administering the conjugate of claim 1 to said subject, wherein said BRM portion binds said target BRM protein, wherein ubiquitin ligase effects degradation of said bound target BRM protein, wherein the level of said BRM target protein is reduced.
